(19) United States
(12) Patent Application Publication
(10) Pub. No.: US 2020/0215113 A1
(43) Pub. Date: Jul. 9, 2020

(54) CHEMICALLY MODIFIED OLIGONUCLEOTIDES

(71) Applicant: Phio Pharmaceuticals Corp., Marlborough, MA (US)

(72) Inventor: Alexey Eliseev, Boston, MA (US)

(73) Assignee: Phio Pharmaceuticals Corp., Marlborough, MA (US)

(21) Appl. No.: 16/637,514

(22) PCT Filed: Aug. 7, 2018
(86) PCT No.: PCT/US2018/045671

§ 371 Date: Feb. 7, 2020

Related U.S. Application Data
(60) Provisional application No. 62/558,183, filed on Sep. 13, 2017.
(60) Provisional application No. 62/542,043, filed on Aug. 7, 2017.

(51) Int. Cl. A61K 35/17; A61K 31/713; C12N 15/113

(52) U.S. Cl. CPC A61K 35/17; A61K 31/713; C12N 15/113; C12N 2310/3515; C12N 2310/315; C12N 2310/321; C12N 2310/322; C12N 2310/14

(57) ABSTRACT

The disclosure relates, in some aspects, to methods and compositions for production of immunogenic compositions. In some embodiments, the disclosure provides host cells which have been treated ex vivo with one or more oligonucleotide agents capable of controlling and/or reducing the differentiation of the host cell. In some embodiments, compositions and methods described by the disclosure are useful as immunogenic modulators for treating cancer.

### RELATED APPLICATIONS

This application claims the benefit under 35 U.S.C. § 119(e) of the filing date of U.S. Provisional Application Ser. No. 62/542,043, filed Aug. 7, 2017, entitled “IMMUNOTHERAPY OF CANCER UTILIZING CHEMICALLY MODIFIED OLIGONUCLEOTIDES”, and 62/558,183, filed Sep. 13, 2017, entitled “CONTROL OF DIFFERENTIATION UTILIZING CHEMICALLY MODIFIED OLIGONUCLEOTIDES IN IMMUNOTHERAPY”, the entire disclosure of each of which is incorporated herein by reference in its entirety.

### FIELD

In some aspects, the disclosure relates to immunogenic compositions and methods of making immunogenic compositions including the use of oligonucleotides to modulate gene targets involved in cellular differentiation and metabolism to improve the population or subsets of therapeutic immune cells. The disclosure further relates to methods of using immunogenic compositions for the treatment of cell proliferative disorders or infectious disease, including, for example, cancer and autoimmune disorders.

### BACKGROUND

A physiologic function of the immune system is to recognize and eliminate neoplastic cells. Therefore, an aspect of tumor progression is the development of immune resistance mechanisms. Once developed, these resistance mechanisms not only prevent the natural immune system from affecting the tumor growth, but also limit the efficacy of any immunotherapeutic approaches to cancer. An immune resistance mechanism involves immune-inhibitory pathways, sometimes referred to as immune checkpoints. The immune-inhibitory pathways play a particularly important role in the interaction between tumor cells and CD8+ cytotoxic T-lymphocytes, including Adoptive Cell Transfer (ACT) therapeutic agents.

Various methods of adoptive cell transfer (ACT) involve ex vivo treatment of cells collected from a patient's samples, such as blood or tumor material. Common steps involved in the preparation of cell-based treatments are isolation of cells from the primary source (e.g., peripheral blood), gene editing (e.g., engineering of chimeric antigen receptor (CAR) T-cells or engineered T-cell receptor (TCR) cells), activation, and expansion.

During the ex vivo processing the cells undergo certain phenotypic changes that may affect their therapeutic properties, such as trafficking to the tumor, proliferative ability and longevity in vivo, and their efficacy in the immunosuppressive environment, among others. For example, the state of T-cell differentiation and maturation typically progresses through the following sequence of subtypes: naïve (T_{N})-stem cell memory (T_{SCM})-central memory (T_{CM}) -effector memory (T_{EM})-terminally differentiated effector T cells (T_{EFF}). It has been observed that phenotypic and functional attributes of early memory T-cells (T_{SCM}/T_{CM}) among CD8+ T cells demonstrate superior in vivo expansion, persistence, and antitumor efficacy than more differentiated effector cells (e.g., T_{EM}, T_{EFF}, etc.).

Immunotherapy of cancer has become increasingly important in clinical practice. Immunotherapies designed to elicit or amplify an immune response can be classified as activation immunotherapies, while immunotherapies that reduce or suppress immune response can be classified as suppression immunotherapies. One activation immunotherapeutic strategy to combat cancer immune resistance mechanisms is inhibiting immune checkpoints (e.g., by using checkpoint-targeting monoclonal antibodies) in order to stimulate or maintain a host immune response.

However, there are a number of drawbacks of using cancer immunotherapeutic agents in combination with checkpoint inhibitors. For example, immune checkpoint blockade can lead to the breaking of immune self-tolerance, thereby inducing a novel syndrome of autoimmune/auto-inflammatory side effects, designated “immune related adverse events.” Additionally, toxicity profiles of checkpoint inhibitors are reportedly different than the toxicity profiles reported for other classes of oncologic agents, and may induce inflammatory events in multiple organ systems, including skin, gastrointestinal, endocrine, pulmonary, hepatic, ocular, and nervous system.

### SUMMARY

In some aspects, the disclosure relates to compositions and methods for controlling the differentiation process of T-cells during production of immunogenic compositions to enhance levels of desired subtypes of therapeutic T cells (e.g., T_{SCM} and T_{CM}). The disclosure is based, in part, on immunomodulatory (e.g., immunogenic) compositions comprising a host cell comprising oligonucleotide molecules that target genes associated with signal transduction/transcription factors, epigenetic, metabolic and co-inhibitory/negative regulatory targets, as well as methods of producing such compositions. In some aspects, the disclosure provides chemically-modified oligonucleotide molecules used in methods of producing immunogenic compositions. In some embodiments, methods and compositions described by the disclosure are useful for the manufacture of immunogenic compositions and for treating a subject having a proliferative or infectious disease.

Accordingly, in some aspects, the disclosure provides a chemically-modified double stranded nucleic acid molecule that targets (e.g., is directed against a gene encoding) Protein Kinase B (PKB, also referred to as AKT), Programmed Cell Death Protein 1 (PD1, also referred to as PDCD1), T cell Immunoreceptor with Ig and ITIM domains (TIGIT), Tumor protein p53 (TP53, also known as p53, cellular tumor antigen, phosphoprotein p53, tumor suppressor p53, antigen NY-CO-13, or transformation-related protein 53 (TRP53)), E3 ubiquitin-protein ligase Cbl-b (Cbl-b), Tet Methylcytosine Dioxygenase 2 (TET2, also known as KIAA1546, Tet Oncongene Family Member 2, Probable Methylcytosine Dioxygenase TET2, Methylcytosine Dioxygenase TET2), PR/SET Domain 1 (Blimp-1, also known as PR Domain Containing 1, With ZNF Domain, PR Domain 1, PRDM1, PRDI-BF1, Beta-interferon Gene Positive-Regulatory Domain I Binding Factor, Positive Regulatory Domain I-Binding Factor 1, B-Lymphocyte-Induced Maturation Protein 1, PR Domain Zinc Finger Protein 1, PR Domain-Containing Protein 1, PRDI-Binding Factor-1), T-Box 21 (TBX21, also known as T-Cell Specific T-Box Transcription Factor T-Bet, Transcription Factor TBLYM, T-Box Protein 21, TBLYM, TBET, T-Box Transcription Factor TBX21, T-Box Expressed in T Cells, T-PET, T-Bet), DNA (cytosine-5)-methyltransferase 3A (DNMT3A), Protein Tyrosine Phosphatase, Non-Receptor Type 6 (PTPN6, also known as SHP-1), or Hexokinase 2 (HK2, also known as Muscle Form Hexokinase).

In some embodiments, a chemically-modified double stranded nucleic acid molecule is directed against a sequence comprising at least 12 contiguous nucleotides of a sequence selected from the sequences within Tables 3-13. In some embodiments, a chemically-modified double stranded nucleic acid molecule is a self-delivering RNA (e.g., sd-rxRNA). In some embodiments, a chemically-modified double stranded nucleic acid molecule (e.g., sd-rxRNA) comprises or consists of, or is targeted to or directed against, a sequence set forth in Tables 3-13, or a fragment thereof.

In some embodiments, a chemically-modified double stranded nucleic acid molecule comprises at least one 2′-O-methyl modification and/or at least one 2′-Fluoro modification, and at least one phosphorothioate modification. In some embodiments, the first nucleotide relative to the 5′end of the guide strand has a 2′-O-methyl modification. In some embodiments, the 2′-O-methyl modification is a 5P-2′O-methyl U modification, or a 5′ vinyl phosphonate 2′-O-methyl U modification.

In some embodiments, a sd-rxRNA is hydrophobically modified. In some embodiments, a sd-rxRNA is linked to one or more hydrophobic conjugates. In some embodiments, the hydrophobic conjugate is cholesterol.

In some aspects, the disclosure provides a sd-rxRNA that is directed against a gene encoding TIGIT, DNMT3A, PTPN6, PDCD1, AKT, P53, Cbl-b, Tet2, Blimp-1, T-Box21, or HK2. In some embodiments, a sd-rxRNA comprises at least 12 contiguous nucleotides of a sequence selected from the sequences within Tables 3-13.

In some aspects, the disclosure provides chemically-modified double stranded nucleic acid molecules that target T-cell Immunoreceptor with Ig and ITIM domains (TIGIT) or Programmed Cell Death Protein 1 (PD1).

In some aspects, the disclosure provides a chemically-modified double stranded nucleic acid molecule that is directed against a gene encoding TIGIT. In some embodiments, the chemically-modified double stranded nucleic acid molecule is directed against a sequence comprising at least 12 contiguous nucleotides selected from the sequences within Table 5. In some embodiments, an sd-rxRNA comprises a sense strand a sense strand having the sequence set forth in SEQ ID NO: 100 (TIGIT 21 sense strand) and/or an antisense strand having the sequence set forth in SEQ ID NO: 101 (TIGIT 21 antisense strand). In some embodiments, an sd-rxRNA comprises a sense strand having the sequence set forth in SEQ ID NO: 100 (TIGIT 21 sense strand) and an antisense strand having the sequence set forth in SEQ ID NO: 101 (TIGIT 21 antisense strand).

In some embodiments, the disclosure provides a chemically-modified double stranded nucleic acid that is directed against PD1. In some embodiments, the chemically-modified double stranded nucleic acid molecule is directed against a sequence comprising at least 12 contiguous nucleotides selected from the sequences within Table 3 or Table 6. In some embodiments, the chemically-modified double stranded nucleic acid molecule comprises a sequence set forth in Table 6. In some embodiments, an sd-rxRNA comprises a sense strand having the sequence set forth in SEQ ID NO: 112 (PD 26 sense strand) and/or an antisense strand having the sequence set forth in SEQ ID NO: 113 (PD 26 antisense strand). In some embodiments, an sd-rxRNA comprises a sense strand having the sequence set forth in SEQ ID NO: 112 (PD 26 sense strand) and an antisense strand having the sequence set forth in SEQ ID NO: 113 (PD 26 antisense strand).

In some embodiments, the disclosure provides a chemically-modified double stranded nucleic acid that is directed against Cbl-b. In some embodiments, the chemically-modified double stranded nucleic acid molecule is directed against a sequence comprising at least 12 contiguous nucleotides selected from the sequences within Table 4 and Table 8. In some embodiments, the chemically-modified double stranded nucleic acid molecule comprises a sequence set forth in Table 8. In some embodiments, a chemically-modified double stranded nucleic acid molecule or a sd-rxRNA as described herein comprises or consists of the sequence set forth in CB 23 sense or antisense strand (SEQ ID NO: 236 or 237) or CB 29 sense or antisense strand (SEQ ID NO: 248 or 249).

In some embodiments, a chemically-modified double stranded nucleic acid molecule or sd-rxRNA as described herein comprises or consists of a sense strand having the sequence set forth in CB 23 sense strand (SEQ ID NO: 236) and/or an antisense strand having the sequence set forth in CB 23 antisense strand (SEQ ID NO: 237). In some embodiments, a chemically-modified double stranded nucleic acid molecule or sd-rxRNA as described herein comprises or consists of a sense strand having the sequence set forth in CB 29 sense strand (SEQ ID NO: 248) and/or an antisense strand having the sequence set forth in CB 29 antisense strand (SEQ ID NO: 249).

In some embodiments, the disclosure provides a chemically-modified double stranded nucleic acid that is directed against HK2. In some embodiments, the chemically-modified double stranded nucleic acid molecule is directed against a sequence comprising at least 12 contiguous nucleotides selected from the sequences within Table 7. In some embodiments, the chemically-modified double stranded nucleic acid molecule comprises a sequence set forth in Table 7.

In some embodiments, the disclosure provides a chemically-modified double stranded nucleic acid that is directed against DNMT3A. In some embodiments, the chemically-modified double stranded nucleic acid molecule is directed against a sequence comprising at least 12 contiguous nucleotides selected from the sequences within Table 9. In some embodiments, the chemically-modified double stranded nucleic acid molecule comprises a sequence set forth in Table 9.

In some embodiments, the disclosure provides a chemically-modified double stranded nucleic acid that is directed against PRDM1. In some embodiments, the chemically-modified double stranded nucleic acid molecule is directed against a sequence comprising at least 12 contiguous nucleotides selected from the sequences within Table 10. In some embodiments, the chemically-modified double stranded nucleic acid molecule comprises a sequence set forth in Table 10.

In some embodiments, the disclosure provides a chemically-modified double stranded nucleic acid that is directed against PTPN6. In some embodiments, the chemically-modified double stranded nucleic acid molecule is directed against a sequence comprising at least 12 contiguous nucleotides selected from the sequences within Table 11. In some embodiments, the chemically-modified double stranded nucleic acid molecule comprises a sequence set forth in Table 11.

In some embodiments, the disclosure provides a chemically-modified double stranded nucleic acid that is directed against TET2. In some embodiments, the chemically-modified double stranded nucleic acid molecule is directed against a sequence comprising at least 12 contiguous nucleotides selected from the sequences within Table 11. In some embodiments, the chemically-modified double stranded nucleic acid molecule comprises a sequence set forth in Table 11.

In some embodiments, the disclosure provides a chemically-modified double stranded nucleic acid that is directed against Tbox21. In some embodiments, the chemically-modified double stranded nucleic acid molecule is directed against a sequence comprising at least 12 contiguous nucleotides selected from the sequences within Table 13. In some embodiments, the chemically-modified double stranded nucleic acid molecule comprises a sequence set forth in Table 13.

In some aspects, the disclosure provides a composition comprising a chemically-modified double stranded nucleic acid molecule or a sd-rxRNA as described herein and a pharmaceutically acceptable excipient.

In some aspects, the disclosure provides a composition (e.g., an immunogenic composition) comprising a chemically-modified double stranded nucleic acid molecule as described by the disclosure (e.g., targeting a sequence set forth in any one of Tables 3-13) or an sd-rxRNA as described by the disclosure (e.g. as set forth in Tables 3-13), and a pharmaceutically acceptable excipient. In some embodiments, the chemically-modified nucleic acid molecule comprises a sequence selected from PD 21 to PD 37 (SEQ ID NOs: 102-135), TIGIT 1 (SEQ ID NO: 60), TIGIT 6 (SEQ ID NO: 65) and TIGIT 21 (SEQ ID NO: 100-101).

In some aspects, the disclosure relates to immunogenic compositions comprising a host cell (e.g., one or more host cells, or a population of host cells) comprising one or more a chemically-modified double stranded nucleic acid molecules as described herein. Examples of host cells include but are not limited to T-cells, NK-cell, antigen-presenting cells (APC), dendritic cells (DC), stem cell (SC), induced pluripotent stem cells (iPSC), and stem central memory T-cells.

In some aspects, the disclosure provides an immunogenic composition comprising a host cell comprising a chemically-modified double stranded nucleic acid molecule that is directed against a TIGIT sequence comprising at least 12 contiguous nucleotides of a sequence selected from the sequences within Table 5.

In some aspects, the disclosure provides an immunogenic composition comprising a host cell comprising an sd-rxRNA that is directed against a gene encoding PD1, wherein the sd-rxRNA comprises at least 12 contiguous nucleotides of a sequence selected from the sequences within Table 3. In some embodiments the sd-rxRNA comprises a sequence set forth in Table 6.

In some embodiments, a chemically-modified double stranded nucleic acid molecule or sd-rxRNA induces at least 50% inhibition of PDCD1 or TIGIT in a host cell.

In some aspects, the disclosure provides an immunogenic composition comprising a host cell comprising an sd-rxRNA that is directed against a gene encoding Cbl-b, wherein the sd-rxRNA comprises at least 12 contiguous nucleotides of a sequence selected from the sequences within Table 4. In some embodiments the sd-rxRNA comprises a sequence set forth in Table 8.

In some aspects, the disclosure provides an immunogenic composition comprising a host cell comprising an sd-rxRNA that is directed against a gene encoding HK2, wherein the sd-rxRNA targets a sequence comprising at least 12 contiguous nucleotides of a sequence selected from the sequences within Table 7. In some embodiments the sd-rxRNA comprises a sequence set forth in Table 7.

In some aspects, the disclosure provides an immunogenic composition comprising a host cell comprising an sd-rxRNA that is directed against a gene encoding DNMT3A, wherein the sd-rxRNA targets a sequence comprising at least 12 contiguous nucleotides of a sequence selected from the sequences within Table 9. In some embodiments the sd-rxRNA comprises a sequence set forth in Table 9.

In some aspects, the disclosure provides an immunogenic composition comprising a host cell comprising an sd-rxRNA that is directed against a gene encoding PRDM1, wherein the sd-rxRNA targets a sequence comprising at least 12 contiguous nucleotides of a sequence selected from the sequences within Table 10. In some embodiments the sd-rxRNA comprises a sequence set forth in Table 10.

In some aspects, the disclosure provides an immunogenic composition comprising a host cell comprising an sd-rxRNA that is directed against a gene encoding PTPN6, wherein the sd-rxRNA targets a sequence comprising at least 12 contiguous nucleotides of a sequence selected from the sequences within Table 11. In some embodiments the sd-rxRNA comprises a sequence set forth in Table 11.

In some aspects, the disclosure provides an immunogenic composition comprising a host cell comprising an sd-rxRNA that is directed against a gene encoding TET2, wherein the sd-rxRNA targets a sequence comprising at least 12 contiguous nucleotides of a sequence selected from the sequences within Table 12. In some embodiments the sd-rxRNA comprises a sequence set forth in Table 12.

In some aspects, the disclosure provides an immunogenic composition comprising a host cell comprising an sd-rxRNA that is directed against a gene encoding Tbox21, wherein the sd-rxRNA targets a sequence comprising at least 12 contiguous nucleotides of a sequence selected from the sequences within Table 13. In some embodiments the sd-rxRNA comprises a sequence set forth in Table 13.

In some aspects, the disclosure provides an immunogenic composition comprising a host cell (e.g., an immune cell, such as a T-cell) which has been treated ex vivo with a chemically-modified double stranded nucleic acid molecule to control and/or reduce the level of differentiation of the host cell (e.g., T-cell) to enable the production of a specific immune cellular population (e.g., a population enriched for a particular T-cell subtype) for administration in a human. In some embodiments, an immunogenic composition comprises a plurality of host cells that are enriched for a particular cell type (e.g. T-cell subtype). For example, in some embodiments, an immunogenic composition comprises at least 50%, at least 60%, at least 70%, at least 80%, at least 90%, at least 95%, at least 99% or 100% (e.g., any percentage between 50% and 100%, inclusive) T-cells of a particular T-cell subtype, such as T_{SCM} or T_{CM} cells.

In some embodiments, an immunogenic composition comprises a host cell comprising a chemically-modified double stranded nucleic acid molecule as described herein (e.g., a chemically-modified double stranded nucleic acid molecule or sd-rxRNA that is directed against a gene encoding DNMT3A, PTPN6, PDCD1, AKT, p53, Cbl-b, Tet2, Blimp-1, T-Box21, or HK2), or a combination of chemically-modified double stranded nucleic acid molecule or sd-rxRNAs directed against one or more genes encoding DNMT3A, PTPN6, PDCD1, AKT, p53, Cbl-b, Tet2, Blimp-1, T-Box21, or HK2. In some embodiments, the chemically-modified double stranded nucleic acid molecule or sd-rxRNA is directed against a sequence comprising at least 12 contiguous nucleotides of a sequence selected from the sequences within Tables 3-13. In some embodiments, a chemically-modified double stranded nucleic acid molecule (e.g., sd-rxRNA) comprises or consists of, or is targeted to or directed against, a sequence set forth in Tables 3-13, or a fragment thereof.

In some embodiments, a host cell is selected from the group of: T-cell, NK-cell, antigen-presenting cell (APC), dendritic cell (DC), stem cell (SC), induced pluripotent stem cell (iPSC), stem cell memory T-cell, and Cytokine-induced Killer cell (CIK). In some embodiments, the host cell is a T-cell. In some embodiments, the T-cell is a CD8+ T-cell. In some embodiments, the T-cell is differentiated into a particular T-cell subtype, such as a T_{SCM} or T_{CM} T-cell after introduction of the chemically-modified double stranded nucleic acid or sd-rxRNA.

In some embodiments, a T-cell comprises one or more transgenes expressing a high affinity T-cell receptor (TCR) and/or a chimeric antigen receptor (CAR).

In some embodiments, a host cell is derived from a healthy donor (e.g., a donor that does not have or is not suspected of having a proliferative disease, such as cancer, or an infectious disease).

In some aspects, the disclosure provides a method for producing an immunogenic composition, the method comprising introducing into a cell one or more chemically-modified double stranded nucleic acid molecules or sd-rxRNAs as described herein. In some embodiments, the chemically-modified double stranded nucleic acid molecules or sd-rxRNA are introduced into the cell ex vivo.

In some embodiments of methods described herein, a cell is a T-cell, NK-cell, antigen-presenting cell (APC), dendritic cell (DC), stem cell (SC), induced pluripotent stem cell (iPSC),stem cell memory T-cell, and Cytokine-induced Killer cell (CIK).

In some embodiments, the T-cell is a CD8+ T-cell. In some embodiments, the T-cell is differentiated into a particular T-cell subtype, such as a T_{SCM} or T_{CM} T-cell after introduction of the chemically-modified double stranded nucleic acid or sd-rxRNA. In some embodiments, the T-cell comprises one or more transgenes expressing a high affinity T-cell receptor (TCR) and/or a chimeric antigen receptor (CAR). In some embodiments, the cell is derived from a healthy donor.

In some aspects, the disclosure provides a method for treating a subject for suffering from a proliferative disease or an infectious disease, the method comprising administering to the subject an immunogenic composition as described herein. In some embodiments, a proliferative disease is cancer. In some embodiments, an infectious disease is a pathogen infection, such as a viral, bacterial, or parasitic infection.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows reduction of PDCD1 mRNA levels utilizing chemically optimized PD-1-targeting sd-rxRNAs in Human Primary T-cells.

FIG. 2 shows dose response curves of chemically optimized sd-rxRNAs targeting PDCD1 in Human Primary T-cells. For each chemically optimized sd-rxRNA, the concentrations tested from left to right were 2 μM, 1 μM, 0.5 μM, 0.25 μM, 0.125 μM and 0.06 μM.

FIG. 3 shows dose response curves of TIGIT-targeting sd-rxRNAs in human primary T-cells. For each sd-rxRNA, the concentrations tested from left to right were 2 μM, 1 μM, 0.5 μM, 0.25 μM, 0.1 μM and 0.04 μM.

FIG. 4 shows a schematic depiction of the progression of the differentiation state of T-cells.

FIG. 5 shows enhanced T central memory (T_{CM}) differentiation from activated human primary T-cells treated with PD-1 and TIGIT-targeting sd-rxRNA in ex vivo culture. Human naïve T cells were activated with CD3/CD28 Dynabeads+IL-2 and treated with 2 μM NTC (non-targeting control) sd-rxRNA, 2 μM PD 1-targeting sd-rxRNA and 2 μM TIGIT-targeting sd-rxRNA. Four days later, cells were harvested and T-cell subsets were analyzed by multi-color flow cytometry. The population of T-cells differentiated to the T_{CM} subtype was enhanced 3.9 fold and 1.7 fold upon PD-1 and TIGIT inhibition, respectively as compared to the control.

FIG. 6 shows two point dose response curves of sd-rxRNAs targeting HK2 in HepG2 cells. For each chemically optimized sd-rxRNA, the concentrations tested were from left to right 1 μM and 0.02 μM.

FIG. 7 shows six point dose response curves of sd-rxRNAs targeting HK2 in Pan-T cells. For each sd-rxRNA, the concentrations tested from left to right were 2 μM, 1 μM, 0.5 μM, 0.25 μM, 0.125 μM and 0.06 μM.

FIG. 8 shows representative data for Cbl-b silencing in T-cells. In the dose response experiment shown in the right-hand caption, for each sd-rxRNA, the concentrations tested from left to right were 2 μM, 1 μM, 0.5 μM, 0.25 μM, 0.1 μM and 0.04 μM.

FIG. 9 shows five point dose response of sd-rxRNAs targeting CBLB in human primary NK cells. For each sd-rxRNA, the concentrations tested from left to right were 2 μM, 1 μM, 0.5 μM, 0.25 μM and 0.125 μM.

FIG. 10 shows three point dose response of sd-rxRNAs targeting DMNT3A in HepG2 cells. For each sd-rxRNA, the concentrations tested from left to right were 1 μM, 0.5 μM and 0.25 μM.

FIG. 11 shows five point dose response curves of sd-rxRNAs targeting DMNT3A in Pan-T cells. For each sd-rxRNA, the concentrations tested from left to right were 2 μM, 1 μM, 0.5 μM, 0.25 μM, 0.125 μM and 0.06 μM.

FIG. 12 shows two point dose response of sd-rxRNAs targeting PRDM1 in A549 cells. For each sd-rxRNA, the concentrations tested were 1 μM (left) and 0.2 μM (right).

FIG. 13 shows six point dose response of sd-rxRNAs targeting PRDM1 in A549 cells. For dose response experiments, for each sd-rxRNA, the concentrations tested from left to right were 1 μM, 0.5 μM, 0.1 μM, 0.05 μM 0.025 μM and 0.01 μM.

FIG. 14 shows two point dose response of sd-rxRNAs targeting PTPN6 in A549 cells. For each sd-rxRNA, the concentrations tested were 1 μM (left) and 0.2 μM (right).

FIG. 15 shows six point dose response of sd-rxRNAs targeting PTPN6 in A549 cells. For dose response experiments, for each sd-rxRNA, the concentrations tested from left to right were 1 μM, 0.5 μM, 0.1 μM, 0.05 μM, 0.025 μM and 0.01 μM.

FIG. 16 shows two point dose response of sd-rxRNAs targeting TET2 in U2OS cells. For each sd-rxRNA, the concentrations tested were 1 μM (left) and 0.2 μM (right).

FIG. 17 shows six point dose response of sd-rxRNAs targeting TET2 in U2OS cells. For dose response experiments, for each sd-rxRNA, the concentrations tested from left to right were 1 μM, 0.5 μM, 0.1 μM, 0.05 μM, 0.025 μM and 0.01 μM.

FIG. 18 shows two point dose response of sd-rxRNAs targeting TBX21 in Pan-T cells. For each sd-rxRNA, the concentrations tested were 1 μM (left) and 0.2 μM (right).

FIG. 19 shows three point dose response of sd-rxRNA targeting TIGIT in human primary NK cells. For each sd-rxRNA, the concentrations tested were 2 μM (left), 1 μM (middle) and 0.5 μM (right).

FIG. 20 shows six point dose response curves of sd-rxRNA targeting AKT1 in human primary T-cells. For each sd-rxRNA, the concentrations tested from left to right were 2 μM, 1 μM, 0.5 μM, 0.25 μM, 0.125 μM and 0.06 μM.

### DETAILED DESCRIPTION

In some aspects, the disclosure relates to compositions and methods for immunotherapy. The disclosure is based, in part, on chemically modified double-stranded nucleic acid molecules (e.g., sd-rxRNAs) targeting genes associated with controlling the differentiation process of T-cells and/or modulation of T-cell expression or activity, such as AKT, PD1, TIGIT, p53, Cbl-b, Tet2, Blimp-1, T-Box 21, or HK2, DNMT3A, PTPN6, etc. sd-rxRNA technology is particularly suitable for controlling the differentiation process of cells, including T-cells, and the production of therapeutic cells rich in the desired subtypes (T_{SCM}/T_{CM}). Several advantages of sd-rxRNA include: (i) sd-rxRNA can be developed in a short period of time and can silence virtually any target including “non-druggable” targets, e.g., those that are difficult to inhibit by small molecules, e.g., transcription factors; (ii) compared to alternative ex vivo siRNA transfection techniques (e.g., lipid mediated transfection or electroporation), sd-rxRNA can transfect a variety of cell types, including T cells with high transfection efficiency retaining a high cell viability; (iii) when added to cell culture media at an early expansion stage, sd-rxRNA compounds provide transient silencing of targets of interest during 8-10 division cycles, allowing the silencing effect to disappear in the final population of cells by the time of their re-infusion into a patient; (iv) sd-rxRNAs can be used in combination to simultaneously silence multiple targets, thus providing considerable flexibility for the use in different types of cell treatment protocols.

Described herein are sd-rxRNA directed to specific targets involved in the differentiation of T-cells, and the beneficial effect of such sd-rxRNAs on the phenotype of T-cells following ex vivo expansion. Also presented is a screening method that can be used to identify sd-rxRNA or combinations of sd-rxRNAs suitable for a specific cell production protocol.

As used herein, “nucleic acid molecule” includes but is not limited to: sd-rxRNA, rxRNAori, oligonucleotides, ASO, siRNA, shRNA, miRNA, ncRNA, cp-lasiRNA, aiRNA, single-stranded nucleic acid molecules, double-stranded nucleic acid molecules, RNA and DNA. In some embodiments, the nucleic acid molecule is a chemically-modified nucleic acid molecule, such as a chemically-modified oligonucleotide. In some embodiments, the nucleic acid molecule is double stranded. In some embodiments, chemically-modified double stranded nucleic acid molecules as described herein are sd-rxRNA molecules.

### Sd-rxRNA Molecules

Aspects of the invention relate to sd-rxRNA molecules that target genes associated with controlling the differentiation process of T-cells and/or modulating T-cell expression or activity, such as DNMT3A, PTPN6, PDCD1, TIGIT, AKT, p53, Cbl-b, Tet2, T-Box 21, Blimp-1 and HK2. In some embodiments, the disclosure provides an sd-rxRNA targeting a gene selected from PDCD1, AKT, p53, Cbl-b, Tet2, T-Box 21, Blimp-1, DNMT3A, PTPN6, and HK2. In some embodiments, a sd-rxRNA described herein comprises or consists of, or is targeted to or directed against, a sequence set forth in Tables 3-13, or a fragment thereof.

As used herein, an “sd-rxRNA” or an “sd-rxRNA molecule” refers to a self-delivering RNA molecule such as those described in, and incorporated by reference from, U.S. Pat. No. 8,796,443, granted on Aug. 5, 2014, entitled “REDUCED SIZE SELF-DELIVERING RNAI COMPOUNDS”, U.S. Pat. No. 9,175,289, granted on Nov. 3, 2015, entitled “REDUCED SIZE SELF-DELIVERING RNAI COMPOUNDS”, and PCT Publication No. WO2010/033247 (Application No. PCT/US2009/005247), filed on Sep. 22, 2009, and entitled “REDUCED SIZE SELF-DELIVERING RNAI COMPOUNDS.” Briefly, an sd-rxRNA, (also referred to as an sd-rxRNA^{nano}) is an isolated asymmetric double stranded nucleic acid molecule comprising a guide strand, with a minimal length of 16 nucleotides, and a passenger strand of 8-18 nucleotides in length, wherein the double stranded nucleic acid molecule has a double stranded region and a single stranded region, the single stranded region having 4-12 nucleotides in length and having at least three nucleotide backbone modifications. In preferred embodiments, the double stranded nucleic acid molecule has one end that is blunt or includes a one or two nucleotide overhang. sd-rxRNA molecules can be optimized through chemical modification, and in some instances through attachment of hydrophobic conjugates. Each of the above-referenced patents and publications are incorporated by reference herein in their entireties.

In some embodiments, an sd-rxRNA comprises an isolated double stranded nucleic acid molecule comprising a guide strand and a passenger strand, wherein the region of the molecule that is double stranded is from 8-15 nucleotides long, wherein the guide strand contains a single stranded region that is 4-12 nucleotides long, wherein the single stranded region of the guide strand contains 3, 4, 5, 6, 7, 8, 9, 10, 11 or 12 phosphorothioate modifications, and wherein at least 40% of the nucleotides of the double stranded nucleic acid are modified.

The nucleic acid molecules of the invention are referred to herein as isolated double stranded or duplex nucleic acids, oligonucleotides or polynucleotides, nano molecules, nano RNA, sd-rxRNA^{nano}, sd-rxRNA or RNA molecules of the invention.

sd-rxRNAs are much more effectively taken up by cells compared to conventional siRNAs. These molecules are highly efficient in silencing of target gene expression and offer significant advantages over previously described RNAi molecules including high activity in the presence of serum, efficient self-delivery, compatibility with a wide variety of linkers, and reduced presence or complete absence of chemical modifications that are associated with toxicity.

In contrast to single-stranded polynucleotides, duplex polynucleotides have traditionally been difficult to deliver to a cell as they have rigid structures and a large number of negative charges which makes membrane transfer difficult. sd-rxRNAs however, although partially double-stranded, are recognized in vivo as single-stranded and, as such, are capable of efficiently being delivered across cell membranes. As a result, the polynucleotides of the invention are capable in many instances of self-delivery. Thus, the polynucleotides of the invention may be formulated in a manner similar to conventional RNAi agents or they may be delivered to the cell or subject alone (or with non-delivery type carriers) and allowed to self-deliver. In one embodiment of the present invention, self-delivering asymmetric double-stranded RNA molecules are provided in which one portion of the molecule resembles a conventional RNA duplex and a second portion of the molecule is single stranded.

The oligonucleotides of the invention in some aspects have a combination of asymmetric structures including a double stranded region and a single stranded region of 5 nucleotides or longer, specific chemical modification patterns and are conjugated to lipophilic or hydrophobic molecules. In some embodiments, this class of RNAi like compounds have superior efficacy in vitro and in vivo. It is believed that the reduction in the size of the rigid duplex region in combination with phosphorothioate modifications applied to a single stranded region contribute to the observed superior efficacy.

In a preferred embodiment, the RNAi compounds of the invention comprise an asymmetric compound comprising a duplex region (required for efficient RISC entry of 8-15 bases long) and single stranded region of 4-12 nucleotides long. In some embodiments, the duplex region is 13 or 14 nucleotides long. A 6 or 7 nucleotide single stranded region is preferred in some embodiments. The single stranded region of the new RNAi compounds also comprises 2-12 phosphorothioate internucleotide linkages (referred to as phosphorothioate modifications). 6-8 phosphorothioate internucleotide linkages are preferred in some embodiments. Additionally, the RNAi compounds of the invention also include a unique chemical modification pattern, which provides stability and is compatible with RISC entry. In some embodiments, the combination of these elements has resulted in unexpected properties which are highly useful for delivery of RNAi reagents in vitro and in vivo.

The chemical modification pattern, which provides stability and is compatible with RISC entry includes modifications to the sense, or passenger, strand as well as the antisense, or guide, strand. For instance the passenger strand can be modified with any chemical entities which confirm stability and do not interfere with activity. Such modifications include 2′ ribo modifications (O-methyl, 2′ F, 2 deoxy and others) and backbone modification like phosphorothioate modifications. A preferred chemical modification pattern in the passenger strand includes O-methyl modification of C and U nucleotides within the passenger strand or alternatively the passenger strand may be completely O-methyl modified.

The guide strand, for example, may also be modified by any chemical modification which confirms stability without interfering with RISC entry. A preferred chemical modification pattern in the guide strand includes the majority of C and U nucleotides being 2′ F modified and the 5′ end being phosphorylated. Another preferred chemical modification pattern in the guide strand includes 2′O-methyl modification of position 1 and C/U in positions 11-18 and 5′ end chemical phosphorylation. Yet another preferred chemical modification pattern in the guide strand includes 2′O-methyl modification of position 1 and C/U in positions 11-18 and 5′ end chemical phosphorylation and 2′F modification of C/U in positions 2-10. In some embodiments the passenger strand and/or the guide strand contains at least one 5-methyl C or U modifications.

In some embodiments, at least 30% of the nucleotides in the sd-rxRNA are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the sd-rxRNA are modified. In some embodiments, 100% of the nucleotides in the sd-rxRNA are modified.

The above-described chemical modification patterns of the oligonucleotides of the invention are well tolerated and actually improve efficacy of asymmetric RNAi compounds. In some embodiments, elimination of any of the described components (guide strand stabilization, phosphorothioate stretch, sense strand stabilization and hydrophobic conjugate) or increase in size in some instances results in sub-optimal efficacy and in some instances complete loss of efficacy. The combination of elements results in development of a compound, which is fully active following passive delivery to cells such as HeLa cells, or T-cells.

The sd-rxRNA can be further improved in some instances by improving the hydrophobicity of compounds using novel types of chemistries. For example, one chemistry is related to use of hydrophobic base modifications. Any base in any position might be modified, as long as modification results in an increase of the partition coefficient of the base. The preferred locations for modification chemistries are positions 4 and 5 of the pyrimidines. The major advantage of these positions is (a) ease of synthesis and (b) lack of interference with base-pairing and A form helix formation, which are essential for RISC complex loading and target recognition. In some embodiments, sd-rxRNA compounds where multiple deoxy Uridines are present without interfering with overall compound efficacy are used. In addition, major improvement in tissue distribution and cellular uptake might be obtained by optimizing the structure of the hydrophobic conjugate. In some of the preferred embodiments, the structure of sterol is modified to alter (increase/decrease) C17 attached chain. This type of modification results in significant increase in cellular uptake and improvement of tissue uptake prosperities in vivo.

In some embodiments, a chemically-modified double stranded nucleic acid molecule is a hydrophobically modified siRNA-antisense hybrid molecule, comprising a double-stranded region of about 13-22 base pairs, with or without a 3′-overhang on each of the sense and antisense strands, and a 3′ single-stranded tail on the antisense strand of about 2-9 nucleotides. In some embodiments, the chemically-modified double stranded nucleic acid molecule contains at least one 2′-O-Methyl modification, at least one 2′-Fluoro modification, and at least one phosphorothioate modification, as well as at least one hydrophobic modification selected from sterol, cholesterol, vitamin D, napthyl, isobutyl, benzyl, indol, tryptophane, phenyl, and the like hydrophobic modifiers. In some embodiments, a chemically-modified double stranded nucleic acid molecule comprises a plurality of such modifications.

In some aspects, the disclosure relates to chemically-modified double stranded nucleic acid molecules that target genes encoding targets related to differentiation of cells (e.g., differentiation of T-cells), such as signal transduction/transcription factor targets, epigenetic targets, metabolic and co-inhibitory/negative regulatory targets. Examples of signal transduction/transcription factors include but are not limited to AKT, Blimp-1, and T-Box21. Examples of epigenetic proteins include but are not limited to Tet2. Examples of Metabolic targets include but are not limited to HK2. Examples of Co-inhibitory/negative regulatory targets include but are not limited to Cbl-b, p53, TIGIT and PD1.

In some embodiments, a chemically-modified double stranded nucleic acid targets a gene encoding DNMT3A, PTPN6, PDCD1, TIGIT, AKT, p53, Tet2, Blimp-1, TBox21 or HK2.

In some aspects, the disclosure relates to chemically-modified double stranded nucleic acid molecules that target genes encoding immune checkpoint proteins. Generally, an immune checkpoint protein is a protein that modulates a host immune response (e.g., by stimulating or suppressing T-cell function). Examples of stimulatory immune checkpoint proteins include but are not limited to CD27, CD28, CD40, CD122, CD137, OX40, glucocortocoid-induced TNFR family related gene (GITR), and inducible T-cell costimulator (ICOS). Examples of inhibitory immune checkpoint proteins include but are not limited to adenosine A2A receptor (A2AR), B7-H3, B7-H4, B and T Lymphocyte Attenuator (BTLA), Cytotoxic T-Lymphocyte-Associated protein 4 (CTLA-4), Indoleamine 2,3-dioxygenase (IDO), Killer-cell Immunoglobulin-like Receptor (KIR), Lymphocyte Activation Gene-3 (LAG3), Programmed Cell Death Protein 1 (PD1), T-cell Immunoglobulin and Mucin Domain 3 (TIM3), T cell immunoreceptor with Ig and ITIM domains (TIGIT) and V-domain Ig suppressor of T-cell Activation (VISTA). In some embodiments, a chemically-modified double stranded nucleic acid targets a gene encoding PDCD1 or TIGIT.

As used herein, “PDCD1” or “PD 1” refers to Programmed Cell Death Protein 1, which is a cell surface receptor that functions to down-regulate the immune system and promote immune self-tolerance by suppressing T-cell-mediated inflammatory activity. In some embodiments, PDCD1 is encoded by a nucleic acid sequence represented by NCBI Reference Sequence Number NM_005018.2.

As used herein, “TIGIT” refers to T-cell Immunoreceptor with Ig and ITIM domains, which is an immune receptor that down-regulates T-cell mediated immunity via the CD226/TIGIT-PVR pathway, for example by increasing interleukin 10 (IL-10) production. In some embodiments, TIGIT is encoded by a nucleic acid sequence represented by NCBI Reference Sequence Number NM_173799.3.

As used herein, “AKT” refers to Protein kinase B, which is a serine/threonine-specific kinase that plays a key role in glucose metabolism, cell proliferation, apoptosis and transcription. In some embodiments, AKT is encoded by a nucleic acid sequence represented by NCBI Reference Sequence Number NM_005163.

As used herein, “p53” refers to Tumor protein p53 (also known as cellular tumor antigen p53, phosphoprotein p53, tumor suppressor p53, antigen NY-CO-13 and transformation-related protein 53), which functions as a tumor suppressor that has been implicated in the regulation of differentiation and development pathways. In some embodiments, p53 is encoded by a nucleic acid sequence represented by NCBI Reference Sequence Number NM_001276761, NM_000546, NM_001126112, NM_001126113, NM_001126114, NM_001127233 or NM_011640.

As used herein, “Cbl-b” refers to E3 ubiquitin-protein ligase Cbl-b, which is an E3-ligase that serves as a negative regulator of T-cell activation. In some embodiments, Cbl-b is encoded by a nucleic acid sequence represented by NCBI Reference Sequence Number NM_170662.

As used herein, “Tet2” refers to Tet Methylcytosine Dioxygenase 2, which is a member of the Tet family, a series of methylcytosine dioxygenase genes which increase cellular levels of 5-Hydroxymethylcytosine (5hmC). In some embodiments, Tet2 is encoded by a nucleic acid sequence represented by NCBI Reference Sequence Number NM_001127208.

As used herein, “Blimp-1” refers to PR/SET Domain 1 (PRDM1), which encodes a protein that acts as a repressor of beta-interferon gene expression. In some embodiments, Blimp-1 is encoded by a nucleic acid sequence represented by NCBI Reference Sequence Number NM_001198.

As used herein, “T-Box 21” refers to T-box transcription factor TBX21, which is a member of a conserved family of genes that share a common DNA-binding domain called the T-box. In some embodiments, T-Box 21 is encoded by a nucleic acid sequence represented by NCBI Reference Sequence Number NM_013351.

As used herein, “HK2” refers to Hexokinase 2, which is an enzyme involved in the phosphorylation of glucose to produce glucose-6-phosphate. In some embodiments, HK2 is encoded by a nucleic acid sequence represented by NCBI Reference Sequence Number NM_000189.

As used herein, “DNMT3A” refers to DNA (cytosine-5)-methyltransferase 3A, which is an enzyme (e.g., a DNA methyltransferase) that catalyzes transfer of methyl groups to specific CpG structures in DNA. In some embodiments, DNMT3A is encoded by a nucleic acid sequence represented by NCBI Reference Sequence Number NM_175629.2.

As used herein, “PTPN6” refers to Tyrosine-protein phosphatase non-receptor type 6, which is also known as Src homology region 2 domain-containing phosphatase 1 (SHP-1). In some embodiments, PTPN6 is encoded by a nucleic acid sequence represented by NCBI Reference Sequence Number NM_002831.5.

Non-limiting examples of PDCD1 and Cbl-b sequences that may be targeted by chemically-modified double stranded nucleic acid molecules of the disclosure are listed in Tables 3-4.

In some embodiments a chemically-modified double stranded nucleic acid molecule comprises at least 12 nucleotides of a sequence within Tables 3-13. In some embodiments, a chemically-modified double stranded nucleic acid molecule comprises at least one sequence within Tables 3-4 (e.g., comprises a sense strand or an antisense strand comprising a sequence as set forth in any one of Tables 3-4). In some embodiments, a chemically-modified double stranded nucleic acid molecule (e.g., sd-rxRNA) comprises or consists of, or is targeted to or directed against, a sequence set forth in Tables 3-13, or a fragment thereof.

In some embodiments, a chemically-modified double stranded nucleic acid molecule (e.g., a sd-rxRNA) comprises a sense strand having the sequence set forth in PD 26 sense strand (SEQ ID NO: 112) and/or an antisense strand having the sequence set forth in PD 26 antisense strand (SEQ ID NO: 113). In some embodiments, a chemically-modified double stranded nucleic acid molecule (e.g., a sd-rxRNA) comprises a sense strand having the sequence set forth in CB 29 sense strand (SEQ ID NO: 248) and/or an antisense strand having the sequence set forth in CB 29 antisense strand (SEQ ID NO:249). In some embodiments, chemically-modified double stranded nucleic acid molecule (e.g., a sd-rxRNA) comprises a sense strand having the sequence set forth in CB 23 sense strand (SEQ ID NO: 236) and/or an antisense strand having the sequence set forth in CB 23 antisense strand (SEQ ID NO: 237).

In some embodiments, a dsRNA formulated according to the invention is a rxRNAori. rxRNAori refers to a class of RNA molecules described in and incorporated by reference from PCT Publication No. WO2009/102427 (Application No. PCT/US2009/000852), filed on Feb. 11, 2009, and entitled, “MODIFIED RNAI POLYNUCLEOTIDES AND USES THEREOF,” and US Patent Publication No. 2011/0039914, filed on Nov. 1, 2010, and entitled “MODIFIED RNAI POLYNUCLEOTIDES AND USES THEREOF.”

In some embodiments, an rxRNAori molecule comprises a double-stranded RNA (dsRNA) construct of 12-35 nucleotides in length, for inhibiting expression of a target gene, comprising: a sense strand having a 5′-end and a 3′-end, wherein the sense strand is highly modified with 2′-modified ribose sugars, and wherein 3-6 nucleotides in the central portion of the sense strand are not modified with 2′-modified ribose sugars and, an antisense strand having a 5′-end and a 3′-end, which hybridizes to the sense strand and to mRNA of the target gene, wherein the dsRNA inhibits expression of the target gene in a sequence-dependent manner.

rxRNAori can contain any of the modifications described herein. In some embodiments, at least 30% of the nucleotides in the rxRNAori are modified. For example, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the rxRNAori are modified. In some embodiments, 100% of the nucleotides in the sd-rxRNA are modified. In some embodiments, only the passenger strand of the rxRNAori contains modifications.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Thus, aspects of the invention relate to isolated double stranded nucleic acid molecules comprising a guide (antisense) strand and a passenger (sense) strand. As used herein, the term “double-stranded” refers to one or more nucleic acid molecules in which at least a portion of the nucleomonomers are complementary and hydrogen bond to form a double-stranded region. In some embodiments, the length of the guide strand ranges from 16-29 nucleotides long. In certain embodiments, the guide strand is 16, 17, 18, 19, 20, 21, 22, 23, 24, 25, 26, 27, 28, or 29 nucleotides long. The guide strand has complementarity to a target gene. Complementarity between the guide strand and the target gene may exist over any portion of the guide strand. Complementarity as used herein may be perfect complementarity or less than perfect complementarity as long as the guide strand is sufficiently complementary to the target that it mediates RNAi. In some embodiments complementarity refers to less than 25%, 20%, 15%, 10%, 5%, 4%, 3%, 2%, or 1% mismatch between the guide strand and the target. Perfect complementarity refers to 100% complementarity. In some embodiments, siRNA sequences with insertions, deletions, and single point mutations relative to the target sequence have also been found to be effective for inhibition. Moreover, not all positions of a siRNA contribute equally to target recognition. Mismatches in the center of the siRNA are most critical and essentially abolish target RNA cleavage. Mismatches upstream of the center or upstream of the cleavage site referencing the antisense strand are tolerated but significantly reduce target RNA cleavage. Mismatches downstream of the center or cleavage site referencing the antisense strand, preferably located near the 3′ end of the antisense strand, e.g. 1, 2, 3, 4, 5 or 6 nucleotides from the 3′ end of the antisense strand, are tolerated and reduce target RNA cleavage only slightly.

While not wishing to be bound by any particular theory, in some embodiments of double stranded nucleic acid molecules described herein, the guide strand is at least 16 nucleotides in length and anchors the Argonaute protein in RISC. In some embodiments, when the guide strand loads into RISC it has a defined seed region and target mRNA cleavage takes place across from position 10-11 of the guide strand. In some embodiments, the 5′ end of the guide strand is or is able to be phosphorylated. The nucleic acid molecules described herein may be referred to as minimum trigger RNA.

In some embodiments of double stranded nucleic acid molecules described herein, the length of the passenger strand ranges from 8-15 nucleotides long. In certain embodiments, the passenger strand is 8, 9, 10, 11, 12, 13, 14 or 15 nucleotides long. The passenger strand has complementarity to the guide strand. Complementarity between the passenger strand and the guide strand can exist over any portion of the passenger or guide strand. In some embodiments, there is 100% complementarity between the guide and passenger strands within the double stranded region of the molecule.

Aspects of the invention relate to double stranded nucleic acid molecules with minimal double stranded regions. In some embodiments the region of the molecule that is double stranded ranges from 8-15 nucleotides long. In certain embodiments, the region of the molecule that is double stranded is 8, 9, 10, 11, 12, 13, 14 or 15 nucleotides long. In certain embodiments the double stranded region is 13 or 14 nucleotides long. In some embodiments, the region of the molecule that is double stranded is 13-22 nucleotides long. In certain embodiments, the region of the molecule that is double stranded is 16, 17, 18, 19, 20, 21 or 22 nucleotides long.

There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. For example the single stranded region can be 4, 5, 6, 7, 8, 9, 10, 11 or 12 nucleotides long. However, in certain embodiments, the single stranded region can also be less than 4 or greater than 12 nucleotides long. In certain embodiments, the single stranded region is at least 6 or at least 7 nucleotides long. In some embodiments, the single stranded region is 2-9 nucleotides long, including 2 or 3 nucleotides long.

RNAi constructs associated with the invention can have a thermodynamic stability (ΔG) of less than −13 kkal/mol. In some embodiments, the thermodynamic stability (ΔG) is less than −20 kkal/mol. In some embodiments there is a loss of efficacy when (ΔG) goes below −21 kkal/mol. In some embodiments a (ΔG) value higher than −13 kkal/mol is compatible with aspects of the invention. Without wishing to be bound by any theory, in some embodiments a molecule with a relatively higher (ΔG) value may become active at a relatively higher concentration, while a molecule with a relatively lower (ΔG) value may become active at a relatively lower concentration. In some embodiments, the (ΔG) value may be higher than −9 kkcal/mol. The gene silencing effects mediated by the RNAi constructs associated with the invention, containing minimal double stranded regions, are unexpected because molecules of almost identical design but lower thermodynamic stability have been demonstrated to be inactive (Rana et al 2004).

Without wishing to be bound by any theory, results described herein suggest that a stretch of 8-10 bp of dsRNA or dsDNA will be structurally recognized by protein components of RISC or co-factors of RISC. Additionally, there is a free energy requirement for the triggering compound that it may be either sensed by the protein components and/or stable enough to interact with such components so that it may be loaded into the Argonaute protein. If optimal thermodynamics are present and there is a double stranded portion that is preferably at least 8 nucleotides then the duplex will be recognized and loaded into the RNAi machinery.

In some embodiments, thermodynamic stability is increased through the use of LNA bases. In some embodiments, additional chemical modifications are introduced. Several non-limiting examples of chemical modifications include: 5′ Phosphate, 2′-O-methyl, 2′-O-ethyl, 2′-fluoro, ribothymidine, C-5 propynyl-dC (pdC) and C-5 propynyl-dU (pdU); C-5 propynyl-C (pC) and C-5 propynyl-U (pU); 5-methyl C, 5-methyl U, 5-methyl dC, 5-methyl dU methoxy, (2,6-diaminopurine), 5′-Dimethoxytrityl-N4-ethyl-2′-deoxyCytidine and MGB (minor groove binder). It should be appreciated that more than one chemical modification can be combined within the same molecule.

Molecules associated with the invention are optimized for increased potency and/or reduced toxicity. For example, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. Specifically, reduction in 2′F content of a molecule is predicted to reduce toxicity of the molecule. Furthermore, the number of phosphorothioate modifications in an RNA molecule can influence the uptake of the molecule into a cell, for example the efficiency of passive uptake of the molecule into a cell. Preferred embodiments of molecules described herein have no 2′F modification and yet are characterized by equal efficacy in cellular uptake and tissue penetration. Such molecules represent a significant improvement over prior art, such as molecules described by Accell and Wolfrum, which are heavily modified with extensive use of 2′F.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contains 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

In some aspects, an optimal passenger strand is approximately 11-14 nucleotides in length. The passenger strand may contain modifications that confer increased stability. One or more nucleotides in the passenger strand can be 2′OMe modified. In some embodiments, one or more of the C and/or U nucleotides in the passenger strand is 2′OMe modified, or all of the C and U nucleotides in the passenger strand are 2′OMe modified. In certain embodiments, all of the nucleotides in the passenger strand are 2′OMe modified. One or more of the nucleotides on the passenger strand can also be phosphate-modified such as phosphorothioate modified. The passenger strand can also contain 2′ ribo, 2′F and 2 deoxy modifications or any combination of the above. Chemical modification patterns on both the guide and passenger strand can be well tolerated and a combination of chemical modifications can lead to increased efficacy and self-delivery of RNA molecules.

Aspects of the invention relate to RNAi constructs that have extended single-stranded regions relative to double stranded regions, as compared to molecules that have been used previously for RNAi. The single stranded region of the molecules may be modified to promote cellular uptake or gene silencing. In some embodiments, phosphorothioate modification of the single stranded region influences cellular uptake and/or gene silencing. The region of the guide strand that is phosphorothioate modified can include nucleotides within both the single stranded and double stranded regions of the molecule. In some embodiments, the single stranded region includes 2-12 phosphorothioate modifications. For example, the single stranded region can include 2, 3, 4, 5, 6, 7, 8, 9, 10, 11, or 12 phosphorothioate modifications. In some instances, the single stranded region contains 6-8 phosphorothioate modifications.

Molecules associated with the invention are also optimized for cellular uptake. In RNA molecules described herein, the guide and/or passenger strands can be attached to a conjugate. In certain embodiments the conjugate is hydrophobic. The hydrophobic conjugate can be a small molecule with a partition coefficient that is higher than 10. The conjugate can be a sterol-type molecule such as cholesterol, or a molecule with an increased length polycarbon chain attached to C17, and the presence of a conjugate can influence the ability of an RNA molecule to be taken into a cell with or without a lipid transfection reagent. The conjugate can be attached to the passenger or guide strand through a hydrophobic linker. In some embodiments, a hydrophobic linker is 5-12C in length, and/or is hydroxypyrrolidine-based. In some embodiments, a hydrophobic conjugate is attached to the passenger strand and the CU residues of either the passenger and/or guide strand are modified. In some embodiments, at least 50%, 55%, 60%, 65%, 70%, 75%, 80%, 85%, 90% or 95% of the CU residues on the passenger strand and/or the guide strand are modified. In some aspects, molecules associated with the invention are self-delivering (sd). As used herein, “self-delivery” refers to the ability of a molecule to be delivered into a cell without the need for an additional delivery vehicle such as a transfection reagent.

Aspects of the invention relate to selecting molecules for use in RNAi. In some embodiments, molecules that have a double stranded region of 8-15 nucleotides can be selected for use in RNAi. In some embodiments, molecules are selected based on their thermodynamic stability (ΔG). In some embodiments, molecules will be selected that have a (ΔG) of less than −13 kkal/mol. For example, the (ΔG) value may be −13, −14, −15, −16, −17, −18, −19, −21, −22 or less than −22 kkal/mol. In other embodiments, the (ΔG) value may be higher than −13 kkal/mol. For example, the (ΔG) value may be −12, −11, −10, −9, −8, −7 or more than −7 kkal/mol. It should be appreciated that AG can be calculated using any method known in the art. In some embodiments AG is calculated using Mfold, available through the Mfold internet site (mfold.bioinfo.rpi.edu/cgi-bin/rna-forml.cgi). Methods for calculating AG are described in, and are incorporated by reference from, the following references: Zuker, M. (2003) Nucleic Acids Res., 31(13):3406-15; Mathews, D. H., Sabina, J., Zuker, M. and Turner, D. H. (1999) J. Mol. Biol. 288:911-940; Mathews, D. H., Disney, M. D., Childs, J. L., Schroeder, S. J., Zuker, M., and Turner, D. H. (2004) Proc. Natl. Acad. Sci. 101:7287-7292; Duan, S., Mathews, D. H., and Turner, D. H. (2006) Biochemistry 45:9819-9832; Wuchty, S., Fontana, W., Hofacker, I. L., and Schuster, P. (1999) Biopolymers 49:145-165.

In certain embodiments, the polynucleotide contains 5′- and/or 3′-end overhangs. The number and/or sequence of nucleotides overhang on one end of the polynucleotide may be the same or different from the other end of the polynucleotide. In certain embodiments, one or more of the overhang nucleotides may contain chemical modification(s), such as phosphorothioate or 2′-OMe modification.

In certain embodiments, the polynucleotide is unmodified. In other embodiments, at least one nucleotide is modified. In further embodiments, the modification includes a 2′-H or 2′-modified ribose sugar at the 2nd nucleotide from the 5′-end of the guide sequence. The “2nd nucleotide” is defined as the second nucleotide from the 5′-end of the polynucleotide.

As used herein, “2′-modified ribose sugar” includes those ribose sugars that do not have a 2′—OH group. “2′-modified ribose sugar” does not include 2′-deoxyribose (found in unmodified canonical DNA nucleotides). For example, the 2′-modified ribose sugar may be 2′-O-alkyl nucleotides, 2′-deoxy-2′-fluoro nucleotides, 2′-deoxy nucleotides, or combination thereof.

In certain embodiments, the 2′-modified nucleotides are pyrimidine nucleotides (e.g., C/U). Examples of 2′-O-alkyl nucleotides include 2′-O-methyl nucleotides, or 2′-O-allyl nucleotides.

In certain embodiments, the sd-rxRNA polynucleotide of the invention with the above-referenced 5′-end modification exhibits significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the RNAi reagent or therapeutics.

As used herein, “off-target” gene silencing refers to unintended gene silencing due to, for example, spurious sequence homology between the antisense (guide) sequence and the unintended target mRNA sequence.

According to this aspect of the invention, certain guide strand modifications further increase nuclease stability, and/or lower interferon induction, without significantly decreasing RNAi activity (or no decrease in RNAi activity at all).

Certain combinations of modifications may result in further unexpected advantages, as partly manifested by enhanced ability to inhibit target gene expression, enhanced serum stability, and/or increased target specificity, etc.

In certain embodiments, the guide strand comprises a 2′-O-methyl modified nucleotide at the 2^{nd} nucleotide on the 5′-end of the guide strand and no other modified nucleotides.

In other aspects, the chemically modified double stranded nucleic acid molecule structures of the present invention mediate sequence-dependent gene silencing by a microRNA mechanism. As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

microRNAs are involved in down-regulating target genes in critical pathways, such as development and cancer, in mice, worms and mammals. Gene silencing through a microRNA mechanism is achieved by specific yet imperfect base-pairing of the miRNA and its target messenger RNA (mRNA). Various mechanisms may be used in microRNA-mediated down-regulation of target mRNA expression.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNAs are expressed by endogenous genes in vivo and are processed from a hairpin or stem-loop precursor (pre-miRNA or pri-miRNAs) by Dicer or other RNAses. miRNAs can exist transiently in vivo as a double-stranded duplex but only one strand is taken up by the RISC complex to direct gene silencing.

In some embodiments a version of chemically modified double stranded nucleic acid compounds, which are effective in cellular uptake and inhibition of miRNA activity are described. Essentially the compounds are similar to RISC entering version but large strand chemical modification patterns are optimized in the way to block cleavage and act as an effective inhibitor of the RISC action. For example, the compound might be completely or mostly O-methyl modified with the phosphorothioate content described previously. For these types of compounds, the 5′ phosphorylation is not necessary in some embodiments. The presence of a double stranded region is preferred as it is promotes cellular uptake and efficient RISC loading.

Another pathway that uses small RNAs as sequence-specific regulators is the RNA interference (RNAi) pathway, which is an evolutionarily conserved response to the presence of double-stranded RNA (dsRNA) in the cell. The dsRNAs are cleaved into −20-base pair (bp) duplexes of small-interfering RNAs (siRNAs) by Dicer. These small RNAs get assembled into multiprotein effector complexes called RNA-induced silencing complexes (RISCs). The siRNAs then guide the cleavage of target mRNAs with perfect complementarity.

Some aspects of biogenesis, protein complexes, and function are shared between the siRNA pathway and the miRNA pathway. Single-stranded polynucleotides may mimic the dsRNA in the siRNA mechanism, or the microRNA in the miRNA mechanism.

In certain embodiments, the modified RNAi constructs may have improved stability in serum and/or cerebral spinal fluid compared to an unmodified RNAi constructs having the same sequence.

In certain embodiments, the structure of the RNAi construct does not induce interferon response in primary cells, such as mammalian primary cells, including primary cells from human, mouse and other rodents, and other non-human mammals. In certain embodiments, the RNAi construct may also be used to inhibit expression of a target gene in an invertebrate organism.

To further increase the stability of the subject constructs in vivo, the 3′-end of the structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety.

The RNAi constructs of the invention are capable of inhibiting the synthesis of any target protein encoded by target gene(s). The invention includes methods to inhibit expression of a target gene either in a cell in vitro, or in vivo. As such, the RNAi constructs of the invention are useful for treating a patient with a disease characterized by the overexpression of a target gene.

The target gene can be endogenous or exogenous (e.g., introduced into a cell by a virus or using recombinant DNA technology) to a cell. Such methods may include introduction of RNA into a cell in an amount sufficient to inhibit expression of the target gene. By way of example, such an RNA molecule may have a guide strand that is complementary to the nucleotide sequence of the target gene, such that the composition inhibits expression of the target gene.

The invention also relates to vectors expressing the nucleic acids of the invention, and cells comprising such vectors or the nucleic acids. The cell may be a mammalian cell in vivo or in culture, such as a human cell.

The invention further relates to compositions comprising the subject RNAi constructs, and a pharmaceutically acceptable carrier or diluent.

The method may be carried out in vitro, ex vivo, or in vivo, in, for example, mammalian cells in culture, such as a human cell in culture.

The target cells (e.g., mammalian cell) may be contacted in the presence of a delivery reagent, such as a lipid (e.g., a cationic lipid) or a liposome.

Another aspect of the invention provides a method for inhibiting the expression of a target gene in a mammalian cell, comprising contacting the mammalian cell with a vector expressing the subject RNAi constructs.

In one aspect of the invention, a longer duplex polynucleotide is provided, including a first polynucleotide that ranges in size from about 16 to about 30 nucleotides; a second polynucleotide that ranges in size from about 26 to about 46 nucleotides, wherein the first polynucleotide (the antisense strand) is complementary to both the second polynucleotide (the sense strand) and a target gene, and wherein both polynucleotides form a duplex and wherein the first polynucleotide contains a single stranded region longer than 6 bases in length and is modified with alternative chemical modification pattern, and/or includes a conjugate moiety that facilitates cellular delivery. In this embodiment, between about 40% to about 90% of the nucleotides of the passenger strand between about 40% to about 90% of the nucleotides of the guide strand, and between about 40% to about 90% of the nucleotides of the single stranded region of the first polynucleotide are chemically modified nucleotides.

In an embodiment, the chemically modified nucleotide in the polynucleotide duplex may be any chemically modified nucleotide known in the art, such as those discussed in detail above. In a particular embodiment, the chemically modified nucleotide is selected from the group consisting of 2′ F modified nucleotides, 2′-O-methyl modified and 2′deoxy nucleotides. In another particular embodiment, the chemically modified nucleotides results from “hydrophobic modifications” of the nucleotide base. In another particular embodiment, the chemically modified nucleotides are phosphorothioates. In an additional particular embodiment, chemically modified nucleotides are combination of phosphorothioates, 2′-O-methyl, 2′deoxy, hydrophobic modifications and phosphorothioates. As these groups of modifications refer to modification of the ribose ring, back bone and nucleotide, it is feasible that some modified nucleotides will carry a combination of all three modification types.

In another embodiment, the chemical modification is not the same across the various regions of the duplex. In a particular embodiment, the first polynucleotide (the passenger strand), has a large number of diverse chemical modifications in various positions. For this polynucleotide up to 90% of nucleotides might be chemically modified and/or have mismatches introduced.

In another embodiment, chemical modifications of the first or second polynucleotide include, but not limited to, 5′ position modification of Uridine and Cytosine (4-pyridyl, 2-pyridyl, indolyl, phenyl (C₆H₅OH); tryptophanyl (C8H6N)CH2CH(NH2)CO), isobutyl, butyl, aminobenzyl; phenyl; naphthyl, etc.), where the chemical modification might alter base pairing capabilities of a nucleotide. For the guide strand an important feature of this aspect of the invention is the position of the chemical modification relative to the 5′ end of the antisense and sequence. For example, chemical phosphorylation of the 5′ end of the guide strand is usually beneficial for efficacy. O-methyl modifications in the seed region of the sense strand (position 2-7 relative to the 5′ end) are not generally well tolerated, whereas 2′F and deoxy are well tolerated. The mid part of the guide strand and the 3′ end of the guide strand are more permissive in a type of chemical modifications applied. Deoxy modifications are not tolerated at the 3′ end of the guide strand.

A unique feature of this aspect of the invention involves the use of hydrophobic modification on the bases. In one embodiment, the hydrophobic modifications are preferably positioned near the 5′ end of the guide strand, in other embodiments, they localized in the middle of the guides strand, in other embodiment they localized at the 3′ end of the guide strand and yet in another embodiment they are distributed thought the whole length of the polynucleotide. The same type of patterns is applicable to the passenger strand of the duplex.

The other part of the molecule is a single stranded region. The single stranded region is expected to range from 7 to 40 nucleotides.

In one embodiment, the single stranded region of the first polynucleotide contains modifications selected from the group consisting of between 40% and 90% hydrophobic base modifications, between 40%-90% phosphorothioates, between 40%-90% modification of the ribose moiety, and any combination of the preceding.

Efficiency of guide strand (first polynucleotide) loading into the RISC complex might be altered for heavily modified polynucleotides, so in one embodiment, the duplex polynucleotide includes a mismatch between nucleotide 9, 11, 12, 13, or 14 on the guide strand (first polynucleotide) and the opposite nucleotide on the sense strand (second polynucleotide) to promote efficient guide strand loading.

More detailed aspects of the invention are described in the sections below.

### Duplex Characteristics

Double-stranded oligonucleotides of the invention may be formed by two separate complementary nucleic acid strands. Duplex formation can occur either inside or outside the cell containing the target gene.

As used herein, the term “duplex” includes the region of the double-stranded nucleic acid molecule(s) that is (are) hydrogen bonded to a complementary sequence. Double-stranded oligonucleotides of the invention may comprise a nucleotide sequence that is sense to a target gene and a complementary sequence that is antisense to the target gene. The sense and antisense nucleotide sequences correspond to the target gene sequence, e.g., are identical or are sufficiently identical to effect target gene inhibition (e.g., are about at least about 98% identical, 96% identical, 94%, 90% identical, 85% identical, or 80% identical) to the target gene sequence.

In certain embodiments, the double-stranded oligonucleotide of the invention is double-stranded over its entire length, i.e., with no overhanging single-stranded sequence at either end of the molecule, i.e., is blunt-ended. In other embodiments, the individual nucleic acid molecules can be of different lengths. In other words, a double-stranded oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). Likewise, when a single nucleic acid molecule is used a portion of the molecule at either end can remain single-stranded.

In one embodiment, a double-stranded oligonucleotide of the invention contains mismatches and/or loops or bulges, but is double-stranded over at least about 70% of the length of the oligonucleotide. In another embodiment, a double-stranded oligonucleotide of the invention is double-stranded over at least about 80% of the length of the oligonucleotide. In another embodiment, a double-stranded oligonucleotide of the invention is double-stranded over at least about 90%-95% of the length of the oligonucleotide. In another embodiment, a double-stranded oligonucleotide of the invention is double-stranded over at least about 96%-98% of the length of the oligonucleotide. In certain embodiments, the double-stranded oligonucleotide of the invention contains at least or up to 1, 2, 3, 4, 5, 6, 7, 8, 9, 10, 11, 12, 13, 14, or 15 mismatches.

### Modifications

The nucleotides of the invention may be modified at various locations, including the sugar moiety, the phosphodiester linkage, and/or the base.

In some embodiments, the base moiety of a nucleoside may be modified. For example, a pyrimidine base may be modified at the 2, 3, 4, 5, and/or 6 position of the pyrimidine ring. In some embodiments, the exocyclic amine of cytosine may be modified. A purine base may also be modified. For example, a purine base may be modified at the 1, 2, 3, 6, 7, or 8 position. In some embodiments, the exocyclic amine of adenine may be modified. In some cases, a nitrogen atom in a ring of a base moiety may be substituted with another atom, such as carbon. A modification to a base moiety may be any suitable modification. Examples of modifications are known to those of ordinary skill in the art. In some embodiments, the base modifications include alkylated purines or pyrimidines, acylated purines or pyrimidines, or other heterocycles.

In some embodiments, a pyrimidine may be modified at the 5 position. For example, the 5 position of a pyrimidine may be modified with an alkyl group, an alkynyl group, an alkenyl group, an acyl group, or substituted derivatives thereof. In other examples, the 5 position of a pyrimidine may be modified with a hydroxyl group or an alkoxyl group or substituted derivative thereof. Also, the N⁴ position of a pyrimidine may be alkylated. In still further examples, the pyrimidine 5-6 bond may be saturated, a nitrogen atom within the pyrimidine ring may be substituted with a carbon atom, and/or the 02 and 0⁴ atoms may be substituted with sulfur atoms. It should be understood that other modifications are possible as well.

In other examples, the N⁷ position and/or N² and/or N³ position of a purine may be modified with an alkyl group or substituted derivative thereof. In further examples, a third ring may be fused to the purine bicyclic ring system and/or a nitrogen atom within the purine ring system may be substituted with a carbon atom. It should be understood that other modifications are possible as well.

Non-limiting examples of pyrimidines modified at the 5 position are disclosed in U.S. Pat. Nos. 5,591,843, 7,205,297, 6,432,963, and 6,020,483; non-limiting examples of pyrimidines modified at the N⁴ position are disclosed in U.S. Pat. No. 5,580,731; non-limiting examples of purines modified at the 8 position are disclosed in U.S. Pat. Nos. 6,355,787 and 5,580,972; non-limiting examples of purines modified at the N⁶ position are disclosed in U.S. Pat. Nos. 4,853,386, 5,789,416, and 7,041,824; and non-limiting examples of purines modified at the 2 position are disclosed in U.S. Pat. Nos. 4,201,860 and 5,587,469, all of which are incorporated herein by reference.

Non-limiting examples of modified bases include N⁴,N⁴-ethanocytosine, 7-deazaxanthosine, 7-deazaguanosine, 8-oxo-N⁶-methyladenine, 4-acetylcytosine, 5-(carboxyhydroxylmethyl) uracil, 5-fluorouracil, 5-bromouracil, 5-carboxymethylaminomethyl-2-thiouracil, 5-carboxymethylaminomethyl uracil, dihydrouracil, inosine, N⁶-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyl uracil, 5-methoxy aminomethyl-2-thiouracil, 5-methoxyuracil, 2-methylthio-N6-isopentenyladenine, pseudouracil, 5-methyl-2-thiouracil, 2-thiouracil, 4-thiouracil, 5-methyluracil, 2-thiocytosine, and 2,6-diaminopurine. In some embodiments, the base moiety may be a heterocyclic base other than a purine or pyrimidine. The heterocyclic base may be optionally modified and/or substituted.

Sugar moieties include natural, unmodified sugars, e.g., monosaccharide (such as pentose, e.g., ribose, deoxyribose), modified sugars and sugar analogs. In general, possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One particularly useful group of modified nucleomonomers are 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the invention may contain both methylated and unmethylated nucleomonomers.

Some exemplary modified nucleomonomers include sugar- or backbone-modified ribonucleotides. Modified ribonucleotides may contain a non-naturally occurring base (instead of a naturally occurring base), such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alxoxy (or OR), R or alkyl, halogen, SH, SR, amino (such as NH₂, NHR, NR₂,), or CN group, wherein R is lower alkyl, alkenyl, or alkynyl.

Modified ribonucleotides may also have the phosphodiester group connecting to adjacent ribonucleotides replaced by a modified group, e.g., of phosphorothioate group. More generally, the various nucleotide modifications may be combined.

Although the antisense (guide) strand may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter antisense gene. In some cases, the antisense strand generally will be substantially identical (although in antisense orientation) to the target gene.

The use of 2′-O-methyl modified RNA may also be beneficial in circumstances in which it is desirable to minimize cellular stress responses. RNA having 2′-O-methyl nucleomonomers may not be recognized by cellular machinery that is thought to recognize unmodified RNA. The use of 2′-O-methylated or partially 2′-O-methylated RNA may avoid the interferon response to double-stranded nucleic acids, while maintaining target RNA inhibition. This may be useful, for example, for avoiding the interferon or other cellular stress responses, both in short RNAi (e.g., siRNA) sequences that induce the interferon response, and in longer RNAi sequences that may induce the interferon response.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary nucleomonomers can be found, e.g., in U.S. Pat. No. 5,849,902, incorporated by reference herein.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

In certain embodiments, oligonucleotides of the invention comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

One of ordinary skill in the art will appreciate that the synthetic methods, as described herein, utilize a variety of protecting groups. By the term “protecting group,” as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenyl acetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethyl silylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxyb enzylideneamine, N-diphenylmethyl eneamine, N-[(2-pyridyl)mesityl]methyleneamine, N—(N′,N′-dimethyl aminomethylene)amine, N,N′-isopropylidenediamine, N-p-nitrob enzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorob enzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethyl silylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “lower alkyl” is used to indicate those alkyl groups (cyclic, acyclic, substituted, unsubstituted, branched, or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments described herein.

The terms “halo” and “halogen” as used herein refer to an atom selected from fluorine, chlorine, bromine, and iodine.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfmyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻ (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The terms “polynucleotide,” “nucleotide sequence,” “nucleic acid,” “nucleic acid molecule,” “nucleic acid sequence,” and “oligonucleotide” refer to a polymer of two or more nucleotides. The polynucleotides can be DNA, RNA, or derivatives or modified versions thereof. The polynucleotide may be single-stranded or double-stranded. The polynucleotide can be modified at the base moiety, sugar moiety, or phosphate backbone, for example, to improve stability of the molecule, its hybridization parameters, etc. The polynucleotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethyl aminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The olynucleotide may comprise a modified sugar moiety (e.g., 2′-fluororibose, ribose, 2′-deoxyribose, 2′-O-methylcytidine, arabinose, and hexose), and/or a modified phosphate moiety (e.g., phosphorothioates and 5′-N-phosphoramidite linkages). A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA, and RNA-RNA hybrids, as well as “protein nucleic acids” (PNA) formed by conjugating bases to an amino acid backbone.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In a preferred embodiment, the nucleomonomers of an oligonucleotide of the invention are RNA nucleotides. In another preferred embodiment, the nucleomonomers of an oligonucleotide of the invention are modified RNA nucleotides. Thus, the oligonucleotides contain modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

The nucleic acid molecules may be associated with a hydrophobic moiety for targeting and/or delivery of the molecule to a cell. In certain embodiments, the hydrophobic moiety is associated with the nucleic acid molecule through a linker. In certain embodiments, the association is through non-covalent interactions. In other embodiments, the association is through a covalent bond. Any linker known in the art may be used to associate the nucleic acid with the hydrophobic moiety. Linkers known in the art are described in published international PCT applications, WO 92/03464, WO 95/23162, WO 2008/021157, WO 2009/021157, WO 2009/134487, WO 2009/126933, U.S. Patent Application Publication 2005/0107325, U.S. Pat. Nos. 5,414,077, 5,419,966, 5,512,667, 5,646,126, and 5,652,359, which are incorporated herein by reference. The linker may be as simple as a covalent bond to a multi-atom linker. The linker may be cyclic or acyclic. The linker may be optionally substituted. In certain embodiments, the linker is capable of being cleaved from the nucleic acid. In certain embodiments, the linker is capable of being hydrolyzed under physiological conditions. In certain embodiments, the linker is capable of being cleaved by an enzyme (e.g., an esterase or phosphodiesterase). In certain embodiments, the linker comprises a spacer element to separate the nucleic acid from the hydrophobic moiety. The spacer element may include one to thirty carbon or heteroatoms. In certain embodiments, the linker and/or spacer element comprises protonatable functional groups. Such protonatable functional groups may promote the endosomal escape of the nucleic acid molecule. The protonatable functional groups may also aid in the delivery of the nucleic acid to a cell, for example, neutralizing the overall charge of the molecule. In other embodiments, the linker and/or spacer element is biologically inert (that is, it does not impart biological activity or function to the resulting nucleic acid molecule).

In certain embodiments, the nucleic acid molecule with a linker and hydrophobic moiety is of the formulae described herein. In certain embodiments, the nucleic acid molecule is of the formula:

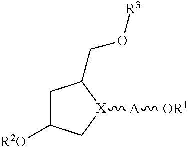

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the molecule is of the formula:

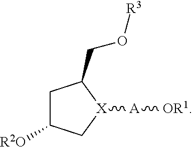

In certain embodiments, the molecule is of the formula:

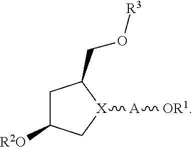

In certain embodiments, the molecule is of the formula:

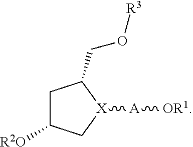

In certain embodiments, the molecule is of the formula:

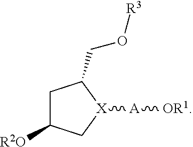

In certain embodiments, X is N. In certain embodiments, X is CH.

In certain embodiments, A is a bond. In certain embodiments, A is substituted or unsubstituted, cyclic or acyclic, branched or unbranched aliphatic. In certain embodiments, A is acyclic, substituted or unsubstituted, branched or unbranched aliphatic. In certain embodiments, A is acyclic, substituted, branched or unbranched aliphatic. In certain embodiments, A is acyclic, substituted, unbranched aliphatic. In certain embodiments, A is acyclic, substituted, unbranched alkyl. In certain embodiments, A is acyclic, substituted, unbranched C₁₋₂₀ alkyl. In certain embodiments, A is acyclic, substituted, unbranched C₁₋₁₂ alkyl. In certain embodiments, A is acyclic, substituted, unbranched C₁₋₁₀ alkyl. In certain embodiments, A is acyclic, substituted, unbranched C₁₋₈ alkyl. In certain embodiments, A is acyclic, substituted, unbranched C₁₋₆ alkyl. In certain embodiments, A is substituted or unsubstituted, cyclic or acyclic, branched or unbranched heteroaliphatic. In certain embodiments, A is acyclic, substituted or unsubstituted, branched or unbranched heteroaliphatic. In certain embodiments, A is acyclic, substituted, branched or unbranched heteroaliphatic. In certain embodiments, A is acyclic, substituted, unbranched heteroaliphatic.

In certain embodiments, A is of the formula:

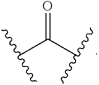 [SMILES: *C(*)=O]

In certain embodiments, A is of one of the formulae:

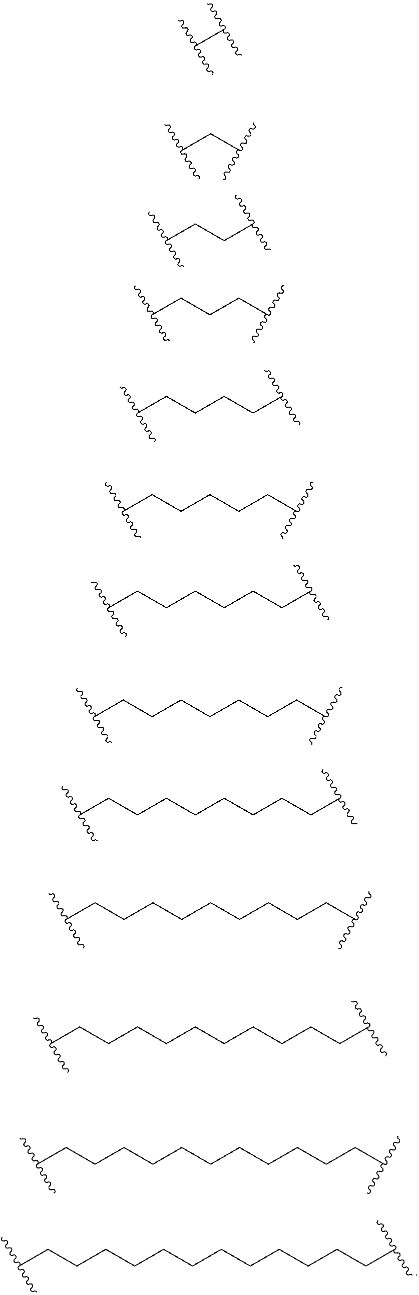

In certain embodiments, A is of one of the formulae:

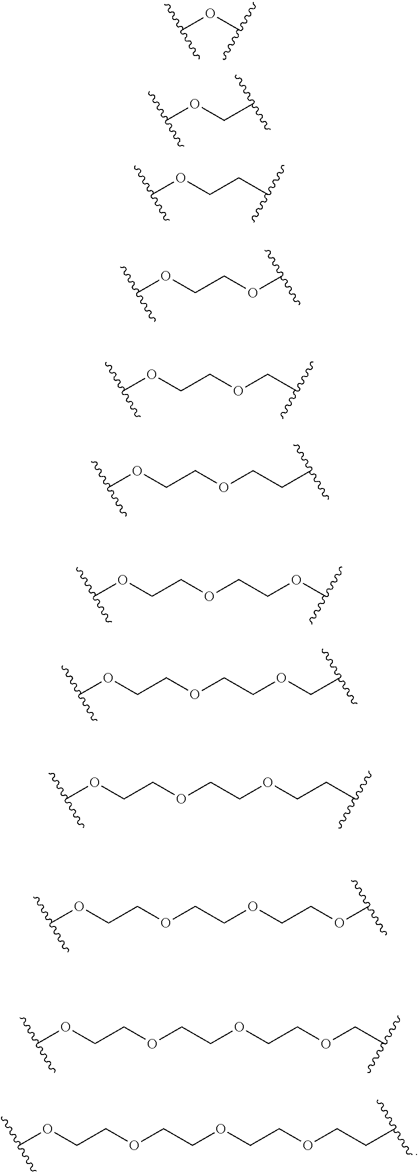

In certain embodiments, A is of one of the formulae:

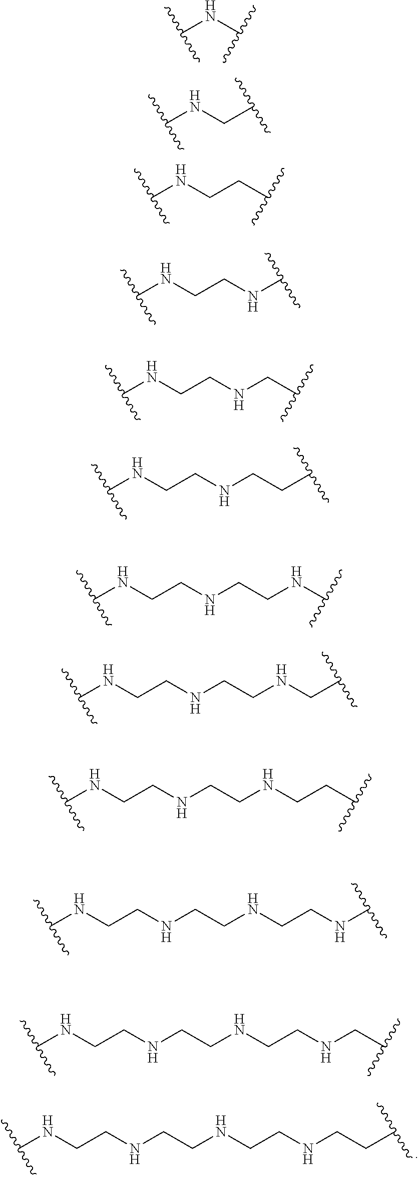

In certain embodiments, A is of the formula:

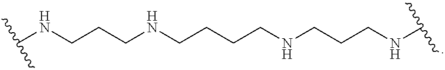 [SMILES: *NCCCNCCCCNCCCN*]

In certain embodiments, A is of the formula:

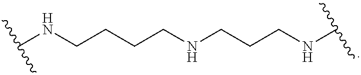 [SMILES: *NCCCCNCCCN*]

In certain embodiments, A is of the formula:

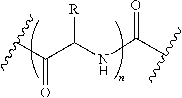

wherein

each occurrence of R is independently the side chain of a natural or unnatural amino acid; and

n is an integer between 1 and 20, inclusive. In certain embodiments, A is of the formula:

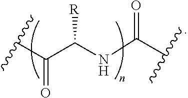

In certain embodiments, each occurrence of R is independently the side chain of a natural amino acid. In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

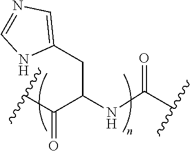

wherein n is an integer between 1 and 20, inclusive. In certain embodiments, A is of the formula:

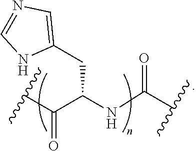

In certain embodiments, n is an integer between 1 and 15, inclusive. In certain embodiments, n is an integer between 1 and 10, inclusive. In certain embodiments, n is an integer between 1 and 5, inclusive.

In certain embodiments, A is of the formula:

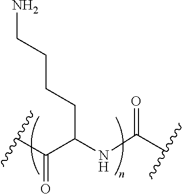

wherein n is an integer between 1 and 20, inclusive. In certain embodiments, A is of the formula:

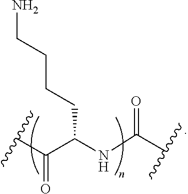

In certain embodiments, n is an integer between 1 and 15, inclusive. In certain embodiments, n is an integer between 1 and 10, inclusive. In certain embodiments, n is an integer between 1 and 5, inclusive.

In certain embodiments, the molecule is of the formula:

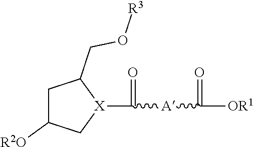

wherein X, R¹, R², and R³ are as defined herein; and

A′ is substituted or unsubstituted, cyclic or acyclic, branched or unbranched aliphatic; or substituted or unsubstituted, cyclic or acyclic, branched or unbranched heteroaliphatic.

In certain embodiments, A′ is of one of the formulae:

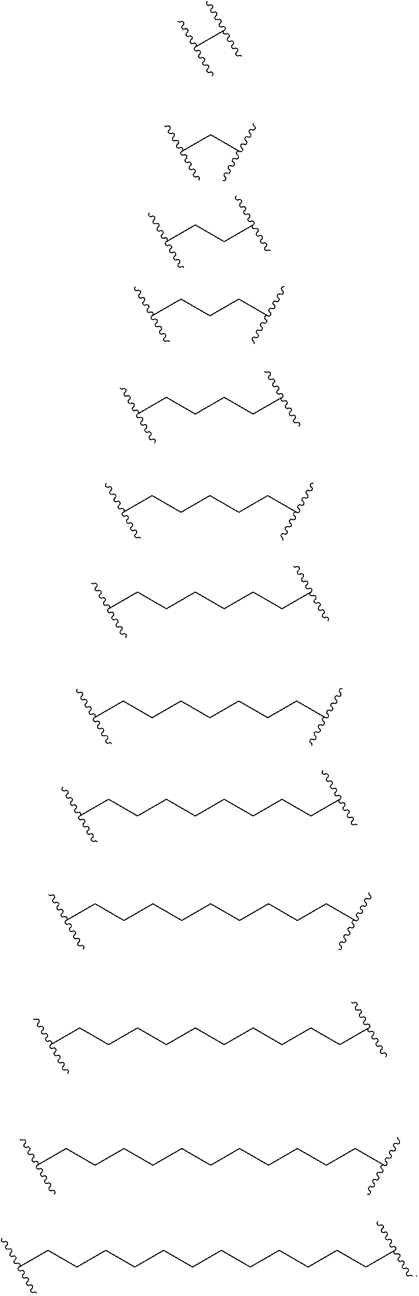

In certain embodiments, A is of one of the formulae:

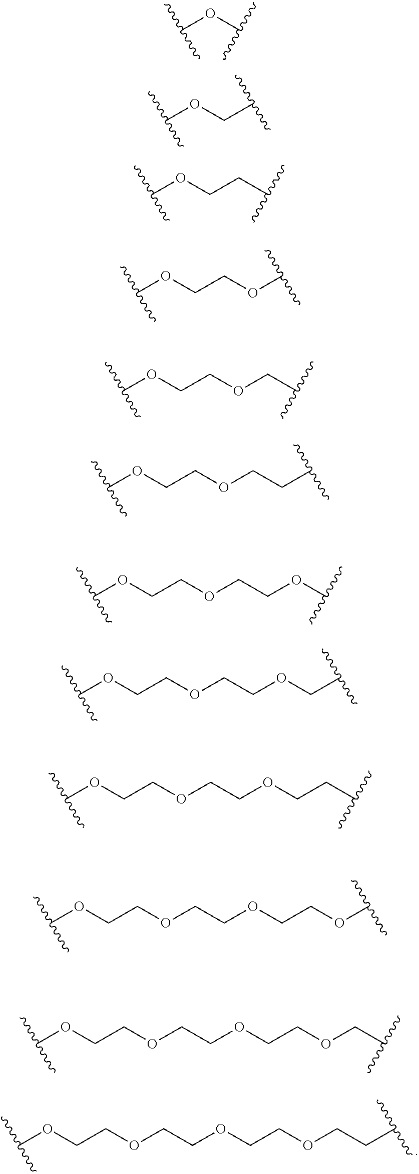

In certain embodiments, A is of one of the formulae:

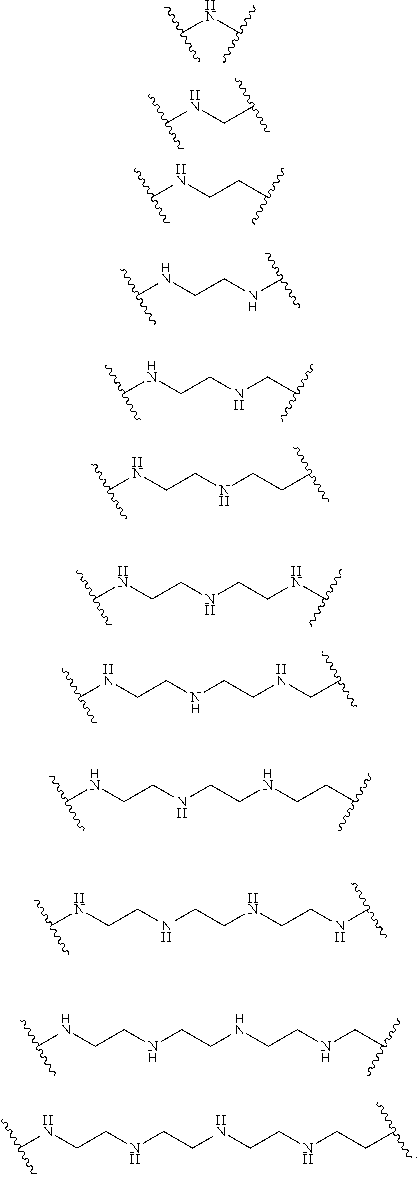

In certain embodiments, A is of the formula:

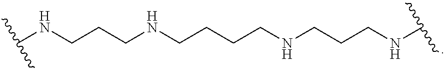 [SMILES: *NCCCNCCCCNCCCN*]

In certain embodiments, A is of the formula:

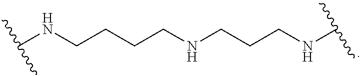 [SMILES: *NCCCCNCCCN*]

In certain embodiments, R¹ is a steroid. In certain embodiments, R¹ is a cholesterol. In certain embodiments, R¹ is a lipophilic vitamin. In certain embodiments, R1 is a vitamin A. In certain embodiments, R¹ is a vitamin E.

In certain embodiments, R¹ is of the formula:

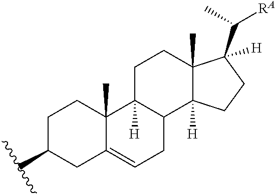

wherein R^{A} is substituted or unsubstituted, cyclic or acyclic, branched or unbranched aliphatic; or substituted or unsubstituted, cyclic or acyclic, branched or unbranched heteroaliphatic.

In certain embodiments, R¹ is of the formula:

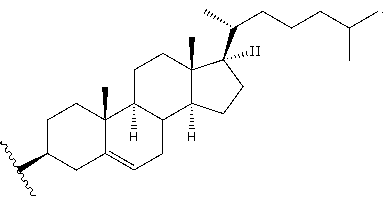

In certain embodiments, R¹ is of the formula:

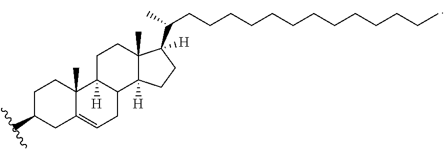

In certain embodiments, R¹ is of the formula:

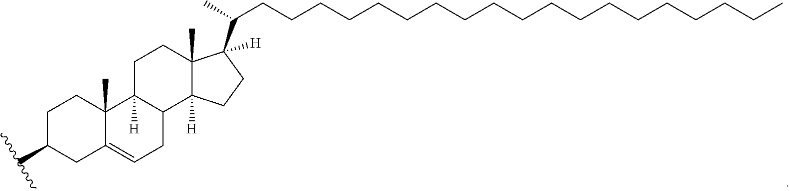

In certain embodiments, R¹ is of the formula:

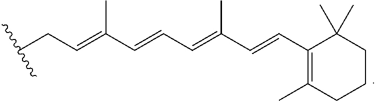 [SMILES: *C/C=C(C)/C=C/C=C(C)/C=C/C1=C(C)CCCC1(C)C]

In certain embodiments, R¹ is of the formula:

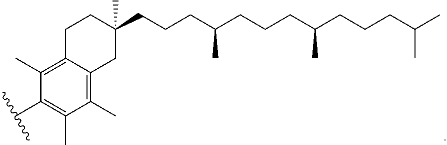 [SMILES: *c1c(C)c(C)c2c(c1C)CC[C@@](C)(CCC[C@H](C)CCC[C@H](C)CCCC(C)C)C2]

In certain embodiments, the nucleic acid molecule is of the formula:

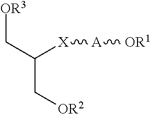

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the nucleic acid molecule is of the formula:

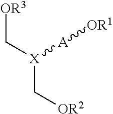

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid.

In certain embodiments, the nucleic acid molecule is of the formula:

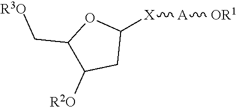

wherein

X is N or CH;

A is a bond; substituted or unsubstituted, cyclic or acyclic, branched or unbranched aliphatic; or substituted or unsubstituted, cyclic or acyclic, branched or unbranched heteroaliphatic;

R¹ is a hydrophobic moiety;

R² is hydrogen; an oxygen-protecting group; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; and

R³ is a nucleic acid. In certain embodiments, the nucleic acid molecule is of the formula:

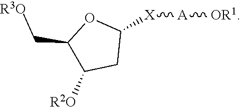

In certain embodiments, the nucleic acid molecule is of the formula:

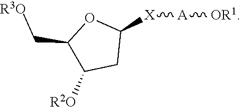

In certain embodiments, the nucleic acid molecule is of the formula:

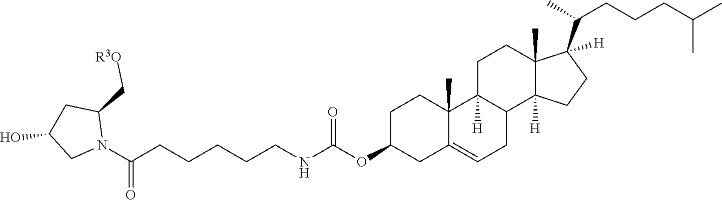

wherein R³ is a nucleic acid.

In certain embodiments, the nucleic acid molecule is of the formula:

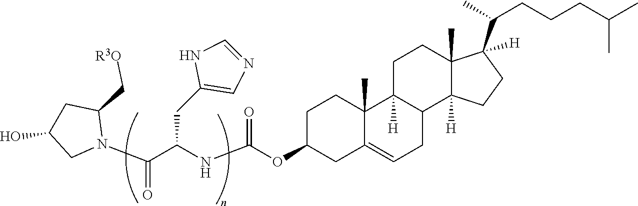

wherein R³ is a nucleic acid; and

n is an integer between 1 and 20, inclusive.

In certain embodiments, the nucleic acid molecule is of the formula:

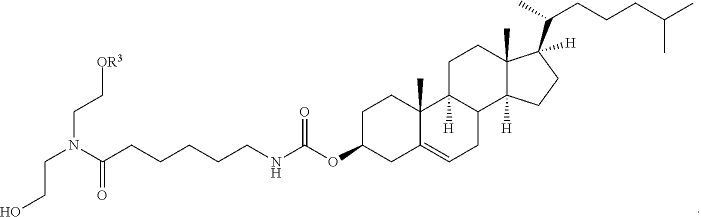

In certain embodiments, the nucleic acid molecule is of the formula:

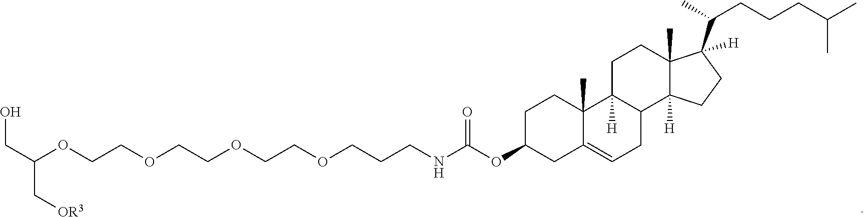

In certain embodiments, the nucleic acid molecule is of the formula:

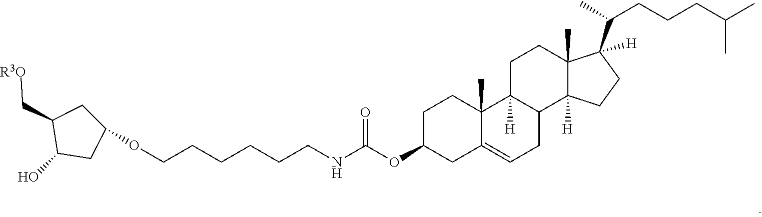

In certain embodiments, the nucleic acid molecule is of the formula:

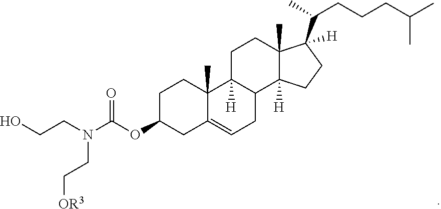

In certain embodiments, the nucleic acid molecule is of the formula:

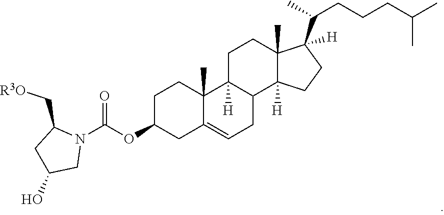

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothiate linkages.

In certain embodiments, oligonucleotides of the invention comprise hydrophobically modified nucleotides or “hydrophobic modifications.” As used herein “hydrophobic modifications” refers to bases that are modified such that (1) overall hydrophobicity of the base is significantly increased, and/or (2) the base is still capable of forming close to regular Watson-Crick interaction. Several non-limiting examples of base modifications include 5-position uridine and cytidine modifications such as phenyl, 4-pyridyl, 2-pyridyl, indolyl, and isobutyl, phenyl (C6H5OH); tryptophanyl (C8H6N)CH2CH(NH2)CO), Isobutyl, butyl, aminobenzyl; phenyl; and naphthyl.

Other types of conjugates that can be attached to the end (3′ or 5′ end), a loop region, or any other parts of a chemically modified double stranded nucleic acid molecule include a sterol, sterol type molecule, peptide, small molecule, protein, etc. In some embodiments, a chemically modified double stranded nucleic acid molecule, such as an sd-rxRNA, may contain more than one conjugate (same or different chemical nature). In some embodiments, the conjugate is cholesterol.

In some embodiments, the first nucleotide relative to the 5′end of the guide strand has a 2′-O-methyl modification, optionally wherein the 2′-O-methyl modification is a 5P-2′O-methyl U modification, or a 5′ vinyl phosphonate 2′-O-methyl U modification. Another way to increase target gene specificity, or to reduce off-target silencing effect, is to introduce a 2′-modification (such as the 2′-0 methyl modification) at a position corresponding to the second 5′-end nucleotide of the guide sequence. Antisense (guide) sequences of the invention can be “chimeric oligonucleotides” which comprise an RNA-like and a DNA-like region.

The language “RNase H activating region” includes a region of an oligonucleotide, e.g., a chimeric oligonucleotide, that is capable of recruiting RNase H to cleave the target RNA strand to which the oligonucleotide binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See, e.g., U.S. Pat. No. 5,849,902). Preferably, the RNase H activating region comprises about nine contiguous deoxyribose containing nucleomonomers.

The language “non-activating region” includes a region of an antisense sequence, e.g., a chimeric oligonucleotide, that does not recruit or activate RNase H. Preferably, a non-activating region does not comprise phosphorothioate DNA. The oligonucleotides of the invention comprise at least one non-activating region. In one embodiment, the non-activating region can be stabilized against nucleases or can provide specificity for the target by being complementary to the target and forming hydrogen bonds with the target nucleic acid molecule, which is to be bound by the oligonucleotide.

In one embodiment, at least a portion of the contiguous polynucleotides are linked by a substitute linkage, e.g., a phosphorothioate linkage.

In certain embodiments, most or all of the nucleotides beyond the guide sequence (2′-modified or not) are linked by phosphorothioate linkages. Such constructs tend to have improved pharmacokinetics due to their higher affinity for serum proteins. The phosphorothioate linkages in the non-guide sequence portion of the polynucleotide generally do not interfere with guide strand activity, once the latter is loaded into RISC. In some embodiments, high levels of phosphorothioate modification can lead to improved delivery. In some embodiments, the guide and/or passenger strand is completely phosphorothioated.

Antisense (guide) sequences of the present invention may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

The chemical modifications described herein are believed to promote single stranded polynucleotide loading into the RISC. Single stranded polynucleotides have been shown to be active in loading into RISC and inducing gene silencing. However, the level of activity for single stranded polynucleotides appears to be 2 to 4 orders of magnitude lower when compared to a duplex polynucleotide.

The present invention provides a description of the chemical modification patterns, which may (a) significantly increase stability of the single stranded polynucleotide (b) promote efficient loading of the polynucleotide into the RISC complex and (c) improve uptake of the single stranded nucleotide by the cell. The chemical modification patterns may include a combination of ribose, backbone, hydrophobic nucleoside and conjugate type of modifications. In addition, in some of the embodiments, the 5′ end of the single polynucleotide may be chemically phosphorylated.

In yet another embodiment, the present invention provides a description of the chemical modification patterns, which improve functionality of RISC inhibiting polynucleotides. Single stranded polynucleotides have been shown to inhibit activity of a preloaded RISC complex through the substrate competition mechanism. For these types of molecules, conventionally called antagomers, the activity usually requires high concentration and in vivo delivery is not very effective. The present invention provides a description of the chemical modification patterns, which may (a) significantly increase stability of the single stranded polynucleotide (b) promote efficient recognition of the polynucleotide by the RISC as a substrate and/or (c) improve uptake of the single stranded nucleotide by the cell. The chemical modification patterns may include a combination of ribose, backbone, hydrophobic nucleoside and conjugate type of modifications.

The modifications provided by the present invention are applicable to all polynucleotides. This includes single stranded RISC entering polynucleotides, single stranded RISC inhibiting polynucleotides, conventional duplexed polynucleotides of variable length (15-40 bp),asymmetric duplexed polynucleotides, and the like. Polynucleotides may be modified with wide variety of chemical modification patterns, including 5′ end, ribose, backbone and hydrophobic nucleoside modifications.

### Synthesis

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. Nos. 5,013,830; 5,214,135; 5,525,719; Kawasaki et al. 1993. J. Med. Chem. 36:831; WO 92/03568; U.S. Pat. Nos. 5,276,019; and 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J Chrom. 599:35.

Other exemplary synthesis techniques are well known in the art (see, e.g., Sambrook et al., Molecular Cloning: a Laboratory Manual, Second Edition (1989); DNA Cloning, Volumes I and II (DN Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject RNAi constructs or at least portions thereof are transcribed from expression vectors encoding the subject constructs. Any art recognized vectors may be use for this purpose. The transcribed RNAi constructs may be isolated and purified, before desired modifications (such as replacing an unmodified sense strand with a modified one, etc.) are carried out.

### Delivery/Carrier

Without wishing to be bound by any particular theory, the inventors believe that the particular patterns of modifications on the passenger strand and guide strand of the double stranded nucleic acid molecules described herein (e.g., sd-rxRNAs) facilitate entry of the guide strand into the nucleus, where the guide strand mediates gene silencing (e.g., silencing of target genes, such as AKT, p53, PDCD1, TIGIT, Cbl-b, Tet2, Blimp-1, T-Box21, DNMT3A, PTPN6, and HK2).

Without wishing to be bound by any theory, several potential mechanisms of action could account for this activity. For example, in some embodiments, the guide strand (e.g., antisense strand) of the nucleic acid molecule (e.g., sd-rxRNA) may dissociate from the passenger strand and enter into the nucleus as a single strand. Once in the nucleus the single stranded guide strand may associate with RNAse H or another ribonuclease and cleave the target (e.g., AKT, p53, PDCD1, TIGIT, Cbl-b, Tet2, Blimp-1, T-Box21, DNMT3A, PTPN6, or HK2) (“Antisense mechanism of action”). In some embodiments, the guide strand (e.g., antisense strand) of the nucleic acid molecule (e.g., sd-rxRNA) may associate with an Argonaute (Ago) protein in the cytoplasm or outside the nucleus, forming a loaded Ago complex. This loaded Ago complex may translocate into the nucleus and then cleave the target (e.g., AKT, p53, PDCD1, TIGIT, Cbl-b, Tet2, Blimp-1, T-Box21, DNMT3A, PTPN6, and HK2). In some embodiments, both strands (e.g. a duplex) of the nucleic acid molecule (e.g., sd-rxRNA) may enter the nucleus and the guide strand may associate with RNAse H, an Ago protein or another ribonuclease and cleaves the target (e.g., AKT, p53, PDCD1, TIGIT, Cbl-b, Tet2, Blimp-1, T-Box21, DNMT3A, PTPN6, and HK2).

The skilled artisan appreciates that the sense strand of the double stranded molecules described herein (e.g., sd-rxRNA sense strand) is not limited to delivery of a guide strand of the double stranded nucleic acid molecule described herein. Rather, in some embodiments, a passenger strand described herein is joined (e.g., covalently bound, non-covalently bound, conjugated, hybridized via a region of complementarity, etc.) to certain molecules (e.g., antisense oligonucleotides, ASO) for the purpose of targeting said other molecule to the nucleus of a cell. In some embodiments, the molecule joined to a sense strand described herein is a synthetic antisense oligonucleotide (ASO). In some embodiments, the sense strand joined to an anti-sense oligonucleotide is between 8-15 nucleotides long, chemically modified, and comprises a hydrophobic conjugate.

Without wishing to be bound by any particular theory, an ASO can be joined to a complementary passenger strand by hydrogen bonding. Accordingly, in some aspects, the disclosure provides a method of delivering a nucleic acid molecule to a cell, the method comprising administering an isolated nucleic acid molecule to a cell, wherein the isolated nucleic acid comprises a sense strand which is complementary to an anti-sense oligonucleotide (ASO), wherein the sense strand is between 8-15 nucleotides in length, comprises at least two phosphorothioate modifications, at least 50% of the pyrimidines in the sense strand are modified, and wherein the molecule comprises a hydrophobic conjugate.

### Uptake of Oligonucleotides by Cells

Oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In some embodiments, the oligonucleotide compositions of the invention are contacted with bacterial cells. In some embodiments, the oligonucleotide compositions of the invention are contacted with eukaryotic cells (e.g., plant cell, mammalian cell, arthropod cell, such as insect cell). In some embodiments, the oligonucleotide compositions of the invention are contacted with stem cells. In some embodiments, the oligonucleotide compositions of the invention are contacted with immune cells, such as T-cells (e.g., CD8+ T-cells). In some embodiments, the T-cells are T_{SCM} or T_{CM} T-cells. In a preferred embodiment, the oligonucleotide compositions of the invention are contacted with human cells.

Oligonucleotide compositions of the invention can be contacted with cells in vitro, e.g., in a test tube or culture dish, (and may or may not be introduced into a subject) or in vivo, e.g., in a subject such as a mammalian subject, or ex vivo. In some embodiments, Oligonucleotides are administered topically or through electroporation. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet 2003 Jan. 19:9; Reichhart J M et al. Genesis. 2002. 34(1-2):1604, Yu et al. 2002. Proc. Natl. Acad Sci. USA 99:6047; Sui et al. 2002. Proc. Natl. Acad Sci. USA 99:5515) viruses, polyamine or polycation conjugates using compounds such as polylysine, protamine, or Ni, N12-bis (ethyl) spermine (see, e.g., Bartzatt, R. et al. 1989. Biotechnol. Appl. Biochem. 11:133; Wagner E. et al. 1992. Proc. Natl. Acad. Sci. 88:4255).

In certain embodiments, the chemically modified double stranded nucleic acid molecules of the invention may be delivered by using various beta-glucan containing particles, referred to as GeRPs (glucan encapsulated RNA loaded particle), described in, and incorporated by reference from, U.S. Provisional Application No. 61/310,611, filed on Mar. 4, 2010 and entitled “Formulations and Methods for Targeted Delivery to Phagocyte Cells.” Such particles are also described in, and incorporated by reference from US Patent Publications US 2005/0281781 A1, and US 2010/0040656, and in PCT publications WO 2006/007372, and WO 2007/050643. The chemically modified double stranded nucleic acid molecule may be hydrophobically modified and optionally may be associated with a lipid and/or amphiphilic peptide. In certain embodiments, the beta-glucan particle is derived from yeast. In certain embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine), etc.

Glucan particles can be derived from insoluble components of fungal cell walls such as yeast cell walls. In some embodiments, the yeast is Baker's yeast. Yeast-derived glucan molecules can include one or more of ß-(1,3)-Glucan, ß-(1,6)-Glucan, mannan and chitin. In some embodiments, a glucan particle comprises a hollow yeast cell wall whereby the particle maintains a three dimensional structure resembling a cell, within which it can complex with or encapsulate a molecule such as an RNA molecule. Some of the advantages associated with the use of yeast cell wall particles are availability of the components, their biodegradable nature, and their ability to be targeted to phagocytic cells.

In some embodiments, glucan particles can be prepared by extraction of insoluble components from cell walls, for example by extracting Baker's yeast (Fleischmann's) with 1M NaOH/pH 4.0 H2O, followed by washing and drying. Methods of preparing yeast cell wall particles are discussed in, and incorporated by reference from U.S. Pat. Nos. 4,810,646, 4,992,540, 5,082,936, 5,028,703, 5,032,401, 5,322,841, 5,401,727, 5,504,079, 5,607,677, 5,968,811, 6,242,594, 6,444,448, 6,476,003, US Patent Publications 2003/0216346, 2004/0014715 and 2010/0040656, and PCT published application WO002/12348.

Protocols for preparing glucan particles are also described in, and incorporated by reference from, the following references: Soto and Ostroff (2008), “Characterization of multilayered nanoparticles encapsulated in yeast cell wall particles for DNA delivery.” Bioconjug Chem 19(4):840-8; Soto and Ostroff (2007), “Oral Macrophage Mediated Gene Delivery System,” Nanotech, Volume 2, Chapter 5 (“Drug Delivery”), pages 378-381; and Li et al. (2007), “Yeast glucan particles activate murine resident macrophages to secrete proinflammatory cytokines via MyD88- and Syk kinase-dependent pathways.” Clinical Immunology 124(2):170-181.

Glucan containing particles such as yeast cell wall particles can also be obtained commercially. Several non-limiting examples include: Nutricell MOS 55 from Biorigin (Sao Paolo, Brazil), SAF-Mannan (SAF Agri, Minneapolis, Minn.), Nutrex (Sensient Technologies, Milwaukee, Wis.), alkali-extracted particles such as those produced by Nutricepts (Nutricepts Inc., Burnsville, Minn.) and ASA Biotech, acid-extracted WGP particles from Biopolymer Engineering, and organic solvent-extracted particles such as Adjuvax™ from Alpha-beta Technology, Inc. (Worcester, Mass.) and microparticulate glucan from Novogen (Stamford, Conn.).

Glucan particles such as yeast cell wall particles can have varying levels of purity depending on the method of production and/or extraction. In some instances, particles are alkali-extracted, acid-extracted or organic solvent-extracted to remove intracellular components and/or the outer mannoprotein layer of the cell wall. Such protocols can produce particles that have a glucan (w/w) content in the range of 50%-90%. In some instances, a particle of lower purity, meaning lower glucan w/w content may be preferred, while in other embodiments, a particle of higher purity, meaning higher glucan w/w content may be preferred.

Glucan particles, such as yeast cell wall particles, can have a natural lipid content. For example, the particles can contain 1%, 2%, 3%, 4%, 5%, 6%, 7%, 8%, 9%, 10%, 11%, 12%, 13%, 14%, 15%, 16%, 17%, 18%, 19%, 20% or more than 20% w/w lipid. In some instances, the presence of natural lipids may assist in complexation or capture of RNA molecules.

Glucan containing particles typically have a diameter of approximately 2-4 microns, although particles with a diameter of less than 2 microns or greater than 4 microns are also compatible with aspects of the invention.

The RNA molecule(s) to be delivered can be complexed or “trapped” within the shell of the glucan particle. The shell or RNA component of the particle can be labeled for visualization, as described in, and incorporated by reference from, Soto and Ostroff (2008) Bioconjug Chem 19:840. Methods of loading GeRPs are discussed further below.

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Immunogenic Compositions and Methods of Producing the Same

In some embodiments, chemically-modified double stranded nucleic acid molecules (e.g., sd-rxRNAs) described herein are useful for producing specific cell subtypes or T-cell subtypes for immunogenic compositions. As used herein, an “immunogenic composition” is a composition comprising a host cell comprising a chemically-modified nucleic acid molecule as described herein, and optionally one or more pharmaceutically acceptable excipients or carriers. Without wishing to be bound by any particular theory, immunogenic compositions as described by the disclosure are characterized by a population of immune cells (e.g., T-cells, NK-cells, antigen-presenting cells (APC), dendritic cells (DC), stem cells (SC), induced pluripotent stem cells (iPSC), etc.) that have been engineered to have an enriched population of a particular cell subtype (e.g., T-cell subtype, such as T_{SCM} or T_{CM} T-cells) and/or reduced (e.g., inhibited) expression or activity of one or more immune checkpoint proteins (e.g., PDCD1, TIGIT, etc.), and are thus useful, in some embodiments, for modulating (e.g., stimulating or inhibiting) the immune response of a subject.

As used herein, a “host cell” is a cell to which one or more chemically-modified double stranded nucleic acid molecules have been introduced. Typically, a host cell is a mammalian cell, for example a human cell, mouse cell, rat cell, pig cell, etc. However, in some embodiments, a host cell is a non-mammalian cell, for example a prokaryotic cell (e.g., bacterial cell), yeast cell, insect cell, etc. Generally, a host cell is derived from a donor, such as a healthy donor (e.g., the cell to which the chemically-modified double stranded nucleic acid is introduced is taken from a donor, such as a healthy donor). For example, a cell or cells may be isolated from a biological sample obtained from a donor, such as a healthy donor, such as bone marrow or blood. As used herein “healthy donor” refers to a subject that does not have, or is not suspected of having, a proliferative disorder or an infectious disease (e.g., a bacterial, viral, or parasitic infection). However, in some embodiments, a host cell is derived from a subject having (or suspected of having) a proliferative disease or an infectious disease, for example in the context of autologous cell therapy.

In some embodiments a cell (e.g., a host cell) is an immune cell, for example a T-cell, B-cell, dentritic cell (DC), granulocyte, natural killer cell, macrophage, etc. In some embodiments, a cell (e.g., a host cell) is a cell that is capable of differentiating into an immune cell, such as a stem cell (SC) or induced pluripotent stem cell (iPSC). In some embodiments, a cell (e.g., a host cell) is a stem cell memory T-cell, for example as described in, and incorporated by reference from, Gattinoni et al. (2017) Nature Medicine 23; 18-27.

In some embodiments, a cell (e.g., a host cell) is a T-cell, such as a killer T-cell, helper T-cell, or a regulatory T-cell. In some embodiments the T-cell is a killer T-cell (e.g., a CD8+ T-cell). In some embodiments, the T-cell is a helper T-cell (e.g., a CD4+ T-cell). In some embodiments, a T-cell is an activated T-cell (e.g., a T-cell that has been presented with a peptide antigen by MHC class II molecules on an antigen presenting cell).

In some embodiments, a T-cell comprises one or more transgenes expressing a high affinity T-cell receptor (TCR) and/or a chimeric antibody receptor (CAR).

In some aspects, the disclosure relates to the discovery that introducing one or more chemically-modified double stranded nucleic acid molecules of the disclosure to a cell (e.g., an immune cell obtained from a donor) to produce a host cell results in a significant reduction of immune checkpoint protein (e.g., TIGIT, PDCD1, etc.) expression or activity in the host cell. In some embodiments, a host cell is characterized by between about 5% and about 50% reduced expression of an immune checkpoint protein relative to a cell (e.g., an immune cell of the same cell type) that does not comprise the chemically-modified double stranded nucleic acid molecules. In some embodiments, a host cell is characterized by greater than 50% (e.g., 51%, 52%, 53%, 54%, 55%, 60%, 70%, 75%, 80%, 85%, 90%, 95%, 99%, 100%, or about any percentage between 51% and 100%) reduced expression of an immune checkpoint protein relative to a cell (e.g., an immune cell of the same cell type) that does not comprise the chemically-modified double stranded nucleic acid molecules (e.g., an immune cell of a subject having or suspected of having a proliferative disease or an infectious disease).

In some aspects, the disclosure relates to the discovery that introducing one or more chemically-modified double stranded nucleic acid molecules (e.g., one or more sd-rxRNAs) of the disclosure to a cell (e.g., an immune cell obtained from a donor) to produce a host cell characterized by a significant reduction of one or more signal transduction/transcription factor, epigenetic, metabolic and/or co-inhibitory/negative regulatory protein (e.g., AKT, p53, PDCD1, TIGIT, Cbl-b, Tet2, Blimp-1, T-Box21, HK2, DNMT3A, PTPN6, etc.) expression or activity in the host cell. In some embodiments, a host cell is characterized by between about 5% and about 50% reduced expression of an immune checkpoint protein relative to a cell (e.g., an immune cell of the same cell type) that does not comprise the chemically-modified double stranded nucleic acid molecules. In some embodiments, a host cell is characterized by greater than 50% (e.g., 51%, 52%, 53%, 54%, 55%, 60%, 70%, 75%, 80%, 85%, 90%, 95%, 99%, 100%, or any percentage between 51% and 100%, including all values in between) reduced expression of a differentiation related target (e.g. signaling molecule, kinase/phosphatase, transcription factor, epigenetic modulator, metabolic and regulatory target) relative to a cell (e.g., an immune cell of the same cell type) that does not comprise the chemically-modified double stranded nucleic acid molecules (e.g., an immune cell of a subject having or suspected of having a proliferative disease or an infectious disease).

In some embodiments, a host cell further comprises one or more additional chemically-modified double stranded nucleic acid molecules that target other differentiation related targets, for example, AKT, p53, PD1, TIGIT, Cbl-b, Tet2, Blimp-1, T-Box21, HK2, DNMT3A, PTPN6, any combination thereof, etc. For example, in some embodiments, an immunogenic composition comprises a host cell engineered to have reduced expression of the following combinations of differentiation related proteins:

### p53 and PD1

### p53 and AKT

### PD1 and AKT

### PD1 and AKT and p53

### Cbl-b and PD1

### Clb-b and AKT

### Clb-b and PD1 and AKT

Or any combination thereof.

In some embodiments, a host cell further comprises one or more additional chemically-modified double stranded nucleic acid molecules that target other immune checkpoint proteins, for example, CTLA-4, BTLA, KIR, B7-H3, B7-H4, TGF32 receptor, etc. For example, in some embodiments, an immunogenic composition comprises a host cell engineered to have reduced expression of the following combinations of immune checkpoint proteins:

### CTLA4 and PD1

### STAT3 and p38

### PD1 and BaxPD1, CTLA4, Lag-1, ILM-3, and TP53

### PD1 and Casp8

### PD1 and IL1OR

### PD1 and TIGIT.

In some embodiments, an immunogenic composition as described by the disclosure comprises a plurality of host cells. In some embodiments, the plurality of host cells is about 10,000 host cells per kilogram, about 50,000 host cells per kilogram, about 100,000 host cells per kilogram, about 250,000 host cells per kilogram, about 500,000 host cells per kilogram, about 1×10⁶ host cells per kilogram, about 5×10⁶ host cells per kilogram, about 1×10⁷ host cells per kilogram, about 1×10⁸ host cells per kilogram, about 1×10⁹ host cells per kilogram, or more than 1×10⁹ host cells per kilogram. In some embodiments, the plurality of host cells is between about 1×10⁵ and 1×10¹⁴ host cells per kilogram.

In some aspects, the disclosure provides methods for producing an immunogenic composition as described by the disclosure. In some embodiments, the methods comprise, introducing into a cell one or more chemically-modified double stranded nucleic acid molecules (e.g., sd-rxRNAs), wherein the one or more chemically-modified double stranded nucleic acid molecules target AKT, p53, PDCD1, TIGIT, Cbl-b, Tet2, Blimp-1, T-Box21, DNMT3A, PTPN6, or HK2, or any combination thereof, thereby producing a host cell with a specific cell subtype or T-cell subtype (e.g., T_{SCM} or T_{CM}).

Methods of producing immunogenic compositions (e.g., producing a host cell or populations of host cells) may be carried out in vitro, ex vivo, or in vivo, in, for example, mammalian cells in culture, such as a human cell in culture. In some embodiments, target cells (e.g., cells obtained from a donor) may be contacted in the presence of a delivery reagent, such as a lipid (e.g., a cationic lipid) or a liposome to facilitate entry of the chemically-modified double stranded nucleic acid molecules into the cell, as described in further detail elsewhere in the disclosure.

### Carriers and Complexing Agents

The disclosure further relates to compositions comprising RNAi constructs as described herein, and a pharmaceutically acceptable carrier or diluent. In some aspects, the disclosure relates to immunogenic compositions comprising the RNAi constructs described herein, and a pharmaceutically acceptable carrier.

As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

For example, in some embodiments, oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types (e.g., immune cells, such as T-cells).

Encapsulating agents entrap oligonucleotides within vesicles. In another embodiment of the invention, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present invention may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some aspects, formulations associated with the invention might be selected for a class of naturally occurring or chemically synthesized or modified saturated and unsaturated fatty acid residues. Fatty acids might exist in a form of triglycerides, diglycerides or individual fatty acids. In another embodiment, the use of well-validated mixtures of fatty acids and/or fat emulsions currently used in pharmacology for parenteral nutrition may be utilized.

Liposome based formulations are widely used for oligonucleotide delivery. However, most of commercially available lipid or liposome formulations contain at least one positively charged lipid (cationic lipids). The presence of this positively charged lipid is believed to be essential for obtaining a high degree of oligonucleotide loading and for enhancing liposome fusogenic properties. Several methods have been performed and published to identify optimal positively charged lipid chemistries. However, the commercially available liposome formulations containing cationic lipids are characterized by a high level of toxicity. In vivo limited therapeutic indexes have revealed that liposome formulations containing positive charged lipids are associated with toxicity (e.g., elevation in liver enzymes) at concentrations only slightly higher than concentration required to achieve RNA silencing.

Nucleic acids associated with the invention can be hydrophobically modified and can be encompassed within neutral nanotransporters. Further description of neutral nanotransporters is incorporated by reference from PCT Application PCT/US2009/005251, filed on Sep. 22, 2009, and entitled “Neutral Nanotransporters.” Such particles enable quantitative oligonucleotide incorporation into non-charged lipid mixtures. The lack of toxic levels of cationic lipids in such neutral nanotransporter compositions is an important feature.

As demonstrated in PCT/US2009/005251, oligonucleotides can effectively be incorporated into a lipid mixture that is free of cationic lipids and such a composition can effectively deliver a therapeutic oligonucleotide to a cell in a manner that it is functional. For example, a high level of activity was observed when the fatty mixture was composed of a phosphatidylcholine base fatty acid and a sterol such as a cholesterol. For instance, one preferred formulation of neutral fatty mixture is composed of at least 20% of DOPC or DSPC and at least 20% of sterol such as cholesterol. Even as low as 1:5 lipid to oligonucleotide ratio was shown to be sufficient to get complete encapsulation of the oligonucleotide in a non-charged formulation.

The neutral nanotransporters compositions enable efficient loading of oligonucleotide into neutral fat formulation. The composition includes an oligonucleotide that is modified in a manner such that the hydrophobicity of the molecule is increased (for example a hydrophobic molecule is attached (covalently or no-covalently) to a hydrophobic molecule on the oligonucleotide terminus or a non-terminal nucleotide, base, sugar, or backbone), the modified oligonucleotide being mixed with a neutral fat formulation (for example containing at least 25% of cholesterol and 25% of DOPC or analogs thereof). A cargo molecule, such as another lipid can also be included in the composition. This composition, where part of the formulation is built into the oligonucleotide itself, enables efficient encapsulation of oligonucleotide in neutral lipid particles.

In some aspects, stable particles ranging in size from 50 to 140 nm can be formed upon complexing of hydrophobic oligonucleotides with preferred formulations. The formulation by itself typically does not form small particles, but rather, forms agglomerates, which are transformed into stable 50-120 nm particles upon addition of the hydrophobic modified oligonucleotide.

In some embodiments, neutral nanotransporter compositions include a hydrophobic modified polynucleotide, a neutral fatty mixture, and optionally a cargo molecule. A “hydrophobic modified polynucleotide” as used herein is a polynucleotide of the invention (e.g., sd-rxRNA) that has at least one modification that renders the polynucleotide more hydrophobic than the polynucleotide was prior to modification. The modification may be achieved by attaching (covalently or non-covalently) a hydrophobic molecule to the polynucleotide. In some instances the hydrophobic molecule is or includes a lipophilic group.

The term “lipophilic group” means a group that has a higher affinity for lipids than its affinity for water. Examples of lipophilic groups include, but are not limited to, cholesterol, a cholesteryl or modified cholesteryl residue, adamantine, dihydrotesterone, long chain alkyl, long chain alkenyl, long chain alkynyl, olely-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g., Cy3 or Cy5), Hoechst 33258 dye, psoralen, or ibuprofen. The cholesterol moiety may be reduced (e.g., as in cholestan) or may be substituted (e.g., by halogen). A combination of different lipophilic groups in one molecule is also possible.

The hydrophobic molecule may be attached at various positions of the polynucleotide. As described above, the hydrophobic molecule may be linked to the terminal residue of the polynucleotide such as the 3′ of 5′-end of the polynucleotide. Alternatively, it may be linked to an internal nucleotide or a nucleotide on a branch of the polynucleotide. The hydrophobic molecule may be attached, for instance to a 2′-position of the nucleotide. The hydrophobic molecule may also be linked to the heterocyclic base, the sugar or the backbone of a nucleotide of the polynucleotide.

The hydrophobic molecule may be connected to the polynucleotide by a linker moiety. Optionally the linker moiety is a non-nucleotidic linker moiety. Non-nucleotidic linkers are e.g. abasic residues (dSpacer), oligoethyleneglycol, such as triethyleneglycol (spacer 9) or hexaethylenegylcol (spacer 18), or alkane-diol, such as butanediol. The spacer units are preferably linked by phosphodiester or phosphorothioate bonds. The linker units may appear just once in the molecule or may be incorporated several times, e.g., via phosphodiester, phosphorothioate, methylphosphonate, or amide linkages.

Typical conjugation protocols involve the synthesis of polynucleotides bearing an aminolinker at one or more positions of the sequence, however, a linker is not required. The amino group is then reacted with the molecule being conjugated using appropriate coupling or activating reagents. The conjugation reaction may be performed either with the polynucleotide still bound to a solid support or following cleavage of the polynucleotide in solution phase. Purification of the modified polynucleotide by HPLC typically results in a pure material.

In some embodiments the hydrophobic molecule is a sterol type conjugate, a PhytoSterol conjugate, cholesterol conjugate, sterol type conjugate with altered side chain length, fatty acid conjugate, any other hydrophobic group conjugate, and/or hydrophobic modifications of the internal nucleoside, which provide sufficient hydrophobicity to be incorporated into micelles.

For purposes of the present invention, the term “sterols”, refers or steroid alcohols are a subgroup of steroids with a hydroxyl group at the 3-position of the A-ring. They are amphipathic lipids synthesized from acetyl-coenzyme A via the HMG-CoA reductase pathway. The overall molecule is quite flat. The hydroxyl group on the A ring is polar. The rest of the aliphatic chain is non-polar. Usually sterols are considered to have an 8 carbon chain at position 17.

For purposes of the present invention, the term “sterol type molecules”, refers to steroid alcohols, which are similar in structure to sterols. The main difference is the structure of the ring and number of carbons in a position 21 attached side chain.

For purposes of the present invention, the term “PhytoSterols” (also called plant sterols) are a group of steroid alcohols, phytochemicals naturally occurring in plants. There are more than 200 different known PhytoSterols

For purposes of the present invention, the term “Sterol side chain” refers to a chemical composition of a side chain attached at the position 17 of sterol-type molecule. In a standard definition sterols are limited to a 4 ring structure carrying a 8 carbon chain at position 17. In this invention, the sterol type molecules with side chain longer and shorter than conventional are described. The side chain may branched or contain double back bones.

Thus, sterols useful in the invention, for example, include cholesterols, as well as unique sterols in which position 17 has attached side chain of 2-7 or longer than 9 carbons. In a particular embodiment, the length of the polycarbon tail is varied between 5 and 9 carbons. Such conjugates may have significantly better in vivo efficacy, in particular delivery to liver. These types of molecules are expected to work at concentrations 5 to 9 fold lower then oligonucleotides conjugated to conventional cholesterols.

Alternatively the polynucleotide may be bound to a protein, peptide or positively charged chemical that functions as the hydrophobic molecule. The proteins may be selected from the group consisting of protamine, dsRNA binding domain, and arginine rich peptides. Exemplary positively charged chemicals include spermine, spermidine, cadaverine, and putrescine.

In another embodiment hydrophobic molecule conjugates may demonstrate even higher efficacy when it is combined with optimal chemical modification patterns of the polynucleotide (as described herein in detail), containing but not limited to hydrophobic modifications, phosphorothioate modifications, and 2′ ribo modifications.

In another embodiment the sterol type molecule may be a naturally occurring PhytoSterols. The polycarbon chain may be longer than 9 and may be linear, branched and/or contain double bonds. Some PhytoSterol containing polynucleotide conjugates may be significantly more potent and active in delivery of polynucleotides to various tissues. Some PhytoSterols may demonstrate tissue preference and thus be used as a way to delivery RNAi specifically to particular tissues.

The hydrophobic modified polynucleotide is mixed with a neutral fatty mixture to form a micelle. The neutral fatty acid mixture is a mixture of fats that has a net neutral or slightly net negative charge at or around physiological pH that can form a micelle with the hydrophobic modified polynucleotide. For purposes of the present invention, the term “micelle” refers to a small nanoparticle formed by a mixture of non-charged fatty acids and phospholipids. The neutral fatty mixture may include cationic lipids as long as they are present in an amount that does not cause toxicity. In preferred embodiments the neutral fatty mixture is free of cationic lipids. A mixture that is free of cationic lipids is one that has less than 1% and preferably 0% of the total lipid being cationic lipid. The term “cationic lipid” includes lipids and synthetic lipids having a net positive charge at or around physiological pH. The term “anionic lipid” includes lipids and synthetic lipids having a net negative charge at or around physiological pH.

The neutral fats bind to the oligonucleotides of the invention by a strong but non-covalent attraction (e.g., an electrostatic, van der Waals, pi-stacking, etc. interaction).

The neutral fat mixture may include formulations selected from a class of naturally occurring or chemically synthesized or modified saturated and unsaturated fatty acid residues. Fatty acids might exist in a form of triglycerides, diglycerides or individual fatty acids. In another embodiment the use of well-validated mixtures of fatty acids and/or fat emulsions currently used in pharmacology for parenteral nutrition may be utilized.

The neutral fatty mixture is preferably a mixture of a choline based fatty acid and a sterol. Choline based fatty acids include for instance, synthetic phosphocholine derivatives such as DDPC, DLPC, DMPC, DPPC, DSPC, DOPC, POPC, and DEPC. DOPC (chemical registry number 4235-95-4) is dioleoylphosphatidylcholine (also known as dielaidoylphosphatidylcholine, dioleoyl-PC, dioleoylphosphocholine, dioleoyl-sn-glycero-3-phosphocholine, dioleylphosphatidylcholine). DSPC (chemical registry number 816-94-4) is distearoylphosphatidylcholine (also known as 1,2-Distearoyl-sn-Glycero-3-phosphocholine).

The sterol in the neutral fatty mixture may be for instance cholesterol. The neutral fatty mixture may be made up completely of a choline based fatty acid and a sterol or it may optionally include a cargo molecule. For instance, the neutral fatty mixture may have at least 20% or 25% fatty acid and 20% or 25% sterol.

For purposes of the present invention, the term “Fatty acids” relates to conventional description of fatty acid. They may exist as individual entities or in a form of two- and triglycerides. For purposes of the present invention, the term “fat emulsions” refers to safe fat formulations given intravenously to subjects who are unable to get enough fat in their diet. It is an emulsion of soy bean oil (or other naturally occurring oils) and egg phospholipids. Fat emulsions are being used for formulation of some insoluble anesthetics. In this disclosure, fat emulsions might be part of commercially available preparations like Intralipid, Liposyn, Nutrilipid, modified commercial preparations, where they are enriched with particular fatty acids or fully de novo-formulated combinations of fatty acids and phospholipids.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

50%-60% of the formulation can optionally be any other lipid or molecule. Such a lipid or molecule is referred to herein as a cargo lipid or cargo molecule. Cargo molecules include but are not limited to intralipid, small molecules, fusogenic peptides or lipids or other small molecules might be added to alter cellular uptake, endosomal release or tissue distribution properties. The ability to tolerate cargo molecules is important for modulation of properties of these particles, if such properties are desirable. For instance the presence of some tissue specific metabolites might drastically alter tissue distribution profiles. For example use of Intralipid type formulation enriched in shorter or longer fatty chains with various degrees of saturation affects tissue distribution profiles of these type of formulations (and their loads).

An example of a cargo lipid useful according to the invention is a fusogenic lipid. For instance, the zwiterionic lipid DOPE (chemical registry number 4004-5-1, 1,2-Dioleoyl-sn-Glycero-3-phosphoethanolamine) is a preferred cargo lipid.

Intralipid may be comprised of the following composition: 1 000 mL contain: purified soybean oil 90 g, purified egg phospholipids 12 g, glycerol anhydrous 22 g, water for injection q.s. ad 1 000 mL. pH is adjusted with sodium hydroxide to pH approximately 8. Energy content/L: 4.6 MJ (190 kcal). Osmolality (approx.): 300 mOsm/kg water. In another embodiment fat emulsion is Liposyn that contains 5% safflower oil, 5% soybean oil, up to 1.2% egg phosphatides added as an emulsifier and 2.5% glycerin in water for injection. It may also contain sodium hydroxide for pH adjustment. pH 8.0 (6.0-9.0). Liposyn has an osmolarity of 276 m Osmol/liter (actual).

Variation in the identity, amounts and ratios of cargo lipids affects the cellular uptake and tissue distribution characteristics of these compounds. For example, the length of lipid tails and level of saturability will affect differential uptake to liver, lung, fat and cardiomyocytes. Addition of special hydrophobic molecules like vitamins or different forms of sterols can favor distribution to special tissues which are involved in the metabolism of particular compounds. In some embodiments, vitamin A or E is used. Complexes are formed at different oligonucleotide concentrations, with higher concentrations favoring more efficient complex formation.

In another embodiment, the fat emulsion is based on a mixture of lipids. Such lipids may include natural compounds, chemically synthesized compounds, purified fatty acids or any other lipids. In yet another embodiment the composition of fat emulsion is entirely artificial. In a particular embodiment, the fat emulsion is more than 70% linoleic acid. In yet another particular embodiment the fat emulsion is at least 1% of cardiolipin. Linoleic acid (LA) is an unsaturated omega-6 fatty acid. It is a colorless liquid made of a carboxylic acid with an 18-carbon chain and two cis double bonds.

In yet another embodiment of the present invention, the alteration of the composition of the fat emulsion is used as a way to alter tissue distribution of hydrophobicly modified polynucleotides. This methodology provides for the specific delivery of the polynucleotides to particular tissues.

In another embodiment the fat emulsions of the cargo molecule contain more than 70% of Linoleic acid (C18H3202) and/or cardiolipin.

Fat emulsions, like intralipid have been used before as a delivery formulation for some non-water soluble drugs (such as Propofol, re-formulated as Diprivan). Unique features of the present invention include (a) the concept of combining modified polynucleotides with the hydrophobic compound(s), so it can be incorporated in the fat micelles and (b) mixing it with the fat emulsions to provide a reversible carrier. After injection into a blood stream, micelles usually bind to serum proteins, including albumin, HDL, LDL and other. This binding is reversible and eventually the fat is absorbed by cells. The polynucleotide, incorporated as a part of the micelle will then be delivered closely to the surface of the cells. After that cellular uptake might be happening though variable mechanisms, including but not limited to sterol type delivery.

Complexing agents bind to the oligonucleotides of the invention by a strong but non-covalent attraction (e.g., an electrostatic, van der Waals, pi-stacking, etc. interaction). In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells. However, as discussed above, formulations free in cationic lipids are preferred in some embodiments.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethyl ammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. Nos. 4,235,871; 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93:3176). Cationic lipids and other complexing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. US.A. 93:3176). In one embodiment, a composition for delivering oligonucleotides of the invention comprises a number of arginine, lysine, histidine or ornithine residues linked to a lipophilic moiety (see e.g., U.S. Pat. No. 5,777,153).

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, a composition for delivering oligonucleotides of the invention comprises a natural or synthetic polypeptide having one or more gamma carboxyglutamic acid residues, or γ-Gla residues. These gamma carboxyglutamic acid residues may enable the polypeptide to bind to each other and to membrane surfaces. In other words, a polypeptide having a series of γ-Gla may be used as a general delivery modality that helps an RNAi construct to stick to whatever membrane to which it comes in contact. This may at least slow RNAi constructs from being cleared from the blood stream and enhance their chance of homing to the target.

The gamma carboxyglutamic acid residues may exist in natural proteins (for example, prothrombin has 10 γ-Gla residues). Alternatively, they can be introduced into the purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated/fine tuned to achieve different levels of “stickiness” of the polypeptide.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J Cell Biol. 128:919). In another embodiment, a Boc-Cys-(Npys)OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀ alkenyl chains, C₂-C₂₀ alkynyl chains, peptides, and heteroatoms (e.g., S, O, NH, etc.). Other exemplary linkers include bifinctional crosslinking agents such as sulfosuccinimidyl-4-(maleimidophenyl)-butyrate (SMPB) (see, e.g., Smith et al. Biochem J 1991.276: 417-2).

In one embodiment, oligonucleotides of the invention are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

Other carriers for in vitro and/or in vivo delivery of RNAi reagents are known in the art, and can be used to deliver the subject RNAi constructs (e.g., to a host cell, such as a T-cell). See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2, just to name a few (all incorporated by reference).

### Therapeutic Methods

In some aspects, the disclosure provides methods of treating a proliferative disease or an infectious disease by administering to a subject (e.g., a subject having or suspected of having a proliferative disease or an infectious disease) an immunogenic composition as described by the disclosure (e.g., an immunogenic composition comprising one or more host cells of a particular cell subtype or T-cell subtype). In some embodiments, immunogenic compositions as described herein are characterized as population of immune cells (e.g., T-cells, NK-cells, antigen-presenting cells (APC), dendritic cells (DC), stem cells (SC), induced pluripotent stem cells (iPSC), etc.) having reduced (e.g., inhibited) expression or activity of one or more genes associated with controlling the differentiation process of T-cells (e.g., AKT, p53, PD1, TIGIT, Cbl-b Tet2, Blimp-1, T-Box21, HK2, DNMT3A, PTPN6, etc.). Without wishing to be bound by any particular theory, immunogenic compositions as described herein are characterized, in some embodiments, by reduced expression of immune checkpoint proteins and are thus useful for stimulating the immune system of a subject having certain proliferative diseases or infectious diseases characterized by increased expression of immune checkpoint proteins.

As used herein, a “proliferative disease” refers to diseases and disorders characterized by excessive proliferation of cells and turnover of cellular matrix, including cancer, atherlorosclerosis, rheumatoid arthritis, psoriasis, idiopathic pulmonary fibrosis, scleroderma, cirrhosis of the liver, etc. Examples of cancers include but are not limited to small cell lung cancer, colon cancer, breast cancer, lung cancer, prostate cancer, ovarian cancer, pancreatic cancer, melanoma, bone cancer (e.g., osteosarcoma, etc.), hematological malignancy such as chronic myeloid leukemia (CML), etc.

As used herein, the term “infectious disease” refers to diseases and disorders that result from infection of a subject with a pathogen. Examples of human pathogens include but are not limited to certain bacteria (e.g., certain strains of E. coli, Salmonella, etc.), viruses (HIV, HCV, influenza, etc.), parasites (protozoans, helminths, amoeba, etc.), yeasts (e.g., certain Candida species, etc.), and fungi (e.g., certain Aspergillus species).

Examples of subjects include mammals, e.g., humans and other primates; cows, pigs, horses, and farming (agricultural) animals; dogs, cats, and other domesticated pets; mice, rats, and transgenic non-human animals.

In some embodiments, immunogenic compositions as described by the disclosure are administered to a subject by adoptive cell transfer (ACT) therapeutic methods. Examples of ACT modalities include but are not limited to autologous cell therapy (e.g., a subject's own cells are removed, genetically-modified, and returned to the subject) and heterologous cell therapy (e.g., cells are removed from a donor, genetically-modified, and placed into a recipient). In some embodiments, cells utilized in ACT therapeutic methods may be genetically-modified to express chimeric antigen receptors (CARs), which are engineered T-cell receptors displaying specificity against a target antigen based on a selected antibody moiety. Accordingly, in some embodiments, CAR T-cells (e.g. CARTs) may be transfected with a chemically-modified double stranded nucleic acid using methods described herein for the purpose of ACT therapy.

With respect to in vivo applications, the formulations of the present invention can be administered to a patient in a variety of forms adapted to the chosen route of administration, e.g., parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

Drug delivery vehicles can be chosen e.g., for in vitro, for systemic administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

Administration of an active amount of an oligonucleotide of the present invention is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically-modified oligonucleotides, e.g., with modification of the phosphate backbone, may require different dosing.

The exact dosage of an immunogenic composition and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regimens may be adjusted to provide the optimum therapeutic response. For example, the immunogenic composition may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject chemically-modified double stranded nucleic acid molecules or immunogenic compositions, whether they are to be administered to cells or to subjects.

Administration of immunogenic compositions, such as through intradermal injection or subcutaneous delivery, can be optimized through testing of dosing regimens. In some embodiments, a single administration is sufficient. To further prolong the effect of the administered immunogenic compositions, the compositions can be administered in a slow-release formulation or device, as would be familiar to one of ordinary skill in the art.

In other embodiments, the chemically-modified double stranded nucleic acid molecules or immunogenic compositions is administered multiple times. In some instances it is administered daily, bi-weekly, weekly, every two weeks, every three weeks, monthly, every two months, every three months, every four months, every five months, every six months or less frequently than every six months. In some instances, it is administered multiple times per day, week, month and/or year. For example, it can be administered approximately every hour, 2 hours, 3 hours, 4 hours, 5 hours, 6 hours, 7 hours, 8 hours, 9 hours 10 hours, 12 hours or more than twelve hours. It can be administered 1, 2, 3, 4, 5, 6, 7, 8, 9, 10 or more than 10 times per day.

Aspects of the invention relate to administering immunogenic compositions to a subject. In some instances the subject is a patient and administering the immunogenic composition involves administering the composition in a doctor's office.

In some embodiments, more than one immunogenic composition is administered simultaneously. For example a composition may be administered that contains 1, 2, 3, 4, 5, 6, 7, 8, 9, 10 or more than 10 different compositions. In certain embodiments, a composition comprises 2 or 3 different immunogenic compositions.

In some embodiments, one or more anticancer agents is administered to a subject in combination with one or more immunogenic compositions as described by the disclosure. An “anticancer agent” can be a small molecule, nucleic acid, protein, peptide, polypeptide (e.g., antibody, antibody fragment, etc.), or any combination of the foregoing. In some embodiments, an anticancer agent is administered to the subject prior to administration of the immunogenic composition. In some embodiments, an anticancer agent is administered to a subject after administration of the immunogenic composition. In some embodiments, an anticancer agent is administered concurrently (e.g., at the same time as) with an immunogenic composition.

Examples of anticancer agents include but are not limited to Abitrexate (Methotrexate), Abraxane (Paclitaxel Albumin-stabilized Nanoparticle Formulation), Ado-Trastuzumab Emtansine, Adriamycin PFS (Doxorubicin Hydrochloride), Adriamycin RDF (Doxorubicin Hydrochloride), Adrucil (Fluorouracil), Afinitor (Everolimus), Anastrozole, Aredia (Pamidronate Disodium), Arimidex (Anastrozole), Aromasin (Exemestane), CapecitabineClafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Herceptin (Trastuzumab), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), Neosar (Cyclophosphamide), Nolvadex (Tamoxifen Citrate), Novaldex (Tamoxifen Citrate), Paclitaxel, Paclitaxel Albumin-stabilized Nanoparticle Formulation, Pamidronate Disodium, Perj eta (Pertuzumab), Pertuzumab, Tamoxifen Citrate, Taxol (Paclitaxel), Taxotere (Docetaxel), Trastuzumab, Toremifene, Tykerb (Lapatinib Ditosylate), Xeloda (Capecitabine), and Zoladex (Goserelin Acetate).

### Self-Delivering RNAi Immunotherapeutic Agents

As described in U.S. Patent Publication No. US 2016/0304873, the entire contents of which are incorporated herein by reference, immunotherapeutic agents were produced by treating cells with particular sd-rxRNA agents designed to target and knock down specific genes involved in immune suppression mechanisms. In particular, the following cells and cell lines, shown in Table 1, have been successfully treated with sd-rxRNA and were shown to knock down at least 70% of targeted gene expression in the specified human cells.

These studies demonstrated utility of these immunogenic agents to suppress expression of target genes in cells normally very resistant to transfection, and suggested the agents are capable of reducing expression of target cells in any cell type.

A number of human genes were selected as candidate target genes due to involvement in immune suppression mechanisms and/or control of T-cell differentiation, including BAX, BAK1, CASP8, ADORA2A, CTLA4, LAG3, TGFBR1, HAVCR2, CCL17, CCL22, DLL2, FASLG, CD274, IDO1, IL1RA, JAG1, JAG2, MAPK14, PDCD1, SOCS1, STAT3, TNFA1P3, TNFSF4, TYRO2, DNMT3A, PTPN6, etc.

TABLE 1

| | | sd-rxRNA | SEQ | % |
|---|---|---|---|---|
| | Target | target | ID | Knock |
| Cell Type | Gene | sequence | NO: | Down |
| Primary human | TP53 | GAGTAGGACA | 1 | >70% |
| T-cells | (P53) | TACCAGCTTA | | 2 uM |
| Primary human | MAP4K4 | AGAGTTCTGT | 2 | >70% |
| T-cells | | GGAAGTCTA | | 2 uM |
| Jurkat T- | MAP4K4 | AGAGTTCTGT | 3 | 100% |
| lymphoma | | GGAAGTCTA | | 1 uM |
| cells | | | | 72 h |
| NK-92 cells | MAP4K4 | AGAGTTCTGT | 4 | 80% |
| | | GGAAGTCTA | | 2 uM |
| | | | | 72 h |
| NK-92 cells | PPIB | ACAGCAAATT | 5 | >75% |
| | | CCATCGTGT | | 2 uM |
| | | | | 72 h |
| NK-92 cells | GADPH | CTGGTAAAGT | 6 | >90% |
| | | GGATATTGTT | | 2 uM |
| | | | | 72 h |
| HeLa Cells | MAP4K4 | AGAGTTCTGT | 7 | >80% |
| | | GGAAGTCTA | | 2 uM |
| | | | | 72 h |

A number of human genes were selected as candidate target genes due to involvement in immune suppression mechanisms, including the genes listed in Table 2 (GenBank Accession Numbers shown in parenthesis):

TABLE 2

| | | |
|---|---|---|
| BAX (NM_004324) | BAK1 (NM_001188) | CASP8 (NM_001228) |
| ADORA2A (NM_000675) | CTLA4 (NM_005214) | LAG3 (NM002286) |
| PDCD1 (NM_NM005018) | TGFBR1 (NM-004612) | HAVCR2 (NM_032782) |
| CCL17 (NM_002987) | CCL22 (NM_002990) | DLL2 (NM_005618) |
| FASLG (NM_000639) | CD274 (NM_001267706) | IDO1 (NM_002164) |
| IL10RA (NM_001558) | JAG1 (NM_000214) | JAG2 (NM_002226) |
| MAPK14 (NM_001315) | SOCS1 (NM_003745) | STAT3 (NM_003150) |
| TNFA1P3 (NM_006290) | TNFSF4 (NM_003326) | TYRO2 (NM_006293) |
| TP53 (NM_000546) | | |

Each of the genes listed in Table 2 above was analyzed using a proprietary algorithm to identify preferred sd-rxRNA targeting sequences and target regions for each gene for prevention of immunosuppression of antigen-presenting cells and T-cells. Non-limiting examples of PDCD1 target sequences are shown in Table 3. Non-limiting examples of Cb1-b target sequences are shown in Table 4.

TABLE 3

| | | | SEQ | | SEQ |
|---|---|---|---|---|---|
| Oligo_ | | | ID | | ID |
| ID | PCDC1 human | Sequence | NO: | Gene region | NO: |
| PD1_1 | PDCD1_NM_005018_ | UAUUAUAUUAUAAUUAUAAU | 8 | CCTTCCCTGTGGTTCTATTATATTATA | 28 |
| | human_2070 | | | ATTATAATTAAATATGAG | |
| PD1_2 | PDCD1_NM_005018_ | UCUAUUAUAUUAUAAUUAUA | 9 | CCCCTTCCCTGTGGTTCTATTATATTAT | 29 |
| | human_2068 | | | AATTATAATTAAATATG | |
| PD1_3 | PDCD1_NM_005018_ | CAUUCCUGAAAUUAUUUAAA | 10 | GCTCTCCTTGGAACCCATTCCTGAAAT | 30 |
| | human_1854 | | | TATTTAAAGGGGTTGGCC | |
| PD1_4 | PDCD1_NM_005018_ | CUAUUAUAUUAUAAUUAUAA | 11 | CCCTTCCCTGTGGTTCTATTATATTAT | 31 |
| | human_2069 | | | AATTATAATTAAATATGA | |
| PD1_5 | PDCD1_NM_005018_ | AGUUUCAGGGAAGGUCAGAA | 12 | CTGCAGGCCTAGAGAAGTTTCAGGGA | 32 |
| | human_1491 | | | AGGTCAGAAGAGCTCCTGG | |
| PD1_6 | PDCD1_NM_005018_ | UGUGGUUCUAUUAUAUUAUA | 13 | GGGATCCCCCTTCCCTGTGGTTCTATT | 33 |
| | human_2062 | | | ATATTATAATTATAATTA | |
| PD1_7 | PDCD1_NM_005018_ | UGUGUUCUCUGUGGACUAUG | 14 | CCCCTCAGCCGTGCCTGTGTTCTCTGT | 34 |
| | human_719 | | | GGACTATGGGGAGCTGGA | |
| PD1_8 | PDCD1_NM_005018_ | CCCAUUCCUGAAAUUAUUUA | 15 | GAGCTCTCCTTGGAACCCATTCCTGAA | 35 |
| | human_1852 | | | ATTATTTAAAGGGGTTGG | |
| PD1_9 | PDCD1_NM_005018_ | UGCCACCAUUGUCUUUCCUA | 16 | TGAGCAGACGGAGTATGCCACCATTG | 36 |
| | human_812 | | | TCTTTCCTAGCGGAATGGG | |
| PD1_10 | PDCD1_NM_005018_ | AAGUUUCAGGGAAGGUCAGA | 17 | CCTGCAGGCCTAGAGAAGTTTCAGGG | 37 |
| | human_1490 | | | AAGGTCAGAAGAGCTCCTG | |
| PD1_11 | PDCD1_NM_005018_ | CUGUGGUUCUAUUAUAUUAU | 18 | AGGGATCCCCCTTCCCTGTGGTTCTAT | 38 |
| | human_2061 | | | TATATTATAATTATAATT | |
| PD1_12 | PDCD1_NM_005018_ | UUCUAUUAUAUUAUAAUUAU | 19 | CCCCCTTCCCTGTGGTTCTATTATATT | 39 |
| | human_2067 | | | ATAATTATAATTAAATAT | |
| PD1_13 | PDCD1_NM_005018_ | UUUCAGGGAAGGUCAGAAGA | 20 | GCAGGCCTAGAGAAGTTTCAGGGAAG | 40 |
| | human_1493 | | | GTCAGAAGAGCTCCTGGCT | |
| PD1_14 | PDCD1_NM_005018_ | CUUGGAACCCAUUCCUGAAA | 21 | ACCCTGGGAGCTCTCCTTGGAACCCAT | 41 |
| | human_1845 | | | TCCTGAAATTATTTAAAG | |
| PD1_15 | PDCD1_NM_005018_ | UCCCUGUGGUUCUAUUAUAU | 22 | ACAAGGGATCCCCCTTCCCTGTGGTTC | 42 |
| | human_2058 | | | TATTATATTATAATTATA | |
| PD1_16 | PDCD1_NM_005018_ | CCUGUGGUUCUAUUAUAUUA | 23 | AAGGGATCCCCCTTCCCTGTGGTTCTA | 43 |
| | human_2060 | | | TTATATTATAATTATAAT | |
| PD1_17 | PDCD1_NM_005018_ | UGGAACCCAUUCCUGAAAUU | 24 | CCTGGGAGCTCTCCTTGGAACCCATTC | 44 |
| | human_1847 | | | CTGAAATTATTTAAAGGG | |
| PD1_18 | PDCD1_NM_005018_ | CCUUCCCUGUGGUUCUAUUA | 25 | GGGACAAGGGATCCCCCTTCCCTGTG | 45 |
| | human_2055 | | | GTTCTATTATATTATAATT | |
| PD1_19 | PDCD1_NM_005018_ | UUCCCUGUGGUUCUAUUAUA | 26 | GACAAGGGATCCCCCTTCCCTGTGGTT | 46 |
| | human_2057 | | | CTATTATATTATAATTAT | |
| PD1_20 | PDCD1_NM_005018_ | CACAGGACUCAUGUCUCAAU | 27 | CAGGCACAGCCCCACCACAGGACTCA | 47 |
| | human_1105 | | | TGTCTCAATGCCCACAGTG | |

TABLE 4

| | | | SEQ |
|---|---|---|---|
| Oligo_ | | | ID |
| ID | Cbl-b human | Sequence | NO: |
| CB-01 | CBLB human | caauugauuu | 48 |
| | NM_170662_978 | aacuugcaau | |
| CB-02 | CBLB human | uuuaacuugc | 49 |
| | NM_170662_985 | aaugauuaca | |
| CB-03 | CBLB human | gaaguuaaag | 50 |
| | NM_170662_1124 | cacgacuaca | |
| CB-04 | CBLB human | aaaguuacac | 51 |
| | NM_170662_1382 | aggaacaaua | |
| CB-05 | CBLB human | uucugucguu | 52 |
| | NM_170662_1550 | gugaaauaaa | |
| CB-06 | CBLB human | cuccuugcau | 53 |
| | NM_170662_1920 | ggugagaaaa | |
| CB-07 | CBLB human | cuguucgguc | 54 |
| | NM_170662_2517 | uugugauaau | |
| CB-08 | CBLB human | ugacuuaagc | 55 |
| | NM_170662_2596 | auauauuuaa | |
| CB-09 | CBLB human | agucucauug | 56 |
| | NM_170662_2813 | aacauucaaa | |
| CB-10 | CBLB human | gguguuuuga | 57 |
| | NM_170662_3618 | uaccuguacu | |
| CB-11 | CBLB human | caacugauca | 58 |
| | NM_170662_3818 | aacuaaugca | |
| CB-12 | CBLB human | agcauuuauu | 59 |
| | NM_170662_3925 | ugucaauaaa | |

For the purposes of the invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Moreover, for the purposes of the present invention, the term “a” or “an” entity refers to one or more of that entity; for example, “a protein” or “a nucleic acid molecule” refers to one or more of those compounds or at least one compound. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds.

According to the present invention, an isolated, or biologically pure, protein or nucleic acid molecule is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

Compositions and methods described herein are further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1: Engineering and Testing of Sd-rxRNAs

Genes listed in Table 1 were analyzed using a proprietary algorithm to identify preferred sd-rxRNA targeting sequences and target regions. Non-limiting examples of PDCD1 and Cbl-b target sequences and/or sd-rxRNA sequences are shown in Table 3, Table 4, Table 6 and Table 8. Representative sequences for analysis of genes encoding AKT, Tet2, Blimp-1, T-Box21, PTPN6, and HK2 are shown in Tables 7 and 9-13.

### Example 2: Self-Delivering RNAi Immunotherapeutic Agents Targeting TIGIT

The gene encoding TIGIT (NCBI GenBank Accession No. NM_173799) was analyzed using a proprietary algorithm to identify preferred sd-rxRNA targeting sequences and target regions for prevention of immunosuppression of antigen-presenting cells and T-cells. Results for TIGIT are shown in Table 5.

TABLE 5

| | | | SEQ | | SEQ |
|---|---|---|---|---|---|
| Oligo_ | | | ID | | ID |
| ID | TIGIT | Sequence | NO: | Gene_region | NO: |
| Tigit 1 | TIGIT_NM_173799_human_ | CUUUUGUCUUUGCUAUUAUA | 60 | CTTCTGGAAGATACACTTTTGTCTTTGCT | 80 |
| | 840 | | | ATTATAGATGAATATA | |
| Tigit 2 | TIGIT_NM_173799_human_ | UAAUUGGUAUAAGCAUAAAA | 61 | CAAGATGTGCTGTTATAATTGGTATAAGC | 81 |
| | 2827 | | | ATAAAATCACACTAGA | |
| Tigit 3 | TIGIT_NM_173799_human_ | CAAAUUGGAAGUGAACUAAA | 62 | ATAGAACACAATTCACAAATTGGAAGTG | 82 |
| | 2436 | | | AACTAAAATGTAATGAC | |
| Tigit 4 | TIGIT_NM_173799_human_ | GUUUGCUGUGGCAGUUUACA | 63 | CGTAAAAATGTTGTTGTTTGCTGTGGCAG | 83 |
| | 2364 | | | TTTACAGCATTTTTCT | |
| Tigit 5 | TIGIT_NM_173799_human_ | GAUCAUAAAUGCAAAAUUAA | 64 | AGTAACGTGGATCTTGATCATAAATGCA | 84 |
| | 1039 | | | AAATTAAAAAATATCTT | |
| Tigit 6 | TIGIT_NM_173799_human_ | CGCGUUGACUAGAAAGAAGA | 65 | AGTCATCGTGGTGGTCGCGTTGACTAGA | 85 |
| | 559 | | | AAGAAGAAAGCCCTCAG | |
| Tigit 7 | TIGIT_NM_173799_human_ | UUUAAAUAGAACUCACUGAA | 66 | TTTGAAAAAAATTTTTTTAAATAGAACTC | 86 |
| | 2666 | | | ACTGAACTAGATTCTC | |
| Tigit 8 | TIGIT_NM_173799_human_ | GCAAAUCUGUUGGAAAUAGA | 67 | TCTTGCAAAATTAGTGCAAATCTGTTGGA | 87 |
| | 2406 | | | AATAGAACACAATTCA | |
| Tigit 9 | TIGIT_NM_173799_human_ | UCUUGCAAAAUUAGUGCAAA | 68 | AGTTTACAGCATTTTTCTTGCAAAATTAG | 88 |
| | 2391 | | | TGCAAATCTGTTGGAA | |
| Tigit 10 | TIGIT_NM_173799_human_ | ACAUAGGAAGAAUGAACUGA | 69 | TCTACCAAATGGGTTACATAGGAAGAAT | 89 |
| | 2284 | | | GAACTGAAATCTGTCCA | |
| Tigit 11 | TIGIT_NM_173799_human_ | UCACUUUUCUACCAAAUGGG | 70 | ATTATTATTATTTTTTCACTTTTCTACCAA | 90 |
| | 2262 | | | ATGGGTTACATAGGA | |
| Tigit 12 | TIGIT_NM_173799_human_ | GUGUUAUUUAACAUAAUUAU | 71 | TGGACTGAGAGTTGGGTGTTATTTAACAT | 91 |
| | 2531 | | | AATTATGGTAATTGGG | |
| Tigit 13 | TIGIT_NM_173799_human_ | UGUGUGUUCAGUUGAGUGAA | 72 | GTGTGTGTATGTGTGTGTGTGTTCAGTTG | 92 |
| | 924 | | | AGTGAATAAATGTCAT | |
| Tigit 14 | TIGIT_NM_173799_human_ | CUUUGCUAUUAUAGAUGAAU | 73 | AAGATACACTTTTGTCTTTGCTATTATAG | 93 |
| | 847 | | | ATGAATATATAAGCAG | |
| Tigit 15 | TIGIT_NM_173799_human_ | GAAAUGGGAUUCAAUUUGAA | 74 | ATGGGTCAGGTTACTGAAATGGGATTCA | 94 |
| | 2637 | | | ATTTGAAAAAAATTTTT | |
| Tigit 16 | TIGIT_NM_173799_human_ | AAAAUGUAAUGACGAAAAGG | 75 | AATTGGAAGTGAACTAAAATGTAATGAC | 95 |
| | 2453 | | | GAAAAGGGAGTAGTGTT | |
| Tigit 17 | TIGIT_NM_173799_human_ | GGUUACAUAGGAAGAAUGAA | 76 | CTTTTCTACCAAATGGGTTACATAGGAAG | 96 |
| | 2280 | | | AATGAACTGAAATCTG | |
| Tigit 18 | TIGIT_NM_173799_human_ | UUUAGCAACAAGACAAUUCA | 77 | GGGGTTGACAATTGTTTTAGCAACAAGA | 97 |
| | 2206 | | | CAATTCAACTATTTCTC | |
| Tigit 19 | TIGIT_NM_173799_human_ | UGCUAUUAUAGAUGAAUAUA | 78 | ATACACTTTTGTCTTTGCTATTATAGATG | 98 |
| | 850 | | | AATATATAAGCAGCTG | |
| Tigit 20 | TIGIT_NM_173799_human_ | GAGAUUUAAUAUGAAUAAUA | 79 | TCACACTAGATTCTGGAGATTTAATATGA | 99 |
| | 2862 | | | ATAATAAGAATACTAT | |

| TIGIT | | | |
|---|---|---|---|
| Optimized | | | |
| sd-rxRNA | Strand ID | | |
| TIGIT | 27384 | fU.mG.fA.mC.fU.mA.fG.mA.fA.mA. | 100 |
| 21 | | fG.mA.fA*mG*fA.TEG-Chl | |
| | 27380 | P.mU.fC.mU.fU.mC.fU.mU.fU.mC.f | 101 |
| | | U.mA.fG.mU.fC*mA*fA*mC*fG*m | |
| | | C*fG | |

### Example 3: Identification of Potent, Chemically-Optimized, Sd-rxRNAs Targeting PDCD1 in Human Primary T-Cells

Primary human T-cells were obtained from AllCells (CA) and cultured in complete RPMI medium containing 10% Fetal Bovine Serum (Gibco) and 1000 IU/ml IL2. Cells were activated with anti-CD3/CD28 Dynabeads (Gibco, 11131) according to the manufacturer's instructions for at least 4 days prior to the transfection. Chemically optimized sd-rxRNA targeting PDCD1 were prepared by separately diluting the sd-rxRNAs to 0.2-2 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 μl/well into the 96-well plate with pre-diluted sd-rxRNAs. Examples of sd-rxRNA targeting PDCD1 are provided in Table 6.

TABLE 6

| PD1 | | | |
|---|---|---|---|
| Optimized | | | |
| sd-rxRNA | | | |
| Duplex | Strand | | SEQ ID |
| ID | ID | Sequence | NO: |
| PD 21 | 27379 | fU.mG.fU.mG.fG.mU.fU.mC.fU.mA.fU.mU.fA*mU*fA-TEG-Chl | 102 |
| | 27383 | P.mU.fA.mU.fA.mA.fU.mA.fG.mA.fA.mC.fC.mA.fC*mA*fG*mG*fG*mA*fA | 103 |
| PD 22 | 27379 | fU.mG.fU.mG.fG.mU.fU.mC.fU.mA.fU.mU.fA*mU*fA-TEG-Chl | 104 |
| | 27678 | P.mU*fA*mU.fA.mA.fU.mA.fG.mA.fA.mC.fC.mA.fC*mA*fG*mG*fG*mA*fA | 105 |
| PD 23 | 27379 | fU.mG.fU.mG.fG.mU.fU.mC.fU.mA.fU.mU.fA*mU*fA-TEG-Chl | 106 |
| | 27679 | P.mU*fA*mU.fA.mA.fU.mA.fG.mA.fA.mC.fC.mA.fC.mA*fG*mG*fG*mA*fA | 107 |
| PD 24 | 27379 | fU.mG.fU.mG.fG.mU.fU.mC.fU.mA.fU.mU.fA*mU*fA-TEG-Chl | 108 |
| | 27680 | P.mU*fA*mU.fA.mA.fU.mA.fG.mA.fA.mC.fC.mA.fC.mA.fG*mG*fG*mA*fA | 109 |
| PD 25 | 27379 | fU.mG.fU.mG.fG.mU.fU.mC.fU.mA.fU.mU.fA*mU*fA-TEG-Chl | 110 |
| | 27681 | S.mU*fA.mU.fA.mA.fU.mA.fG.mA.fA.mC.fC.mA.fC*mA*fG*mG*fG*mA*fA | 111 |
| PD 26 | 27379 | fU.mG.fU.mG.fG.mU.fU.mC.fU.mA.fU.mU.fA*mU*fA-TEG-Chl | 112 |
| | 27683 | P.mU.fA.fU.mA.mA.fU.mA.mG.mA.fA.fC.fC.mA.fC*mA*mG*mG*mG*mA*mA | 113 |
| PD 27 | 27379 | fU.mG.fU.mG.fG.mU.fU.mC.fU.mA.fU.mU.fA*mU*fA-TEG-Chl | 114 |
| | 27684 | P.mU.fA.mU.fA.mA.fU.mA.fG.mA.fA.fC.fC.mA.fC*mA*fG*mG*fG*mA*fA | 115 |
| PD 28 | 27379 | fU.mG.fU.mG.fG.mU.fU.mC.fU.mA.fU.mU.fA*mU*fA-TEG-Chl | 116 |
| | 27685 | P.mU.fA.mU.fA.mA.fU.mA.fG.mA.fA.fC.mC.mA.fC*mA*fG*mG*fG*mA*fA | 117 |
| PD 29 | 27379 | fU.mG.fU.mG.fG.mU.fU.mC.fU.mA.fU.mU.fA*mU*fA-TEG-Chl | 118 |
| | 27687 | P.mU.fA.mU.A.A.fU.A.G.A.fA.mC.fC.A.fC*A*G*mG*G*mA*fA | 119 |
| PD 30 | 27379 | fU.mG.fU.mG.fG.mU.fU.mC.fU.mA.fU.mU.fA*mU*fA-TEG-Chl | 120 |
| | 27686 | P.mY.fA.mY.fA.mA.fY.mA.fG.mA.fA.mX.fX.mA.fX*mA*fG*mG*fG*mA*fA | 121 |
| PD 31 | 27379 | fU.mG.fU.mG.fG.mU.fU.mC.fU.mA.fU.mU.fA*mU*fA-TEG-Chl | 122 |
| | 27681 | VP.mU*fA.mU.fA.mA.fU.mA.fG.mA.fA.mC.fC.mA.fC*mA*fG*mG*fG* | 123 |
| | | mA*fA | |
| PD 32 | 27688 | mU.mG.mU.mG.mG.mU.mU.mC.mU.mA.mU.mU.mA*mU*mA-TEG-Chl | 124 |
| | 27383 | P.mU.fA.mU.fA.mA.fU.mA.fG.mA.fA.mC.fC.mA.fC*mA*fG*mG*fG*mA*fA | 125 |
| PD 33 | 27689 | fU*mG*fU.mG.fG.mU.fU.mC.fU.mA.fU.mU.fA*mU*fA-TEG-Chl | 126 |
| | 27383 | P.mU.fA.mU.fA.mA.fU.mA.fG.mA.fA.mC.fC.mA.fC*mA*fG*mG*fG*mA*fA | 127 |
| PD 34 | 27690 | fU.mG.fU.G.G.mU.fU.mC.fU.A.fU.mU.A*mU*fA-TEG-Chl | 128 |
| | 27383 | P.mU.fA.mU.fA.mA.fU.mA.fG.mA.fA.mC.fC.mA.fC*mA*fG*mG*fG*mA*fA | 129 |
| PD 35 | 27379 | fU.mG.fU.mG.fG.mU.fU.mC.fU.mA.fU.mU.fA*mU*fA.TEG-Chl | 130 |
| | 27686 | P.mY.fA.mY.fA.mA.fY.mA.fG.mA.fA.mX.fX.mA.fX*mA*fG*mG*fG*mA*fA | 131 |
| PD 36 | 27683 | P.mU.fA.fU.mA.mA.fU.mA.mG.mA.fA.fC.fC.mA.fC*mA*mG*mG*mG*mA*mA | 132 |
| | 27690 | fU.mG.fU.G.G.mU.fU.mC.fU.A.fU.mU.A*mU*fA-TEG-Chl | 133 |
| PD 37 | 27684 | P.mU.fA.mU.fA.mA.fU.mA.fG.mA.fA.fC.fC.mA.fC*mA*fG*mG*fG*mA*fA | 134 |
| | 27690 | fU.mG.fU.G.G.mU.fU.mC.fU.A.fU.mU.A*mU*fA-TEG-Chl | 135 |

Key
A = adenosine
G = guanosine
U = uridine
C = cytodine
m = 2′-O-methyl nucleotide
f = 2′ fluoro nucleotide
Y = 5 methyl uridine
X = 5 methyl cytodine
*= phosphorothioate linkage
.= phosphodiester linkage
TEG-CHl = cholesterol-TEG-Glyceryl
P = 5′ inorganic Phosphate
VP - 5′ Vinyl Phosphonate
S - 5′ Thiophosphate

72 h later, the transfected cells were spun down for 10 minutes at 300×g. The media was removed and the cells were resuspended in 40 μL of Phosphate Buffered Saline (Gibco). Cells were then transferred to Invitrogen mRNA Catcher plates and RNA was isolated as according to Manufacturer's instructions. Taqman gene expression assays were used in the following combinations: human PDCD1-FAM (Taqman, Hs01550088_ml)/human PPIB-FAM (Taqman HS00168719_m1). Reaction volumes were prepared for triplicates however each sample was run in duplicate. A volume of 45 μl/well of each reaction mix was combined with 15 μl RNA per well from the previously isolated RNA. The samples were amplified using the Taqman RNA to CT 1-step kit as per manufactures instructions.

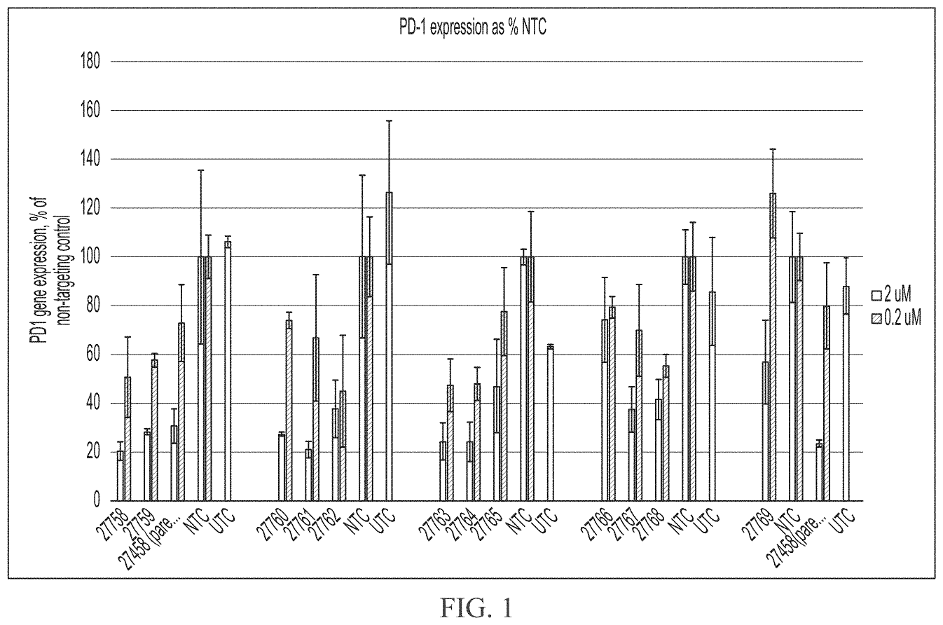

Results shown in FIG. 1 demonstrate significant silencing of PDCD1-targetingsd-rxRNA agents delivered to T-cells, obtaining greater than 60-70% inhibition of gene expression with 2 μM sd-rxRNA.

### Example 4: Six Point Dose Response Curves of Chemically-Optimized, Sd-rxRNAs Targeting PDCD1 in Human Primary T-Cells

Primary human T-cells were obtained from AllCells (CA) and cultured in complete RPMI medium containing 10% Fetal Bovine Serum (Gibco) and 1000 IU/mL IL2. Cells were activated with anti-CD3/CD28 Dynabeads (Gibco, 11131) according to the manufacturer's instructions for at least 4 days prior to the transfection. Chemically optimized sd-rxRNA targeting PDCD1 were prepared by separately diluting the sd-rxRNAs to 0.06-2 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 μl/well into the 96-well plate with pre-diluted sd-rxRNAs.

72 h later, the transfected cells were spun down for 10 minutes at 300×g. The media was removed and the cells were resuspended in 40 μL of Phosphate Buffered Saline (Gibco). Cells were then transferred to Invitrogen mRNA Catcher plates and RNA was isolated according to the manufacturer's instructions. Taqman gene expression assays were used in the following combinations: human PDCD1-FAM (Taqman, Hs01550088_m1)/human PPIB-FAM (Taqman, Hs00168719_m1). A volume of 45 μl/well of each reaction mix was combined with 15 μl RNA per well from the previously isolated RNA. The samples were amplified as described in Example 3.

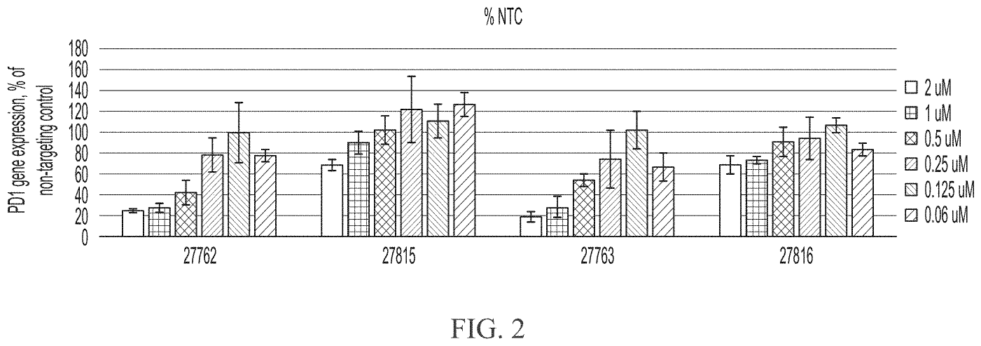

Results shown in FIG. 2 demonstrate significant silencing of PDCD1-targeting sd-rxRNA agents PD26 and PD27 delivered to T-cells, obtaining greater than 60-70% inhibition of gene expression with 2 μM sd-rxRNA.

### Example 5: Silencing Activity of Sd-rxRNAs Targeting TIGIT in Human Primary T-Cells

Primary human T-cells were obtained from AllCells (CA) and cultured in complete RPMI medium containing 1000 IU/ml IL2. Cells were activated with anti-CD3/CD28 Dynabeads (Gibco, 11131) according to the manufacturer's instructions for at least 4 days prior to the transfection. Cells were collected by brief vortexing to dislodge the beads from cells and separating them using the designated magnet. Chemically optimized sd-rxRNA targeting TIGIT were prepared by separately diluting the sd-rxRNAs to 0.04-2 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 μl/well into the 96-well plate with pre-diluted sd-rxRNAs. Examples of sd-rxRNA targeting TIGIT are provided in Table 4.

72 h later, the transfected cells were washed once with 100 μl/well PBS and processed with FastLane Cell Multiplex Kit reagents according to the manufacturer's instructions. Taqman gene expression assays were used in the following combinations: human TIGIT-FAM (Taqman, Hs00545087_m1_m1)/GAPDH-VIC. A volume of 18 μl/well of each reaction mix was combined with 2 μl lysates per well from the previously prepared lysates. The samples were amplified as described in Example 2.

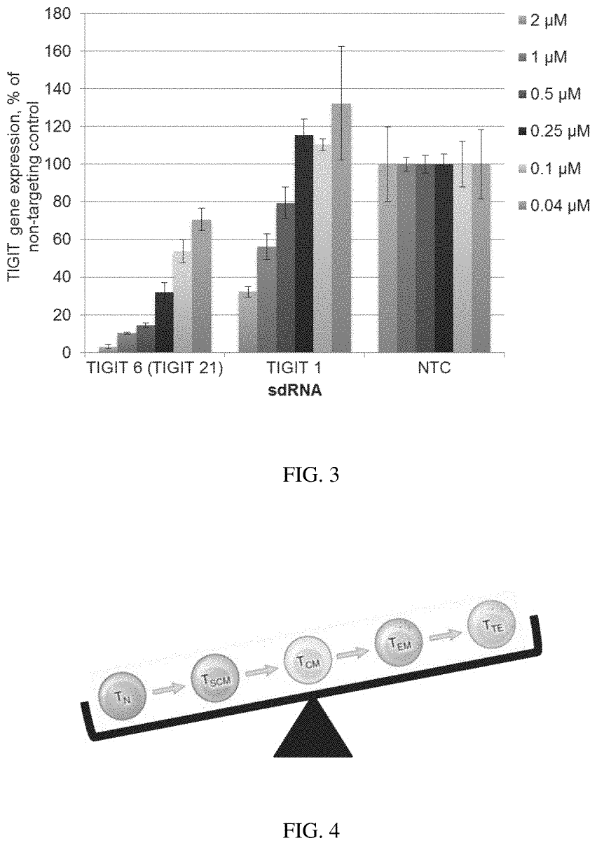

Results shown in FIG. 3 demonstrate significant silencing of TIGIT-targeting sd-rxRNA agents TIGIT 6 and TIGIT 1 delivered to T-cells, obtaining greater than 60-70% inhibition of gene expression with 2 μM sd-rxRNA.

### Example 6: Enhanced T Central Memory (T_{CM}) Differentiation from Activated Human Primary T Cells Treated with PD-1 and TIGIT Targeting Sd-rxRNA in Ex Vivo Culture

This example describes the modification of T-cells with sd-rxRNA to achieve a balance between antitumor efficacy and self-renewal properties of the T-cells. FIG. 4 shows a schematic depiction of the effect of sd-rxRNA treatment on progression of differentiation state of T-cells. Briefly, treatment of T-cells with sd-rxRNA affects cell differentiation during manufacturing of cell-based therapies (e.g., production of ACTs). Additionally, treatment with a plurality of sd-rxRNAs targeting different genes enables simultaneous modulation of multiple differentiation mechanisms, such as signaling pathways, transcription factors, metabolic targets and epigenetic regulators. Treatment of T-cells with sd-rxRNA also allows targeting of “non-druggable” mechanisms.

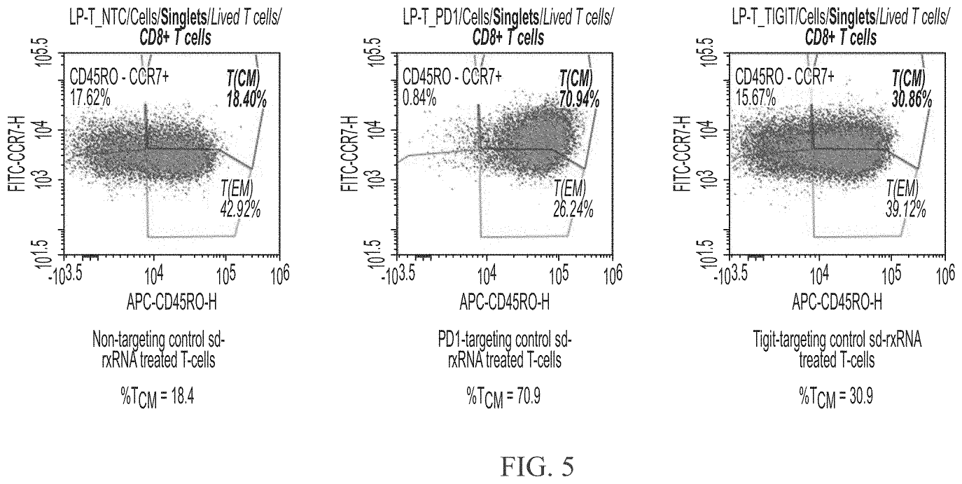

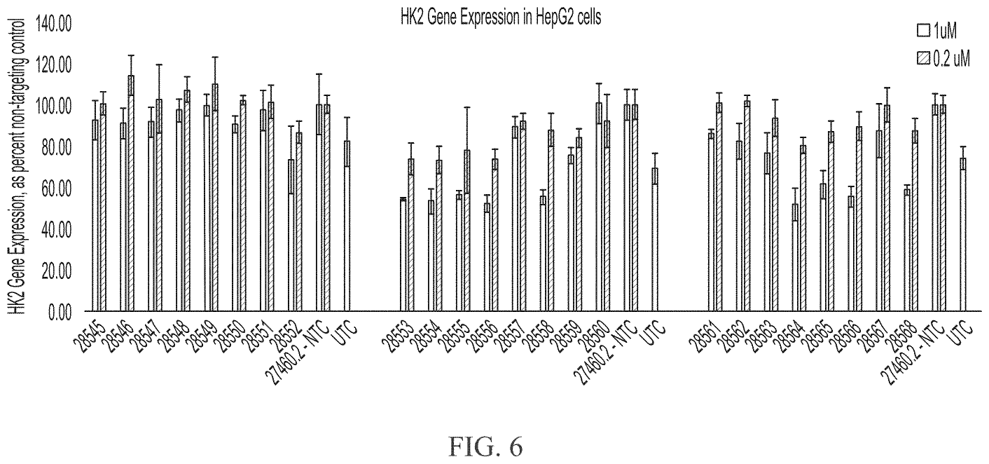

Peripheral blood of a healthy donor was obtained from Stem Express (Arlington, Mass.). Naïve T cells were purified with EasySep™ Human Naïve Pan T Cell Isolation kit from Stem Cell Technologies (Cambridge, Mass.) according to the manufacturer's instructions. Purified naïve T-cells were then activated with CD3/CD28 Dynabeads (ThermoFisher Scientific, Waltham, Mass.) in a 1:1 beads to cells ratio in AIM-V medium+5% FBS+10 ng/mL hIL2 (GeneScript, Piscataway, N.J.). Chemically optimized sd-rxRNA targeting PDCD1 (PD-1), TIGIT, and sd-rxRNA non-targeting control were added to the culture at 2 μM. Four days later, Cells were harvested and stained with Live/Dead fixable Aqua Dead Cell stain kit (ThermoFisher Scientific, Waltham, Mass.), APC-H7 conjugated anti-human CD3, Pacific Blue conjugated anti-human CD8, FITC conjugated anti-human CCR7 and APC conjugated anti-human CD45RO (BD Bioscience, San Jose, Calif. and BioLegend, San Diego, Calif.). As shown in FIG. 5, the T-cell differentiation to the CD8⁺ T_{CM} (CCR7⁺ CD45RO⁻) subtype was enhanced 3.9 fold and 1.7 fold upon PD-1 and TIGIT inhibition, respectively as compared to the control.

### Example 7: Two Point Dose Response Curves of Sd-rxRNAs Targeting HK2 in HepG2 Cells

HepG2 cells were obtained from ATCC (VA) and cultured in complete EMEM medium containing 10% Fetal Bovine Serum (Gibco). Twenty-four hours prior to transfection, cells were seeded at 10,000 cells per well into 96-well plates. sd-rxRNA compounds targeting HK2 (e.g., as set forth in Table 5) were prepared by separately diluting the sd-rxRNAs to 0.25-1 μM in Accell Media (Dharmacon, CO) per sample (well) and aliquoted at 100 i 1/well of the pre-seeded 96-well plates. 48 h post administration, the transected cells were lysed and mRNA levels determined by the Quantigene branched DNA assay according to the manufacture's protocol using gene-specific probes (Affymetrix). FIG. 6 demonstrates the HK2-targeting sd-rxRNAs reduce target gene mRNA levels in vitro in HepG2 cells. Data were normalized to a house keeping gene (PPIB) and graphed with respect to the non-targeting control. Error bars represent the standard deviation from the mean of biological triplicates.

### Example 8: Six Point Dose Response Curves of Sd-rxRNAs Targeting HK2 in Pan-T Cells

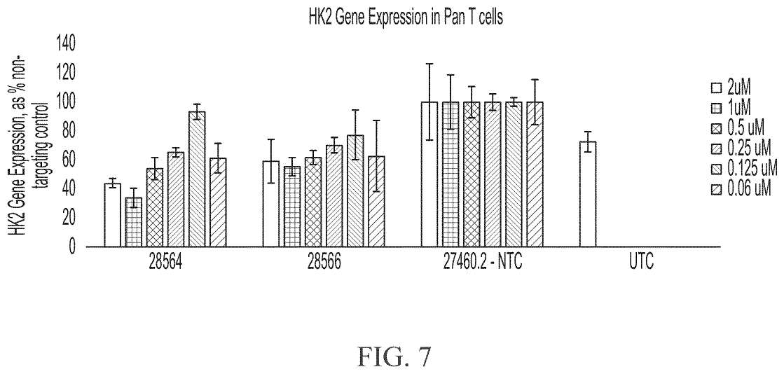

Primary human T-cells were obtained from AllCells (CA) and cultured in complete RPMI medium containing 10% Fetal Bovine Serum (Gibco) and containing 1000 IU/ml IL2. Cells were activated with anti-CD3/CD28 Dynabeads (Gibco, 11131) according to the manufacturer's instructions for at least 4 days prior to the transfection. sd-rxRNA compounds targeting HK2 and a non-targeting control sd-rxRNA (#28599) were prepared by separately diluting the sd-rxRNAs to 0.04-2 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 μl/well into the 96-well plate with pre-diluted sd-rxRNAs. Examples of sd-rxRNA targeting HK2 sequence are provided in Table 7. 72 h post administration, cells were lysed and mRNA levels determined by the Quantigene branched DNA assay according to the manufacture's protocol using gene-specific probes (Affymetrix). FIG. 7 demonstrates the HK2-targeting sd-rxRNAs reduce target gene mRNA levels in vitro in human Pan T cells. Data were normalized to a house keeping gene (PPIB) and graphed with respect to the non-targeting control. Error bars represent the standard deviation from the mean of biological triplicates.

### Example 9: Cbl-b Silencing in T Cells

T-cells were cultured in complete RPMI medium containing 10% Fetal Bovine Serum (Gibco) and containing 1000 IU/ml 1L2. Cells were activated with anti-CD3/CD28 Dynabeads (Gibco, 11131) according to the manufacturer's instructions for at least 4 days prior to the transfection. sd-rxRNA compounds targeting Cbl-b or a non-targeting control (NTC) sd-rxRNA were prepared by separately diluting the sd-rxRNAs to 2 uM or 0.04-2 μM in serum-free RPMI per sample (well) and aliquoted at 100 l/well of 96-well plate. Cells were prepared RPMI medium containing 4% FBS and L2 2000 U/ml at 1,000,000 cells/ml and seeded at 100 l/well into the 96-well plate with pre-diluted sd-rxRNAs. Examples of sd-rxRNA targeting Cbl-b sequence are provided in Table 8. At the end of the transfection incubation period, the plated transfected cells were washed once with 100 l/well PBS and processed with FastLane Cell Multiplex Kit reagents according to the manufacturer's instructions. Taqman gene expression assays were used in the following combinations: human Cbl-b-FAM/GAPDH-VIC. A volume of 18 l/well of each reaction mix was combined with 2 μl lysates per well from the previously prepared lysates. The samples were amplified as according to manufacturer's instructions.

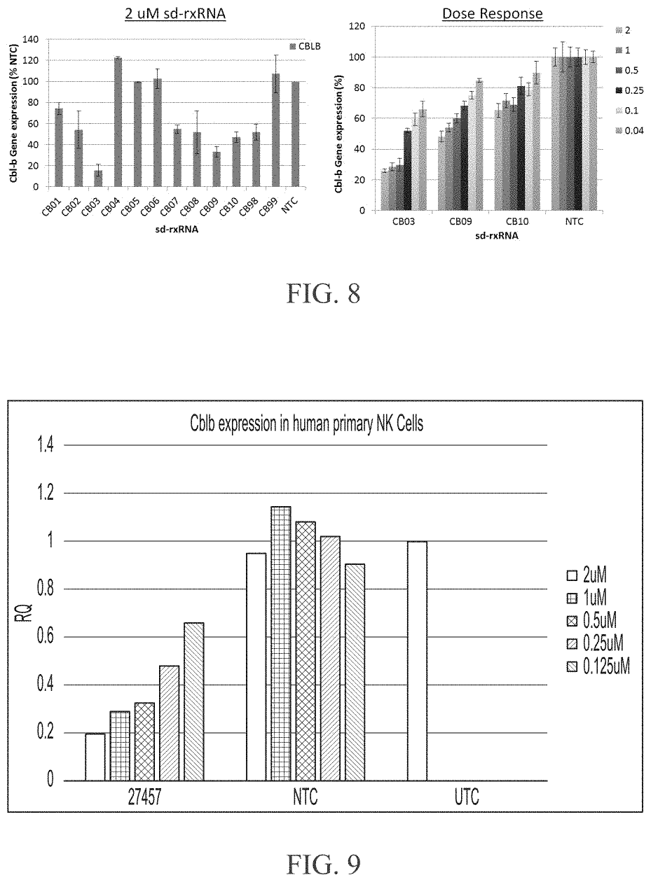

The results in FIG. 8 demonstrate significant silencing of both Cbl-b by sd-rxRNA compounds transfected into T-cells, reaching 70-80% inhibition of gene expression with 1-2 μM sd-rxRNA.

### Example 10: Six point dose response of sd-rxRNAs Targeting CBLB in human primary NK Cells

A peripheral blood leukopak was obtained from StemCell Technologies. Primary NK cells were isolated using a negative selection kit (Miltenyi) and cells were cultured in X-Vivo 10 (Lonza)+1 ng/ml IL-15. Cells were collected for transfection and the cell concentration was adjusted to ˜1×10⁶ cells/mL in X-vivo media containing IL-15. Cells were seeded directly into 24-well plates containing sd-rxRNAs ranging in final concentration from 0.125 μM to 2 μM. After 72 hour incubation, the transfected cells were collected and RNA was isolated using the RNEasy RNA isolation kit (Qiagen) as per manufacturer's protocol. Taqman gene expression assays were used in the following combination: human Cblb-FAM (Taqman, Hs00180288_m1)/human TBP-FAM (Taqman, Hs00427620_ml). A volume of 15 l/well of each reaction mix was combined with 5 μL RNA per well from the previously isolated RNA. The samples were amplified following the RNA to Ct 1-step protocol (ThermoFisher).

Results shown in FIG. 9 demonstrate silencing of Cblb-targeting sd-rxRNA agent 27457 delivered to human primary NK cells, obtaining greater than 80% inhibition of gene expression with 2 μM sd-rxRNA.

### Example 11: Three Point Dose Response Curves of Sd-rxRNAs Targeting DMNT3A in HepG2 Cells

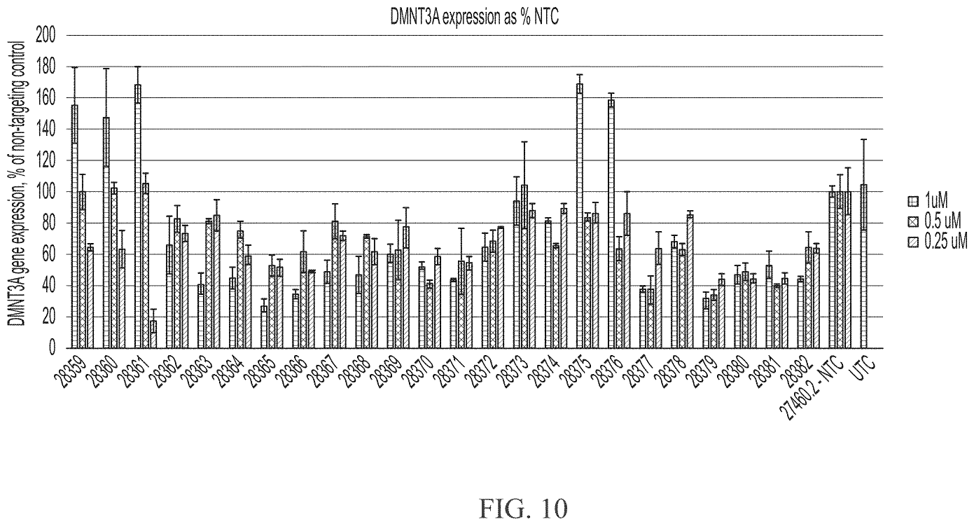

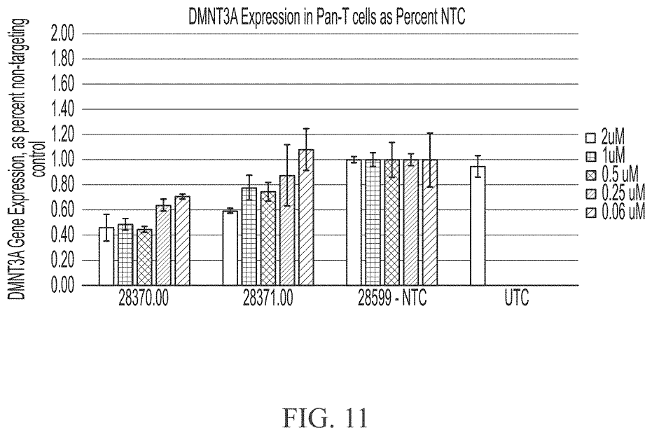

HepG2 cells were obtained from ATCC (VA) and cultured in complete EMEM medium containing 10% Fetal Bovine Serum (Gibco). Twenty-four hours prior to transfection, cells were seeded at 10,000 cells per well into 96-well plates. sd-rxRNA compounds targeting DMNT3A were prepared by separately diluting the sd-rxRNAs to 0.25-1 μM in Accell Media (Dharmacon, CO) per sample (well) and aliquoted at 100 μl/well of the pre-seeded 96-well plates. 48 h post administration, the transected cells were lysed and mRNA levels determined by the Quantigene branched DNA assay according to the manufacturer's protocol using gene-specific probes (Affymetrix). FIG. 10 demonstrates the DMNT3A-targeting sd-rxRNAs reduce target gene mRNA levels in vitro in HepG2 cells. Data were normalized to a house keeping gene (PPIB) and graphed with respect to the non-targeting control. Error bars represent the standard deviation from the mean of biological triplicates.

### Example 12: Five Point Dose Response Curves Sd-rxRNAs Targeting DMNT3A in Pan-T Cells

Primary human T-cells were obtained from AllCells (CA) and cultured in complete ImmunoCult- XF T Cell Expansion Medium (Stem Cell Technologies, Vancouver, BC) containing 1000 IU/ml IL2. Cells were activated with anti-CD3/CD28 Dynabeads (Gibco, 11131) according to the manufacturer's instructions for at least 4 days prior to the transfection. sd-rxRNA compounds targeting DMNT3A and a non-targeting control sd-rxRNA (#28599) were prepared by separately diluting the sd-rxRNAs to 0.04-2 μM in complete Immunocult per sample (well) and aliquoted at 100 μl/well of 96-well plate. Cells were prepared in complete Immunocult medium and seeded at 100 μl/well into the 96-well plate with pre-diluted sd-rxRNAs. Examples of sd-rxRNA sequences targeting DMNT3A are provided in Table 9. 72 h post administration, cells were lysed and mRNA levels determined by the Quantigene branched DNA assay according to the manufacture's protocol using gene-specific probes (Affymetrix). FIG. 11 demonstrates the DMNT3A-targeting sd-rxRNAs reduce target gene mRNA levels in vitro in human Pan T cells. Data were normalized to a house keeping gene (PPIB) and graphed with respect to the non-targeting control. Error bars represent the standard deviation from the mean of biological triplicates.

### Example 13: Two Point Dose Response of Sd-rxRNAs Targeting PRDM1 in A549 Cells

A549 cells were obtained from ATCC (VA) and cultured in complete ATCC-formulated F-12K medium containing 10% Fetal Bovine Serum (Gibco). Twenty-four hours prior to transfection, cells were seeded at 10,000 cells per well into 96-well plates. sd-rxRNA compounds targeting PRDM1 were prepared by separately diluting the sd-rxRNAs to 0.2-2 μM in Accell Media (Dharmacon) per sample (well) and aliquoted at 100 l/well of the pre-seeded 96-well plates. After 72 hours incubation, the transfected cells were lysed and mRNA levels determined by the Quantigene branched DNA assay according to the manufacture's protocol using gene-specific probes (Affymetrix).

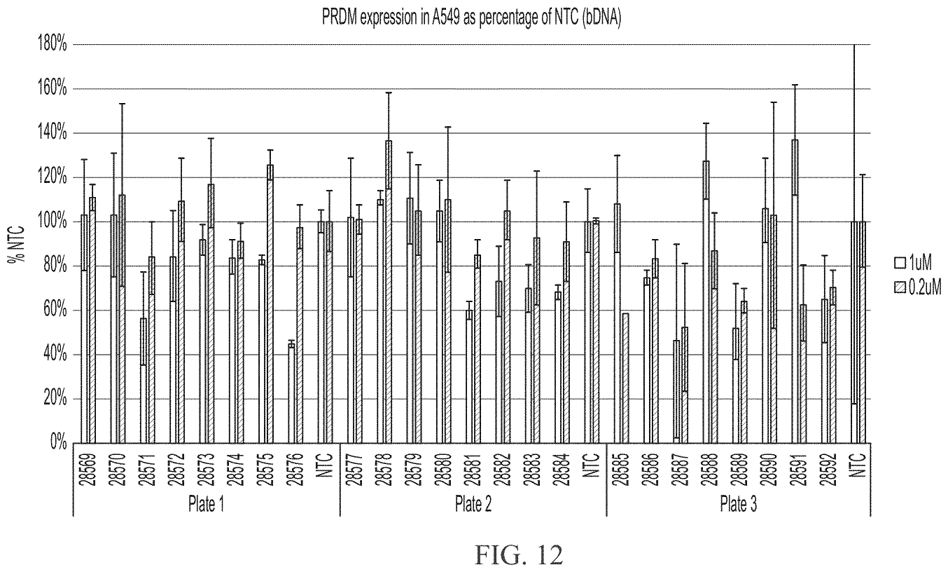

Results shown in FIG. 12 demonstrate silencing of PRDM1-targeting sd-rxRNA agents delivered to A549 cells, obtaining greater than 40% inhibition of gene expression with 2 μM sd-rxRNA. Data were normalized to a house keeping gene (HPRT) and graphed with respect to the non-targeting control. Error bars represent the standard deviation from the mean of biological triplicates.

### Example 14: Six Point Dose Response of Sd-rxRNAs Targeting PRDM1 in A549 Cells

A549 cells were obtained from ATCC (VA) and cultured in complete ATCC-formulated F-12K medium containing 10% Fetal Bovine Serum (Gibco). Twenty-four hours prior to transfection, cells were seeded at 10,000 cells per well into 96-well plates. sd-rxRNA compounds targeting PRDM1 were prepared by separately diluting the sd-rxRNAs to 0.2-2 μM in Accell Media (Dharmacon) per sample (well) and aliquoted at 100 l/well of the pre-seeded 96-well plates. Examples of sd-rxRNA sequences targeting PRDM1 are provided in Table 10. After 72 hours incubation, the transfected cells were lysed and mRNA levels determined by the Quantigene branched DNA assay according to the manufacture's protocol using gene-specific probes (Affymetrix).

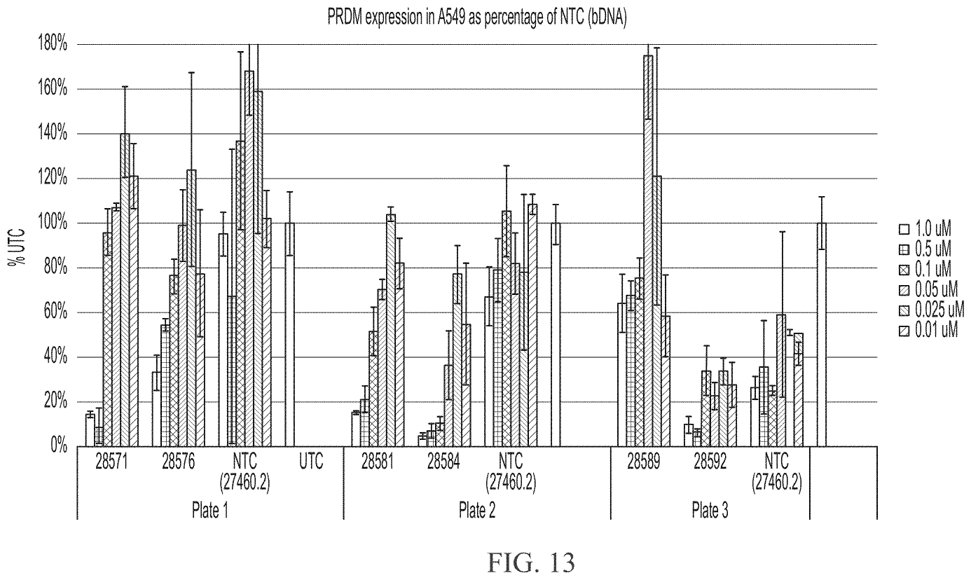

Results shown in FIG. 13 demonstrate silencing of PRDM1-targeting sd-rxRNA agents delivered to A549 cells, obtaining greater than 80% inhibition of gene expression with 2 μM sd-rxRNA. Data were normalized to a house keeping gene (HPRT) and graphed with respect to the non-targeting control. Error bars represent the standard deviation from the mean of biological triplicates.

### Example 15: Six Point Dose Response of Sd-rxRNAs Targeting PTPN6 in A549 Cells

A549 cells were obtained from ATCC (VA) and cultured in complete ATCC-formulated F-12K medium containing 10% Fetal Bovine Serum (Gibco). Twenty-four hours prior to transfection, cells were seeded at 10,000 cells per well into 96-well plates. sd-rxRNA compounds targeting PTPN6 were prepared by separately diluting the sd-rxRNAs to 0.2-2 μM in Accell Media (Dharmacon) per sample (well) and aliquoted at 100 l/well of the pre-seeded 96-well plates. After 72 hour incubation, the transfected cells were lysed and mRNA levels determined by the Quantigene branched DNA assay according to the manufacture's protocol using gene-specific probes (Affymetrix).

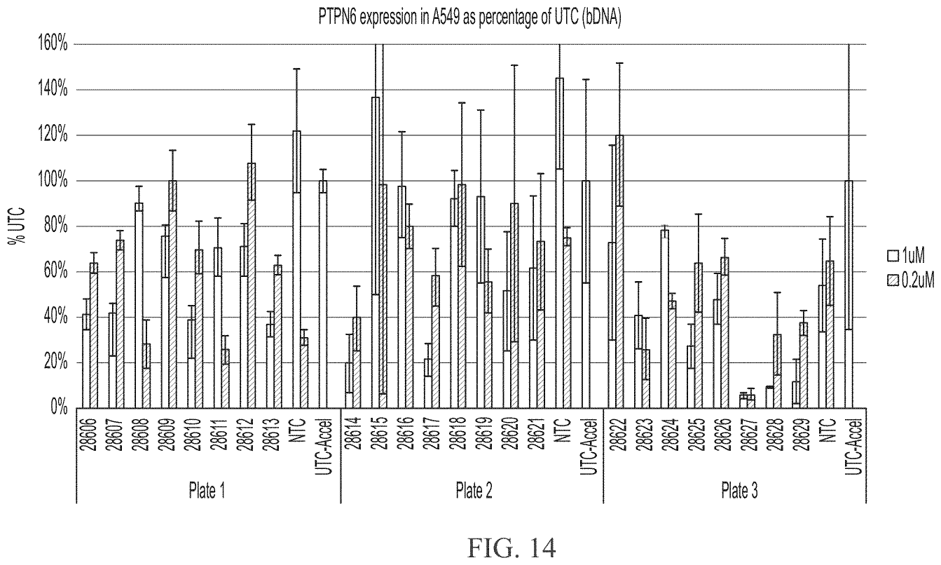

Results shown in FIG. 14 demonstrate silencing of PTPN6-targeting sd-rxRNA agents delivered to A549 cells, obtaining greater than 40% inhibition of gene expression with 2 μM sd-rxRNA. Data were normalized to a house keeping gene (TFRC) and graphed with respect to the untransfected control. Error bars represent the standard deviation from the mean of biological triplicates.

### Example 16: Six Point Dose Response of Sd-rxRNAs Targeting PTPN6 in A549 Cells

A549 cells were obtained from ATCC (VA) and cultured in complete ATCC-formulated F-12K medium containing 10% Fetal Bovine Serum (Gibco). Twenty-four hours prior to transfection, cells were seeded at 10,000 cells per well into 96-well plates. sd-rxRNA compounds targeting PTPN6 were prepared by separately diluting the sd-rxRNAs to 0.2-2 μM in Accell Media (Dharmacon) per sample (well) and aliquoted at 100 l/well of the pre-seeded 96-well plates. Examples of sd-rxRNA sequences targeting PTPN6 are provided in Table 11. After 72 hour incubation, the transfected cells were lysed and mRNA levels determined by the Quantigene branched DNA assay according to the manufacture's protocol using gene-specific probes (Affymetrix).

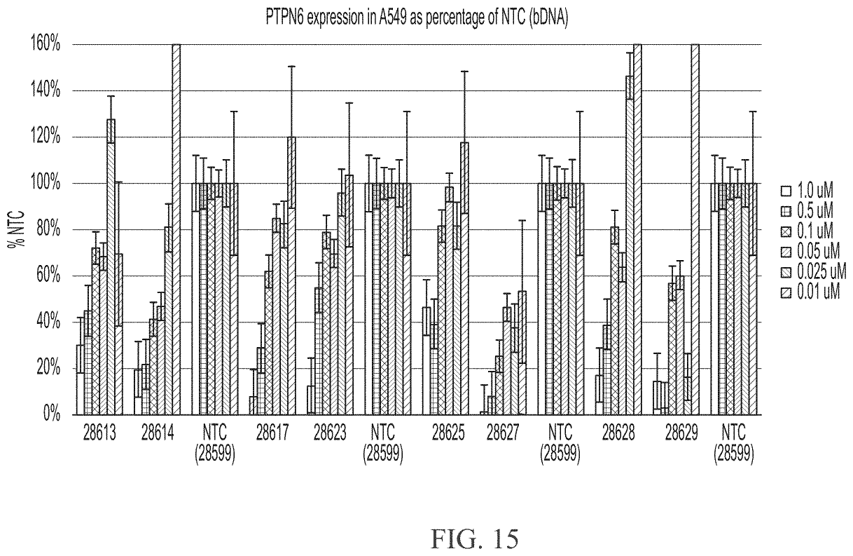

Results shown in FIG. 15 demonstrate silencing of PTPN6-targeting sd-rxRNA agents 28613, 28614, 28617, 28623, 28627, 28628, and 28629 delivered to A549 cells, obtaining greater than 80% inhibition of gene expression with 2 μM sd-rxRNA. Data were normalized to a house keeping gene (TFRC) and graphed with respect to the non-targeting control. Error bars represent the standard deviation from the mean of biological triplicates.

### Example 17: Two Point Dose Response of Sd-rxRNAs Targeting TET2 in U20S Cells

U20S cells were obtained from ATCC (VA) and cultured in complete ATCC-formulated McCoy's 5a Medium containing 10% Fetal Bovine Serum (Gibco). Twenty-four hours prior to transfection, cells were seeded at 10,000 cells per well into 96-well plates. sd-rxRNA compounds targeting TET2 were prepared by separately diluting the sd-rxRNAs to 0.2-2 μM in Accell Media (Dharmacon) per sample (well) and aliquoted at 100 l/well of the pre-seeded 96-well plates. After 72 hours incubation, the transfected cells were lysed and mRNA levels determined by the Quantigene branched DNA assay according to the manufacture's protocol using gene-specific probes (Affymetrix).

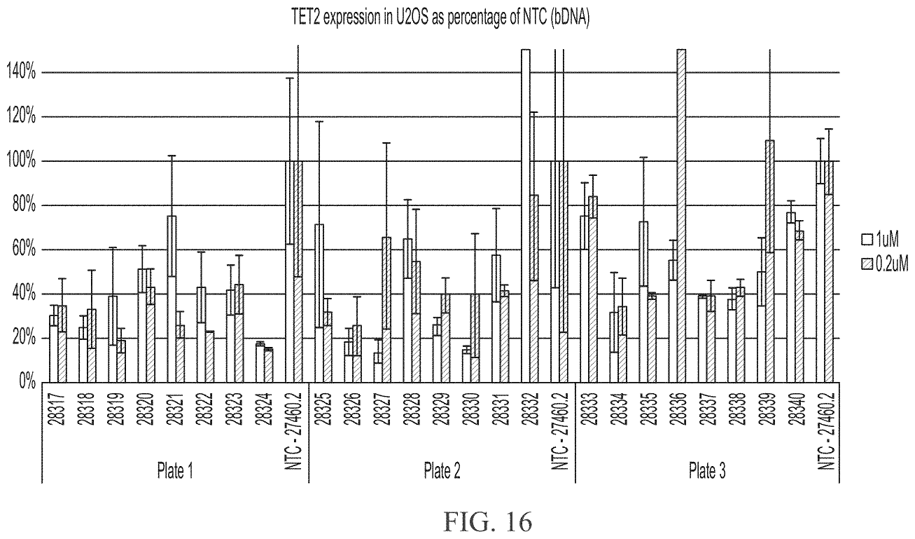

Results shown in FIG. 16 demonstrate silencing of TET2-targeting sd-rxRNA agents delivered to A549 cells, obtaining greater than 80% inhibition of gene expression with 2 μM sd-rxRNA. Data were normalized to a house keeping gene (PPIB) and graphed with respect to the non-targeting control. Error bars represent the standard deviation from the mean of biological triplicates.

### Example 18: Six Point Dose Response of Sd-rxRNAs Targeting TET2 in U2OSCells

U2OS cells were obtained from ATCC (VA) and cultured in complete ATCC-formulated F-12K medium containing 10% Fetal Bovine Serum (Gibco). Twenty-four hours prior to transfection, cells were seeded at 10,000 cells per well into 96-well plates. sd-rxRNA compounds targeting TET2 were prepared by separately diluting the sd-rxRNAs to 0.2-2 μM in Accell Media (Dharmacon) per sample (well) and aliquoted at 100 l/well of the pre-seeded 96-well plates. Examples of sd-rxRNA sequences targeting TET2 are provided in Table 12. After 72 hour incubation, the transfected cells were lysed and mRNA levels determined by the Quantigene branched DNA assay according to the manufacture's protocol using gene-specific probes (Affymetrix).

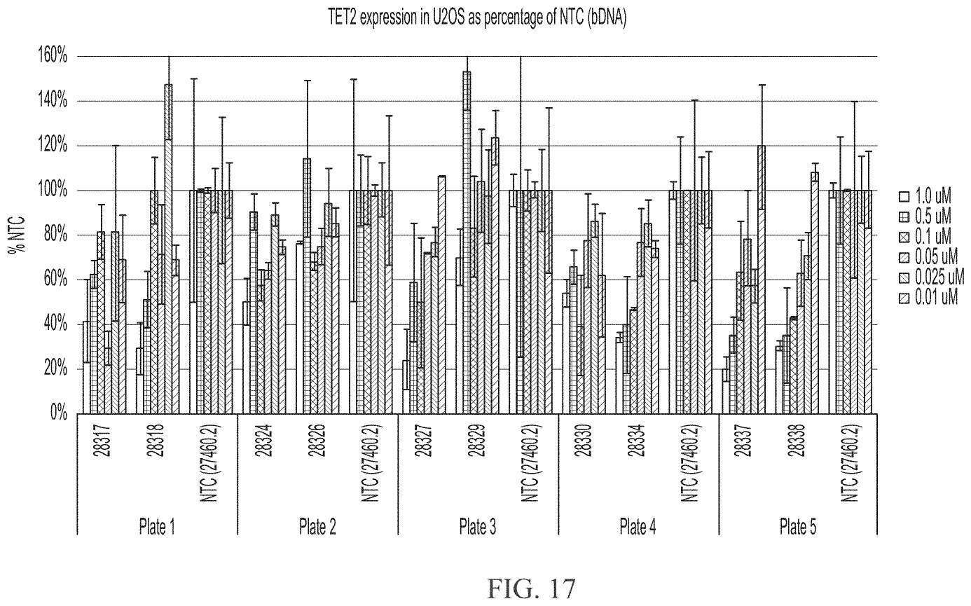

Results shown in FIG. 17 demonstrate silencing of TET2-targeting sd-rxRNA agents delivered to U20S cells, obtaining greater than 60% inhibition of gene expression with 2 μM sd-rxRNA. Data were normalized to a house keeping gene (PPIB) and graphed with respect to the non-targeting control. Error bars represent the standard deviation from the mean of biological triplicates.

### Example 19: Two Point Dose Response Curves of Sd-rxRNAs Targeting TBX21 in Pan T Cells

Primary human T-cells were obtained from AllCells (CA) and cultured in complete RPMI medium containing 10% Fetal Bovine Serum (Gibco) and containing 1000 IU/ml IL2. Cells were activated with anti-CD3/CD28 Dynabeads (Gibco, 11131) according to the manufacturer's instructions for at least 4 days prior to the transfection. sd-rxRNA compounds targeting TBX21 were prepared by separately diluting the sd-rxRNAs to 0.2 and 1 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 μl/well into the 96-well plate with pre-diluted sd-rxRNAs. Examples of sd-rxRNA sequences targeting TET2 are provided in Table 13. 72 h post administration, the transected cells were lysed and mRNA levels determined by the Quantigene branched DNA assay according to the manufacture's protocol using gene-specific probes (Affymetrix).

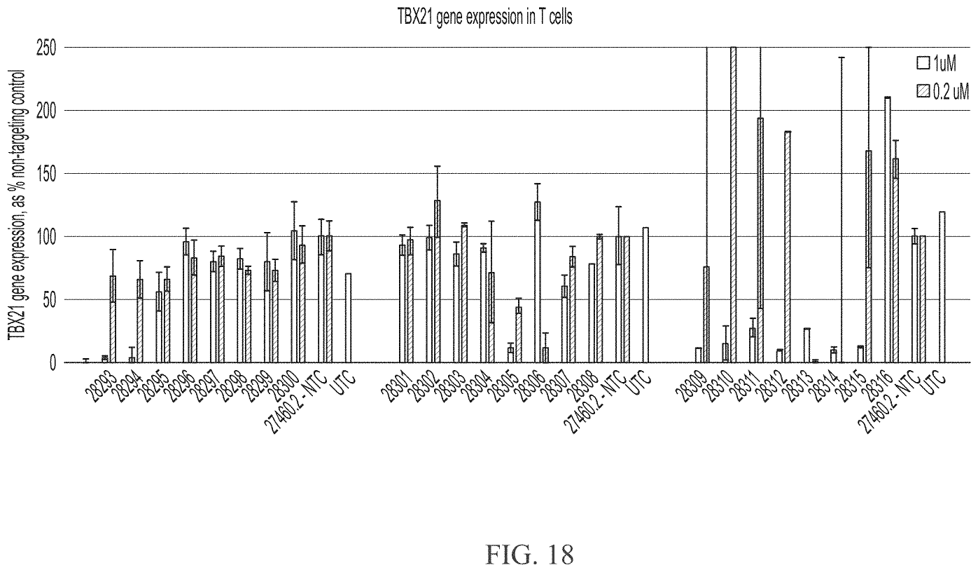

FIG. 18 demonstrates the TBX21-targeting sd-rxRNAs reduce target gene mRNA levels in vitro in Pan T cells. Data were normalized to a house keeping gene (PPIB) and graphed with respect to the non-targeting control. Error bars represent the standard deviation from the mean of biological triplicates.

### Example 20. Three Point Dose Response of Sd-rxRNAs Targeting TIGIT in Human Primary NK Cells

A peripheral blood leukopak was obtained from StemCell Technologies. Primary NK cells were isolated using a negative selection kit (Miltenyi) and cells were cultured in RPMI containing 10% FBS (Gibco) and 100 IU/ml IL-2.

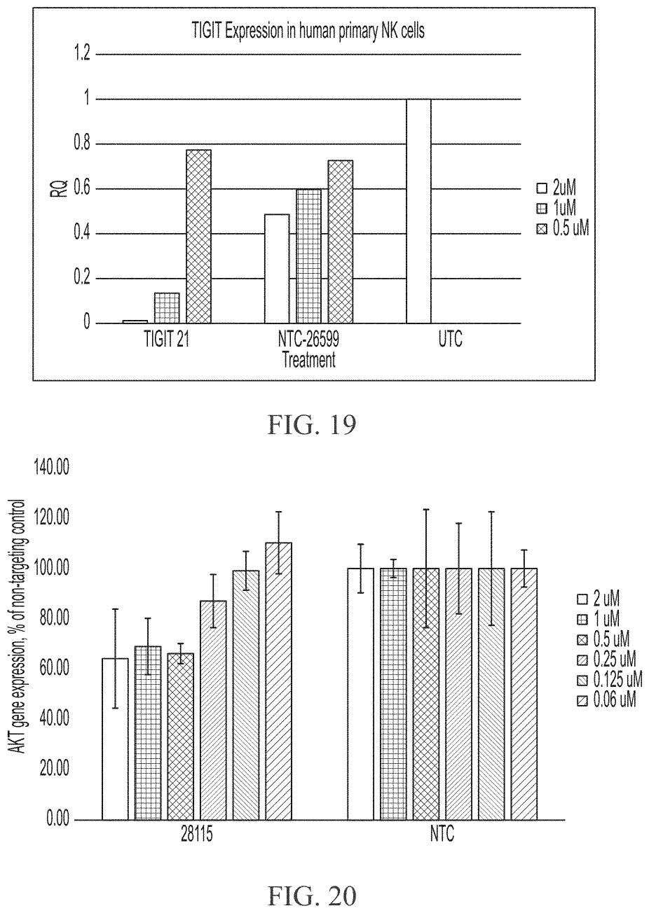

Cells were collected for transfection and the cell concentration was adjusted to ˜1×106 cells/mL in RPMI containing 5% FBS (Gibco) and 100 IU/ml IL-2. Cells were seeded directly into 24-well plates containing sd-rxRNAs ranging in final concentration from 0.5 μM to 2 μM. Examples of sd-rxRNA sequences targeting TIGIT are provided in Table 5. After 72 hour incubation, the transfected cells were collected and RNA was isolated using the RNEasy RNA isolation kit (Qiagen) as per manufacturer's protocol. Taqman gene expression assays were used in the following combination: human TIGIT-FAM (Taqman, Hs00545087_ml)/human TBP-FAM (Taqman, Hs00427620_ml). A volume of 15 μl/well of each reaction mix was combined with 5 μL RNA per well from the previously isolated RNA. The samples were amplified following the RNA to Ct 1-step protocol (ThermoFisher) Results shown in FIG. 19 demonstrate silencing of TIGIT-targeting sd-rxRNA agent 27459 delivered to human primary NK cells, obtaining greater than 80% inhibition of gene expression with 2 μM sd-rxRNA.

### Example 21: Six Point Dose Response Curves of Sd-rxRNAs Targeting AKT1 in Human Primary T-Cells

Primary human T-cells were obtained from AllCells (CA) and cultured in complete RPMI medium containing 10% Fetal Bovine Serum (Gibco) and 1000 IU/ml IL2. Cells were activated with anti-CD3/CD28 Dynabeads (Gibco, 11131) according to the manufacturer's instructions for at least 4 days prior to the transfection. sd-rxRNA compounds targeting AKT1 were prepared by separately diluting the sd-rxRNAs to 0.06-2 μM in serum-free RPMI per sample (well) and aliquoted at 100 μl/well of 96-well plate. Cells were prepared in RPMI medium containing 4% FBS and IL2 2000 U/ml at 1,000,000 cells/ml and seeded at 100 l/well into the 96-well plate with pre-diluted sd-rxRNAs. An example of an sd-rxRNA sequence targeting AKT1 is provided in Table 11. 72 h later, the transfected cells were spun down for 10 minutes at 300×g. The media was removed and the cells were resuspended in 40 uL of Phosphate Buffered Saline (Gibco). Cells were then transferred to Invitrogen mRNA Catcher plates and RNA was isolated according to manufacturer's instructions. Taqman gene expression assays were used in the following combinations: human AKT1-FAM (Taqman, Hs0178289_m1)/human PPIB-FAM (Taqman, Hs00168719_ml). A volume of 45 l/well of each reaction mix was combined with 15 μl RNA per well from the previously isolated RNA. The samples were amplified according to manufacturer's instructions.

Results shown in FIG. 20 demonstrate silencing of AKT1-targeting sd-rxRNA agent 28115 delivered to T-cells, obtaining greater than 40% inhibition of gene expression with 2 μM sd-rxRNA.

### Listing of Tables:

Table 1 shows examples of genes successfully silenced using sd-rxRNAs.
Table 2 shows examples of candidate genes for silencing with sd-rxRNAs.
Table 3 shows examples of PD1 targeting sequences.
Table 4 shows examples of Cbl-b targeting sequences.
Table 5 shows examples of TIGIT targeting sequences and sd-rxRNAs.
Table 6 shows examples of PD1 sd-rxRNAs.
Table 7 shows examples of HK2 target sequences and sd-rxRNAs.
Table 8 shows examples of Cbl-b sd-rxRNAs.
Table 9 shows examples of DNMT3A target sequences and sd-rxRNAs.
Table 10 shows examples of PRDM1 target sequences and sd-rxRNAs.
Table 11 shows examples of PTPN6 target sequences and sd-rxRNAs.
Table 12 shows examples of TET2 target sequences and sd-rxRNAs.
Table 13 shows examples of Tbox21 target sequences and sd-rxRNAs.

TABLE 7

| Oligo | | | Start | | | Passenger | Guide |
|---|---|---|---|---|---|---|---|
| ID | Gene | Accession number | Site | Sequence | Gene region | sequence | Sequence |
| 28545 | HK2 | NM_000189.4 | 2298 | CCAGCUGUU | gcaguggcacCCAGC | UGUUUGAC | UAAUGUGGU |
| | | | | UGACCACAU | UGUUUGACCACA | CACAUUA | CAAACAGCU |
| | | | | UG (SEQ ID | UUGccgaaugccug | (SEQ ID NO: | GG (SEQ ID |
| | | | | NO: 136) | (SEQ ID NO: 160) | 184) | NO: 208) |
| 28546 | HK2 | NM_000189.4 | 2302 | CUGUUUGAC | uggcacccagCUGUU | UGACCACA | UCGGCAAUG |
| | | | | CACAUUGCC | UGACCACAUUGC | UUGCCGA | UGGUCAAAC |
| | | | | GA (SEQ ID | CGAaugccuggcua | (SEQ ID NO: | AG (SEQ ID |
| | | | | NO: 137) | (SEQ ID NO: 161) | 185) | NO: 209) |
| 28547 | HK2 | NM_000189.4 | 2305 | UUUGACCAC | cacccagcugUUUGA | CCACAUUGC | UAUUCGGCA |
| | | | | AUUGCCGAA | CCACAUUGCCGA | CGAAUA | AUGUGGUCA |
| | | | | UG (SEQ ID | AUGccuggcuaacu | (SEQ ID NO: | AA (SEQ ID |
| | | | | NO: 138) | (SEQ ID NO: 162) | 186) | NO: 210) |
| 28548 | HK2 | NM_000189.4 | 3858 | AGAGGAGUU | uccaccggcgAGAGG | AGUUUGAC | UAUCCAGGU |
| | | | | UGACCUGGA | AGUUUGACCUGG | CUGGAUA | CAAACUCCU |
| | | | | UG (SEQ ID | AUGugguugcugug | (SEQ ID NO: | CU (SEQ ID |
| | | | | NO: 139) | (SEQ ID NO: 163) | 187) | NO: 211) |
| 28549 | HK2 | NM_000189.4 | 3939 | CUGUGAAGU | aagacccucaCUGUG | AAGUUGGC | UAAUGAGGC |
| | | | | UGGCCUCAU | AAGUUGGCCUCA | CUCAUUA | CAACUUCAC |
| | | | | UG (SEQ ID | UUGuuggcacgggc | (SEQ ID NO: | AG (SEQ ID |
| | | | | NO: 140) | (SEQ ID NO: 164) | 188) | NO: 212) |
| 28550 | HK2 | NM_000189.4 | 3973 | AAUGCCUGC | cacgggcagcAAUGC | CUGCUACA | UCCUCCAUG |
| | | | | UACAUGGAG | CUGCUACAUGGA | UGGAGGA | UAGCAGGCA |
| | | | | GA (SEQ ID | GGAgaugcgcaacg | (SEQ ID NO: | UU (SEQ ID |
| | | | | NO: 141) | (SEQ ID NO: 165) | 189) | NO: 213) |
| 28551 | HK2 | NM_000189.4 | 4363 | UGUGACGAC | ugagagcaccUGUGA | CGACAGCA | UCAAUGAUG |
| | | | | AGCAUCAUU | CGACAGCAUCAU | UCAUUGA | CUGUCGUCA |
| | | | | GU (SEQ ID | UGUuaaggaggugu | (SEQ ID NO: | CA (SEQ ID |
| | | | | NO: 142) | (SEQ ID NO: 166) | 190) | NO: 214) |
| 28552 | HK2 | NM_000189.4 | 2597 | GUGAGAUUG | gaccacaacuGUGAG | AUUGGUCU | UACAAUGAG |
| | | | | GUCUCAUUG | AUUGGUCUCAUU | CAUUGUA | ACCAAUCUC |
| | | | | UG (SEQ ID | GUGggcacgggcag | (SEQ ID NO: | AC (SEQ ID |
| | | | | NO: 143) | (SEQ ID NO: 167) | 191) | NO: 215) |
| 28553 | HK2 | NM_000189.4 | 4287 | GUCUCAGAU | ccaaguucuuGUCUC | AGAUUGAG | UGUCACUCU |
| | | | | UGAGAGUGA | AGAUUGAGAGUG | AGUGACA | CAAUCUGAG |
| | | | | CU (SEQ ID | ACUgccuggcccug | (SEQ ID | AC (SEQ ID |
| | | | | NO: 144) | (SEQ ID NO: 168) | NO: 192) | NO: 216) |
| 28554 | HK2 | NM_000189.4 | 4289 | CUCAGAUUG | aaguucuuguCUCAG | AUUGAGAG | UCAGUCACU |
| | | | | AGAGUGACU | AUUGAGAGUGAC | UGACUGA | CUCAAUCUG |
| | | | | GC (SEQ ID | UGCcuggcccugcu | (SEQ ID NO: | AG (SEQ ID |
| | | | | NO: 145) | (SEQ ID NO: 169) | 193) | NO: 217) |
| 28555 | HK2 | NM_000189.4 | 4544 | AAGUCAUGC | cacuuugccaAAGUC | AUGCAUGA | UACUGUCUC |
| | | | | AUGAGACAG | AUGCAUGAGACA | GACAGUA | AUGCAUGAC |
| | | | | UG (SEQ ID | GUGaaggaccuggc | (SEQ ID NO: | UU (SEQ ID |
| | | | | NO: 146) | (SEQ ID NO: 170) | 194) | NO: 218) |
| 28556 | HK2 | NM_000189.4 | 6985 | UGAGCACUC | cucaaauccuUGAGC | ACUCAGUC | UUCACUAGA |
| | | | | AGUCUAGUG | ACUCAGUCUAGU | UAGUGAA | CUGAGUGCU |
| | | | | AA (SEQ ID | GAAgauguugucau | (SEQ ID NO: | CA (SEQ ID |
| | | | | NO: 147) | (SEQ ID NO: 171) | 195) | NO: 219) |
| 28557 | HK2 | NM_000189.4 | 2187 | GACCAACUU | aucuuggaggGACCA | ACUUCCGU | UAAGCACAC |
| | | | | CCGUGUGCU | ACUUCCGUGUGC | GUGCUUA | GGAAGUUGG |
| | | | | UU (SEQ ID | UUUgggugaaagua | (SEQ ID NO: | UC (SEQ ID |
| | | | | NO: 148) | (SEQ ID NO: 172) | 196) | NO: 220) |
| 28558 | HK2 | NM_000189.4 | 4154 | AAAUGAUCA | agguucgagaAAAUG | AUCAGUGG | UUACAUUCC |
| | | | | GUGGAAUGU | AUCAGUGGAAUG | AAUGUAA | ACUGAUCAU |
| | | | | AC (SEQ ID | UACcugggugagau | (SEQ ID NO: | UU (SEQ ID |
| | | | | NO: 149) | (SEQ ID NO: 173) | 197) | NO: 221) |
| 28559 | HK2 | NM_000189.4 | 6982 | CCUUGAGCA | caucucaaauCCUUG | AGCACUCA | UCUAGACUG |
| | | | | CUCAGUCUA | AGCACUCAGUCU | GUCUAGA | AGUGCUCAA |
| | | | | GU (SEQ ID | AGUgaagauguugu | (SEQ ID NO: | GG (SEQ ID |
| | | | | NO: 150) | (SEQ ID NO: 174) | 198) | NO: 222) |
| 28560 | HK2 | NM_000189.4 | 4525 | CUACAUCCU | ccucuacaagCUACA | UCCUCACUU | UUGGCAAAG |
| | | | | CACUUUGCC | UCCUCACUUUGC | UGCCAA | UGAGGAUGU |
| | | | | AA (SEQ ID | CAAagucaugcaug | (SEQ ID NO: | AG (SEQ ID |
| | | | | NO: 151) | (SEQ ID NO: 175) | 199) | NO: 223) |
| 28561 | HK2 | NM_000189.4 | 3516 | CUUGGACCU | acuucuuggcCUUGG | ACCUUGGA | UUGUUCCUC |
| | | | | UGGAGGAAC | ACCUUGGAGGAA | GGAACAA | CAAGGUCCA |
| | | | | AA (SEQ ID | CAAauuuccggguc | (SEQ ID NO: | AG (SEQ ID |
| | | | | NO: 152) | (SEQ ID NO: 176) | 200) | NO: 224) |
| 28562 | HK2 | NM_000189.4 | 3586 | GAGAUGCAC | ggguggagugGAGAU | GCACAACA | UAGAUCUUG |
| | | | | AACAAGAUC | GCACAACAAGAU | AGAUCUA | UUGUGCAUC |
| | | | | UA (SEQ ID | CUAcgccaucccgc | (SEQ ID NO: | UC (SEQ ID |
| | | | | NO: 153) | (SEQ ID NO: 177) | 201) | NO: 225) |
| 28563 | HK2 | NM_000189.4 | 3905 | GAACUAUGA | gacacagucgGAACU | AUGAUGAC | UCCACAGGU |
| | | | | UGACCUGUG | AUGAUGACCUGU | CUGUGGA | CAUCAUAGU |
| | | | | GC (SEQ ID | GGCuuugaagaccc | (SEQ ID NO: | UC (SEQ ID |
| | | | | NO: 154) | (SEQ ID NO: 178) | 202) | NO: 226) |
| 28564 | HK2 | NM_000189.4 | 1985 | AGAAGGUUG | gaccaagugcAGAAG | GUUGACCA | UAGAUACUG |
| | | | | ACCAGUAUC | GUUGACCAGUAU | GUAUCUA | GUCAACCUU |
| | | | | UC (SEQ ID | CUCuaccacaugcg | (SEQ ID NO: | CU (SEQ ID |
| | | | | NO: 155) | (SEQ ID NO: 179) | 203) | NO: 227) |
| 28565 | HK2 | NM_000189.4 | 1987 | AAGGUUGAC | ccaagugcagAAGGU | UGACCAGU | UAGAGAUAC |
| | | | | CAGUAUCUC | UGACCAGUAUCU | AUCUCUA | UGGUCAACC |
| | | | | UA (SEQ ID | CUAccacaugcgcc | (SEQ ID NO: | UU (SEQ ID |
| | | | | NO: 156) | (SEQ ID NO: 180) | 204) | NO: 228) |
| 28566 | HK2 | NM_000189.4 | 2927 | UUGAGACCA | accggucgcuUUGAG | ACCAAAGA | UGAGAUGUC |
| | | | | AAGACAUCU | ACCAAAGACAUC | CAUCUCA | UUUGGUCUC |
| | | | | CA (SEQ ID | UCAgacauugaagg | (SEQ ID NO: | AA (SEQ ID |
| | | | | NO: 157) | (SEQ ID NO: 181) | 205) | NO: 229) |
| 28567 | HK2 | NM_000189.4 | 3164 | ACGGUUCCG | auuggggucgACGGU | UCCGUCUAC | UUUCUUGUA |
| | | | | UCUACAAGA | UCCGUCUACAAG | AAGAAA | GACGGAACC |
| | | | | AA (SEQ ID | AAAcacccccauuu | (SEQ ID NO: | GU (SEQ ID |
| | | | | NO: 158) | (SEQ ID NO: 182) | 206) | NO: 230) |
| 28568 | HK2 | NM_000189.4 | 1991 | UUGACCAGU | gugcagaaggUUGAC | CAGUAUCU | UUGGUAGAG |
| | | | | AUCUCUACC | CAGUAUCUCUAC | CUACCAA | AUACUGGUC |
| | | | | AC (SEQ ID | CACaugcgccucuc | (SEQ ID NO: | AA (SEQ ID |
| | | | | NO: 159) | (SEQ ID NO: 183) | 207) | NO: 231) |

TABLE 8
Cbl-b sd-rxRNA

| Cbl-b | | | |
|---|---|---|---|
| sd- | | | |
| rxRNA | | | SEQ |
| Duplex | Start | | ID |
| ID | Site | Sequence | NO: |
| CB 21 | 978 | fG.mA.fU.mU.fU.mA.fA.mC.fU.mU.fG.mC.fA*mA*fA-TEG- | 232 |
| | | Chl | |
| | | PmU.fU.mU.fG.mC.fA.mA.fG.mU.fU.mA.fA.mA.fU*mC*fA* | 233 |
| | | mA*fU*mU*fG | |
| CB 22 | 985 | fC.mU.fU.mG.fC.mA.fA.mU.fG.mA.fU.mU.fA*mC*fA-TEG- | 234 |
| | | Chl | |
| | | PmU.fG.mU.fA.mA.fU.mC.fA.mU.fU.mG.fC.mA.fA*mG*fU* | 235 |
| | | mU*fA*mA*fA | |
| CB 23 | 1124 | fU.mA.fA.mA.fG.mC.fA.mC.fG.mA.fC.mU.fA*mC*fA-TEG- | 236 |
| | | Chl | |
| | | PmU.fG.mU.fA.mG.fU.mC.fG.mU.fG.mC.fU.mU.fU*mA*fA* | 237 |
| | | mC*fU*mU*fC | |
| CB 24 | 1382 | fU.mA.fC.mA.fC.mA.fG.mG.fA.mA.fC.mA.fA*mU*fA-TEG- | 238 |
| | | Chl | |
| | | PmU.fA.mU.fU.mG.fU.mU.fC.mC.fU.mG.fU.mG.fU*mA*fA* | 239 |
| | | mC*fU*mU*fU | |
| CB 25 | 1550 | fU.mC.fG.mU.fU.mG.fU.mG.fA.mA.fA.mU.fA*mA*fA-TEG- | 240 |
| | | Chl | |
| | | PmU.fU.mU.fA.mU.fU.mU.fC.mA.fC.mA.fA.mC.fG*mA*fC* | 241 |
| | | mA*fG*mA*fA | |
| CB 26 | 1920 | fU.mG.fC.mA.fU.mG.fG.mU.fG.mA.fG.mA.fA*mA*fA-TEG- | 242 |
| | | Chl | |
| | | PmUSU.mU.fU.mCSU.mCSA.mCSC.mA.fU.mG.fC*mA*fA*mG*f | 243 |
| | | G*mA*fG | |
| CB 27 | 2517 | fC.mG.fG.mU.fC.mU.fU.mG.fU.mG.fA.mU.fA*mA*fA-TEG- | 244 |
| | | Chl | |
| | | PmU.fU.mU.fA.mU.fC.mA.fC.mA.fA.mG.fA.mC.fC*mG*fA* | 245 |
| | | mA*fC*mA*fG | |
| CB 28 | 2596 | fU.mA.fA.mG.fC.mA.fU.mA.fU.mA.fU.mU.fU*mA*fA-TEG- | 246 |
| | | Chl | |
| | | PmU.fU.mA.fA.mA.fU.mA.fU.mA.fU.mG.fC.mU.fU*mA*fA* | 247 |
| | | mG*fU*mC*fA | |
| CB 29 | 2813 | fC.mA.fU.mU.fG.mA.fA.mC.fA.mU.fU.mC.fA*mA*fA-TEG- | 248 |
| | | Chl | |
| | | PmU.fU.mU.fG.mA.fA.mU.fG.mU.fU.mC.fA.mA.fU*mG*fA* | 249 |
| | | mG*fA*mC*fU | |
| CB 30 | 3618 | fU.mU.fU.mG.fA.mU.fA.mC.fC.mU.fG.mU.fA*mC*fA-TEG- | 250 |
| | | Chl | |
| | | PmU.fG.mU.fA.mC.fA.mG.fG.mU.fA.mU.fC.mA.fA*mA*fA* | 251 |
| | | mC*fA*mC*fC | |
| CB 31 | 3818 | fG.mA.fU.mC.fA.mA.fA.mC.fU.mA.fA.mU.fG*mC*fA-TEG- | 252 |
| | | Chl | |
| | | PmUSG.mCSA.mUSU.mASG.mUSU.mUSG.mASU*mC*fA*mG* | 253 |
| | | fU*mU*fG | |
| CB 32 | 3925 | fU.mU.fA.mU.fU.mU.fG.mU.fC.mA.fA.mU.fA*mA*fA-TEG- | 254 |
| | | Chl | |
| | | PmU.fU.mU.fA.mU.fU.mG.fA.mC.fA.mA.fA.mU.fA*mA*fA* | 255 |
| | | mU*fG*mC*fU | |

Key
A = adenosine
G = guanosine
U = uridine
C = cytodine
m = 2′-O-methyl nucleotide
f = 2′ fluoro nucleotide
Y = 5 methyl uridine
X = 5 methyl cytodine
*= phosphorothioate linkage
.= phosphodiester linkage
TEG-Chl = cholesterol-TEG-Glyceryl
P = 5′ inorganic Phosphate

TABLE 9

| Oligo | | | Start | | | Passenger | Guide |
|---|---|---|---|---|---|---|---|
| ID | Gene | Accession number | Site | Sequence | Gene region | sequence | Sequence |
| 28359 | DNMT3A | NM_175629.2 | 1747 | UUAUUGAUG | gucaaggagaUUA | GAUGAGCG | UCUUGUGCG |
| | | | | AGCGCACAA | UUGAUGAGCG | CACAAGA | CUCAUCAAU |
| | | | | GA (SEQ ID | CACAAGAgagc | (SEQ ID NO: | AA (SEQ ID |
| | | | | NO: 256) | ggcuggu (SEQ ID | 304) | NO: 328) |
| | | | | | NO: 280) | | |
| 28360 | DNMT3A | NM_175629.2 | 1266 | AUUGUGUCU | gccaggccgcAUU | GUCUUGGU | UUCAUCCAC |
| | | | | UGGUGGAUG | GUGUCUUGGU | GGAUGAA | CAAGACACA |
| | | | | AC (SEQ ID | GGAUGACgggc | (SEQ ID NO: | AU (SEQ ID |
| | | | | NO: 257) | cggagcc (SEQ ID | 305) | NO: 329) |
| | | | | | NO: 281) | | |
| 28361 | DNMT3A | NM_175629.2 | 1428 | AUGUACC | GCcaagcagcccAUG | CCGCAAAG | UAGAUGGCU |
| | | | | AAAGCCAUC | UACCGCAAAG | CCAUCUA | UUGCGGUAC |
| | | | | UA (SEQ ID | CCAUCUAcgagg | (SEQ ID NO: | AU (SEQ ID |
| | | | | NO: 258) | uccugc (SEQ ID | 306) | NO: 330) |
| | | | | | NO: 282) | | |
| 28362 | DNMT3A | NM_175629.2 | 1988 | AAACAACAA | ucaugugcggAAA | ACAACUGC | UCCUGCAGC |
| | | | | CUGCUGCAG | CAACAACUGC | UGCAGGA | AGUUGUUGU |
| | | | | GU (SEQ ID | UGCAGGUgcuu | (SEQ ID NO: | UU (SEQ ID |
| | | | | NO: 259) | uugcgug (SEQ ID | 307) | NO: 331) |
| | | | | | NO: 283) | | |
| 28363 | DNMT3A | NM_175629.2 | 2411 | ACAGAAGCA | gcagcgucacACA | AGCAUAUC | UCUCCUGGA |
| | | | | UAUCCAGGA | GAAGCAUAUC | CAGGAGA | UAUGCUUCU |
| | | | | GU (SEQ ID | CAGGAGUgggg | (SEQ ID NO: | GU (SEQ ID |
| | | | | NO: 260) | cccauuc (SEQ ID | 308) | NO: 332) |
| | | | | | NO: 284) | | |
| 28364 | DNMT3A | NM_175629.2 | 2530 | UCUUUGAGU | ggccggcucuUCU | GAGUUCUA | UAGGCGGUA |
| | | | | UCUACCGCC | UUGAGUUCUA | CCGCCUA | GAACUCAAA |
| | | | | UC (SEQ ID | CCGCCUCcugca | (SEQ ID NO: | GA (SEQ ID |
| | | | | NO: 261) | ugaugc (SEQ ID | 309) | NO: 333) |
| | | | | | NO: 285) | | |
| 28365 | DNMT3A | NM_175629.2 | 3899 | UAAAAGGUA | gauauauauaUAA | GGUACUGU | UUAGUUAAC |
| | | | | CUGUUAACU | AAGGUACUGU | UAACUAA | AGUACCUUU |
| | | | | AC (SEQ ID | UAACUACugua | (SEQ ID NO: | UA (SEQ ID |
| | | | | NO: 262) | caacccg (SEQ ID | 310) | NO: 334) |
| | | | | | NO: 286) | | |
| 28366 | DNMT3A | NM_175629.2 | 3622 | CUGAUCAGA | ugucucuagcCUG | CAGAUAGG | UUGUGCUCC |
| | | | | UAGGAGCAC | AUCAGAUAGG | AGCACAA | UAUCUGAUC |
| | | | | AA (SEQ ID | AGCACAAgcag | (SEQ ID NO: | AG (SEQ ID |
| | | | | NO: 263) | gggacgg (SEQ ID | 311) | NO: 335) |
| | | | | | NO: 287) | | |
| 28367 | DNMT3A | NM_175629.2 | 2913 | UUAUGGUGC | agaggacaucUUA | GUGCACUG | UCCAUUUCA |
| | | | | ACUGAAAUG | UGGUGCACUG | AAAUGGA | GUGCACCAU |
| | | | | GA (SEQ ID | AAAUGGAaagg | (SEQ ID NO: | AA (SEQ ID |
| | | | | NO: 264) | guauuug (SEQ ID | 312) | NO: 336) |
| | | | | | NO: 288) | | |
| 28368 | DNMT3A | NM_175629.2 | 2821 | GCAAAGUGA | gccaaguucaGCA | GUGAGGAC | UGUAAUGGU |
| | | | | GGACCAUUA | AAGUGAGGAC | CAUUACA | CCUCACUUU |
| | | | | CU (SEQ ID | CAUUACUacgag | (SEQ ID NO: | GC (SEQ ID |
| | | | | NO: 265) | gucaaa (SEQ ID | 313) | NO: 337) |
| | | | | | NO: 289) | | |
| 28369 | DNMT3A | NM_175629.2 | 3843 | CGCUGUUAC | uucuagaagcCGC | UUACCUCU | UUAAACAAG |
| | | | | CUCUUGUUU | UGUUACCUCU | UGUUUAA | AGGUAACAG |
| | | | | AC (SEQ ID | UGUUUACaguu | (SEQ ID NO: | CG (SEQ ID |
| | | | | NO: 266) | uauauau (SEQ ID | 314) | NO: 338) |
| | | | | | NO: 290) | | |
| 28370 | DNMT3A | NM_175629.2 | 3804 | CCACACAGG | caggugccuaCCA | CAGGAAAC | UUUCAAGGU |
| | | | | AAACCUUGA | CACAGGAAAC | CUUGAAA | UUCCUGUGU |
| | | | | AG (SEQ ID | CUUGAAGaaaau | (SEQ ID NO: | GG (SEQ ID |
| | | | | NO: 267) | caguuu (SEQ ID | 315) | NO: 339) |
| | | | | | NO: 291) | | |
| 28371 | DNMT3A | NM_175629.2 | 3418 | CUUGCUGUG | gguuuuguuuCUU | UGUGACUG | UUUGUUUCA |
| | | | | ACUGAAACA | GCUGUGACUG | AAACAAA | GUCACAGCA |
| | | | | AG (SEQ ID | AAACAAGaagg | (SEQ ID NO: | AG (SEQ ID |
| | | | | NO: 268) | uuauugc (SEQ ID | 316) | NO: 340) |
| | | | | | NO: 292) | | |
| 28372 | DNMT3A | NM_175629.2 | 2670 | GUGAUGAUU | guccaacccuGUG | GAUUGAUG | UCUUUGGCA |
| | | | | GAUGCCAAA | AUGAUUGAUG | CCAAAGA | UCAAUCAUC |
| | | | | GA (SEQ ID | CCAAAGAagug | (SEQ ID NO: | AC (SEQ ID |
| | | | | NO: 269) | ucagcug (SEQ ID | 317) | NO: 341) |
| | | | | | NO: 293) | | |
| 28373 | DNMT3A | NM_175629.2 | 2169 | AAUAACCAC | guucuucgcuAAU | CCACGACC | UAUUCCUGG |
| | | | | GACCAGGAA | AACCACGACC | AGGAAUA | UCGUGGUUA |
| | | | | UU (SEQ ID | AGGAAUUugac | (SEQ ID NO: | UU (SEQ ID |
| | | | | NO: 270) | ccuccaa (SEQ ID | 318) | NO: 342) |
| | | | | | NO: 294) | | |
| 28374 | DNMT3A | NM_175629.2 | 1386 | UUUUGCAGU | gcugagcucgUUU | CAGUGCGU | UGGUGGAAC |
| | | | | GCGUUCCAC | UGCAGUGCGU | UCCACCA | GCACUGCAA |
| | | | | CA (SEQ ID | UCCACCAggcca | (SEQ ID NO: | AA (SEQ ID |
| | | | | NO271) | cguaca (SEQ ID | 319) | NO: 343) |
| | | | | | NO: 295) | | |
| 28375 | DNMT3A | NM_175629.2 | 1935 | UACCAGUCC | cgacgacggcUAC | GUCCUACU | UUGGUGCAG |
| | | | | UACUGCACC | CAGUCCUACU | GCACCAA | UAGGACUGG |
| | | | | AU (SEQ ID | GCACCAUcugcu | (SEQ ID NO: | UA (SEQ ID |
| | | | | NO: 272) | gugggg (SEQ ID | 320) | NO: 344) |
| | | | | | NO: 296) | | |
| 28376 | DNMT3A | NM_175629.2 | 2302 | UUCAGGUGG | gacuugggcaUUC | GUGGACCG | UAUGUAGCG |
| | | | | ACCGCUACA | AGGUGGACCG | CUACAUA | GUCCACCUG |
| | | | | UU (SEQ ID | CUACAUUgccuc | (SEQ ID NO: | AA (SEQ ID |
| | | | | NO: 273) | ggaggu (SEQ ID | 321) | NO: 345) |
| | | | | | NO: 297) | | |
| 28377 | DNMT3A | NM_175629.2 | 2642 | CAUCUCGCG | acaagagggaCAU | CGCGAUUU | UCUCGAGAA |
| | | | | AUUUCUCGA | CUCGCGAUUU | CUCGAGA | AUCGCGAGA |
| | | | | GU (SEQ ID | CUCGAGUccaac | (SEQ ID NO: | UG (SEQ ID |
| | | | | NO: 274) | ccugug (SEQ ID | 322) | NO: 346) |
| | | | | | NO: 298) | | |
| 28378 | DNMT3A | NM_175629.2 | 3046 | CUCCGCUGA | caccucuucgCUC | CUGAAGGA | UAAAUACUC |
| | | | | AGGAGUAUU | CGCUGAAGGA | GUAUUUA | CUUCAGCGG |
| | | | | UU (SEQ ID | GUAUUUUgcgu | (SEQ ID NO: | AG (SEQ ID |
| | | | | NO: 275) | gugugua (SEQ ID | 323) | NO: 347) |
| | | | | | NO: 299) | | |
| 28379 | DNMT3A | NM_175629.2 | 2154 | CAGAUGUUC | cucccggcucCAG | GUUCUUCG | UUAUUAGCG |
| | | | | UUCGCUAAU | AUGUUCUUCG | CUAAUAA | AAGAACAUC |
| | | | | AA (SEQ ID | CUAAUAAccacg | (SEQ ID NO: | UG (SEQ ID |
| | | | | NO: 276) | accagg (SEQ ID | 324) | NO: 348) |
| | | | | | NO: 300) | | |
| 28380 | DNMT3A | NM_175629.2 | 1874 | AAUGUGCCA | ucguuggaggAAU | GCCAAAAC | UCUUGCAGU |
| | | | | AAACUGCAA | GUGCCAAAAC | UGCAAGA | UUUGGCACA |
| | | | | GA (SEQ ID | UGCAAGAacug | (SEQ ID NO: | UU (SEQ ID |
| | | | | NO: 277) | cuuucug (SEQ ID | 325) | NO: 349) |
| | | | | | NO: 301) | | |
| 28381 | DNMT3A | NM_175629.2 | 1329 | UUCGGAGAC | ggucauguggUUC | AGACGGCA | UAGAAUUUG |
| | | | | GGCAAAUUC | GGAGACGGCA | AAUUCUA | CCGUCUCCG |
| | | | | UC (SEQ ID | AAUUCUCagug | (SEQ ID NO: | AA (SEQ ID |
| | | | | NO: 278) | gugugug (SEQ ID | 326) | NO: 350) |
| | | | | | NO: 302) | | |
| 28382 | DNMT3A | NM_175629.2 | 1326 | UGGUUCGGA | cugggucaugUGG | CGGAGACG | UAUUUGCCG |
| | | | | GACGGCAAA | UUCGGAGACG | GCAAAUA | UCUCCGAAC |
| | | | | UU (SEQ ID | GCAAAUUcuca | (SEQ ID NO: | CA (SEQ ID |
| | | | | NO: 279) | guggugu (SEQ ID | 327) | NO: 351) |
| | | | | | NO: 303) | | |

TABLE 10

| Oligo | | | Start | | | Passenger | Guide |
|---|---|---|---|---|---|---|---|
| ID | Gene | Accession number | Site | Sequence | Gene region | sequence | Sequence |
| 28569 | PRDM1 | NM_001198.3 | 970 | AGAGAGUAC | ugucccaaagAGA | GUACAGCG | UCUUUCAC |
| | | | | AGCGUGAAA | GAGUACAGCG | UGAAAGA | GCUGUACU |
| | | | | GA (SEQ ID | UGAAAGAaauc | (SEQ ID NO: | CUCU (SEQ |
| | | | | NO: 352) | cuaaaau (SEQ ID | 400) | ID NO: 424) |
| | | | | | NO: 376) | | |
| 28570 | PRDM1 | NM_001198.3 | 972 | AGAGUACAG | ucccaaagagAGA | ACAGCGUG | UUUCUUUC |
| | | | | CGUGAAAGA | GUACAGCGUG | AAAGAAA | ACGCUGUA |
| | | | | AA (SEQ ID | AAAGAAAuccu | (SEQ ID NO: | CUCU (SEQ |
| | | | | NO: 353) | aaaauug (SEQ ID | 401) | ID NO: 425) |
| | | | | | NO: 377) | | |
| 28571 | PRDM1 | NM_001198.3 | 815 | AGGAACUUC | ccugccaaccAGG | CUUCUUGU | UUACCACAC |
| | | | | UUGUGUGGU | AACUUCUUGU | GUGGUAA | AAGAAGUU |
| | | | | AU (SEQ ID | GUGGUAUuguc | (SEQ ID NO: | CCU (SEQ ID |
| | | | | NO: 354) | gggacuu (SEQ ID | 402) | NO: 426) |
| | | | | | NO: 378) | | |
| 28572 | PRDM1 | NM_001198.3 | 2135 | AGGUCUGCC | caugaaugccAGG | UGCCACAA | UAAUCUCU |
| | | | | ACAAGAGAU | UCUGCCACAA | GAGAUUA | UGUGGCAG |
| | | | | UU (SEQ ID | GAGAUUUagca | (SEQ ID NO: | ACCU (SEQ |
| | | | | NO: 355) | gcaccag (SEQ ID | 403) | ID NO: 427) |
| | | | | | NO: 379) | | |
| 28573 | PRDM1 | NM_001198.3 | 2137 | GUCUGCCAC | ugaaugccagGUC | CCACAAGA | UUAAAUCU |
| | | | | AAGAGAUUU | UGCCACAAGA | GAUUUAA | CUUGUGGC |
| | | | | AG (SEQ ID | GAUUUAGcagc | (SEQ ID NO: | AGAC (SEQ |
| | | | | NO: 356) | accagca (SEQ ID | 404) | ID NO: 428) |
| | | | | | NO: 380) | | |
| 28574 | PRDM1 | NM_001198.3 | 4266 | ACCACUUAA | uauauuuauaACC | UUAAAUUG | UGGCUCAC |
| | | | | AUUGUGAGC | ACUUAAAUUG | UGAGCCA | AAUUUAAG |
| | | | | CA (SEQ ID | UGAGCCAagcca | (SEQ ID NO: | UGGU (SEQ |
| | | | | NO: 357) | uguaaa (SEQ ID | 405) | ID NO: 429) |
| | | | | | NO: 381) | | |
| 28575 | PRDM1 | NM_001198.3 | 4276 | UUGUGAGCC | accacuuaaaUUG | AGCCAAGC | UUACAUGG |
| | | | | AAGCCAUGU | UGAGCCAAGC | CAUGUAA | CUUGGCUC |
| | | | | AA (SEQ ID | CAUGUAAaaga | (SEQ ID NO: | ACAA (SEQ |
| | | | | NO: 358) | ucuacuu (SEQ ID | 406) | ID NO: 430) |
| | | | | | NO: 382) | | |
| 28576 | PRDM1 | NM_001198.3 | 2669 | CUGUAAAGG | ccucugguacCUG | AAGGUCAA | UUCUUGUU |
| | | | | UCAAACAAG | UAAAGGUCAA | ACAAGAA | UGACCUUU |
| | | | | AA (SEQ ID | ACAAGAAacag | (SEQ ID NO: | ACAG (SEQ |
| | | | | NO: 359) | uugaacc (SEQ ID | 407) | ID NO: 431) |
| | | | | | NO: 383) | | |
| 28577 | PRDM1 | NM_001198.3 | 5052 | UUUACUUUG | uuacuggcuuUUU | UUUGCUAG | UGUUGUUC |
| | | | | CUAGAACAA | ACUUUGCUAG | AACAACA | UAGCAAAG |
| | | | | CA (SEQ ID | AACAACAaacua | (SEQ ID NO: | UAAA (SEQ |
| | | | | NO: 360) | ucuuau (SEQ ID | 408) | ID NO: 432) |
| | | | | | NO: 384) | | |
| 28578 | PRDM1 | NM_001198.3 | 5055 | ACUUUGCUA | cuggcuuuuuACU | GCUAGAAC | UUUUGUUG |
| | | | | GAACAACAA | UUGCUAGAAC | AACAAAA | UUCUAGCA |
| | | | | AC (SEQ ID | AACAAACuauc | (SEQ ID NO: | AAGU (SEQ |
| | | | | NO: 361) | uuauguu (SEQ ID | 409) | ID NO: 433) |
| | | | | | NO: 385) | | |
| 28579 | PRDM1 | NM_001198.3 | 968 | AGAGAGAGU | aaugucccaaAGA | GAGUACAG | UUUCACGC |
| | | | | ACAGCGUGA | GAGAGUACAG | CGUGAAA | UGUACUCU |
| | | | | AA (SEQ ID | CGUGAAAgaaa | (SEQ ID NO: | CUCU (SEQ |
| | | | | NO: 362) | uccuaaa (SEQ ID | 410) | ID NO: 434) |
| | | | | | NO: 386) | | |
| 28580 | PRDM1 | NM_001198.3 | 771 | GAUGAACAU | gucagaacggGAU | ACAUCUAC | UGUAGAAG |
| | | | | CUACUUCUA | GAACAUCUAC | UUCUACA | UAGAUGUU |
| | | | | CA (SEQ ID | UUCUACAccauu | (SEQ ID NO: | CAUC (SEQ |
| | | | | NO: 363) | aagccc (SEQ ID | 411) | ID NO: 435) |
| | | | | | NO: 387) | | |
| 28581 | PRDM1 | NM_001198.3 | 819 | ACUUCUUGU | ccaaccaggaACU | UUGUGUGG | UACAAUAC |
| | | | | GUGGUAUUG | UCUUGUGUGG | UAUUGUA | CACACAAG |
| | | | | UC (SEQ ID | UAUUGUCggga | (SEQ ID NO: | AAGU (SEQ |
| | | | | NO: 364) | cuuugca (SEQ ID | 412) | ID NO: 436) |
| | | | | | NO: 388) | | |
| 28582 | PRDM1 | NM_001198.3 | 1867 | GAAGCCAUG | cagcagcgacGAA | CAUGAAUC | UUAAUGAG |
| | | | | AAUCUCAUU | GCCAUGAAUC | UCAUUAA | AUUCAUGG |
| | | | | AA (SEQ ID | UCAUUAAaaaca | (SEQ ID NO: | CUUC (SEQ |
| | | | | NO: 365) | aaagaa (SEQ ID | 413) | ID NO: 437) |
| | | | | | NO: 389) | | |
| 28583 | PRDM1 | NM_001198.3 | 3117 | AAAGUUUAC | aauaauuaaaAAA | UUACAAUG | UUCCAGUC |
| | | | | AAUGACUGG | GUUUACAAUG | ACUGGAA | AUUGUAAA |
| | | | | AA (SEQ ID | ACUGGAAagau | (SEQ ID NO: | CUUU (SEQ |
| | | | | NO: 366) | uccuugu (SEQ ID | 414) | ID NO: 438) |
| | | | | | NO: 390) | | |
| 28584 | PRDM1 | NM_001198.3 | 1999 | AAUCUGAAG | ccagcucuccAAU | GAAGGUCC | UUCAGGUG |
| | | | | GUCCACCUG | CUGAAGGUCC | ACCUGAA | GACCUUCA |
| | | | | AG (SEQ ID | ACCUGAGagug | (SEQ ID NO: | GAUU (SEQ |
| | | | | NO: 367) | cacagug (SEQ ID | 415) | ID NO: 439) |
| | | | | | NO: 391) | | |
| 28585 | PRDM1 | NM_001198.3 | 2027 | GUGGAGAAC | agagugcacaGUG | GAACGGCC | UUUGAAAG |
| | | | | GGCCUUUCA | GAGAACGGCC | UUUCAAA | GCCGUUCUC |
| | | | | AA (SEQ ID | UUUCAAAuguc | (SEQ ID NO: | CAC (SEQ ID |
| | | | | NO: 368) | agacuug (SEQ ID | 416) | NO: 440) |
| | | | | | NO: 392) | | |
| 28586 | PRDM1 | NM_001198.3 | 3494 | AAGGCUUUA | gggugacaggAAG | UUUACCAA | UGACAGGU |
| | | | | CCAACCUGU | GCUUUACCAA | CCUGUCA | UGGUAAAG |
| | | | | CU (SEQ ID | CCUGUCUcuccc | (SEQ ID NO: | CCUU (SEQ |
| | | | | NO: 369) | uccaaa (SEQ ID | 417) | ID NO: 441) |
| | | | | | NO: 393) | | |
| 28587 | PRDM1 | NM_001198.3 | 699 | AAGCAACUG | augaagagaaAAG | ACUGGAUG | UAUAGCGC |
| | | | | GAUGCGCUA | CAACUGGAUG | CGCUAUA | AUCCAGUU |
| | | | | UG (SEQ ID | CGCUAUGugaa | (SEQ ID NO: | GCUU (SEQ |
| | | | | NO: 370) | uccagca (SEQ ID | 418) | ID NO: 442) |
| | | | | | NO: 394) | | |
| 28588 | PRDM1 | NM_001198.3 | 2335 | AGCCUCAAG | ccaucucuguAGC | CAAGGUUC | UUCAGGUG |
| | | | | GUUCACCUG | CUCAAGGUUC | ACCUGAA | AACCUUGA |
| | | | | AA (SEQ ID | ACCUGAAaggg | (SEQ ID NO: | GGCU (SEQ |
| | | | | NO: 371) | aacugcg (SEQ ID | 419) | ID NO: 443) |
| | | | | | NO: 395) | | |
| 28589 | PRDM1 | NM_001198.3 | 2314 | AAGAACUAC | ccagugccacAAG | CUACAUCC | UAGAGAUG |
| | | | | AUCCAUCUC | AACUACAUCC | AUCUCUA | GAUGUAGU |
| | | | | UG (SEQ ID | AUCUCUGuagcc | (SEQ ID NO: | UCUU (SEQ |
| | | | | NO: 372) | ucaagg (SEQ ID | 420) | ID NO: 444) |
| | | | | | NO: 396) | | |
| 28590 | PRDM1 | NM_001198.3 | 2246 | UUGUGCACC | uucacccaguUUG | CACCUGAA | UUGCAGUU |
| | | | | UGAAACUGC | UGCACCUGAA | ACUGCAA | UCAGGUGC |
| | | | | AC (SEQ ID | ACUGCACaagcg | (SEQ ID NO: | ACAA (SEQ |
| | | | | NO: 373) | ucugca (SEQ ID | 421) | ID NO: 445) |
| | | | | | NO: 397) | | |
| 28591 | PRDM1 | NM_001198.3 | 2000 | AUCUGAAGG | cagcucuccaAUC | AAGGUCCA | UCUCAGGU |
| | | | | UCCACCUGA | UGAAGGUCCA | CCUGAGA | GGACCUUC |
| | | | | GA (SEQ ID | CCUGAGAgugc | (SEQ ID NO: | AGAU (SEQ |
| | | | | NO: 374) | acagugg (SEQ ID | 422) | ID NO: 446) |
| | | | | | NO: 398) | | |
| 28592 | PRDM1 | NM_001198.3 | 1939 | CAGAACGGC | gcugaagaagCAG | CGGCAAGA | UACUUGAU |
| | | | | AAGAUCAAG | AACGGCAAGA | UCAAGUA | CUUGCCGU |
| | | | | UA (SEQ ID | UCAAGUAcgaa | (SEQ ID NO: | UCUG (SEQ |
| | | | | NO: 375) | ugcaacg (SEQ ID | 423) | ID NO: 447) |
| | | | | | NO: 399) | | |

TABLE 11

| Oligo | | | Start | | | Passenger | Guide |
|---|---|---|---|---|---|---|---|
| ID | Gene | Accession number | Site | Sequence | Gene region | sequence | Sequence |
| 28606 | PTPN6 | NM_080549.3 | 1057 | GCAAGAACC | gagaacaaggGCA | AACCGCUA | UUUCUUGU |
| | | | | GCUACAAGA | AGAACCGCUA | CAAGAAA | AGCGGUUC |
| | | | | AC (SEQ ID | CAAGAACauuc | (SEQ ID NO: | UUGC (SEQ |
| | | | | NO: 448) | uccccuu (SEQ ID | 496) | ID NO: 520) |
| | | | | | NO: 472) | | |
| 28607 | PTPN6 | NM_080549.3 | 1059 | AAGAACCGC | gaacaagggcAAG | CCGCUACA | UUGUUCUU |
| | | | | UACAAGAAC | AACCGCUACA | AGAACAA | GUAGCGGU |
| | | | | AU (SEQ ID | AGAACAUucuc | (SEQ ID NO: | UCUU (SEQ |
| | | | | NO: 449) | cccuuug (SEQ ID | 497) | ID NO: 521) |
| | | | | | NO: 473) | | |
| 28608 | PTPN6 | NM_080549.3 | 1305 | GAGAAAGGC | ccgagaggugGAG | AGGCCGGA | UAUUUGUU |
| | | | | CGGAACAAA | AAAGGCCGGA | ACAAAUA | CCGGCCUUU |
| | | | | UG (SEQ ID | ACAAAUGcguc | (SEQ ID NO: | CUC (SEQ ID |
| | | | | NO: 450) | ccauacu (SEQ ID | 498) | NO: 522) |
| | | | | | NO: 474) | | |
| 28609 | PTPN6 | NM_080549.3 | 1303 | UGGAGAAAG | acccgagaggUGG | AAAGGCCG | UUUGUUCC |
| | | | | GCCGGAACA | AGAAAGGCCG | GAACAAA | GGCCUUUC |
| | | | | AA (SEQ ID | GAACAAAugcg | (SEQ ID NO: | UCCA (SEQ |
| | | | | NO: 451) | ucccaua (SEQ ID | 499) | ID NO: 523) |
| | | | | | NO: 475) | | |
| 28610 | PTPN6 | NM_080549.3 | 393 | CAUAUUCGG | ucaggugaccCAU | UCGGAUCC | UAGUUCUG |
| | | | | AUCCAGAAC | AUUCGGAUCC | AGAACUA | GAUCCGAA |
| | | | | UC (SEQ ID | AGAACUCaggg | (SEQ ID NO: | UAUG (SEQ |
| | | | | NO: 452) | gauuucu (SEQ ID | 500) | ID NO: 524) |
| | | | | | NO: 476) | | |
| 28611 | PTPN6 | NM_080549.3 | 395 | UAUUCGGAU | aggugacccaUAU | GGAUCCAG | UUGAGUUC |
| | | | | CCAGAACUC | UCGGAUCCAG | AACUCAA | UGGAUCCG |
| | | | | AG (SEQ ID | AACUCAGggga | (SEQ ID NO: | AAUA (SEQ |
| | | | | NO: 453) | uuucuau (SEQ ID | 501) | ID NO: 525) |
| | | | | | NO: 477) | | |
| 28612 | PTPN6 | NM_080549.3 | 1239 | AAUGACUUC | ggccacggucAAU | CUUCUGGC | UCCAUCUGC |
| | | | | UGGCAGAUG | GACUUCUGGC | AGAUGGA | CAGAAGUC |
| | | | | GC (SEQ ID | AGAUGGCgugg | (SEQ ID NO: | AUU (SEQ ID |
| | | | | NO: 454) | caggaga (SEQ ID | 502) | NO: 526) |
| | | | | | NO: 478) | | |
| 28613 | PTPN6 | NM_080549.3 | 1140 | GACUACAUC | ccccggguccGAC | CAUCAAUG | UAGUUGGC |
| | | | | AAUGCCAAC | UACAUCAAUG | CCAACUA | AUUGAUGU |
| | | | | UA (SEQ ID | CCAACUAcauca | (SEQ ID NO: | AGUC (SEQ |
| | | | | NO: 455) | agaacc (SEQ ID | 503) | ID NO: 527) |
| | | | | | NO: 479) | | |
| 28614 | PTPN6 | NM_080549.3 | 1060 | AGAACCGCU | aacaagggcaAGA | CGCUACAA | UAUGUUCU |
| | | | | ACAAGAACA | ACCGCUACAA | GAACAUA | UGUAGCGG |
| | | | | UU (SEQ ID | GAACAUUcuccc | (SEQ ID NO: | UUCU (SEQ |
| | | | | NO: 456) | cuuuga (SEQ ID | 504) | ID NO: 528) |
| | | | | | NO: 480) | | |
| 28615 | PTPN6 | NM_080549.3 | 1473 | CAUUACCAG | ggagaucuggCAU | CCAGUACC | UAGCUCAG |
| | | | | UACCUGAGC | UACCAGUACC | UGAGCUA | GUACUGGU |
| | | | | UG (SEQ ID | UGAGCUGgccc | (SEQ ID NO: | AAUG (SEQ |
| | | | | NO: 457) | gaccaug (SEQ ID | 505) | ID NO: 529) |
| | | | | | NO: 481) | | |
| 28616 | PTPN6 | NM_080549.3 | 1086 | UUUGACCAC | cauucuccccUUU | CCACAGCC | UUCACUCG |
| | | | | AGCCGAGUG | GACCACAGCC | GAGUGAA | GCUGUGGU |
| | | | | AU (SEQ ID | GAGUGAUccug | (SEQ ID NO: | CAAA (SEQ |
| | | | | NO: 458) | cagggac (SEQ ID | 506) | ID NO: 530) |
| | | | | | NO: 482) | | |
| 28617 | PTPN6 | NM_080549.3 | 1690 | ACAUCCAGA | ugugacauugACA | CAGAAGAC | UUGGAUGG |
| | | | | AGACCAUCC | UCCAGAAGAC | CAUCCAA | UCUUCUGG |
| | | | | AG (SEQ ID | CAUCCAGaugg | (SEQ ID NO: | AUGU (SEQ |
| | | | | NO: 459) | ugcgggc (SEQ ID | 507) | ID NO: 531) |
| | | | | | NO: 483) | | |
| 28618 | PTPN6 | NM_080549.3 | 1470 | UGGCAUUAC | ucgggagaucUGG | UUACCAGU | UUCAGGUA |
| | | | | CAGUACCUG | CAUUACCAGU | ACCUGAA | CUGGUAAU |
| | | | | AG (SEQ ID | ACCUGAGcugg | (SEQ ID NO: | GCCA (SEQ |
| | | | | NO: 460) | cccgacc (SEQ ID | 508) | ID NO: 532) |
| | | | | | NO: 484) | | |
| 28619 | PTPN6 | NM_080549.3 | 1188 | GAGAACGCU | aggcccugauGAG | CGCUAAGA | UUGUAGGU |
| | | | | AAGACCUAC | AACGCUAAGA | CCUACAA | CUUAGCGU |
| | | | | AU (SEQ ID | CCUACAUcgcca | (SEQ ID NO: | UCUC (SEQ |
| | | | | NO: 461) | gccagg (SEQ ID | 509) | ID NO: 533) |
| | | | | | NO: 485) | | |
| 28620 | PTPN6 | NM_080549.3 | 1191 | AACGCUAAG | cccugaugagAAC | UAAGACCU | UCGAUGUA |
| | | | | ACCUACAUC | GCUAAGACCU | ACAUCGA | GGUCUUAG |
| | | | | GC (SEQ ID | ACAUCGCcagcc | (SEQ ID NO: | CGUU (SEQ |
| | | | | NO: 462) | agggcu (SEQ ID | 510) | ID NO: 534) |
| | | | | | NO: 486) | | |
| 28621 | PTPN6 | NM_080549.3 | 1755 | UACAAGUUC | ggaggcgcagUAC | GUUCAUCU | UCCACGUA |
| | | | | AUCUACGUG | AAGUUCAUCU | ACGUGGA | GAUGAACU |
| | | | | GC (SEQ ID | ACGUGGCcaucg | (SEQ ID NO: | UGUA (SEQ |
| | | | | NO: 463) | cccagu (SEQ ID | 511) | ID NO: 535) |
| | | | | | NO: 487) | | |
| 28622 | PTPN6 | NM_080549.3 | 1393 | AGCAUGACA | aacugcggggAGC | GACACAAC | UUAUUCGG |
| | | | | CAACCGAAU | AUGACACAAC | CGAAUAA | UUGUGUCA |
| | | | | AC (SEQ ID | CGAAUACaaacu | (SEQ ID NO: | UGCU (SEQ |
| | | | | NO: 464) | ccguac (SEQ ID | 512) | ID NO: 536) |
| | | | | | NO: 488) | | |
| 28623 | PTPN6 | NM_080549.3 | 2060 | CACAAGGAG | cggcugcagaCAC | GGAGGAUG | UCAUACAC |
| | | | | GAUGUGUAU | AAGGAGGAUG | UGUAUGA | AUCCUCCUU |
| | | | | GA (SEQ ID | UGUAUGAgaac | (SEQ ID NO: | GUG (SEQ ID |
| | | | | NO: 465) | cugcaca (SEQ ID | 513) | NO: 537) |
| | | | | | NO: 489) | | |
| 28624 | PTPN6 | NM_080549.3 | 894 | GUGAAUGCG | ugccacgaggGUG | UGCGGCUG | UCAAUGUC |
| | | | | GCUGACAUU | AAUGCGGCUG | ACAUUGA | AGCCGCAU |
| | | | | GA (SEQ ID | ACAUUGAgaacc | (SEQ ID NO: | UCAC (SEQ |
| | | | | NO: 466) | gagugu (SEQ ID | 514) | ID NO: 538) |
| | | | | | NO: 490) | | |
| 28625 | PTPN6 | NM_080549.3 | 739 | ACAUCAAGG | agggucacccACA | AAGGUCAU | UUCGCACA |
| | | | | UCAUGUGCG | UCAAGGUCAU | GUGCGAA | UGACCUUG |
| | | | | AG (SEQ ID | GUGCGAGggug | (SEQ ID NO: | AUGU (SEQ |
| | | | | NO: 467) | gacgcua (SEQ ID | 515) | ID NO: 539) |
| | | | | | NO: 491) | | |
| 28626 | PTPN6 | NM_080549.3 | 1746 | GAGGCGCAG | ggugcagacgGAG | GCAGUACA | UUGAACUU |
| | | | | UACAAGUUC | GCGCAGUACA | AGUUCAA | GUACUGCG |
| | | | | AU (SEQ ID | AGUUCAUcuac | (SEQ ID NO: | CCUC (SEQ |
| | | | | NO: 468) | guggcca (SEQ ID | 516) | ID NO: 540) |
| | | | | | NO: 492) | | |
| 28627 | PTPN6 | NM_080549.3 | 910 | UUGAGAACC | gcggcugacaUUG | AACCGAGU | UUCCAACAC |
| | | | | GAGUGUUGG | AGAACCGAGU | GUUGGAA | UCGGUUCU |
| | | | | AA (SEQ ID | GUUGGAAcuga | (SEQ ID NO: | CAA (SEQ ID |
| | | | | NO: 469) | acaagaa (SEQ ID | 517) | NO: 541) |
| | | | | | NO: 493) | | |
| 28628 | PTPN6 | NM_080549.3 | 2222 | CAUUUCGCG | ccuguggaagCAU | CGCGAUGG | UGUCUGUC |
| | | | | AUGGACAGA | UUCGCGAUGG | ACAGACA | CAUCGCGA |
| | | | | CU (SEQ ID | ACAGACUcacaa | (SEQ ID NO: | AAUG (SEQ |
| | | | | NO: 470) | ccugaa (SEQ ID | 518) | ID NO: 542) |
| | | | | | NO: 494) | | |
| 28629 | PTPN6 | NM_080549.3 | 633 | UGGACGUUU | gggcgagcccUGG | GUUUCUUG | UCACGCACA |
| | | | | CUUGUGCGU | ACGUUUCUUG | UGCGUGA | AGAAACGU |
| | | | | GA (SEQ ID | UGCGUGAgagc | (SEQ ID NO: | CCA (SEQ ID |
| | | | | NO: 471) | cucagcc (SEQ ID | 519) | NO: 543) |
| | | | | | NO: 495) | | |

TABLE 12

| Oligo | | | Start | | | Passenger | Guide |
|---|---|---|---|---|---|---|---|
| ID | Gene | Accession number | Site | Sequence | Gene region | sequence | Sequence |
| 28317 | TET2 | NM_001127208.2 | 1104 | UAAUGCCU | aaggcagugcUAA | CCUAAUGG | UGUAGCAC |
| | | | | AAUGGUGC | UGCCUAAUGG | UGCUACA | CAUUAGGC |
| | | | | UACA (SEQ | UGCUACAguuu | (SEQ ID NO: | AUUA (SEQ |
| | | | | ID NO: 544) | cugccuc (SEQ ID | 592) | ID NO: 616) |
| | | | | | NO: 568) | | |
| 28318 | TET2 | NM_001127208.2 | 3551 | AAGAGCAU | ugugcagcaaAAG | CAUCAUUG | UUGGUCUC |
| | | | | CAUUGAGA | AGCAUCAUUG | AGACCAA | AAUGAUGC |
| | | | | CCAU (SEQ | AGACCAUggag | (SEQ ID NO: | UCUU (SEQ |
| | | | | ID NO: 545) | cagcauc (SEQ ID | 593) | ID NO: 617) |
| | | | | | NO: 569) | | |
| 28319 | TET2 | NM_001127208.2 | 1107 | UGCCUAAU | gcagugcuaaUGC | AAUGGUGC | UACUGUAG |
| | | | | GGUGCUAC | CUAAUGGUGC | UACAGUA | CACCAUUA |
| | | | | AGUU (SEQ | UACAGUUucug | (SEQ ID NO: | GGCA (SEQ |
| | | | | ID NO: 546) | ccucuuc (SEQ ID | 594) | ID NO: 618) |
| | | | | | NO: 570) | | |
| 28320 | TET2 | NM_001127208.2 | 3554 | AGCAUCAU | gcagcaaaagAGC | CAUUGAGA | UCCAUGGU |
| | | | | UGAGACCA | AUCAUUGAGA | CCAUGGA | CUCAAUGA |
| | | | | UGGA (SEQ | CCAUGGAgcagc | (SEQ ID NO: | UGCU (SEQ |
| | | | | ID NO: 547) | aucuga (SEQ ID | 595) | ID NO: 619) |
| | | | | | NO: 571) | | |
| 28321 | TET2 | NM_001127208.2 | 477 | AAGCAAGC | gauggccccgAAG | AGCCUGAU | UUGUUCCA |
| | | | | CUGAUGGA | CAAGCCUGAU | GGAACAA | UCAGGCUU |
| | | | | ACAG (SEQ | GGAACAGgaua | (SEQ ID NO: | GCUU (SEQ |
| | | | | ID NO: 548) | gaaccaa (SEQ ID | 596) | ID NO: 620) |
| | | | | | NO: 572) | | |
| 28322 | TET2 | NM_001127208.2 | 1386 | AUGCUGAU | gaugcugaugAUG | GAUAAUGC | UUUACUGG |
| | | | | AAUGCCAG | CUGAUAAUGC | CAGUAAA | CAUUAUCA |
| | | | | UAAA (SEQ | CAGUAAAcuag | (SEQ ID NO: | GCAU (SEQ |
| | | | | ID NO: 549) | cugcaau (SEQ ID | 597) | ID NO: 621) |
| | | | | | NO: 573) | | |
| 28323 | TET2 | NM_001127208.2 | 631 | AAAUGGAG | auccagaaguAAA | GAGACACC | UCCACUUG |
| | | | | ACACCAAG | UGGAGACACC | AAGUGGA | GUGUCUCC |
| | | | | UGGC (SEQ | AAGUGGCacuc | (SEQ ID NO: | AUUU (SEQ |
| | | | | ID NO: 550) | uuucaaa (SEQ ID | 598) | ID NO: 622) |
| | | | | | NO: 574) | | |
| 28324 | TET2 | NM_001127208.2 | 1384 | UGAUGCUG | gugaugcugaUGA | CUGAUAAU | UACUGGCA |
| | | | | AUAAUGCC | UGCUGAUAAU | GCCAGUA | UUAUCAGC |
| | | | | AGUA (SEQ | GCCAGUAaacua | (SEQ ID NO: | AUCA (SEQ |
| | | | | ID NO: 551) | gcugca (SEQ ID | 599) | ID NO: 623) |
| | | | | | NO: 575) | | |
| 28325 | TET2 | NM_001127208.2 | 2376 | AGUCACAA | cuggagcacaAGU | CAAAUGUA | UACUUGGU |
| | | | | AUGUACCA | CACAAAUGUA | CCAAGUA | ACAUUUGU |
| | | | | AGUU (SEQ | CCAAGUUgaaau | (SEQ ID NO: | GACU (SEQ |
| | | | | ID NO: 552) | gaauca (SEQ ID | 600) | ID NO: 624) |
| | | | | | NO: 576) | | |
| 28326 | TET2 | NM_001127208.2 | 1613 | AUGAAUGG | acaaaaugaaAUG | UGGUGCUU | UUGAAGUA |
| | | | | UGCUUACU | AAUGGUGCUU | ACUUCAA | AGCACCAU |
| | | | | UCAA (SEQ | ACUUCAAgcaaa | (SEQ ID NO: | UCAU (SEQ |
| | | | | ID NO: 553) | gcucag (SEQ ID | 601) | ID NO: 625) |
| | | | | | NO: 577) | | |
| 28327 | TET2 | NM_001127208.2 | 768 | UAAAACGC | aauggaggaaUAA | CGCACAGU | UUCACUAA |
| | | | | ACAGUUAG | AACGCACAGU | UAGUGAA | CUGUGCGU |
| | | | | UGAA (SEQ | UAGUGAAccuu | (SEQ ID NO: | UUUA (SEQ |
| | | | | ID NO: 554) | cucucuc (SEQ ID | 602) | ID NO: 626) |
| | | | | | NO: 578) | | |
| 28328 | TET2 | NM_001127208.2 | 1618 | UGGUGCUU | augaaaugaaUGG | CUUACUUC | UUUGCUUG |
| | | | | ACUUCAAG | UGCUUACUUC | AAGCAAA | AAGUAAGC |
| | | | | CAAA (SEQ | AAGCAAAgcuc | (SEQ ID NO: | ACCA (SEQ |
| | | | | ID NO: 555) | aguguuc (SEQ ID | 603) | ID NO: 627) |
| | | | | | NO: 579) | | |
| 28329 | TET2 | NM_001127208.2 | 1620 | GUGCUUAC | gaaaugaaugGUG | UACUUCAA | UCUUUGCU |
| | | | | UUCAAGCA | CUUACUUCAA | GCAAAGA | UGAAGUAA |
| | | | | AAGC (SEQ | GCAAAGCucag | (SEQ ID NO: | GCAC (SEQ |
| | | | | ID NO: 556) | uguucac (SEQ ID | 604) | ID NO: 628) |
| | | | | | NO: 580) | | |
| 28330 | TET2 | NM_001127208.2 | 3314 | CAGAAGGA | gaccccucccCAG | GGACACUC | UGCUUUUG |
| | | | | CACUCAAA | AAGGACACUC | AAAAGCA | AGUGUCCU |
| | | | | AGCA (SEQ | AAAAGCAugcu | (SEQ ID NO: | UCUG (SEQ |
| | | | | ID NO: 557) | gcucuaa (SEQ ID | 605) | ID NO: 629) |
| | | | | | NO: 581) | | |
| 28331 | TET2 | NM_001127208.2 | 1184 | UAUUAUCC | acugucucaaUAU | UCCAGAUU | UAAACACA |
| | | | | AGAUUGUG | UAUCCAGAUU | GUGUUUA | AUCUGGAU |
| | | | | UUUC (SEQ | GUGUUUCcauu | (SEQ ID NO: | AAUA (SEQ |
| | | | | ID NO: 558) | gcggugc (SEQ ID | 606) | ID NO: 630) |
| | | | | | NO: 582) | | |
| 28332 | TET2 | NM_001127208.2 | 3318 | AGGACACU | ccuccccagaAGG | ACUCAAAA | UGCAUGCU |
| | | | | CAAAAGCA | ACACUCAAAA | GCAUGCA | UUUGAGUG |
| | | | | UGCU (SEQ | GCAUGCUgcuc | (SEQ ID NO: | UCCU (SEQ |
| | | | | ID NO: 559) | uaaggug (SEQ ID | 607) | ID NO: 631) |
| | | | | | NO: 583) | | |
| 28333 | TET2 | NM_001127208.2 | 1240 | CAUUAACA | acauaaaugcCAU | ACAGUCAG | UAGUAGCC |
| | | | | GUCAGGCU | UAACAGUCAG | GCUACUA | UGACUGUU |
| | | | | ACUA (SEQ | GCUACUAauga | (SEQ ID NO: | AAUG (SEQ |
| | | | | ID NO: 560) | guugucc (SEQ ID | 608) | ID NO: 632) |
| | | | | | NO: 584) | | |
| 28334 | TET2 | NM_001127208.2 | 2580 | UGUUGAAA | auguccccagUGU | AAACAGCA | UUUCAAGU |
| | | | | CAGCACUU | UGAAACAGCA | CUUGAAA | GCUGUUUC |
| | | | | GAAU (SEQ | CUUGAAUcaaca | (SEQ ID NO: | AACA (SEQ |
| | | | | ID NO: 561) | ggcuuc (SEQ ID | 609) | ID NO: 633) |
| | | | | | NO: 585) | | |
| 28335 | TET2 | NM_001127208.2 | 2814 | UUGGCCAG | ggaucauucuUUG | CAGACUAA | UUCCACUU |
| | | | | ACUAAAGU | GCCAGACUAA | AGUGGAA | UAGUCUGG |
| | | | | GGAA (SEQ | AGUGGAAgaau | (SEQ ID NO: | CCAA (SEQ |
| | | | | ID NO: 562) | guuuuca (SEQ ID | 610) | ID NO: 634) |
| | | | | | NO: 586) | | |
| 28336 | TET2 | NM_001127208.2 | 1579 | UGGCAGCU | aagcuccuggUGG | GCUCUGAA | UAUACCGU |
| | | | | CUGAACGG | CAGCUCUGAA | CGGUAUA | UCAGAGCU |
| | | | | UAUU (SEQ | CGGUAUUuaaaa | (SEQ ID NO: | GCCA (SEQ |
| | | | | ID NO: 563) | caaaau (SEQ ID | 611) | ID NO: 635) |
| | | | | | NO: 587) | | |
| 28337 | TET2 | NM_001127208.2 | 3237 | AAAGGUAC | cuugcucagcAAA | UACUUGAU | UUUAUGUA |
| | | | | UUGAUACA | GGUACUUGAU | ACAUAAA | UCAAGUAC |
| | | | | UAAC (SEQ | ACAUAACcaugc | (SEQ ID NO: | CUUU (SEQ |
| | | | | ID NO: 564) | aaaugu (SEQ ID | 612) | ID NO: 636) |
| | | | | | NO: 588) | | |
| 28338 | TET2 | NM_001127208.2 | 2993 | AACAAUAC | uucuuguucaAAC | UACACACC | UAAACUAG |
| | | | | ACACCUAG | AAUACACACC | UAGUUUA | GUGUGUAU |
| | | | | UUUC (SEQ | UAGUUUCagag | (SEQ ID NO: | UGUU (SEQ |
| | | | | ID NO: 565) | aauaaag (SEQ ID | 613) | ID NO: 637) |
| | | | | | NO: 589) | | |
| 28339 | TET2 | NM_001127208.2 | 2631 | ACUCACACC | ccauuuucaaACU | CACCUUUU | UUGUUGCA |
| | | | | UUUUGCAA | CACACCUUUU | GCAACAA | AAAGGUGU |
| | | | | CAU (SEQ ID | GCAACAUaagcc | (SEQ ID NO: | GAGU (SEQ |
| | | | | NO: 566) | ucauaa (SEQ ID | 614) | ID NO: 638) |
| | | | | | NO: 590) | | |
| 28340 | TET2 | NM_001127208.2 | 1874 | CCUAAUCCA | uucccagaguCCU | UCCAUCUA | UCAUGUGU |
| | | | | UCUACACA | AAUCCAUCUA | CACAUGA | AGAUGGAU |
| | | | | UGU (SEQ ID | CACAUGUaugca | (SEQ ID NO: | UAGG (SEQ |
| | | | | NO: 567) | gcccuu (SEQ ID | 615) | ID NO: 639) |
| | | | | | NO: 591) | | |

TABLE 13

| Oligo | | | Start | | | Passenger | Guide |
|---|---|---|---|---|---|---|---|
| ID | Gene | Accession number | Site | Sequence | Gene region | sequence | Sequence |
| 28293 | Tbox21 | NM_013351.1 | 641 | CACCUGUUG | gcucaacaacCACC | GUUGUGGU | UACUUGGA |
| | | | | UGGUCCAAG | UGUUGUGGUC | CCAAGUA | CCACAACAG |
| | | | | UU (SEQ ID | CAAGUUuaauca | (SEQ ID NO: | GUG (SEQ ID |
| | | | | NO: 640) | gcacc (SEQ ID | 690) | NO: 715) |
| | | | | | NO: 665) | | |
| 28294 | Tbox21 | NM_013351.1 | 755 | CACUACAGG | gcccaccagcCAC | CAGGAUGU | UCCACAAAC |
| | | | | AUGUUUGUG | UACAGGAUGU | UUGUGGA | AUCCUGUA |
| | | | | GA (SEQ ID | UUGUGGAcgug | (SEQ ID NO: | GUG (SEQ ID |
| | | | | NO: 641) | gucuugg (SEQ ID | 691) | NO: 716) |
| | | | | | NO: 666) | | |
| 28295 | Tbox21 | NM_013351.1 | 2506 | CUGAGAGUG | auuuauuguaCUG | AGUGGUGU | UAUCCAGA |
| | | | | GUGUCUGGA | AGAGUGGUGU | CUGGAUA | CACCACUCU |
| | | | | UA (SEQ ID | CUGGAUAuauu | (SEQ ID NO: | CAG (SEQ ID |
| | | | | NO: 642) | ccuuuug (SEQ ID | 692) | NO: 717) |
| | | | | | NO: 667) | | |
| 28296 | Tbox21 | NM_013351.1 | 1723 | CUAUCCUUC | gcguguccccCUA | CUUCCAGU | UGUCACCAC |
| | | | | CAGUGGUGA | UCCUUCCAGU | GGUGACA | UGGAAGGA |
| | | | | CA (SEQ ID | GGUGACAgcuc | (SEQ ID NO: | UAG (SEQ ID |
| | | | | NO: 643) | cuccccu (SEQ ID | 693) | NO: 718) |
| | | | | | NO: 668) | | |
| 28297 | Tbox21 | NM_013351.1 | 1133 | GCCGAGAUU | cuaccagaauGCC | GAUUACUC | UUCAGCUG |
| | | | | ACUCAGCUG | GAGAUUACUC | AGCUGAA | AGUAAUCU |
| | | | | AA (SEQ ID | AGCUGAAaauu | (SEQ ID NO: | CGGC (SEQ |
| | | | | NO: 644) | gauaaua (SEQ ID | 694) | ID NO: 719) |
| | | | | | NO: 669) | | |
| 28298 | Tbox21 | NM_013351.1 | 1070 | UCCAACACG | cugcaacgcuUCC | CACGCAUA | UUAAAGAU |
| | | | | CAUAUCUUU | AACACGCAUA | UCUUUAA | AUGCGUGU |
| | | | | AC (SEQ ID | UCUUUACuuuc | (SEQ ID NO: | UGGA (SEQ |
| | | | | NO: 645) | caagaaa (SEQ ID | 695) | ID NO: 720) |
| | | | | | NO: 670) | | |
| 28299 | Tbox21 | NM_013351.1 | 1415 | GUCAGCAUG | guuucgagcaGUC | CAUGAAGC | uAUGCAGGC |
| | | | | AAGCCUGCA | AGCAUGAAGC | CUGCAUA | UUCAUGCU |
| | | | | UU (SEQ ID | CUGCAUUcuug | (SEQ ID NO: | GAC (SEQ ID |
| | | | | NO: 646) | cccucug (SEQ ID | 696) | NO: 721) |
| | | | | | NO: 671) | | |
| 28300 | Tbox21 | NM_013351.1 | 1692 | AAGGAGACU | ggacugggcgAAG | GACUCUAA | UCUCCUCUU |
| | | | | CUAAGAGGA | GAGACUCUAA | GAGGAGA | AGAGUCUC |
| | | | | GG (SEQ ID | GAGGAGGcgcg | (SEQ ID NO: | CUU (SEQ ID |
| | | | | NO: 647) | ugucccc (SEQ ID | 697) | NO: 722) |
| | | | | | NO: 672) | | |
| 28301 | Tbox21 | NM_013351.1 | 2058 | UUUACCUGG | acuacagucgUUU | CUGGUGCU | UAGACGCA |
| | | | | UGCUGCGUC | ACCUGGUGCU | GCGUCUA | GCACCAGG |
| | | | | UU (SEQ ID | GCGUCUUgcuu | (SEQ ID NO: | UAAA (SEQ |
| | | | | NO: 648) | uugguuu (SEQ ID | 698) | ID NO: 723) |
| | | | | | NO: 673) | | |
| 28302 | Tbox21 | NM_013351.1 | 1019 | CUGCAUAUC | ccagccccggCUG | UAUCGUUG | UUCACCUCA |
| | | | | GUUGAGGUG | CAUAUCGUUG | AGGUGAA | ACGAUAUG |
| | | | | AA (SEQ ID | AGGUGAAcgac | (SEQ ID NO: | CAG (SEQ ID |
| | | | | NO: 649) | ggagagc (SEQ ID | 699) | NO: 724) |
| | | | | | NO: 674) | | |
| 28303 | Tbox21 | NM_013351.1 | 2196 | CUUCCUUUG | acuccacuuuCUU | UUUGUACA | UAGUUACU |
| | | | | UACAGUAAC | CCUUUGUACA | GUAACUA | GUACAAAG |
| | | | | UU (SEQ ID | GUAACUUucaac | (SEQ ID NO: | GAAG (SEQ |
| | | | | NO: 650) | cuuuuc (SEQ ID | 700) | ID NO: 725) |
| | | | | | NO: 675) | | |
| 28304 | Tbox21 | NM_013351.1 | 1929 | CUCUGUUUA | ucuggcccuuCUC | UUUAGUAG | UAACCAAC |
| | | | | GUAGUUGGU | UGUUUAGUAG | UUGGUUA | UACUAAAC |
| | | | | UG (SEQ ID | UUGGUUGggga | (SEQ ID NO: | AGAG (SEQ |
| | | | | NO: 651) | agugggg (SEQ ID | 701) | ID NO: 726) |
| | | | | | NO: 676) | | |
| 28305 | Tbox21 | NM_013351.1 | 1884 | GAAACGGAU | acuaauuuggGAA | GGAUGAAG | UUCAGUCC |
| | | | | GAAGGACUG | ACGGAUGAAG | GACUGAA | UUCAUCCG |
| | | | | AG (SEQ ID | GACUGAGaagg | (SEQ ID NO: | UUUC (SEQ |
| | | | | NO: 652) | cccccgc (SEQ ID | 702) | ID NO: 727) |
| | | | | | NO: 677) | | |
| 28306 | Tbox21 | NM_013351.1 | 1861 | UUGGAGGAC | uguuauuaggUUG | GGACACCG | UAUUAGUC |
| | | | | ACCGACUAA | GAGGACACCG | ACUAAUA | GGUGUCCU |
| | | | | UU (SEQ ID | ACUAAUUuggg | (SEQ ID NO: | CCAA (SEQ |
| | | | | NO: 653) | aaacgga (SEQ ID | 703) | ID NO: 728) |
| | | | | | NO: 678) | | |
| 28307 | Tbox21 | NM_013351.1 | 2202 | UUGUACAGU | cuuucuuccuUUG | CAGUAACU | UGUUGAAA |
| | | | | AACUUUCAA | UACAGUAACU | UUCAACA | GUUACUGU |
| | | | | CC (SEQ ID | UUCAACCuuuu | (SEQ ID NO: | ACAA (SEQ |
| | | | | NO: 654) | cguuggc (SEQ ID | 704) | ID NO: 729) |
| | | | | | NO: 679) | | |
| 28308 | Tbox21 | NM_013351.1 | 976 | CCAGAUGAU | acaaugugacCCA | UGAUUGUG | UCUGGAGC |
| | | | | UGUGCUCCA | GAUGAUUGUG | CUCCAGA | ACAAUCAU |
| | | | | GU (SEQ ID | CUCCAGUcccuc | (SEQ ID NO: | CUGG (SEQ |
| | | | | NO: 655) | cauaag (SEQ ID | 705) | ID NO: 730) |
| | | | | | NO: 680) | | |
| 28309 | Tbox21 | NM_013351.1 | 1633 | CUUGGUGUG | agggucccccCUU | UGUGGACU | UAAUCUCA |
| | | | | GACUGAGAU | GGUGUGGACU | GAGAUUA | GUCCACACC |
| | | | | UG (SEQ ID | GAGAUUGccccc | (SEQ ID NO: | AAG (SEQ ID |
| | | | | NO: 656) | auccgg (SEQ ID | 706) | NO: 731) |
| | | | | | NO: 681) | | |
| 28310 | Tbox21 | NM_013351.1 | 2047 | AACUACAGU | ccucugcccuAAC | CAGUCGUU | UCAGGUAA |
| | | | | CGUUUACCU | UACAGUCGUU | UACCUGA | ACGACUGU |
| | | | | GG (SEQ ID | UACCUGGugcu | (SEQ ID NO: | AGUU (SEQ |
| | | | | NO: 657) | gcgucuu (SEQ ID | 707) | ID NO: 732) |
| | | | | | NO: 682) | | |
| 28311 | Tbox21 | NM_013351.1 | 2304 | GAAAGGACU | cagggucaggGAA | GACUCACC | UAAGUCAG |
| | | | | CACCUGACU | AGGACUCACC | UGACUUA | GUGAGUCC |
| | | | | UU (SEQ ID | UGACUUUggac | (SEQ ID NO: | UUUC (SEQ |
| | | | | NO: 658) | agcuggc (SEQ ID | 708) | ID NO: 733) |
| | | | | | NO: 683) | | |
| 28312 | Tbox21 | NM_013351.1 | 644 | CUGUUGUGG | caacaaccacCUGU | GUGGUCCA | UUAAACUU |
| | | | | UCCAAGUUU | UGUGGUCCAA | AGUUUAA | GGACCACA |
| | | | | AA (SEQ ID | GUUUAAucagca | (SEQ ID NO: | ACAG (SEQ |
| | | | | NO: 659) | ccaga (SEQ ID | 709) | ID NO: 734) |
| | | | | | NO: 684) | | |
| 28313 | Tbox21 | NM_013351.1 | 653 | UCCAAGUUU | ccuguuguggUCC | GUUUAAUC | UGGUGCUG |
| | | | | AAUCAGCAC | AAGUUUAAUC | AGCACCA | AUUAAACU |
| | | | | CA (SEQ ID | AGCACCAgacag | (SEQ ID NO: | UGGA (SEQ |
| | | | | NO: 660) | agauga (SEQ ID | 710) | ID NO: 735) |
| | | | | | NO: 685) | | |
| 28314 | Tbox21 | NM_013351.1 | 767 | UUUGUGGAC | cuacaggaugUUU | GGACGUGG | UCCAAGACC |
| | | | | GUGGUCUUG | GUGGACGUGG | UCUUGGA | ACGUCCACA |
| | | | | GU (SEQ ID | UCUUGGUggac | (SEQ ID NO: | AA (SEQ ID |
| | | | | NO: 661) | cagcacc (SEQ ID | 711) | NO: 736) |
| | | | | | NO: 686) | | |
| 28315 | Tbox21 | NM_013351.1 | 1205 | UACACAUCU | ugaguccaugUAC | AUCUGUUG | UUGGUGUC |
| | | | | GUUGACACC | ACAUCUGUUG | ACACCAA | AACAGAUG |
| | | | | AG (SEQ ID | ACACCAGcaucc | (SEQ ID NO: | UGUA (SEQ |
| | | | | NO: 662) | ccuccc (SEQ ID | 712) | ID NO: 737) |
| | | | | | NO: 687) | | |
| 28316 | Tbox21 | NM_013351.1 | 2259 | GAACAAAUA | gauccaaaaaGAA | AAUACACG | UAACAUAC |
| | | | | CACGUAUGU | CAAAUACACG | UAUGUUA | GUGUAUUU |
| | | | | UA (SEQ ID | UAUGUUAuaac | (SEQ ID NO: | GUUC (SEQ |
| | | | | NO: 663) | caucagc (SEQ ID | 713) | ID NO: 738) |
| | | | | | NO: 688) | | |
| 28115 | AKT1 | NM_005163.2 | 2625 | UAUUGUGUA | uaaauuuguuUAU | GUAUUAUG | UGAACAAC |
| | | | | UUAUGUUGU | UGUGUAUUAU | UUGUUCA | AUAAUACA |
| | | | | UC (SEQ ID | GUUGUUCaaau | (SEQ ID NO: | CAAU (SEQ |
| | | | | NO: 664) | gcauuu (SEQ ID | 714) | ID NO: 739) |
| | | | | | NO: 689) | | |

Those skilled in the art will recognize, or be able to ascertain using no more than routine experimentation, many equivalents to the specific embodiments of the invention described herein. Such equivalents are intended to be encompassed by the following claims.

What is claimed is:

1. A chemically-modified double stranded nucleic acid molecule that is directed against a gene encoding TIGIT, PDCD1, AKT, p53, Cbl-b, Tet2, Blimp-1, T-Box21, DNM3A, PTPN6, or HK2, optionally wherein the chemically-modified double stranded nucleic acid molecule is directed against a sequence comprising at least 12 contiguous nucleotides of a sequence selected from the sequences within Tables 3-13.

2. The chemically-modified double stranded nucleic acid molecule of claim 1, wherein the chemically-modified double stranded nucleic acid molecule is an sd-rxRNA.

3. The chemically-modified double stranded nucleic acid molecule of claim 1 or 2, wherein the chemically-modified double stranded nucleic acid molecule comprises at least one 2′-O-methyl modification and/or at least one 2′-Fluoro modification, and at least one phosphorothioate modification.

4. An sd-rxRNA that is directed against a gene encoding TIGIT, PDCD1, AKT, P53, Cbl-b, Tet2, Blimp-1, T-Box21, DNMT3A, PTPN6, or HK2, wherein the sd-rxRNA comprises at least 12 contiguous nucleotides of a sequence selected from the sequences within Tables 3-13.

5. The sd-rxRNA of claim 4, wherein the sd-rxRNA is hydrophobically modified.

6. The sd-rxRNA of claim 4 or 5, wherein the sd-rxRNA is linked to one or more hydrophobic conjugates, optionally wherein the hydrophobic conjugate is cholesterol.

7. A composition comprising a chemically-modified double stranded nucleic acid molecule of any one of claims 1 to 3 and a pharmaceutically acceptable excipient.

8. The composition of claim 7, wherein the chemically-modified double stranded nucleic acid molecule comprises or consists of at least 12 contiguous nucleotides of a sequence selected from Table 3, 4, 5, 6, or 8, optionally wherein chemically-modified double stranded nucleic acid molecule comprises the sequence set forth in TIGIT 1 (SEQ ID NO: 60), TIGIT 6 (SEQ ID NO: 65), TIGIT 21 (SEQ ID NOs: 100 and 101), PD 26 (SEQ ID NOs: 112 and 113), CB 23 (SEQ ID NOs: 236 and 237), or CB 29 (SEQ ID NOs: 248 and 249).

9. A composition comprising the sd-rxRNA of any one of claims 4 to 6 and a pharmaceutically acceptable excipient.

10. The composition of claim 9, wherein the sd-rxRNA comprises or consists of the sequence set forth in CB 23 (SEQ ID NO: 236 or 237) or CB 29 (SEQ ID NO: 248 or 249).

11. The composition of claim 9, wherein the chemically-modified double stranded nucleic acid molecule comprises a sense strand having the sequence set forth in PD 26 sense strand (SEQ ID NO: 112) and/or an antisense strand having the sequence set forth in PD 26 antisense strand (SEQ ID NO: 113).

12. The composition of claim 11, wherein the chemically-modified double stranded nucleic acid molecule or the sd-rxRNA comprises or consists of a sense strand having the sequence set forth in PD 26 sense strand (SEQ ID NO: 112) and an antisense strand having the sequence set forth in PD 26 antisense strand (SEQ ID NO: 113).

13. The composition of any one of claims 7 to 9, wherein the chemically-modified double stranded nucleic acid molecule or the sd-rxRNA comprises a sense strand having the sequence set forth in CB 23 sense strand (SEQ ID NO: 236) and/or an antisense strand having the sequence set forth in CB 23 antisense strand (SEQ ID NO: 237).

14. The composition of claim 13, wherein the chemically-modified double stranded nucleic acid molecule or the sd-rxRNA consists of a sense strand having the sequence set forth in CB 23 sense strand (SEQ ID NO: 236) and an antisense strand having the sequence set forth in CB 23 antisense strand (SEQ ID NO: 237).

15. The composition of any one of claims 7 to 9, wherein the chemically-modified double stranded nucleic acid molecule or the sd-rxRNA comprises a sense strand having the sequence set forth in CB 29 sense strand (SEQ ID NO: 248) and/or an antisense strand having the sequence set forth in CB 29 antisense strand (SEQ ID NO: 249).

16. The composition of claim 15, wherein the chemically-modified double stranded nucleic acid molecule or the sd-rxRNA consists of a sense strand having the sequence set forth in CB 29 sense strand (SEQ ID NO: 248) and an antisense strand having the sequence set forth in CB 29 antisense strand (SEQ ID NO: 249).

17. The composition of any one of claims 7 to 9, wherein the chemically-modified double stranded nucleic acid molecule comprises a sense strand having the sequence set forth in TIGIT 21 sense strand (SEQ ID NO: 100) and/or an antisense strand having the sequence set forth in TIGIT 21 antisense strand (SEQ ID NO: 101).

18. The composition of claim 17, wherein the chemically-modified double stranded nucleic acid molecule comprises a sense strand having the sequence set forth in TIGIT 21 sense strand (SEQ ID NO: 100) and an antisense strand having the sequence set forth in TIGIT 21 antisense strand (SEQ ID NO: 101).

19. An immunogenic composition comprising a host cell which was treated ex vivo with a chemically-modified double stranded nucleic acid molecule to control and/or reduce the level of differentiation of the host cell to enable the production of a specific immune cellular population for administration in a human.

20. The immunogenic composition of claim 19, wherein the host cell comprises a chemically-modified double stranded nucleic acid molecule that is directed against a gene encoding PDCD1, AKT, p53, Cbl-b, Tet2, Blimp-1, T-Box21, DNMT3A, PTPN6, or HK2, optionally wherein the chemically-modified double stranded nucleic acid molecule is directed against a sequence comprising at least 12 contiguous nucleotides of a sequence selected from the sequences within Tables 3-13, further optionally wherein the chemically-modified double stranded nucleic acid molecule is directed against PDCD1 and comprises at least 12 contiguous nucleotides of a sequence selected from Table 3 or 6.

21. The immunogenic composition of claim 19 or 20, wherein the chemically-modified double stranded nucleic acid molecule comprises at least one 2′-O-methyl modification and/or at least one 2′-Fluoro modification, and at least one phosphorothioate modification.

22. The immunogenic composition of claim 21, wherein the chemically-modified double stranded nucleic acid molecule is hydrophobically modified.

23. The immunogenic composition of claim 22, wherein the chemically-modified double stranded nucleic acid molecule is linked to one or more hydrophobic conjugates, optionally wherein the hydrophobic conjugate is cholesterol.

24. The immunogenic composition of any one of claims 19 to 23, wherein the host cell is selected from the group of: T-cell, NK-cell, antigen-presenting cell (APC), dendritic cell (DC), stem cell (SC), induced pluripotent stem cell (iPSC),stem cell memory T-cell, and Cytokine-induced Killer cell (CIK).

25. The immunogenic composition of claim 24, wherein the host cell is a T-cell.

26. The immunogenic composition of claim 24 or 25, wherein the T-cell is a CD8+ T-cell, optionally wherein the T-cell is differentiated into a T_{SCM} or T_{CM} after introduction of the chemically-modified double stranded nucleic acid molecule or the sd-rxRNA, further optionally wherein the immunogenic composition comprises at least 50%, at least 60%, at least 70%, at least 80%, at least 90%, at least 95%, at least 99% or 100% T_{SCM} or T_{CM} cells.

27. The immunogenic composition of any one of claims 24 to 26, wherein the T-cell comprises one or more transgenes expressing a high affinity T-cell receptor (TCR) and/or a chimeric antigen receptor (CAR).

28. The immunogenic composition of any one of claims 19 to 27, wherein the host cell is derived from a healthy donor.

29. A method for producing an immunogenic composition, the method comprising introducing into a cell one or more chemically-modified double stranded nucleic acid molecules, wherein the one or more chemically-modified nucleic acid molecules target PDCD1, AKT, p53, Cbl-b, Tet2, Blimp-1, T-Box21, DNMT3A, PTPN6, and/or HK2, thereby producing a host cell.

30. A method for producing an immunogenic composition, the method comprising introducing into a cell the chemically-modified double stranded nucleic acid molecule or the sd-rxRNA of any one of claims 1 to 6.

31. The method of claim 29 or 30, wherein the cell is a T-cell, NK-cell, antigen-presenting cell (APC), dendritic cell (DC), stem cell (SC), induced pluripotent stem cell (iPSC),stem cell memory T-cell, and Cytokine-induced Killer cell (CIK).

32. The method of claim 30, wherein the T-cell is a CD8+ T-cell, optionally wherein the T-cell is differentiated into a T_{SCM} or T_{CM} after introduction of the chemically-modified double stranded nucleic acid or sd-rxRNA, further optionally wherein the immunogenic composition comprises at least 50%, at least 60%, at least 70%, at least 80%, at least 90%, at least 95%, at least 99% or 100% T_{SCM} or T_{CM} cells.

33. The method of claim 31 or 32, wherein the T-cell comprises one or more transgenes expressing a high affinity T-cell receptor (TCR) and/or a chimeric antigen receptor (CAR).

34. The method of any one of claims 29 to 33, wherein the cell is derived from a healthy donor.

35. A method for treating a subject suffering from a proliferative disease or infectious disease, the method comprising administering to the subject the immunogenic composition of any one of claims 19 to 28.

36. The method of claim 35, wherein the proliferative disease is cancer.

37. The method of claim 35, wherein the infectious disease is a pathogen infection.

38. The method of claim 37, wherein the pathogen infection is a bacterial infection, viral infection, or parasitic infection.

40. An immunogenic composition comprising a host cell comprising a chemically-modified double stranded nucleic acid molecule that is directed against a TIGIT sequence comprising at least 12 contiguous nucleotides of a sequence selected from the sequences within Table 5.

41. An immunogenic composition comprising a host cell comprising a chemically-modified double stranded nucleic acid molecule that is directed against a PDCD1 sequence comprising at least 12 contiguous nucleotides of a sequence selected from the sequences within Table 3 or 6.

42. The immunogenic composition of claim 40 or 41, wherein the chemically-modified double stranded nucleic acid molecule is an sd-rxRNA.

43. The immunogenic composition of any one of claims 40 to 42, wherein the host cell comprises a first chemically-modified double stranded nucleic acid molecule or sd-rxRNA targeting PDCD1 and a second chemically-modified double stranded nucleic acid molecule or sd-rxRNA targeting TIGIT.

44. The immunogenic composition of any one of claims 40 to 43, wherein the chemically-modified double stranded nucleic acid molecule or sd-rxRNA induces at least 50% inhibition of PDCD1 or TIGIT in the host cell.

45. A method for treating a subject suffering from a proliferative disease or infectious disease, the method comprising administering to the subject the immunogenic composition of any one of claims 40 to 44.


SEQUENCE LISTING (739 sequences)

SEQ ID NO: 1 - DNA, 20 bp, Homo sapiens
gagtaggacataccagctta

SEQ ID NO: 2 - DNA, 19 bp, Homo sapiens
agagttctgtggaagtcta

SEQ ID NO: 3 - DNA, 19 bp, Homo sapiens
agagttctgtggaagtcta

SEQ ID NO: 4 - DNA, 19 bp, Homo sapiens
agagttctgtggaagtcta

SEQ ID NO: 5 - DNA, 19 bp, Homo sapiens
acagcaaattccatcgtgt

SEQ ID NO: 6 - DNA, 20 bp, Homo sapiens
ctggtaaagtggatattgtt

SEQ ID NO: 7 - DNA, 19 bp, Homo sapiens
agagttctgtggaagtcta

SEQ ID NO: 8 - RNA, 20 nt, Homo sapiens
uauuauauuauaauuauaau

SEQ ID NO: 9 - RNA, 20 nt, Homo sapiens
ucuauuauauuauaauuaua

SEQ ID NO: 10 - RNA, 20 nt, Homo sapiens
cauuccugaaauuauuuaaa

SEQ ID NO: 11 - RNA, 20 nt, Homo sapiens
cuauuauauuauaauuauaa

SEQ ID NO: 12 - RNA, 20 nt, Homo sapiens
aguuucagggaaggucagaa

SEQ ID NO: 13 - RNA, 20 nt, Homo sapiens
ugugguucuauuauauuaua

SEQ ID NO: 14 - RNA, 20 nt, Homo sapiens
uguguucucuguggacuaug

SEQ ID NO: 15 - RNA, 20 nt, Homo sapiens
cccauuccugaaauuauuua

SEQ ID NO: 16 - RNA, 20 nt, Homo sapiens
ugccaccauugucuuuccua

SEQ ID NO: 17 - RNA, 20 nt, Homo sapiens
aaguuucagggaaggucaga

SEQ ID NO: 18 - RNA, 20 nt, Homo sapiens
cugugguucuauuauauuau

SEQ ID NO: 19 - RNA, 20 nt, Homo sapiens
uucuauuauauuauaauuau

SEQ ID NO: 20 - RNA, 20 nt, Homo sapiens
uuucagggaaggucagaaga

SEQ ID NO: 21 - RNA, 20 nt, Homo sapiens
cuuggaacccauuccugaaa

SEQ ID NO: 22 - RNA, 20 nt, Homo sapiens
ucccugugguucuauuauau

SEQ ID NO: 23 - RNA, 20 nt, Homo sapiens
ccugugguucuauuauauua

SEQ ID NO: 24 - RNA, 20 nt, Homo sapiens
uggaacccauuccugaaauu

SEQ ID NO: 25 - RNA, 20 nt, Homo sapiens
ccuucccugugguucuauua

SEQ ID NO: 26 - RNA, 20 nt, Homo sapiens
uucccugugguucuauuaua

SEQ ID NO: 27 - RNA, 20 nt, Homo sapiens
cacaggacucaugucucaau

SEQ ID NO: 28 - DNA, 45 bp, Homo sapiens
ccttccctgtggttctattatattataattataattaaatatgag

SEQ ID NO: 29 - DNA, 45 bp, Homo sapiens
ccccttccctgtggttctattatattataattataattaaatatg

SEQ ID NO: 30 - DNA, 45 bp, Homo sapiens
gctctccttggaacccattcctgaaattatttaaaggggttggcc

SEQ ID NO: 31 - DNA, 45 bp, Homo sapiens
cccttccctgtggttctattatattataattataattaaatatga

SEQ ID NO: 32 - DNA, 45 bp, Homo sapiens
ctgcaggcctagagaagtttcagggaaggtcagaagagctcctgg

SEQ ID NO: 33 - DNA, 45 bp, Homo sapiens
gggatcccccttccctgtggttctattatattataattataatta

SEQ ID NO: 34 - DNA, 45 bp, Homo sapiens
cccctcagccgtgcctgtgttctctgtggactatggggagctgga

SEQ ID NO: 35 - DNA, 45 bp, Homo sapiens
gagctctccttggaacccattcctgaaattatttaaaggggttgg

SEQ ID NO: 36 - DNA, 45 bp, Homo sapiens
tgagcagacggagtatgccaccattgtctttcctagcggaatggg

SEQ ID NO: 37 - DNA, 45 bp, Homo sapiens
cctgcaggcctagagaagtttcagggaaggtcagaagagctcctg

SEQ ID NO: 38 - DNA, 45 bp, Homo sapiens
agggatcccccttccctgtggttctattatattataattataatt

SEQ ID NO: 39 - DNA, 45 bp, Homo sapiens
cccccttccctgtggttctattatattataattataattaaatat

SEQ ID NO: 40 - DNA, 45 bp, Homo sapiens
gcaggcctagagaagtttcagggaaggtcagaagagctcctggct

SEQ ID NO: 41 - DNA, 45 bp, Homo sapiens
accctgggagctctccttggaacccattcctgaaattatttaaag

SEQ ID NO: 42 - DNA, 45 bp, Homo sapiens
acaagggatcccccttccctgtggttctattatattataattata

SEQ ID NO: 43 - DNA, 45 bp, Homo sapiens
aagggatcccccttccctgtggttctattatattataattataat

SEQ ID NO: 44 - DNA, 45 bp, Homo sapiens
cctgggagctctccttggaacccattcctgaaattatttaaaggg

SEQ ID NO: 45 - DNA, 45 bp, Homo sapiens
gggacaagggatcccccttccctgtggttctattatattataatt

SEQ ID NO: 46 - DNA, 45 bp, Homo sapiens
gacaagggatcccccttccctgtggttctattatattataattat

SEQ ID NO: 47 - DNA, 45 bp, Homo sapiens
caggcacagccccaccacaggactcatgtctcaatgcccacagtg

SEQ ID NO: 48 - RNA, 20 nt, Homo sapiens
caauugauuuaacuugcaau

SEQ ID NO: 49 - RNA, 20 nt, Homo sapiens
uuuaacuugcaaugauuaca

SEQ ID NO: 50 - RNA, 20 nt, Homo sapiens
gaaguuaaagcacgacuaca

SEQ ID NO: 51 - RNA, 20 nt, Homo sapiens
aaaguuacacaggaacaaua

SEQ ID NO: 52 - RNA, 20 nt, Homo sapiens
uucugucguugugaaauaaa

SEQ ID NO: 53 - RNA, 20 nt, Homo sapiens
cuccuugcauggugagaaaa

SEQ ID NO: 54 - RNA, 20 nt, Homo sapiens
cuguucggucuugugauaau

SEQ ID NO: 55 - RNA, 20 nt, Homo sapiens
ugacuuaagcauauauuuaa

SEQ ID NO: 56 - RNA, 20 nt, Homo sapiens
agucucauugaacauucaaa

SEQ ID NO: 57 - RNA, 20 nt, Homo sapiens
gguguuuugauaccuguacu

SEQ ID NO: 58 - RNA, 20 nt, Homo sapiens
caacugaucaaacuaaugca

SEQ ID NO: 59 - RNA, 20 nt, Homo sapiens
agcauuuauuugucaauaaa

SEQ ID NO: 60 - RNA, 20 nt, Homo sapiens
cuuuugucuuugcuauuaua

SEQ ID NO: 61 - RNA, 20 nt, Homo sapiens
uaauugguauaagcauaaaa

SEQ ID NO: 62 - RNA, 20 nt, Homo sapiens
caaauuggaagugaacuaaa

SEQ ID NO: 63 - RNA, 20 nt, Homo sapiens
guuugcuguggcaguuuaca

SEQ ID NO: 64 - RNA, 20 nt, Homo sapiens
gaucauaaaugcaaaauuaa

SEQ ID NO: 65 - RNA, 20 nt, Homo sapiens
cgcguugacuagaaagaaga

SEQ ID NO: 66 - RNA, 20 nt, Homo sapiens
uuuaaauagaacucacugaa

SEQ ID NO: 67 - RNA, 20 nt, Homo sapiens
gcaaaucuguuggaaauaga

SEQ ID NO: 68 - RNA, 20 nt, Homo sapiens
ucuugcaaaauuagugcaaa

SEQ ID NO: 69 - RNA, 20 nt, Homo sapiens
acauaggaagaaugaacuga

SEQ ID NO: 70 - RNA, 20 nt, Homo sapiens
ucacuuuucuaccaaauggg

SEQ ID NO: 71 - RNA, 20 nt, Homo sapiens
guguuauuuaacauaauuau

SEQ ID NO: 72 - RNA, 20 nt, Homo sapiens
uguguguucaguugagugaa

SEQ ID NO: 73 - RNA, 20 nt, Homo sapiens
cuuugcuauuauagaugaau

SEQ ID NO: 74 - RNA, 20 nt, Homo sapiens
gaaaugggauucaauuugaa

SEQ ID NO: 75 - RNA, 20 nt, Homo sapiens
aaaauguaaugacgaaaagg

SEQ ID NO: 76 - RNA, 20 nt, Homo sapiens
gguuacauaggaagaaugaa

SEQ ID NO: 77 - RNA, 20 nt, Homo sapiens
uuuagcaacaagacaauuca

SEQ ID NO: 78 - RNA, 20 nt, Homo sapiens
ugcuauuauagaugaauaua

SEQ ID NO: 79 - RNA, 20 nt, Homo sapiens
gagauuuaauaugaauaaua

SEQ ID NO: 80 - DNA, 45 bp, Homo sapiens
cttctggaagatacacttttgtctttgctattatagatgaatata

SEQ ID NO: 81 - DNA, 45 bp, Homo sapiens
caagatgtgctgttataattggtataagcataaaatcacactaga

SEQ ID NO: 82 - DNA, 45 bp, Homo sapiens
atagaacacaattcacaaattggaagtgaactaaaatgtaatgac

SEQ ID NO: 83 - DNA, 45 bp, Homo sapiens
cgtaaaaatgttgttgtttgctgtggcagtttacagcatttttct

SEQ ID NO: 84 - DNA, 45 bp, Homo sapiens
agtaacgtggatcttgatcataaatgcaaaattaaaaaatatctt

SEQ ID NO: 85 - DNA, 45 bp, Homo sapiens
agtcatcgtggtggtcgcgttgactagaaagaagaaagccctcag

SEQ ID NO: 86 - DNA, 45 bp, Homo sapiens
tttgaaaaaaatttttttaaatagaactcactgaactagattctc

SEQ ID NO: 87 - DNA, 45 bp, Homo sapiens
tcttgcaaaattagtgcaaatctgttggaaatagaacacaattca

SEQ ID NO: 88 - DNA, 45 bp, Homo sapiens
agtttacagcatttttcttgcaaaattagtgcaaatctgttggaa

SEQ ID NO: 89 - DNA, 45 bp, Homo sapiens
tctaccaaatgggttacataggaagaatgaactgaaatctgtcca

SEQ ID NO: 90 - DNA, 45 bp, Homo sapiens
attattattattttttcacttttctaccaaatgggttacatagga

SEQ ID NO: 91 - DNA, 45 bp, Homo sapiens
tggactgagagttgggtgttatttaacataattatggtaattggg

SEQ ID NO: 92 - DNA, 45 bp, Homo sapiens
gtgtgtgtatgtgtgtgtgtgttcagttgagtgaataaatgtcat

SEQ ID NO: 93 - DNA, 45 bp, Homo sapiens
aagatacacttttgtctttgctattatagatgaatatataagcag

SEQ ID NO: 94 - DNA, 45 bp, Homo sapiens
atgggtcaggttactgaaatgggattcaatttgaaaaaaattttt

SEQ ID NO: 95 - DNA, 45 bp, Homo sapiens
aattggaagtgaactaaaatgtaatgacgaaaagggagtagtgtt

SEQ ID NO: 96 - DNA, 45 bp, Homo sapiens
cttttctaccaaatgggttacataggaagaatgaactgaaatctg

SEQ ID NO: 97 - DNA, 45 bp, Homo sapiens
ggggttgacaattgttttagcaacaagacaattcaactatttctc

SEQ ID NO: 98 - DNA, 45 bp, Homo sapiens
atacacttttgtctttgctattatagatgaatatataagcagctg

SEQ ID NO: 99 - DNA, 45 bp, Homo sapiens
tcacactagattctggagatttaatatgaataataagaatactat

SEQ ID NO: 100 - RNA, 15 nt, Artificial Sequence
  Synthetic Polynucleotide
  misc_feature (1)..(1): 2′ fluoro uridine
  misc_feature (1)..(13): modified by a phosphodiester linkage
  misc_feature (2)..(2): 2′-O-methyl guanine
  misc_feature (3)..(3): 2′ fluoro adenine
  misc_feature (4)..(4): 2′-O-methyl cytodine
  misc_feature (5)..(5): 2′ fluoro uridine
  misc_feature (6)..(6): 2′-O-methyl adenine
  misc_feature (7)..(7): 2′ fluoro guanine
  misc_feature (8)..(8): 2′-O-methyl adenine
  misc_feature (9)..(9): 2′ fluoro adenine
  misc_feature (10)..(10): 2′-O-methyl adenine
  misc_feature (11)..(11): 2′ fluoro guanine
  misc_feature (12)..(12): 2′-O-methyl adenine
  misc_feature (13)..(13): 2′ fluoro adenine
  misc_feature (13)..(15): modified by a phosphothioate linkage
  misc_feature (14)..(14): 2′-O-methyl guanine
  misc_feature (15)..(15): 2′ fluoro adenine
  misc_feature (15)..(15): modified by a phosphodiester linkage
  misc_feature (15)..(15): modified by TEG-Chl
ugacuagaaagaaga

SEQ ID NO: 101 - RNA, 20 nt, Artificial Sequence
  Synthetic Polynucleotide
  misc_feature (1)..(1): modified by 5′ inorganic Phosphate
  misc_feature (1)..(14): modified by a phosphodiester linkage
  misc_feature (1)..(1): 2′-O-methyl uridine
  misc_feature (2)..(2): 2′ fluoro cytidine
  misc_feature (3)..(3): 2′-O-methyl uridine
  misc_feature (4)..(4): 2′ fluoro uridine
  misc_feature (5)..(5): 2′-O-methyl cytidine
  misc_feature (6)..(6): 2′ fluoro uridine
  misc_feature (7)..(7): 2′-O-methyl uridine
  misc_feature (8)..(8): 2′ fluoro uridine
  misc_feature (9)..(9): 2′-O-methyl cytidine
  misc_feature (10)..(10): 2′ fluoro uridine
  misc_feature (11)..(11): 2′-O-methyl adenine
  misc_feature (12)..(12): 2′ fluoro guanine
  misc_feature (13)..(13): 2′-O-methyl uridine
  misc_feature (14)..(20): modified by a phosphothioate linkage
  misc_feature (14)..(14): 2′ fluoro cytidine
  misc_feature (15)..(15): 2′-O-methyl adenine
  misc_feature (16)..(16): 2′ fluoro adenine
  misc_feature (17)..(17): 2′-O-methyl cytidine
  misc_feature (18)..(18): 2′ fluoro guanine
  misc_feature (19)..(19): 2′-O-methyl cytidine
  misc_feature (20)..(20): 2′ fluoro guanine
ucuucuuucuagucaacgcg

SEQ ID NO: 102 - RNA, 15 nt, Artificial Sequence
  Synthetic Polynucleotide
ugugguucuauuaua

SEQ ID NO: 103 - RNA, 20 nt, Artificial Sequence
  Synthetic Polynucleotide
uauaauagaaccacagggaa

SEQ ID NO: 104 - RNA, 15 nt, Artificial Sequence
  Synthetic Polynucleotide
ugugguucuauuaua

SEQ ID NO: 105 - RNA, 20 nt, Artificial Sequence
  Synthetic Polynucleotide
uauaauagaaccacagggaa

SEQ ID NO: 106 - RNA, 15 nt, Artificial Sequence
  Synthetic Polynucleotide
ugugguucuauuaua

SEQ ID NO: 107 - RNA, 20 nt, Artificial Sequence
  Synthetic Polynucleotide
uauaauagaaccacagggaa

SEQ ID NO: 108 - RNA, 15 nt, Artificial Sequence
  Synthetic Polynucleotide
ugugguucuauuaua

SEQ ID NO: 109 - RNA, 20 nt, Artificial Sequence
  Synthetic Polynucleotide
uauaauagaaccacagggaa

SEQ ID NO: 110 - RNA, 15 nt, Artificial Sequence
  Synthetic Polynucleotide
ugugguucuauuaua

SEQ ID NO: 111 - RNA, 21 nt, Artificial Sequence
  Synthetic Polynucleotide
suauaauagaaccacagggaa

SEQ ID NO: 112 - RNA, 15 nt, Artificial Sequence
  Synthetic Polynucleotide
  misc_feature (1)..(1): 2′ fluoro uridine
  misc_feature (1)..(13): modified by a phosphodiester linkage
  misc_feature (2)..(2): 2′-O-methyl guanine
  misc_feature (3)..(3): 2′ fluoro uridine
  misc_feature (4)..(4): 2′-O-methyl guanine
  misc_feature (5)..(5): 2′ fluoro guanine
  misc_feature (6)..(6): 2′-O-methyl uridine
  misc_feature (7)..(7): 2′ fluoro uridine
  misc_feature (8)..(8): 2′-O-methyl cytidine
  misc_feature (9)..(9): 2′ fluoro uridine
  misc_feature (10)..(10): 2′-O-methyl adenine
  misc_feature (11)..(11): 2′ fluoro uridine
  misc_feature (12)..(12): 2′-O-methyl uridine
  misc_feature (13)..(13): 2′ fluoro adenine
  misc_feature (13)..(15): modified by a phosphorothioate linkage
  misc_feature (14)..(14): 2′-O-methyl uridine
  misc_feature (15)..(15): 2′ fluoro adenine
  misc_feature (15)..(15): modified by TEG-Chl
ugugguucuauuaua

SEQ ID NO: 113 - RNA, 20 nt, Artificial Sequence
  Synthetic Polynucleotide
  misc_feature (1)..(1): modified by 5′ inorganic Phosphate
  misc_feature (1)..(14): modified by a phosphodiester linkage
  misc_feature (1)..(1): 2′-O-methyl uridine
  misc_feature (2)..(2): 2′ fluoro adenine
  misc_feature (3)..(3): 2′ fluoro uridine
  misc_feature (4)..(4): 2′-O-methyl adenine
  misc_feature (5)..(5): 2′-O-methyl adenine
  misc_feature (6)..(6): 2′ fluoro uridine
  misc_feature (7)..(7): 2′-O-methyl adenine
  misc_feature (8)..(8): 2′-O-methyl guanine
  misc_feature (9)..(9): 2′-O-methyl adenine
  misc_feature (10)..(10): 2′ fluoro adenine
  misc_feature (11)..(11): 2′ fluoro cytodine
  misc_feature (12)..(12): 2′ fluoro cytodine
  misc_feature (13)..(13): 2′-O-methyl adenine
  misc_feature (14)..(20): modified by a phosphothioate linkage
  misc_feature (14)..(14): 2′ fluoro cytidine
  misc_feature (15)..(15): 2′-O-methyl adenine
  misc_feature (16)..(16): 2′-O-methyl guanine
  misc_feature (17)..(17): 2′-O-methyl guanine
  misc_feature (18)..(18): 2′-O-methyl guanine
  misc_feature (19)..(19): 2′-O-methyl adenine
  misc_feature (20)..(20): 2′-O-methyl adenine
uauaauagaaccacagggaa

SEQ ID NO: 114 - RNA, 15 nt, Artificial Sequence
  Synthetic Polynucleotide
ugugguucuauuaua

SEQ ID NO: 115 - RNA, 20 nt, Artificial Sequence
  Synthetic Polynucleotide
uauaauagaaccacagggaa

SEQ ID NO: 116 - RNA, 15 nt, Artificial Sequence
  Synthetic Polynucleotide
ugugguucuauuaua

SEQ ID NO: 117 - RNA, 20 nt, Artificial Sequence
  Synthetic Polynucleotide
uauaauagaaccacagggaa

SEQ ID NO: 118 - RNA, 15 nt, Artificial Sequence
  Synthetic Polynucleotide
ugugguucuauuaua

SEQ ID NO: 119 - RNA, 20 nt, Artificial Sequence
  Synthetic Polynucleotide
uauaauagaaccacagggaa

SEQ ID NO: 120 - RNA, 15 nt, Artificial Sequence
  Synthetic Polynucleotide
ugugguucuauuaua

SEQ ID NO: 121 - RNA, 20 nt, Artificial Sequence
  Synthetic Polynucleotide
uauaauagaaccacagggaa

SEQ ID NO: 122 - RNA, 15 nt, Artificial Sequence
  Synthetic Polynucleotide
ugugguucuauuaua

SEQ ID NO: 123 - RNA, 20 nt, Artificial Sequence
  Synthetic Polynucleotide
uauaauagaaccacagggaa

SEQ ID NO: 124 - RNA, 15 nt, Artificial Sequence
  Synthetic Polynucleotide
ugugguucuauuaua

SEQ ID NO: 125 - RNA, 20 nt, Artificial Sequence
  Synthetic Polynucleotide
uauaauagaaccacagggaa

SEQ ID NO: 126 - RNA, 15 nt, Artificial Sequence
  Synthetic Polynucleotide
ugugguucuauuaua

SEQ ID NO: 127 - RNA, 20 nt, Artificial Sequence
  Synthetic Polynucleotide
uauaauagaaccacagggaa

SEQ ID NO: 128 - RNA, 15 nt, Artificial Sequence
  Synthetic Polynucleotide
ugugguucuauuaua

SEQ ID NO: 129 - RNA, 20 nt, Artificial Sequence
  Synthetic Polynucleotide
uauaauagaaccacagggaa

SEQ ID NO: 130 - RNA, 15 nt, Artificial Sequence
  Synthetic Polynucleotide
ugugguucuauuaua

SEQ ID NO: 131 - RNA, 20 nt, Artificial Sequence
  Synthetic Polynucleotide
uauaauagaaccacagggaa

SEQ ID NO: 132 - RNA, 20 nt, Artificial Sequence
  Synthetic Polynucleotide
uauaauagaaccacagggaa

SEQ ID NO: 133 - RNA, 15 nt, Artificial Sequence
  Synthetic Polynucleotide
ugugguucuauuaua

SEQ ID NO: 134 - RNA, 20 nt, Artificial Sequence
  Synthetic Polynucleotide
uauaauagaaccacagggaa

SEQ ID NO: 135 - RNA, 15 nt, Artificial Sequence
  Synthetic Polynucleotide
ugugguucuauuaua

SEQ ID NO: 136 - RNA, 20 nt, Homo sapiens
ccagcuguuugaccacauug

SEQ ID NO: 137 - RNA, 20 nt, Homo sapiens
cuguuugaccacauugccga

SEQ ID NO: 138 - RNA, 20 nt, Homo sapiens
uuugaccacauugccgaaug

SEQ ID NO: 139 - RNA, 20 nt, Homo sapiens
agaggaguuugaccuggaug

SEQ ID NO: 140 - RNA, 20 nt, Homo sapiens
cugugaaguuggccucauug

SEQ ID NO: 141 - RNA, 20 nt, Homo sapiens
aaugccugcuacauggagga

SEQ ID NO: 142 - RNA, 20 nt, Homo sapiens
ugugacgacagcaucauugu

SEQ ID NO: 143 - RNA, 20 nt, Homo sapiens
gugagauuggucucauugug

SEQ ID NO: 144 - RNA, 20 nt, Homo sapiens
gucucagauugagagugacu

SEQ ID NO: 145 - RNA, 20 nt, Homo sapiens
cucagauugagagugacugc

SEQ ID NO: 146 - RNA, 20 nt, Homo sapiens
aagucaugcaugagacagug

SEQ ID NO: 147 - RNA, 20 nt, Homo sapiens
ugagcacucagucuagugaa

SEQ ID NO: 148 - RNA, 20 nt, Homo sapiens
gaccaacuuccgugugcuuu

SEQ ID NO: 149 - RNA, 20 nt, Homo sapiens
aaaugaucaguggaauguac

SEQ ID NO: 150 - RNA, 20 nt, Homo sapiens
ccuugagcacucagucuagu

SEQ ID NO: 151 - RNA, 20 nt, Homo sapiens
cuacauccucacuuugccaa

SEQ ID NO: 152 - RNA, 20 nt, Homo sapiens
cuuggaccuuggaggaacaa

SEQ ID NO: 153 - RNA, 20 nt, Homo sapiens
gagaugcacaacaagaucua

SEQ ID NO: 154 - RNA, 20 nt, Homo sapiens
gaacuaugaugaccuguggc

SEQ ID NO: 155 - RNA, 20 nt, Homo sapiens
agaagguugaccaguaucuc

SEQ ID NO: 156 - RNA, 20 nt, Homo sapiens
aagguugaccaguaucucua

SEQ ID NO: 157 - RNA, 20 nt, Homo sapiens
uugagaccaaagacaucuca

SEQ ID NO: 158 - RNA, 20 nt, Homo sapiens
acgguuccgucuacaagaaa

SEQ ID NO: 159 - RNA, 20 nt, Homo sapiens
uugaccaguaucucuaccac

SEQ ID NO: 160 - RNA, 41 nt, Homo sapiens
gcaguggcacccagcuguuugaccacauugccgaaugccug

SEQ ID NO: 161 - RNA, 41 nt, Homo sapiens
uggcacccagcuguuugaccacauugccgaaugccuggcua

SEQ ID NO: 162 - RNA, 41 nt, Homo sapiens
cacccagcuguuugaccacauugccgaaugccuggcuaacu

SEQ ID NO: 163 - RNA, 41 nt, Homo sapiens
uccaccggcgagaggaguuugaccuggaugugguugcugug

SEQ ID NO: 164 - RNA, 41 nt, Homo sapiens
aagacccucacugugaaguuggccucauuguuggcacgggc

SEQ ID NO: 165 - RNA, 41 nt, Homo sapiens
cacgggcagcaaugccugcuacauggaggagaugcgcaacg

SEQ ID NO: 166 - RNA, 41 nt, Homo sapiens
ugagagcaccugugacgacagcaucauuguuaaggaggugu

SEQ ID NO: 167 - RNA, 41 nt, Homo sapiens
gaccacaacugugagauuggucucauugugggcacgggcag

SEQ ID NO: 168 - RNA, 41 nt, Homo sapiens
ccaaguucuugucucagauugagagugacugccuggcccug

SEQ ID NO: 169 - RNA, 41 nt, Homo sapiens
aaguucuugucucagauugagagugacugccuggcccugcu

SEQ ID NO: 170 - RNA, 41 nt, Homo sapiens
cacuuugccaaagucaugcaugagacagugaaggaccuggc

SEQ ID NO: 171 - RNA, 41 nt, Homo sapiens
cucaaauccuugagcacucagucuagugaagauguugucau

SEQ ID NO: 172 - RNA, 41 nt, Homo sapiens
aucuuggagggaccaacuuccgugugcuuugggugaaagua

SEQ ID NO: 173 - RNA, 41 nt, Homo sapiens
agguucgagaaaaugaucaguggaauguaccugggugagau

SEQ ID NO: 174 - RNA, 41 nt, Homo sapiens
caucucaaauccuugagcacucagucuagugaagauguugu

SEQ ID NO: 175 - RNA, 41 nt, Homo sapiens
ccucuacaagcuacauccucacuuugccaaagucaugcaug

SEQ ID NO: 176 - RNA, 41 nt, Homo sapiens
acuucuuggccuuggaccuuggaggaacaaauuuccggguc

SEQ ID NO: 177 - RNA, 41 nt, Homo sapiens
ggguggaguggagaugcacaacaagaucuacgccaucccgc

SEQ ID NO: 178 - RNA, 41 nt, Homo sapiens
gacacagucggaacuaugaugaccuguggcuuugaagaccc

SEQ ID NO: 179 - RNA, 41 nt, Homo sapiens
gaccaagugcagaagguugaccaguaucucuaccacaugcg

SEQ ID NO: 180 - RNA, 41 nt, Homo sapiens
ccaagugcagaagguugaccaguaucucuaccacaugcgcc

SEQ ID NO: 181 - RNA, 41 nt, Homo sapiens
accggucgcuuugagaccaaagacaucucagacauugaagg

SEQ ID NO: 182 - RNA, 41 nt, Homo sapiens
auuggggucgacgguuccgucuacaagaaacacccccauuu

SEQ ID NO: 183 - RNA, 41 nt, Homo sapiens
gugcagaagguugaccaguaucucuaccacaugcgccucuc

SEQ ID NO: 184 - RNA, 15 nt, Homo sapiens
uguuugaccacauua

SEQ ID NO: 185 - RNA, 15 nt, Homo sapiens
ugaccacauugccga

SEQ ID NO: 186 - RNA, 15 nt, Homo sapiens
ccacauugccgaaua

SEQ ID NO: 187 - RNA, 15 nt, Homo sapiens
aguuugaccuggaua

SEQ ID NO: 188 - RNA, 15 nt, Homo sapiens
aaguuggccucauua

SEQ ID NO: 189 - RNA, 15 nt, Homo sapiens
cugcuacauggagga

SEQ ID NO: 190 - RNA, 15 nt, Homo sapiens
cgacagcaucauuga

SEQ ID NO: 191 - RNA, 15 nt, Homo sapiens
auuggucucauugua

SEQ ID NO: 192 - RNA, 15 nt, Homo sapiens
agauugagagugaca

SEQ ID NO: 193 - RNA, 15 nt, Homo sapiens
auugagagugacuga

SEQ ID NO: 194 - RNA, 15 nt, Homo sapiens
augcaugagacagua

SEQ ID NO: 195 - RNA, 15 nt, Homo sapiens
acucagucuagugaa

SEQ ID NO: 196 - RNA, 15 nt, Homo sapiens
acuuccgugugcuua

SEQ ID NO: 197 - RNA, 15 nt, Homo sapiens
aucaguggaauguaa

SEQ ID NO: 198 - RNA, 15 nt, Homo sapiens
agcacucagucuaga

SEQ ID NO: 199 - RNA, 15 nt, Homo sapiens
uccucacuuugccaa

SEQ ID NO: 200 - RNA, 15 nt, Homo sapiens
accuuggaggaacaa

SEQ ID NO: 201 - RNA, 15 nt, Homo sapiens
gcacaacaagaucua

SEQ ID NO: 202 - RNA, 15 nt, Homo sapiens
augaugaccugugga

SEQ ID NO: 203 - RNA, 15 nt, Homo sapiens
guugaccaguaucua

SEQ ID NO: 204 - RNA, 15 nt, Homo sapiens
ugaccaguaucucua

SEQ ID NO: 205 - RNA, 15 nt, Homo sapiens
accaaagacaucuca

SEQ ID NO: 206 - RNA, 15 nt, Homo sapiens
uccgucuacaagaaa

SEQ ID NO: 207 - RNA, 15 nt, Homo sapiens
caguaucucuaccaa

SEQ ID NO: 208 - RNA, 20 nt, Artificial Sequence
  Synthetic Polynucleotide
uaauguggucaaacagcugg

SEQ ID NO: 209 - RNA, 20 nt, Artificial Sequence
  Synthetic Polynucleotide
ucggcaauguggucaaacag

SEQ ID NO: 210 - RNA, 20 nt, Artificial Sequence
  Synthetic Polynucleotide
uauucggcaauguggucaaa

SEQ ID NO: 211 - RNA, 20 nt, Artificial Sequence
  Synthetic Polynucleotide
uauccaggucaaacuccucu

SEQ ID NO: 212 - RNA, 20 nt, Artificial Sequence
  Synthetic Polynucleotide
uaaugaggccaacuucacag

SEQ ID NO: 213 - RNA, 20 nt, Artificial Sequence
  Synthetic Polynucleotide
uccuccauguagcaggcauu

SEQ ID NO: 214 - RNA, 20 nt, Artificial Sequence
  Synthetic Polynucleotide
ucaaugaugcugucgucaca

SEQ ID NO: 215 - RNA, 20 nt, Artificial Sequence
  Synthetic Polynucleotide
uacaaugagaccaaucucac

SEQ ID NO: 216 - RNA, 20 nt, Artificial Sequence
  Synthetic Polynucleotide
ugucacucucaaucugagac

SEQ ID NO: 217 - RNA, 20 nt, Artificial Sequence
  Synthetic Polynucleotide
ucagucacucucaaucugag

SEQ ID NO: 218 - RNA, 20 nt, Artificial Sequence
  Synthetic Polynucleotide
uacugucucaugcaugacuu

SEQ ID NO: 219 - RNA, 20 nt, Artificial Sequence
  Synthetic Polynucleotide
uucacuagacugagugcuca

SEQ ID NO: 220 - RNA, 20 nt, Artificial Sequence
  Synthetic Polynucleotide
uaagcacacggaaguugguc

SEQ ID NO: 221 - RNA, 20 nt, Artificial Sequence
  Synthetic Polynucleotide
uuacauuccacugaucauuu

SEQ ID NO: 222 - RNA, 20 nt, Artificial Sequence
  Synthetic Polynucleotide
ucuagacugagugcucaagg

SEQ ID NO: 223 - RNA, 20 nt, Artificial Sequence
  Synthetic Polynucleotide
uuggcaaagugaggauguag

SEQ ID NO: 224 - RNA, 20 nt, Artificial Sequence
  Synthetic Polynucleotide
uuguuccuccaagguccaag

SEQ ID NO: 225 - RNA, 20 nt, Artificial Sequence
  Synthetic Polynucleotide
uagaucuuguugugcaucuc

SEQ ID NO: 226 - RNA, 20 nt, Artificial Sequence
  Synthetic Polynucleotide
uccacaggucaucauaguuc

SEQ ID NO: 227 - RNA, 20 nt, Artificial Sequence
  Synthetic Polynucleotide
uagauacuggucaaccuucu

SEQ ID NO: 228 - RNA, 20 nt, Artificial Sequence
  Synthetic Polynucleotide
uagagauacuggucaaccuu

SEQ ID NO: 229 - RNA, 20 nt, Artificial Sequence
  Synthetic Polynucleotide
ugagaugucuuuggucucaa

SEQ ID NO: 230 - RNA, 20 nt, Artificial Sequence
  Synthetic Polynucleotide
uuucuuguagacggaaccgu

SEQ ID NO: 231 - RNA, 20 nt, Artificial Sequence
  Synthetic Polynucleotide
uugguagagauacuggucaa

SEQ ID NO: 232 - RNA, 15 nt, Artificial Sequence
  Synthetic Polynucleotide
gauuuaacuugcaaa

SEQ ID NO: 233 - RNA, 20 nt, Artificial Sequence
  Synthetic Polynucleotide
uuugcaaguuaaaucaauug

SEQ ID NO: 234 - RNA, 15 nt, Artificial Sequence
  Synthetic Polynucleotide
cuugcaaugauuaca

SEQ ID NO: 235 - RNA, 20 nt, Artificial Sequence
  Synthetic Polynucleotide
uguaaucauugcaaguuaaa

SEQ ID NO: 236 - RNA, 15 nt, Artificial Sequence
  Synthetic Polynucleotide
  misc_feature (1)..(1): 2′ fluoro uridine
  misc_feature (1)..(13): modified by a phosphodiester linkage
  misc_feature (2)..(2): 2′-O-methyl adenine
  misc_feature (3)..(3): 2′ fluoro adenine
  misc_feature (4)..(4): 2′-O-methyl adenine
  misc_feature (5)..(5): 2′ fluoro guanine
  misc_feature (6)..(6): 2′-O-methyl cytidine
  misc_feature (7)..(7): 2′ fluoro adenine
  misc_feature (8)..(8): 2′-O-methyl cytidine
  misc_feature (9)..(9): 2′ fluoro guanine
  misc_feature (10)..(10): 2′-O-methyl adenine
  misc_feature (11)..(11): 2′ fluoro cytidine
  misc_feature (12)..(12): 2′-O-methyl uridine
  misc_feature (13)..(13): 2′ fluoro adenine
  misc_feature (13)..(15): modified by a phosphorothioate linkage
  misc_feature (14)..(14): 2′-O-methyl cytidine
  misc_feature (15)..(15): 2′ fluoro adenine
  misc_feature (15)..(15): modified by TEG-Chl
uaaagcacgacuaca

SEQ ID NO: 237 - RNA, 20 nt, Artificial Sequence
  Synthetic Polynucleotide
  misc_feature (1)..(1): modified by 5′ inorganic Phosphate
  misc_feature (1)..(14): modified by a phosphodiester linkage
  misc_feature (1)..(1): 2′-O-methyl uridine
  misc_feature (2)..(2): 2′ fluoro guanine
  misc_feature (3)..(3): 2′-O-methyl uridine
  misc_feature (4)..(4): 2′ fluoro adenine
  misc_feature (5)..(5): 2′-O-methyl guanine
  misc_feature (6)..(6): 2′ fluoro uridine
  misc_feature (7)..(7): 2′-O-methyl cytidine
  misc_feature (8)..(8): 2′ fluoro guanine
  misc_feature (9)..(9): 2′-O-methyl uridine
  misc_feature (10)..(10): 2′ fluoro guanine
  misc_feature (11)..(11): 2′-O-methyl cytidine
  misc_feature (12)..(12): 2′ fluoro uridine
  misc_feature (13)..(13): 2′-O-methyl uridine
  misc_feature (14)..(20): modified by a phosphothioate linkage
  misc_feature (14)..(14): 2′ fluoro uridine
  misc_feature (15)..(15): 2′-O-methyl adenine
  misc_feature (16)..(16): 2′ fluoro adenine
  misc_feature (17)..(17): 2′-O-methyl cytidine
  misc_feature (18)..(18): 2′ fluoro uridine
  misc_feature (19)..(19): 2′-O-methyl uridine
  misc_feature (20)..(20): 2′ fluoro cytidine
uguagucgugcuuuaacuuc

SEQ ID NO: 238 - RNA, 15 nt, Artificial Sequence
  Synthetic Polynucleotide
uacacaggaacaaua

SEQ ID NO: 239 - RNA, 20 nt, Artificial Sequence
  Synthetic Polynucleotide
uauuguuccuguguaacuuu

SEQ ID NO: 240 - RNA, 15 nt, Artificial Sequence
  Synthetic Polynucleotide
ucguugugaaauaaa

SEQ ID NO: 241 - RNA, 20 nt, Artificial Sequence
  Synthetic Polynucleotide
uuuauuucacaacgacagaa

SEQ ID NO: 242 - RNA, 15 nt, Artificial Sequence
  Synthetic Polynucleotide
ugcauggugagaaaa

SEQ ID NO: 243 - RNA, 20 nt, Artificial Sequence
  Synthetic Polynucleotide
uuuucucaccaugcaaggag

SEQ ID NO: 244 - RNA, 15 nt, Artificial Sequence
  Synthetic Polynucleotide
cggucuugugauaaa

SEQ ID NO: 245 - RNA, 20 nt, Artificial Sequence
  Synthetic Polynucleotide
uuuaucacaagaccgaacag

SEQ ID NO: 246 - RNA, 15 nt, Artificial Sequence
  Synthetic Polynucleotide
uaagcauauauuuaa

SEQ ID NO: 247 - RNA, 20 nt, Artificial Sequence
  Synthetic Polynucleotide
uuaaauauaugcuuaaguca

SEQ ID NO: 248 - RNA, 15 nt, Artificial Sequence
  Synthetic Polynucleotide
  misc_feature (1)..(1): 2′ fluoro cytidine
  misc_feature (1)..(13): modified by a phosphodiester linkage
  misc_feature (2)..(2): 2′-O-methyl adenine
  misc_feature (3)..(3): 2′ fluoro uridine
  misc_feature (4)..(4): 2′-O-methyl uridine
  misc_feature (5)..(5): 2′ fluoro guanine
  misc_feature (6)..(6): 2′-O-methyl adenine
  misc_feature (7)..(7): 2′ fluoro adenine
  misc_feature (8)..(8): 2′-O-methyl cytidine
  misc_feature (9)..(9): 2′ fluoro adenine
  misc_feature (10)..(10): 2′-O-methyl uridine
  misc_feature (11)..(11): 2′ fluoro uridine
  misc_feature (12)..(12): 2′-O-methyl cytidine
  misc_feature (13)..(13): 2′ fluoro adenine
  misc_feature (13)..(15): modified by a phosphorothioate linkage
  misc_feature (14)..(14): 2′-O-methyl adenine
  misc_feature (15)..(15): 2′ fluoro adenine
  misc_feature (15)..(15): modified by TEG-Chl
cauugaacauucaaa

SEQ ID NO: 249 - RNA, 20 nt, Artificial Sequence
  Synthetic Polynucleotide
  misc_feature (1)..(1): modified by 5′ inorganic Phosphate
  misc_feature (1)..(14): modified by a phosphodiester linkage
  misc_feature (1)..(1): 2′-O-methyl uridine
  misc_feature (2)..(2): 2′ fluoro uridine
  misc_feature (3)..(3): 2′-O-methyl uridine
  misc_feature (4)..(4): 2′ fluoro guanine
  misc_feature (5)..(5): 2′-O-methyl adenine
  misc_feature (6)..(6): 2′ fluoro adenine
  misc_feature (7)..(7): 2′-O-methyl uridine
  misc_feature (8)..(8): 2′ fluoro guanine
  misc_feature (9)..(9): 2′-O-methyl uridine
  misc_feature (10)..(10): 2′ fluoro uridine
  misc_feature (11)..(11): 2′-O-methyl cytidine
  misc_feature (12)..(12): 2′ fluoro adenine
  misc_feature (13)..(13): 2′-O-methyl adenine
  misc_feature (14)..(20): modified by a phosphothioate linkage
  misc_feature (14)..(14): 2′ fluoro uridine
  misc_feature (15)..(15): 2′-O-methyl guanine
  misc_feature (16)..(16): 2′ fluoro adenine
  misc_feature (17)..(17): 2′-O-methyl guanine
  misc_feature (18)..(18): 2′ fluoro adenine
  misc_feature (19)..(19): 2′-O-methyl cytidine
  misc_feature (20)..(20): 2′ fluoro uridine
uuugaauguucaaugagacu

SEQ ID NO: 250 - RNA, 15 nt, Artificial Sequence
  Synthetic Polynucleotide
uuugauaccuguaca

SEQ ID NO: 251 - RNA, 20 nt, Artificial Sequence
  Synthetic Polynucleotide
uguacagguaucaaaacacc

SEQ ID NO: 252 - RNA, 15 nt, Artificial Sequence
  Synthetic Polynucleotide
gaucaaacuaaugca

SEQ ID NO: 253 - RNA, 20 nt, Artificial Sequence
  Synthetic Polynucleotide
ugcauuaguuugaucaguug

SEQ ID NO: 254 - RNA, 15 nt, Artificial Sequence
  Synthetic Polynucleotide
uuauuugucaauaaa

SEQ ID NO: 255 - RNA, 20 nt, Artificial Sequence
  Synthetic Polynucleotide
uuuauugacaaauaaaugcu

SEQ ID NO: 256 - RNA, 20 nt, Homo sapiens
uuauugaugagcgcacaaga

SEQ ID NO: 257 - RNA, 20 nt, Homo sapiens
auugugucuugguggaugac

SEQ ID NO: 258 - RNA, 20 nt, Homo sapiens
auguaccgcaaagccaucua

SEQ ID NO: 259 - RNA, 20 nt, Homo sapiens
aaacaacaacugcugcaggu

SEQ ID NO: 260 - RNA, 20 nt, Homo sapiens
acagaagcauauccaggagu

SEQ ID NO: 261 - RNA, 20 nt, Homo sapiens
ucuuugaguucuaccgccuc

SEQ ID NO: 262 - RNA, 20 nt, Homo sapiens
uaaaagguacuguuaacuac

SEQ ID NO: 263 - RNA, 20 nt, Homo sapiens
cugaucagauaggagcacaa

SEQ ID NO: 264 - RNA, 20 nt, Homo sapiens
uuauggugcacugaaaugga

SEQ ID NO: 265 - RNA, 20 nt, Homo sapiens
gcaaagugaggaccauuacu

SEQ ID NO: 266 - RNA, 20 nt, Homo sapiens
cgcuguuaccucuuguuuac

SEQ ID NO: 267 - RNA, 20 nt, Homo sapiens
ccacacaggaaaccuugaag

SEQ ID NO: 268 - RNA, 20 nt, Homo sapiens
cuugcugugacugaaacaag

SEQ ID NO: 269 - RNA, 20 nt, Homo sapiens
gugaugauugaugccaaaga

SEQ ID NO: 270 - RNA, 20 nt, Homo sapiens
aauaaccacgaccaggaauu

SEQ ID NO: 271 - RNA, 20 nt, Homo sapiens
uuuugcagugcguuccacca

SEQ ID NO: 272 - RNA, 20 nt, Homo sapiens
uaccaguccuacugcaccau

SEQ ID NO: 273 - RNA, 20 nt, Homo sapiens
uucagguggaccgcuacauu

SEQ ID NO: 274 - RNA, 20 nt, Homo sapiens
caucucgcgauuucucgagu

SEQ ID NO: 275 - RNA, 20 nt, Homo sapiens
cuccgcugaaggaguauuuu

SEQ ID NO: 276 - RNA, 20 nt, Homo sapiens
cagauguucuucgcuaauaa

SEQ ID NO: 277 - RNA, 20 nt, Homo sapiens
aaugugccaaaacugcaaga

SEQ ID NO: 278 - RNA, 20 nt, Homo sapiens
uucggagacggcaaauucuc

SEQ ID NO: 279 - RNA, 20 nt, Homo sapiens
ugguucggagacggcaaauu

SEQ ID NO: 280 - RNA, 41 nt, Homo sapiens
gucaaggagauuauugaugagcgcacaagagagcggcuggu

SEQ ID NO: 281 - RNA, 41 nt, Homo sapiens
gccaggccgcauugugucuugguggaugacgggccggagcc

SEQ ID NO: 282 - RNA, 41 nt, Homo sapiens
caagcagcccauguaccgcaaagccaucuacgagguccugc

SEQ ID NO: 283 - RNA, 41 nt, Homo sapiens
ucaugugcggaaacaacaacugcugcaggugcuuuugcgug

SEQ ID NO: 284 - RNA, 41 nt, Homo sapiens
gcagcgucacacagaagcauauccaggaguggggcccauuc

SEQ ID NO: 285 - RNA, 41 nt, Homo sapiens
ggccggcucuucuuugaguucuaccgccuccugcaugaugc

SEQ ID NO: 286 - RNA, 41 nt, Homo sapiens
gauauauauauaaaagguacuguuaacuacuguacaacccg

SEQ ID NO: 287 - RNA, 41 nt, Homo sapiens
ugucucuagccugaucagauaggagcacaagcaggggacgg

SEQ ID NO: 288 - RNA, 41 nt, Homo sapiens
agaggacaucuuauggugcacugaaauggaaaggguauuug

SEQ ID NO: 289 - RNA, 41 nt, Homo sapiens
gccaaguucagcaaagugaggaccauuacuacgaggucaaa

SEQ ID NO: 290 - RNA, 41 nt, Homo sapiens
uucuagaagccgcuguuaccucuuguuuacaguuuauauau

SEQ ID NO: 291 - RNA, 41 nt, Homo sapiens
caggugccuaccacacaggaaaccuugaagaaaaucaguuu

SEQ ID NO: 292 - RNA, 41 nt, Homo sapiens
gguuuuguuucuugcugugacugaaacaagaagguuauugc

SEQ ID NO: 293 - RNA, 41 nt, Homo sapiens
guccaacccugugaugauugaugccaaagaagugucagcug

SEQ ID NO: 294 - RNA, 41 nt, Homo sapiens
guucuucgcuaauaaccacgaccaggaauuugacccuccaa

SEQ ID NO: 295 - RNA, 41 nt, Homo sapiens
gcugagcucguuuugcagugcguuccaccaggccacguaca

SEQ ID NO: 296 - RNA, 41 nt, Homo sapiens
cgacgacggcuaccaguccuacugcaccaucugcugugggg

SEQ ID NO: 297 - RNA, 41 nt, Homo sapiens
gacuugggcauucagguggaccgcuacauugccucggaggu

SEQ ID NO: 298 - RNA, 41 nt, Homo sapiens
acaagagggacaucucgcgauuucucgaguccaacccugug

SEQ ID NO: 299 - RNA, 41 nt, Homo sapiens
caccucuucgcuccgcugaaggaguauuuugcgugugugua

SEQ ID NO: 300 - RNA, 41 nt, Homo sapiens
cucccggcuccagauguucuucgcuaauaaccacgaccagg

SEQ ID NO: 301 - RNA, 41 nt, Homo sapiens
ucguuggaggaaugugccaaaacugcaagaacugcuuucug

SEQ ID NO: 302 - RNA, 41 nt, Homo sapiens
ggucaugugguucggagacggcaaauucucaguggugugug

SEQ ID NO: 303 - RNA, 41 nt, Homo sapiens
cugggucaugugguucggagacggcaaauucucaguggugu

SEQ ID NO: 304 - RNA, 15 nt, Homo sapiens
gaugagcgcacaaga

SEQ ID NO: 305 - RNA, 15 nt, Homo sapiens
gucuugguggaugaa

SEQ ID NO: 306 - RNA, 15 nt, Homo sapiens
ccgcaaagccaucua

SEQ ID NO: 307 - RNA, 15 nt, Homo sapiens
acaacugcugcagga

SEQ ID NO: 308 - RNA, 15 nt, Homo sapiens
agcauauccaggaga

SEQ ID NO: 309 - RNA, 15 nt, Homo sapiens
gaguucuaccgccua

SEQ ID NO: 310 - RNA, 15 nt, Homo sapiens
gguacuguuaacuaa

SEQ ID NO: 311 - RNA, 15 nt, Homo sapiens
cagauaggagcacaa

SEQ ID NO: 312 - RNA, 15 nt, Homo sapiens
gugcacugaaaugga

SEQ ID NO: 313 - RNA, 15 nt, Homo sapiens
gugaggaccauuaca

SEQ ID NO: 314 - RNA, 15 nt, Homo sapiens
uuaccucuuguuuaa

SEQ ID NO: 315 - RNA, 15 nt, Homo sapiens
caggaaaccuugaaa

SEQ ID NO: 316 - RNA, 15 nt, Homo sapiens
ugugacugaaacaaa

SEQ ID NO: 317 - RNA, 15 nt, Homo sapiens
gauugaugccaaaga

SEQ ID NO: 318 - RNA, 15 nt, Homo sapiens
ccacgaccaggaaua

SEQ ID NO: 319 - RNA, 15 nt, Homo sapiens
cagugcguuccacca

SEQ ID NO: 320 - RNA, 15 nt, Homo sapiens
guccuacugcaccaa

SEQ ID NO: 321 - RNA, 15 nt, Homo sapiens
guggaccgcuacaua

SEQ ID NO: 322 - RNA, 15 nt, Homo sapiens
cgcgauuucucgaga

SEQ ID NO: 323 - RNA, 15 nt, Homo sapiens
cugaaggaguauuua

SEQ ID NO: 324 - RNA, 15 nt, Homo sapiens
guucuucgcuaauaa

SEQ ID NO: 325 - RNA, 15 nt, Homo sapiens
gccaaaacugcaaga

SEQ ID NO: 326 - RNA, 15 nt, Homo sapiens
agacggcaaauucua

SEQ ID NO: 327 - RNA, 15 nt, Homo sapiens
cggagacggcaaaua

SEQ ID NO: 328 - RNA, 20 nt, Artificial Sequence
  Synthetic Polynucleotide
ucuugugcgcucaucaauaa

SEQ ID NO: 329 - RNA, 20 nt, Artificial Sequence
  Synthetic Polynucleotide
uucauccaccaagacacaau

SEQ ID NO: 330 - RNA, 20 nt, Artificial Sequence
  Synthetic Polynucleotide
uagauggcuuugcgguacau

SEQ ID NO: 331 - RNA, 20 nt, Artificial Sequence
  Synthetic Polynucleotide
uccugcagcaguuguuguuu

SEQ ID NO: 332 - RNA, 20 nt, Artificial Sequence
  Synthetic Polynucleotide
ucuccuggauaugcuucugu

SEQ ID NO: 333 - RNA, 20 nt, Artificial Sequence
  Synthetic Polynucleotide
uaggcgguagaacucaaaga

SEQ ID NO: 334 - RNA, 20 nt, Artificial Sequence
  Synthetic Polynucleotide
uuaguuaacaguaccuuuua

SEQ ID NO: 335 - RNA, 20 nt, Artificial Sequence
  Synthetic Polynucleotide
uugugcuccuaucugaucag

SEQ ID NO: 336 - RNA, 20 nt, Artificial Sequence
  Synthetic Polynucleotide
uccauuucagugcaccauaa

SEQ ID NO: 337 - RNA, 20 nt, Artificial Sequence
  Synthetic Polynucleotide
uguaaugguccucacuuugc

SEQ ID NO: 338 - RNA, 20 nt, Artificial Sequence
  Synthetic Polynucleotide
uuaaacaagagguaacagcg

SEQ ID NO: 339 - RNA, 20 nt, Artificial Sequence
  Synthetic Polynucleotide
uuucaagguuuccugugugg

SEQ ID NO: 340 - RNA, 20 nt, Artificial Sequence
  Synthetic Polynucleotide
uuuguuucagucacagcaag

SEQ ID NO: 341 - RNA, 20 nt, Artificial Sequence
  Synthetic Polynucleotide
ucuuuggcaucaaucaucac

SEQ ID NO: 342 - RNA, 20 nt, Artificial Sequence
  Synthetic Polynucleotide
uauuccuggucgugguuauu

SEQ ID NO: 343 - RNA, 20 nt, Artificial Sequence
  Synthetic Polynucleotide
ugguggaacgcacugcaaaa

SEQ ID NO: 344 - RNA, 20 nt, Artificial Sequence
  Synthetic Polynucleotide
uuggugcaguaggacuggua

SEQ ID NO: 345 - RNA, 20 nt, Artificial Sequence
  Synthetic Polynucleotide
uauguagcgguccaccugaa

SEQ ID NO: 346 - RNA, 20 nt, Artificial Sequence
  Synthetic Polynucleotide
ucucgagaaaucgcgagaug

SEQ ID NO: 347 - RNA, 20 nt, Artificial Sequence
  Synthetic Polynucleotide
uaaauacuccuucagcggag

SEQ ID NO: 348 - RNA, 20 nt, Artificial Sequence
  Synthetic Polynucleotide
uuauuagcgaagaacaucug

SEQ ID NO: 349 - RNA, 20 nt, Artificial Sequence
  Synthetic Polynucleotide
ucuugcaguuuuggcacauu

SEQ ID NO: 350 - RNA, 20 nt, Artificial Sequence
  Synthetic Polynucleotide
uagaauuugccgucuccgaa

SEQ ID NO: 351 - RNA, 20 nt, Artificial Sequence
  Synthetic Polynucleotide
uauuugccgucuccgaacca

SEQ ID NO: 352 - RNA, 20 nt, Homo sapiens
agagaguacagcgugaaaga

SEQ ID NO: 353 - RNA, 20 nt, Homo sapiens
agaguacagcgugaaagaaa

SEQ ID NO: 354 - RNA, 20 nt, Homo sapiens
aggaacuucuugugugguau

SEQ ID NO: 355 - RNA, 20 nt, Homo sapiens
aggucugccacaagagauuu

SEQ ID NO: 356 - RNA, 20 nt, Homo sapiens
gucugccacaagagauuuag

SEQ ID NO: 357 - RNA, 20 nt, Homo sapiens
accacuuaaauugugagcca

SEQ ID NO: 358 - RNA, 20 nt, Homo sapiens
uugugagccaagccauguaa

SEQ ID NO: 359 - RNA, 20 nt, Homo sapiens
cuguaaaggucaaacaagaa

SEQ ID NO: 360 - RNA, 20 nt, Homo sapiens
uuuacuuugcuagaacaaca

SEQ ID NO: 361 - RNA, 20 nt, Homo sapiens
acuuugcuagaacaacaaac

SEQ ID NO: 362 - RNA, 20 nt, Homo sapiens
agagagaguacagcgugaaa

SEQ ID NO: 363 - RNA, 20 nt, Homo sapiens
gaugaacaucuacuucuaca

SEQ ID NO: 364 - RNA, 20 nt, Homo sapiens
acuucuugugugguauuguc

SEQ ID NO: 365 - RNA, 20 nt, Homo sapiens
gaagccaugaaucucauuaa

SEQ ID NO: 366 - RNA, 20 nt, Homo sapiens
aaaguuuacaaugacuggaa

SEQ ID NO: 367 - RNA, 20 nt, Homo sapiens
aaucugaagguccaccugag

SEQ ID NO: 368 - RNA, 20 nt, Homo sapiens
guggagaacggccuuucaaa

SEQ ID NO: 369 - RNA, 20 nt, Homo sapiens
aaggcuuuaccaaccugucu

SEQ ID NO: 370 - RNA, 20 nt, Homo sapiens
aagcaacuggaugcgcuaug

SEQ ID NO: 371 - RNA, 20 nt, Homo sapiens
agccucaagguucaccugaa

SEQ ID NO: 372 - RNA, 20 nt, Homo sapiens
aagaacuacauccaucucug

SEQ ID NO: 373 - RNA, 20 nt, Homo sapiens
uugugcaccugaaacugcac

SEQ ID NO: 374 - RNA, 20 nt, Homo sapiens
aucugaagguccaccugaga

SEQ ID NO: 375 - RNA, 20 nt, Homo sapiens
cagaacggcaagaucaagua

SEQ ID NO: 376 - RNA, 41 nt, Homo sapiens
ugucccaaagagagaguacagcgugaaagaaauccuaaaau

SEQ ID NO: 377 - RNA, 41 nt, Homo sapiens
ucccaaagagagaguacagcgugaaagaaauccuaaaauug

SEQ ID NO: 378 - RNA, 41 nt, Homo sapiens
ccugccaaccaggaacuucuugugugguauugucgggacuu

SEQ ID NO: 379 - RNA, 41 nt, Homo sapiens
caugaaugccaggucugccacaagagauuuagcagcaccag

SEQ ID NO: 380 - RNA, 41 nt, Homo sapiens
ugaaugccaggucugccacaagagauuuagcagcaccagca

SEQ ID NO: 381 - RNA, 41 nt, Homo sapiens
uauauuuauaaccacuuaaauugugagccaagccauguaaa

SEQ ID NO: 382 - RNA, 41 nt, Homo sapiens
accacuuaaauugugagccaagccauguaaaagaucuacuu

SEQ ID NO: 383 - RNA, 41 nt, Homo sapiens
ccucugguaccuguaaaggucaaacaagaaacaguugaacc

SEQ ID NO: 384 - RNA, 41 nt, Homo sapiens
uuacuggcuuuuuacuuugcuagaacaacaaacuaucuuau

SEQ ID NO: 385 - RNA, 41 nt, Homo sapiens
cuggcuuuuuacuuugcuagaacaacaaacuaucuuauguu

SEQ ID NO: 386 - RNA, 41 nt, Homo sapiens
aaugucccaaagagagaguacagcgugaaagaaauccuaaa

SEQ ID NO: 387 - RNA, 41 nt, Homo sapiens
gucagaacgggaugaacaucuacuucuacaccauuaagccc

SEQ ID NO: 388 - RNA, 41 nt, Homo sapiens
ccaaccaggaacuucuugugugguauugucgggacuuugca

SEQ ID NO: 389 - RNA, 41 nt, Homo sapiens
cagcagcgacgaagccaugaaucucauuaaaaacaaaagaa

SEQ ID NO: 390 - RNA, 41 nt, Homo sapiens
aauaauuaaaaaaguuuacaaugacuggaaagauuccuugu

SEQ ID NO: 391 - RNA, 41 nt, Homo sapiens
ccagcucuccaaucugaagguccaccugagagugcacagug

SEQ ID NO: 392 - RNA, 41 nt, Homo sapiens
agagugcacaguggagaacggccuuucaaaugucagacuug

SEQ ID NO: 393 - RNA, 41 nt, Homo sapiens
gggugacaggaaggcuuuaccaaccugucucucccuccaaa

SEQ ID NO: 394 - RNA, 41 nt, Homo sapiens
augaagagaaaagcaacuggaugcgcuaugugaauccagca

SEQ ID NO: 395 - RNA, 41 nt, Homo sapiens
ccaucucuguagccucaagguucaccugaaagggaacugcg

SEQ ID NO: 396 - RNA, 41 nt, Homo sapiens
ccagugccacaagaacuacauccaucucuguagccucaagg

SEQ ID NO: 397 - RNA, 41 nt, Homo sapiens
uucacccaguuugugcaccugaaacugcacaagcgucugca

SEQ ID NO: 398 - RNA, 41 nt, Homo sapiens
cagcucuccaaucugaagguccaccugagagugcacagugg

SEQ ID NO: 399 - RNA, 41 nt, Homo sapiens
gcugaagaagcagaacggcaagaucaaguacgaaugcaacg

SEQ ID NO: 400 - RNA, 15 nt, Homo sapiens
guacagcgugaaaga

SEQ ID NO: 401 - RNA, 15 nt, Homo sapiens
acagcgugaaagaaa

SEQ ID NO: 402 - RNA, 15 nt, Homo sapiens
cuucuugugugguaa

SEQ ID NO: 403 - RNA, 15 nt, Homo sapiens
ugccacaagagauua

SEQ ID NO: 404 - RNA, 15 nt, Homo sapiens
ccacaagagauuuaa

SEQ ID NO: 405 - RNA, 15 nt, Homo sapiens
uuaaauugugagcca

SEQ ID NO: 406 - RNA, 15 nt, Homo sapiens
agccaagccauguaa

SEQ ID NO: 407 - RNA, 15 nt, Homo sapiens
aaggucaaacaagaa

SEQ ID NO: 408 - RNA, 15 nt, Homo sapiens
uuugcuagaacaaca

SEQ ID NO: 409 - RNA, 15 nt, Homo sapiens
gcuagaacaacaaaa

SEQ ID NO: 410 - RNA, 15 nt, Homo sapiens
gaguacagcgugaaa

SEQ ID NO: 411 - RNA, 15 nt, Homo sapiens
acaucuacuucuaca

SEQ ID NO: 412 - RNA, 15 nt, Homo sapiens
uugugugguauugua

SEQ ID NO: 413 - RNA, 15 nt, Homo sapiens
caugaaucucauuaa

SEQ ID NO: 414 - RNA, 15 nt, Homo sapiens
uuacaaugacuggaa

SEQ ID NO: 415 - RNA, 15 nt, Homo sapiens
gaagguccaccugaa

SEQ ID NO: 416 - RNA, 15 nt, Homo sapiens
gaacggccuuucaaa

SEQ ID NO: 417 - RNA, 15 nt, Homo sapiens
uuuaccaaccuguca

SEQ ID NO: 418 - RNA, 15 nt, Homo sapiens
acuggaugcgcuaua

SEQ ID NO: 419 - RNA, 15 nt, Homo sapiens
caagguucaccugaa

SEQ ID NO: 420 - RNA, 15 nt, Homo sapiens
cuacauccaucucua

SEQ ID NO: 421 - RNA, 15 nt, Homo sapiens
caccugaaacugcaa

SEQ ID NO: 422 - RNA, 15 nt, Homo sapiens
aagguccaccugaga

SEQ ID NO: 423 - RNA, 15 nt, Homo sapiens
cggcaagaucaagua

SEQ ID NO: 424 - RNA, 20 nt, Artificial Sequence
  Synthetic Polynucleotide
ucuuucacgcuguacucucu

SEQ ID NO: 425 - RNA, 20 nt, Artificial Sequence
  Synthetic Polynucleotide
uuucuuucacgcuguacucu

SEQ ID NO: 426 - RNA, 20 nt, Artificial Sequence
  Synthetic Polynucleotide
uuaccacacaagaaguuccu

SEQ ID NO: 427 - RNA, 20 nt, Artificial Sequence
  Synthetic Polynucleotide
uaaucucuuguggcagaccu

SEQ ID NO: 428 - RNA, 20 nt, Artificial Sequence
  Synthetic Polynucleotide
uuaaaucucuuguggcagac

SEQ ID NO: 429 - RNA, 20 nt, Artificial Sequence
  Synthetic Polynucleotide
uggcucacaauuuaaguggu

SEQ ID NO: 430 - RNA, 20 nt, Artificial Sequence
  Synthetic Polynucleotide
uuacauggcuuggcucacaa

SEQ ID NO: 431 - RNA, 20 nt, Artificial Sequence
  Synthetic Polynucleotide
uucuuguuugaccuuuacag

SEQ ID NO: 432 - RNA, 20 nt, Artificial Sequence
  Synthetic Polynucleotide
uguuguucuagcaaaguaaa

SEQ ID NO: 433 - RNA, 20 nt, Artificial Sequence
  Synthetic Polynucleotide
uuuuguuguucuagcaaagu

SEQ ID NO: 434 - RNA, 20 nt, Artificial Sequence
  Synthetic Polynucleotide
uuucacgcuguacucucucu

SEQ ID NO: 435 - RNA, 20 nt, Artificial Sequence
  Synthetic Polynucleotide
uguagaaguagauguucauc

SEQ ID NO: 436 - RNA, 20 nt, Artificial Sequence
  Synthetic Polynucleotide
uacaauaccacacaagaagu

SEQ ID NO: 437 - RNA, 20 nt, Artificial Sequence
  Synthetic Polynucleotide
uuaaugagauucauggcuuc

SEQ ID NO: 438 - RNA, 20 nt, Artificial Sequence
  Synthetic Polynucleotide
uuccagucauuguaaacuuu

SEQ ID NO: 439 - RNA, 20 nt, Artificial Sequence
  Synthetic Polynucleotide
uucagguggaccuucagauu

SEQ ID NO: 440 - RNA, 20 nt, Artificial Sequence
  Synthetic Polynucleotide
uuugaaaggccguucuccac

SEQ ID NO: 441 - RNA, 20 nt, Artificial Sequence
  Synthetic Polynucleotide
ugacagguugguaaagccuu

SEQ ID NO: 442 - RNA, 20 nt, Artificial Sequence
  Synthetic Polynucleotide
uauagcgcauccaguugcuu

SEQ ID NO: 443 - RNA, 20 nt, Artificial Sequence
  Synthetic Polynucleotide
uucaggugaaccuugaggcu

SEQ ID NO: 444 - RNA, 20 nt, Artificial Sequence
  Synthetic Polynucleotide
uagagauggauguaguucuu

SEQ ID NO: 445 - RNA, 20 nt, Artificial Sequence
  Synthetic Polynucleotide
uugcaguuucaggugcacaa

SEQ ID NO: 446 - RNA, 20 nt, Artificial Sequence
  Synthetic Polynucleotide
ucucagguggaccuucagau

SEQ ID NO: 447 - RNA, 20 nt, Artificial Sequence
  Synthetic Polynucleotide
uacuugaucuugccguucug

SEQ ID NO: 448 - RNA, 20 nt, Homo sapiens
gcaagaaccgcuacaagaac

SEQ ID NO: 449 - RNA, 20 nt, Homo sapiens
aagaaccgcuacaagaacau

SEQ ID NO: 450 - RNA, 20 nt, Homo sapiens
gagaaaggccggaacaaaug

SEQ ID NO: 451 - RNA, 20 nt, Homo sapiens
uggagaaaggccggaacaaa

SEQ ID NO: 452 - RNA, 20 nt, Homo sapiens
cauauucggauccagaacuc

SEQ ID NO: 453 - RNA, 20 nt, Homo sapiens
uauucggauccagaacucag

SEQ ID NO: 454 - RNA, 20 nt, Homo sapiens
aaugacuucuggcagauggc

SEQ ID NO: 455 - RNA, 20 nt, Homo sapiens
gacuacaucaaugccaacua

SEQ ID NO: 456 - RNA, 20 nt, Homo sapiens
agaaccgcuacaagaacauu

SEQ ID NO: 457 - RNA, 20 nt, Homo sapiens
cauuaccaguaccugagcug

SEQ ID NO: 458 - RNA, 20 nt, Homo sapiens
uuugaccacagccgagugau

SEQ ID NO: 459 - RNA, 20 nt, Homo sapiens
acauccagaagaccauccag

SEQ ID NO: 460 - RNA, 20 nt, Homo sapiens
uggcauuaccaguaccugag

SEQ ID NO: 461 - RNA, 20 nt, Homo sapiens
gagaacgcuaagaccuacau

SEQ ID NO: 462 - RNA, 20 nt, Homo sapiens
aacgcuaagaccuacaucgc

SEQ ID NO: 463 - RNA, 20 nt, Homo sapiens
uacaaguucaucuacguggc

SEQ ID NO: 464 - RNA, 20 nt, Homo sapiens
agcaugacacaaccgaauac

SEQ ID NO: 465 - RNA, 20 nt, Homo sapiens
cacaaggaggauguguauga

SEQ ID NO: 466 - RNA, 20 nt, Homo sapiens
gugaaugcggcugacauuga

SEQ ID NO: 467 - RNA, 20 nt, Homo sapiens
acaucaaggucaugugcgag

SEQ ID NO: 468 - RNA, 20 nt, Homo sapiens
gaggcgcaguacaaguucau

SEQ ID NO: 469 - RNA, 20 nt, Homo sapiens
uugagaaccgaguguuggaa

SEQ ID NO: 470 - RNA, 20 nt, Homo sapiens
cauuucgcgauggacagacu

SEQ ID NO: 471 - RNA, 20 nt, Homo sapiens
uggacguuucuugugcguga

SEQ ID NO: 472 - RNA, 41 nt, Homo sapiens
gagaacaagggcaagaaccgcuacaagaacauucuccccuu

SEQ ID NO: 473 - RNA, 41 nt, Homo sapiens
gaacaagggcaagaaccgcuacaagaacauucuccccuuug

SEQ ID NO: 474 - RNA, 41 nt, Homo sapiens
ccgagagguggagaaaggccggaacaaaugcgucccauacu

SEQ ID NO: 475 - RNA, 41 nt, Homo sapiens
acccgagagguggagaaaggccggaacaaaugcgucccaua

SEQ ID NO: 476 - RNA, 41 nt, Homo sapiens
ucaggugacccauauucggauccagaacucaggggauuucu

SEQ ID NO: 477 - RNA, 41 nt, Homo sapiens
aggugacccauauucggauccagaacucaggggauuucuau

SEQ ID NO: 478 - RNA, 41 nt, Homo sapiens
ggccacggucaaugacuucuggcagauggcguggcaggaga

SEQ ID NO: 479 - RNA, 41 nt, Homo sapiens
ccccggguccgacuacaucaaugccaacuacaucaagaacc

SEQ ID NO: 480 - RNA, 41 nt, Homo sapiens
aacaagggcaagaaccgcuacaagaacauucuccccuuuga

SEQ ID NO: 481 - RNA, 41 nt, Homo sapiens
ggagaucuggcauuaccaguaccugagcuggcccgaccaug

SEQ ID NO: 482 - RNA, 41 nt, Homo sapiens
cauucuccccuuugaccacagccgagugauccugcagggac

SEQ ID NO: 483 - RNA, 41 nt, Homo sapiens
ugugacauugacauccagaagaccauccagauggugcgggc

SEQ ID NO: 484 - RNA, 41 nt, Homo sapiens
ucgggagaucuggcauuaccaguaccugagcuggcccgacc

SEQ ID NO: 485 - RNA, 41 nt, Homo sapiens
aggcccugaugagaacgcuaagaccuacaucgccagccagg

SEQ ID NO: 486 - RNA, 41 nt, Homo sapiens
cccugaugagaacgcuaagaccuacaucgccagccagggcu

SEQ ID NO: 487 - RNA, 41 nt, Homo sapiens
ggaggcgcaguacaaguucaucuacguggccaucgcccagu

SEQ ID NO: 488 - RNA, 41 nt, Homo sapiens
aacugcggggagcaugacacaaccgaauacaaacuccguac

SEQ ID NO: 489 - RNA, 41 nt, Homo sapiens
cggcugcagacacaaggaggauguguaugagaaccugcaca

SEQ ID NO: 490 - RNA, 41 nt, Homo sapiens
ugccacgagggugaaugcggcugacauugagaaccgagugu

SEQ ID NO: 491 - RNA, 41 nt, Homo sapiens
agggucacccacaucaaggucaugugcgaggguggacgcua

SEQ ID NO: 492 - RNA, 41 nt, Homo sapiens
ggugcagacggaggcgcaguacaaguucaucuacguggcca

SEQ ID NO: 493 - RNA, 41 nt, Homo sapiens
gcggcugacauugagaaccgaguguuggaacugaacaagaa

SEQ ID NO: 494 - RNA, 41 nt, Homo sapiens
ccuguggaagcauuucgcgauggacagacucacaaccugaa

SEQ ID NO: 495 - RNA, 41 nt, Homo sapiens
gggcgagcccuggacguuucuugugcgugagagccucagcc

SEQ ID NO: 496 - RNA, 15 nt, Homo sapiens
aaccgcuacaagaaa

SEQ ID NO: 497 - RNA, 15 nt, Homo sapiens
ccgcuacaagaacaa

SEQ ID NO: 498 - RNA, 15 nt, Homo sapiens
aggccggaacaaaua

SEQ ID NO: 499 - RNA, 15 nt, Homo sapiens
aaaggccggaacaaa

SEQ ID NO: 500 - RNA, 15 nt, Homo sapiens
ucggauccagaacua

SEQ ID NO: 501 - RNA, 15 nt, Homo sapiens
ggauccagaacucaa

SEQ ID NO: 502 - RNA, 15 nt, Homo sapiens
cuucuggcagaugga

SEQ ID NO: 503 - RNA, 15 nt, Homo sapiens
caucaaugccaacua

SEQ ID NO: 504 - RNA, 15 nt, Homo sapiens
cgcuacaagaacaua

SEQ ID NO: 505 - RNA, 15 nt, Homo sapiens
ccaguaccugagcua

SEQ ID NO: 506 - RNA, 15 nt, Homo sapiens
ccacagccgagugaa

SEQ ID NO: 507 - RNA, 15 nt, Homo sapiens
cagaagaccauccaa

SEQ ID NO: 508 - RNA, 15 nt, Homo sapiens
uuaccaguaccugaa

SEQ ID NO: 509 - RNA, 15 nt, Homo sapiens
cgcuaagaccuacaa

SEQ ID NO: 510 - RNA, 15 nt, Homo sapiens
uaagaccuacaucga

SEQ ID NO: 511 - RNA, 15 nt, Homo sapiens
guucaucuacgugga

SEQ ID NO: 512 - RNA, 15 nt, Homo sapiens
gacacaaccgaauaa

SEQ ID NO: 513 - RNA, 15 nt, Homo sapiens
ggaggauguguauga

SEQ ID NO: 514 - RNA, 15 nt, Homo sapiens
ugcggcugacauuga

SEQ ID NO: 515 - RNA, 15 nt, Homo sapiens
aaggucaugugcgaa

SEQ ID NO: 516 - RNA, 15 nt, Homo sapiens
gcaguacaaguucaa

SEQ ID NO: 517 - RNA, 15 nt, Homo sapiens
aaccgaguguuggaa

SEQ ID NO: 518 - RNA, 15 nt, Homo sapiens
cgcgauggacagaca

SEQ ID NO: 519 - RNA, 15 nt, Homo sapiens
guuucuugugcguga

SEQ ID NO: 520 - RNA, 20 nt, Artificial Sequence
  Synthetic Polynucleotide
uuucuuguagcgguucuugc

SEQ ID NO: 521 - RNA, 20 nt, Artificial Sequence
  Synthetic Polynucleotide
uuguucuuguagcgguucuu

SEQ ID NO: 522 - RNA, 20 nt, Artificial Sequence
  Synthetic Polynucleotide
uauuuguuccggccuuucuc

SEQ ID NO: 523 - RNA, 20 nt, Artificial Sequence
  Synthetic Polynucleotide
uuuguuccggccuuucucca

SEQ ID NO: 524 - RNA, 20 nt, Artificial Sequence
  Synthetic Polynucleotide
uaguucuggauccgaauaug

SEQ ID NO: 525 - RNA, 20 nt, Artificial Sequence
  Synthetic Polynucleotide
uugaguucuggauccgaaua

SEQ ID NO: 526 - RNA, 20 nt, Artificial Sequence
  Synthetic Polynucleotide
uccaucugccagaagucauu

SEQ ID NO: 527 - RNA, 20 nt, Artificial Sequence
  Synthetic Polynucleotide
uaguuggcauugauguaguc

SEQ ID NO: 528 - RNA, 20 nt, Artificial Sequence
  Synthetic Polynucleotide
uauguucuuguagcgguucu

SEQ ID NO: 529 - RNA, 20 nt, Artificial Sequence
  Synthetic Polynucleotide
uagcucagguacugguaaug

SEQ ID NO: 530 - RNA, 20 nt, Artificial Sequence
  Synthetic Polynucleotide
uucacucggcuguggucaaa

SEQ ID NO: 531 - RNA, 20 nt, Artificial Sequence
  Synthetic Polynucleotide
uuggauggucuucuggaugu

SEQ ID NO: 532 - RNA, 20 nt, Artificial Sequence
  Synthetic Polynucleotide
uucagguacugguaaugcca

SEQ ID NO: 533 - RNA, 20 nt, Artificial Sequence
  Synthetic Polynucleotide
uuguaggucuuagcguucuc

SEQ ID NO: 534 - RNA, 20 nt, Artificial Sequence
  Synthetic Polynucleotide
ucgauguaggucuuagcguu

SEQ ID NO: 535 - RNA, 20 nt, Artificial Sequence
  Synthetic Polynucleotide
uccacguagaugaacuugua

SEQ ID NO: 536 - RNA, 20 nt, Artificial Sequence
  Synthetic Polynucleotide
uuauucgguugugucaugcu

SEQ ID NO: 537 - RNA, 20 nt, Artificial Sequence
  Synthetic Polynucleotide
ucauacacauccuccuugug

SEQ ID NO: 538 - RNA, 20 nt, Artificial Sequence
  Synthetic Polynucleotide
ucaaugucagccgcauucac

SEQ ID NO: 539 - RNA, 20 nt, Artificial Sequence
  Synthetic Polynucleotide
uucgcacaugaccuugaugu

SEQ ID NO: 540 - RNA, 20 nt, Artificial Sequence
  Synthetic Polynucleotide
uugaacuuguacugcgccuc

SEQ ID NO: 541 - RNA, 20 nt, Artificial Sequence
  Synthetic Polynucleotide
uuccaacacucgguucucaa

SEQ ID NO: 542 - RNA, 20 nt, Artificial Sequence
  Synthetic Polynucleotide
ugucuguccaucgcgaaaug

SEQ ID NO: 543 - RNA, 20 nt, Artificial Sequence
  Synthetic Polynucleotide
ucacgcacaagaaacgucca

SEQ ID NO: 544 - RNA, 20 nt, Homo sapiens
uaaugccuaauggugcuaca

SEQ ID NO: 545 - RNA, 20 nt, Homo sapiens
aagagcaucauugagaccau

SEQ ID NO: 546 - RNA, 20 nt, Homo sapiens
ugccuaauggugcuacaguu

SEQ ID NO: 547 - RNA, 20 nt, Homo sapiens
agcaucauugagaccaugga

SEQ ID NO: 548 - RNA, 20 nt, Homo sapiens
aagcaagccugauggaacag

SEQ ID NO: 549 - RNA, 20 nt, Homo sapiens
augcugauaaugccaguaaa

SEQ ID NO: 550 - RNA, 20 nt, Homo sapiens
aaauggagacaccaaguggc

SEQ ID NO: 551 - RNA, 20 nt, Homo sapiens
ugaugcugauaaugccagua

SEQ ID NO: 552 - RNA, 20 nt, Homo sapiens
agucacaaauguaccaaguu

SEQ ID NO: 553 - RNA, 20 nt, Homo sapiens
augaauggugcuuacuucaa

SEQ ID NO: 554 - RNA, 20 nt, Homo sapiens
uaaaacgcacaguuagugaa

SEQ ID NO: 555 - RNA, 20 nt, Homo sapiens
uggugcuuacuucaagcaaa

SEQ ID NO: 556 - RNA, 20 nt, Homo sapiens
gugcuuacuucaagcaaagc

SEQ ID NO: 557 - RNA, 20 nt, Homo sapiens
cagaaggacacucaaaagca

SEQ ID NO: 558 - RNA, 20 nt, Homo sapiens
uauuauccagauuguguuuc

SEQ ID NO: 559 - RNA, 20 nt, Homo sapiens
aggacacucaaaagcaugcu

SEQ ID NO: 560 - RNA, 20 nt, Homo sapiens
cauuaacagucaggcuacua

SEQ ID NO: 561 - RNA, 20 nt, Homo sapiens
uguugaaacagcacuugaau

SEQ ID NO: 562 - RNA, 20 nt, Homo sapiens
uuggccagacuaaaguggaa

SEQ ID NO: 563 - RNA, 20 nt, Homo sapiens
uggcagcucugaacgguauu

SEQ ID NO: 564 - RNA, 20 nt, Homo sapiens
aaagguacuugauacauaac

SEQ ID NO: 565 - RNA, 20 nt, Homo sapiens
aacaauacacaccuaguuuc

SEQ ID NO: 566 - RNA, 20 nt, Homo sapiens
acucacaccuuuugcaacau

SEQ ID NO: 567 - RNA, 20 nt, Homo sapiens
ccuaauccaucuacacaugu

SEQ ID NO: 568 - RNA, 41 nt, Homo sapiens
aaggcagugcuaaugccuaauggugcuacaguuucugccuc

SEQ ID NO: 569 - RNA, 41 nt, Homo sapiens
ugugcagcaaaagagcaucauugagaccauggagcagcauc

SEQ ID NO: 570 - RNA, 41 nt, Homo sapiens
gcagugcuaaugccuaauggugcuacaguuucugccucuuc

SEQ ID NO: 571 - RNA, 41 nt, Homo sapiens
gcagcaaaagagcaucauugagaccauggagcagcaucuga

SEQ ID NO: 572 - RNA, 41 nt, Homo sapiens
gauggccccgaagcaagccugauggaacaggauagaaccaa

SEQ ID NO: 573 - RNA, 41 nt, Homo sapiens
gaugcugaugaugcugauaaugccaguaaacuagcugcaau

SEQ ID NO: 574 - RNA, 41 nt, Homo sapiens
auccagaaguaaauggagacaccaaguggcacucuuucaaa

SEQ ID NO: 575 - RNA, 41 nt, Homo sapiens
gugaugcugaugaugcugauaaugccaguaaacuagcugca

SEQ ID NO: 576 - RNA, 41 nt, Homo sapiens
cuggagcacaagucacaaauguaccaaguugaaaugaauca

SEQ ID NO: 577 - RNA, 41 nt, Homo sapiens
acaaaaugaaaugaauggugcuuacuucaagcaaagcucag

SEQ ID NO: 578 - RNA, 41 nt, Homo sapiens
aauggaggaauaaaacgcacaguuagugaaccuucucucuc

SEQ ID NO: 579 - RNA, 41 nt, Homo sapiens
augaaaugaauggugcuuacuucaagcaaagcucaguguuc

SEQ ID NO: 580 - RNA, 41 nt, Homo sapiens
gaaaugaauggugcuuacuucaagcaaagcucaguguucac

SEQ ID NO: 581 - RNA, 41 nt, Homo sapiens
gaccccuccccagaaggacacucaaaagcaugcugcucuaa

SEQ ID NO: 582 - RNA, 41 nt, Homo sapiens
acugucucaauauuauccagauuguguuuccauugcggugc

SEQ ID NO: 583 - RNA, 41 nt, Homo sapiens
ccuccccagaaggacacucaaaagcaugcugcucuaaggug

SEQ ID NO: 584 - RNA, 41 nt, Homo sapiens
acauaaaugccauuaacagucaggcuacuaaugaguugucc

SEQ ID NO: 585 - RNA, 41 nt, Homo sapiens
auguccccaguguugaaacagcacuugaaucaacaggcuuc

SEQ ID NO: 586 - RNA, 41 nt, Homo sapiens
ggaucauucuuuggccagacuaaaguggaagaauguuuuca

SEQ ID NO: 587 - RNA, 41 nt, Homo sapiens
aagcuccugguggcagcucugaacgguauuuaaaacaaaau

SEQ ID NO: 588 - RNA, 41 nt, Homo sapiens
cuugcucagcaaagguacuugauacauaaccaugcaaaugu

SEQ ID NO: 589 - RNA, 41 nt, Homo sapiens
uucuuguucaaacaauacacaccuaguuucagagaauaaag

SEQ ID NO: 590 - RNA, 41 nt, Homo sapiens
ccauuuucaaacucacaccuuuugcaacauaagccucauaa

SEQ ID NO: 591 - RNA, 41 nt, Homo sapiens
uucccagaguccuaauccaucuacacauguaugcagcccuu

SEQ ID NO: 592 - RNA, 15 nt, Homo sapiens
ccuaauggugcuaca

SEQ ID NO: 593 - RNA, 15 nt, Homo sapiens
caucauugagaccaa

SEQ ID NO: 594 - RNA, 15 nt, Homo sapiens
aauggugcuacagua

SEQ ID NO: 595 - RNA, 15 nt, Homo sapiens
cauugagaccaugga

SEQ ID NO: 596 - RNA, 15 nt, Homo sapiens
agccugauggaacaa

SEQ ID NO: 597 - RNA, 15 nt, Homo sapiens
gauaaugccaguaaa

SEQ ID NO: 598 - RNA, 15 nt, Homo sapiens
gagacaccaagugga

SEQ ID NO: 599 - RNA, 15 nt, Homo sapiens
cugauaaugccagua

SEQ ID NO: 600 - RNA, 15 nt, Homo sapiens
caaauguaccaagua

SEQ ID NO: 601 - RNA, 15 nt, Homo sapiens
uggugcuuacuucaa

SEQ ID NO: 602 - RNA, 15 nt, Homo sapiens
cgcacaguuagugaa

SEQ ID NO: 603 - RNA, 15 nt, Homo sapiens
cuuacuucaagcaaa

SEQ ID NO: 604 - RNA, 15 nt, Homo sapiens
uacuucaagcaaaga

SEQ ID NO: 605 - RNA, 15 nt, Homo sapiens
ggacacucaaaagca

SEQ ID NO: 606 - RNA, 15 nt, Homo sapiens
uccagauuguguuua

SEQ ID NO: 607 - RNA, 15 nt, Homo sapiens
acucaaaagcaugca

SEQ ID NO: 608 - RNA, 15 nt, Homo sapiens
acagucaggcuacua

SEQ ID NO: 609 - RNA, 15 nt, Homo sapiens
aaacagcacuugaaa

SEQ ID NO: 610 - RNA, 15 nt, Homo sapiens
cagacuaaaguggaa

SEQ ID NO: 611 - RNA, 15 nt, Homo sapiens
gcucugaacgguaua

SEQ ID NO: 612 - RNA, 15 nt, Homo sapiens
uacuugauacauaaa

SEQ ID NO: 613 - RNA, 15 nt, Homo sapiens
uacacaccuaguuua

SEQ ID NO: 614 - RNA, 15 nt, Homo sapiens
caccuuuugcaacaa

SEQ ID NO: 615 - RNA, 15 nt, Homo sapiens
uccaucuacacauga

SEQ ID NO: 616 - RNA, 20 nt, Artificial Sequence
  Synthetic Polynucleotide
uguagcaccauuaggcauua

SEQ ID NO: 617 - RNA, 20 nt, Artificial Sequence
  Synthetic Polynucleotide
uuggucucaaugaugcucuu

SEQ ID NO: 618 - RNA, 20 nt, Artificial Sequence
  Synthetic Polynucleotide
uacuguagcaccauuaggca

SEQ ID NO: 619 - RNA, 20 nt, Artificial Sequence
  Synthetic Polynucleotide
uccauggucucaaugaugcu

SEQ ID NO: 620 - RNA, 20 nt, Artificial Sequence
  Synthetic Polynucleotide
uuguuccaucaggcuugcuu

SEQ ID NO: 621 - RNA, 20 nt, Artificial Sequence
  Synthetic Polynucleotide
uuuacuggcauuaucagcau

SEQ ID NO: 622 - RNA, 20 nt, Artificial Sequence
  Synthetic Polynucleotide
uccacuuggugucuccauuu

SEQ ID NO: 623 - RNA, 20 nt, Artificial Sequence
  Synthetic Polynucleotide
uacuggcauuaucagcauca

SEQ ID NO: 624 - RNA, 20 nt, Artificial Sequence
  Synthetic Polynucleotide
uacuugguacauuugugacu

SEQ ID NO: 625 - RNA, 20 nt, Artificial Sequence
  Synthetic Polynucleotide
uugaaguaagcaccauucau

SEQ ID NO: 626 - RNA, 20 nt, Artificial Sequence
  Synthetic Polynucleotide
uucacuaacugugcguuuua

SEQ ID NO: 627 - RNA, 20 nt, Artificial Sequence
  Synthetic Polynucleotide
uuugcuugaaguaagcacca

SEQ ID NO: 628 - RNA, 20 nt, Artificial Sequence
  Synthetic Polynucleotide
ucuuugcuugaaguaagcac

SEQ ID NO: 629 - RNA, 20 nt, Artificial Sequence
  Synthetic Polynucleotide
ugcuuuugaguguccuucug

SEQ ID NO: 630 - RNA, 20 nt, Artificial Sequence
  Synthetic Polynucleotide
uaaacacaaucuggauaaua

SEQ ID NO: 631 - RNA, 20 nt, Artificial Sequence
  Synthetic Polynucleotide
ugcaugcuuuugaguguccu

SEQ ID NO: 632 - RNA, 20 nt, Artificial Sequence
  Synthetic Polynucleotide
uaguagccugacuguuaaug

SEQ ID NO: 633 - RNA, 20 nt, Artificial Sequence
  Synthetic Polynucleotide
uuucaagugcuguuucaaca

SEQ ID NO: 634 - RNA, 20 nt, Artificial Sequence
  Synthetic Polynucleotide
uuccacuuuagucuggccaa

SEQ ID NO: 635 - RNA, 20 nt, Artificial Sequence
  Synthetic Polynucleotide
uauaccguucagagcugcca

SEQ ID NO: 636 - RNA, 20 nt, Artificial Sequence
  Synthetic Polynucleotide
uuuauguaucaaguaccuuu

SEQ ID NO: 637 - RNA, 20 nt, Artificial Sequence
  Synthetic Polynucleotide
uaaacuagguguguauuguu

SEQ ID NO: 638 - RNA, 20 nt, Artificial Sequence
  Synthetic Polynucleotide
uuguugcaaaaggugugagu

SEQ ID NO: 639 - RNA, 20 nt, Artificial Sequence
  Synthetic Polynucleotide
ucauguguagauggauuagg

SEQ ID NO: 640 - RNA, 20 nt, Homo sapiens
caccuguugugguccaaguu

SEQ ID NO: 641 - RNA, 20 nt, Homo sapiens
cacuacaggauguuugugga

SEQ ID NO: 642 - RNA, 20 nt, Homo sapiens
cugagaguggugucuggaua

SEQ ID NO: 643 - RNA, 20 nt, Homo sapiens
cuauccuuccaguggugaca

SEQ ID NO: 644 - RNA, 20 nt, Homo sapiens
gccgagauuacucagcugaa

SEQ ID NO: 645 - RNA, 20 nt, Homo sapiens
uccaacacgcauaucuuuac

SEQ ID NO: 646 - RNA, 20 nt, Homo sapiens
gucagcaugaagccugcauu

SEQ ID NO: 647 - RNA, 20 nt, Homo sapiens
aaggagacucuaagaggagg

SEQ ID NO: 648 - RNA, 20 nt, Homo sapiens
uuuaccuggugcugcgucuu

SEQ ID NO: 649 - RNA, 20 nt, Homo sapiens
cugcauaucguugaggugaa

SEQ ID NO: 650 - RNA, 20 nt, Homo sapiens
cuuccuuuguacaguaacuu

SEQ ID NO: 651 - RNA, 20 nt, Homo sapiens
cucuguuuaguaguugguug

SEQ ID NO: 652 - RNA, 20 nt, Homo sapiens
gaaacggaugaaggacugag

SEQ ID NO: 653 - RNA, 20 nt, Homo sapiens
uuggaggacaccgacuaauu

SEQ ID NO: 654 - RNA, 20 nt, Homo sapiens
uuguacaguaacuuucaacc

SEQ ID NO: 655 - RNA, 20 nt, Homo sapiens
ccagaugauugugcuccagu

SEQ ID NO: 656 - RNA, 20 nt, Homo sapiens
cuugguguggacugagauug

SEQ ID NO: 657 - RNA, 20 nt, Homo sapiens
aacuacagucguuuaccugg

SEQ ID NO: 658 - RNA, 20 nt, Homo sapiens
gaaaggacucaccugacuuu

SEQ ID NO: 659 - RNA, 20 nt, Homo sapiens
cuguugugguccaaguuuaa

SEQ ID NO: 660 - RNA, 20 nt, Homo sapiens
uccaaguuuaaucagcacca

SEQ ID NO: 661 - RNA, 20 nt, Homo sapiens
uuuguggacguggucuuggu

SEQ ID NO: 662 - RNA, 20 nt, Homo sapiens
uacacaucuguugacaccag

SEQ ID NO: 663 - RNA, 20 nt, Homo sapiens
gaacaaauacacguauguua

SEQ ID NO: 664 - RNA, 20 nt, Homo sapiens
uauuguguauuauguuguuc

SEQ ID NO: 665 - RNA, 41 nt, Homo sapiens
gcucaacaaccaccuguugugguccaaguuuaaucagcacc

SEQ ID NO: 666 - RNA, 41 nt, Homo sapiens
gcccaccagccacuacaggauguuuguggacguggucuugg

SEQ ID NO: 667 - RNA, 41 nt, Homo sapiens
auuuauuguacugagaguggugucuggauauauuccuuuug

SEQ ID NO: 668 - RNA, 41 nt, Homo sapiens
gcgugucccccuauccuuccaguggugacagcuccuccccu

SEQ ID NO: 669 - RNA, 41 nt, Homo sapiens
cuaccagaaugccgagauuacucagcugaaaauugauaaua

SEQ ID NO: 670 - RNA, 41 nt, Homo sapiens
cugcaacgcuuccaacacgcauaucuuuacuuuccaagaaa

SEQ ID NO: 671 - RNA, 41 nt, Homo sapiens
guuucgagcagucagcaugaagccugcauucuugcccucug

SEQ ID NO: 672 - RNA, 41 nt, Homo sapiens
ggacugggcgaaggagacucuaagaggaggcgcgugucccc

SEQ ID NO: 673 - RNA, 41 nt, Homo sapiens
acuacagucguuuaccuggugcugcgucuugcuuuugguuu

SEQ ID NO: 674 - RNA, 41 nt, Homo sapiens
ccagccccggcugcauaucguugaggugaacgacggagagc

SEQ ID NO: 675 - RNA, 41 nt, Homo sapiens
acuccacuuucuuccuuuguacaguaacuuucaaccuuuuc

SEQ ID NO: 676 - RNA, 41 nt, Homo sapiens
ucuggcccuucucuguuuaguaguugguuggggaagugggg

SEQ ID NO: 677 - RNA, 41 nt, Homo sapiens
acuaauuugggaaacggaugaaggacugagaaggcccccgc

SEQ ID NO: 678 - RNA, 41 nt, Homo sapiens
uguuauuagguuggaggacaccgacuaauuugggaaacgga

SEQ ID NO: 679 - RNA, 41 nt, Homo sapiens
cuuucuuccuuuguacaguaacuuucaaccuuuucguuggc

SEQ ID NO: 680 - RNA, 41 nt, Homo sapiens
acaaugugacccagaugauugugcuccagucccuccauaag

SEQ ID NO: 681 - RNA, 41 nt, Homo sapiens
aggguccccccuugguguggacugagauugcccccauccgg

SEQ ID NO: 682 - RNA, 41 nt, Homo sapiens
ccucugcccuaacuacagucguuuaccuggugcugcgucuu

SEQ ID NO: 683 - RNA, 41 nt, Homo sapiens
cagggucagggaaaggacucaccugacuuuggacagcuggc

SEQ ID NO: 684 - RNA, 41 nt, Homo sapiens
caacaaccaccuguugugguccaaguuuaaucagcaccaga

SEQ ID NO: 685 - RNA, 41 nt, Homo sapiens
ccuguugugguccaaguuuaaucagcaccagacagagauga

SEQ ID NO: 686 - RNA, 41 nt, Homo sapiens
cuacaggauguuuguggacguggucuugguggaccagcacc

SEQ ID NO: 687 - RNA, 41 nt, Homo sapiens
ugaguccauguacacaucuguugacaccagcauccccuccc

SEQ ID NO: 688 - RNA, 41 nt, Homo sapiens
gauccaaaaagaacaaauacacguauguuauaaccaucagc

SEQ ID NO: 689 - RNA, 40 nt, Homo sapiens
uaaauuuguuuauuguguauuauguuguucaaaugcauuu

SEQ ID NO: 690 - RNA, 15 nt, Homo sapiens
guugugguccaagua

SEQ ID NO: 691 - RNA, 15 nt, Homo sapiens
caggauguuugugga

SEQ ID NO: 692 - RNA, 15 nt, Homo sapiens
aguggugucuggaua

SEQ ID NO: 693 - RNA, 15 nt, Homo sapiens
cuuccaguggugaca

SEQ ID NO: 694 - RNA, 15 nt, Homo sapiens
gauuacucagcugaa

SEQ ID NO: 695 - RNA, 15 nt, Homo sapiens
cacgcauaucuuuaa

SEQ ID NO: 696 - RNA, 15 nt, Homo sapiens
caugaagccugcaua

SEQ ID NO: 697 - RNA, 15 nt, Homo sapiens
gacucuaagaggaga

SEQ ID NO: 698 - RNA, 15 nt, Homo sapiens
cuggugcugcgucua

SEQ ID NO: 699 - RNA, 15 nt, Homo sapiens
uaucguugaggugaa

SEQ ID NO: 700 - RNA, 15 nt, Homo sapiens
uuuguacaguaacua

SEQ ID NO: 701 - RNA, 15 nt, Homo sapiens
uuuaguaguugguua

SEQ ID NO: 702 - RNA, 15 nt, Homo sapiens
ggaugaaggacugaa

SEQ ID NO: 703 - RNA, 15 nt, Homo sapiens
ggacaccgacuaaua

SEQ ID NO: 704 - RNA, 15 nt, Homo sapiens
caguaacuuucaaca

SEQ ID NO: 705 - RNA, 15 nt, Homo sapiens
ugauugugcuccaga

SEQ ID NO: 706 - RNA, 15 nt, Homo sapiens
uguggacugagauua

SEQ ID NO: 707 - RNA, 15 nt, Homo sapiens
cagucguuuaccuga

SEQ ID NO: 708 - RNA, 15 nt, Homo sapiens
gacucaccugacuua

SEQ ID NO: 709 - RNA, 15 nt, Homo sapiens
gugguccaaguuuaa

SEQ ID NO: 710 - RNA, 15 nt, Homo sapiens
guuuaaucagcacca

SEQ ID NO: 711 - RNA, 15 nt, Homo sapiens
ggacguggucuugga

SEQ ID NO: 712 - RNA, 15 nt, Homo sapiens
aucuguugacaccaa

SEQ ID NO: 713 - RNA, 15 nt, Homo sapiens
aauacacguauguua

SEQ ID NO: 714 - RNA, 15 nt, Homo sapiens
guauuauguuguuca

SEQ ID NO: 715 - RNA, 20 nt, Artificial Sequence
  Synthetic Polynucleotide
uacuuggaccacaacaggug

SEQ ID NO: 716 - RNA, 20 nt, Artificial Sequence
  Synthetic Polynucleotide
uccacaaacauccuguagug

SEQ ID NO: 717 - RNA, 20 nt, Artificial Sequence
  Synthetic Polynucleotide
uauccagacaccacucucag

SEQ ID NO: 718 - RNA, 20 nt, Artificial Sequence
  Synthetic Polynucleotide
ugucaccacuggaaggauag

SEQ ID NO: 719 - RNA, 20 nt, Artificial Sequence
  Synthetic Polynucleotide
uucagcugaguaaucucggc

SEQ ID NO: 720 - RNA, 20 nt, Artificial Sequence
  Synthetic Polynucleotide
uuaaagauaugcguguugga

SEQ ID NO: 721 - RNA, 20 nt, Artificial Sequence
  Synthetic Polynucleotide
uaugcaggcuucaugcugac

SEQ ID NO: 722 - RNA, 20 nt, Artificial Sequence
  Synthetic Polynucleotide
ucuccucuuagagucuccuu

SEQ ID NO: 723 - RNA, 20 nt, Artificial Sequence
  Synthetic Polynucleotide
uagacgcagcaccagguaaa

SEQ ID NO: 724 - RNA, 20 nt, Artificial Sequence
  Synthetic Polynucleotide
uucaccucaacgauaugcag

SEQ ID NO: 725 - RNA, 20 nt, Artificial Sequence
  Synthetic Polynucleotide
uaguuacuguacaaaggaag

SEQ ID NO: 726 - RNA, 20 nt, Artificial Sequence
  Synthetic Polynucleotide
uaaccaacuacuaaacagag

SEQ ID NO: 727 - RNA, 20 nt, Artificial Sequence
  Synthetic Polynucleotide
uucaguccuucauccguuuc

SEQ ID NO: 728 - RNA, 20 nt, Artificial Sequence
  Synthetic Polynucleotide
uauuagucgguguccuccaa

SEQ ID NO: 729 - RNA, 20 nt, Artificial Sequence
  Synthetic Polynucleotide
uguugaaaguuacuguacaa

SEQ ID NO: 730 - RNA, 20 nt, Artificial Sequence
  Synthetic Polynucleotide
ucuggagcacaaucaucugg

SEQ ID NO: 731 - RNA, 20 nt, Artificial Sequence
  Synthetic Polynucleotide
uaaucucaguccacaccaag

SEQ ID NO: 732 - RNA, 20 nt, Artificial Sequence
  Synthetic Polynucleotide
ucagguaaacgacuguaguu

SEQ ID NO: 733 - RNA, 20 nt, Artificial Sequence
  Synthetic Polynucleotide
uaagucaggugaguccuuuc

SEQ ID NO: 734 - RNA, 20 nt, Artificial Sequence
  Synthetic Polynucleotide
uuaaacuuggaccacaacag

SEQ ID NO: 735 - RNA, 20 nt, Artificial Sequence
  Synthetic Polynucleotide
uggugcugauuaaacuugga

SEQ ID NO: 736 - RNA, 20 nt, Artificial Sequence
  Synthetic Polynucleotide
uccaagaccacguccacaaa

SEQ ID NO: 737 - RNA, 20 nt, Artificial Sequence
  Synthetic Polynucleotide
uuggugucaacagaugugua

SEQ ID NO: 738 - RNA, 20 nt, Artificial Sequence
  Synthetic Polynucleotide
uaacauacguguauuuguuc

SEQ ID NO: 739 - RNA, 20 nt, Artificial Sequence
  Synthetic Polynucleotide
ugaacaacauaauacacaau